(19) United States
(12) Patent Application Publication
(10) Pub. No.: US 2025/0269059 A1
(43) Pub. Date: Aug. 28, 2025

(54) RT EDITING COMPOSITIONS AND METHODS

(71) Applicant: CRISPR Therapeutics AG, Zug (CH)

(72) Inventors: Christof Fellmann, South Boston, MA (US); Aamir Mir, South Boston, MA (US); Parbir Singh Grewal, South Boston, MA (US); Megha Jayachandran, South Boston, MA (US); Victoria Barnhouse, South Boston, MA (US); Bhakti Narendra Kadam, South Boston, MA (US); Victor Chen, South Boston, MA (US); Qichen Yuan, South Boston, MA (US); Nassim Ajami, South Boston, MA (US); Cristian Loaiza, South Boston, MA (US); Benjamin Thomas Freeman, South Boston, MA (US)

(21) Appl. No.: 19/064,068

(22) Filed: Feb. 26, 2025

Related U.S. Application Data
(60) Provisional application No. 63/558,338, filed on Feb. 27, 2024.
(60) Provisional application No. 63/649,882, filed on May 20, 2024.
(60) Provisional application No. 63/718,994, filed on Nov. 11, 2024.

(51) Int. Cl. A61K 48/00; A61K 9/1272; A61K 9/51; A61P 5/00; A61P 11/00; C12N 9/12; C12N 9/22; C12N 15/11; C12N 15/86; C12N 15/88

(52) U.S. Cl. CPC A61K 48/005; A61K 9/1272; A61K 9/5123; A61P 5/00; A61P 11/00; C12N 9/1276; C12N 9/22; C12N 15/111; C12N 15/86; C12N 15/88; C07K 2319/09; C07K 2319/80; C12N 2310/20; C12N 2750/14143; C12N 2800/22; C12N 2830/48; C12Y 207/07049

(57) ABSTRACT

Disclosed herein include methods, compositions, and kits suitable for use in gene editing. RT editing systems are provided herein comprising a fusion protein comprising a Cas9 nickase and a reverse transcriptase, and a template armed guide RNA (tagRNA) comprising from 5′ to 3′ a spacer sequence, a scaffold sequence, an editing template and a flap binding sequence. In some embodiments, the RT editing system further comprises an enhancer guide RNA (egRNA).

### RELATED APPLICATIONS

The present application claims priority to U.S. Provisional Application No. 63/558,338, filed Feb. 27, 2024; U.S. Provisional Application No. 63/649,882, filed May 20, 2024; and U.S. Provisional Application No. 63/718,994, filed Nov. 11, 2024. The entire contents of these applications are hereby expressly incorporated by reference in their entireties.

### REFERENCE TO SEQUENCE LISTING

The present application is being filed along with a Sequence Listing in electronic format. The Sequence Listing is provided as a file entitled 80EM-341785-US_SeqListing, created Feb. 25, 2025, which is 10,377 kilobytes in size. The information in the electronic format of the Sequence Listing is incorporated herein by reference in its entirety.

### BACKGROUND

### Field

The present disclosure relates generally to the field of gene editing.

### Description of the Related Art

Many diseases and disorders have a genetic component, including those that involve pathogenic single nucleotide mutations. There is a need for nucleic acid editing compositions and methods with greater editing efficiency and specificity. Existing gene editing approaches wherein components are engineered to be expressed within target cells suffer from insufficient levels of expression, or inadequate efficacy in their action, to be therapeutically effective; or lack specificity in directing a precise editing outcome. There is a need for compositions and methods with improved strength, mechanistic efficacy, and duration of gene editing component expression and employment for in vivo applications.

### SUMMARY

Disclosed herein are reverse transcriptase (RT)-based editing systems. In some embodiments, the RT editing system comprises: a fusion protein comprising a Cas9 nickase and a reverse transcriptase, and a template armed guide RNA (tagRNA) comprising from 5′ to 3′ a spacer sequence, a scaffold sequence, an editing template and a flap binding sequence. In some embodiments, the RT editing system further comprises an enhancer guide RNA (egRNA).

Disclosed herein are reverse transcriptase (RT) editors. In some embodiments, the RT editor comprises: a DNA binding domain, a DNA endonuclease domain and a DNA polymerase domain, wherein the DNA polymerase domain and the DNA endonuclease domain are fused or linked to form a fusion protein, wherein the DNA polymerase domain comprises a reverse transcriptase, optionally a reverse transcriptase comprising an amino acid sequence that is at least 80% identical, at least 85% identical, at least 90% identical, or at least 95%, or 100% identical to any one of SEQ ID NOs: 141, 250-269, 315-319, 899-909, and 1015-1018, optionally an amino acid sequence having at least one, at least two, at least three, at least four, or at least five mismatches relative to the sequence of any one of SEQ ID NOs: 141, 250-269, and 315-319, 899-909, and 1015-1018, and wherein the DNA binding domain, DNA endonuclease domain comprises a Cas9 nickase, optionally a Cas9 nickase comprising an amino acid sequence that is at least 80% identical, at least 85% identical, at least 90% identical, or at least 95%, or 100% identical to any one of SEQ ID NOs: 142-163 and 270-314, optionally an amino acid sequence having at least one, at least two, at least three, at least four, or at least five mismatches relative to the sequence of any one of SEQ ID NOs: 142-163 and 270-314.

Disclosed herein include template armed guide RNAs (tagRNAs). In some embodiments, the tagRNA comprises: a spacer that is complementary to a search target sequence on a first strand of a double stranded target DNA; an editing template that comprises a region of complementarity to an editing target sequence on a second strand of the double stranded target DNA; and a scaffold sequence that associates with a reverse transcriptase (RT) editor comprising a DNA binding domain, a DNA endonuclease domain and a DNA polymerase domain, and wherein the first strand and the second strand of the double-stranded target DNA are complementary to each other.

In some embodiments, the tagRNA comprises a flap binding sequence at least partially complementary to the spacer. In some embodiments, the scaffold sequence is between the spacer and the editing template. In some embodiments, the tagRNA comprises from 5′ to 3′: the spacer, the scaffold sequence, the editing template, and the flap binding sequence. In some embodiments, the spacer, the scaffold sequence, the editing template, and the flap binding sequence form a contiguous sequence in a single molecule. In some embodiments, the editing template comprises an intended nucleotide edit compared to the double stranded target DNA. In some embodiments, the editing template comprises several intended nucleotide edits compared to the double stranded target DNA. In some embodiments, the tagRNA guides the RT editor to incorporate the intended nucleotide edit or edits into the double stranded target DNA when the tagRNA is contacted with the double stranded target DNA. In some embodiments, the RT editor synthesizes a single stranded DNA encoded by the editing template, wherein the single stranded DNA replaces the editing target sequence and results in incorporation of the intended nucleotide edit into a region corresponding to the editing target in the double stranded target DNA. In some embodiments, the search target sequence is complementary to a protospacer sequence in the double stranded target DNA, and wherein the protospacer sequence is adjacent to a protospacer adjacent motif (PAM) in the double stranded target DNA. In some embodiments, the tagRNA results in incorporation of a nucleotide edit or nucleotide edits in the PAM when contacted with the double stranded target DNA. In some embodiments, the tagRNA results in incorporation of a nucleotide edit or nucleotide edits outside the PAM when contacted with the double-stranded target DNA. In some embodiments, the spacer of the tagRNA is from 16 to 25 nucleotides in length, optionally 20 nucleotides in length or 21-23 nucleotides in length. In some embodiments, the flap binding sequence is about 2 to 20 nucleotides in length, optionally about 8 to 16 nucleotides in length or 6 nucleotides in length. In some embodiments, the editing template is about 4 to 30 nucleotides in length, optionally about 10 to 30 nucleotides in length, further optionally 6 to 9 nucleotides in length.

In some embodiments, the tagRNA results in incorporation of the intended nucleotide edit about 0 to 30 base pairs downstream of the nickase cleavage site. In some embodiments, the intended nucleotide edit comprises a single nucleotide substitution compared to the region corresponding to the editing target sequence in the double stranded target DNA, optionally, the single nucleotide substitution is a T>G substitution or a T>C substitution. In some embodiments, the intended nucleotide edit comprises more than one nucleotide substitution compared to the region corresponding to the editing target sequence. In some embodiments, the intended nucleotide edit or edits comprises an insertion compared to the region corresponding to the editing target in the double stranded target DNA (e.g., an insertion of a nucleotide sequence at least 50, at least 45, at least 40, at least 35, at least 30, at least 25, at least 20, at least 15, at least 10, or at least 5, nucleotides in length). In some embodiments, the intended nucleotide edit or edits comprises a deletion compared to the region corresponding to the editing target in the double stranded target DNA. In some embodiments, the editing template comprises one or more silent nucleotide edits compared to the region corresponding to the editing target in the double stranded target DNA. In some embodiments, the editing template comprises a wild type DNA sequence. In some embodiments, the tagRNA results in correction of a mutation when contacted with the double stranded target DNA. In some embodiments, the tagRNA comprises any one of the sequences of SEQ ID NOs: 16-47, 99-130, 164-181, 320-497, 563-682, 791-792, 1046-1053, and 1089-1203, or a sequence that exhibits at least about 80%, at least about 85%, at least 90%, at least 95%, or 100% identity to any one of the sequences of SEQ ID NOs: 16-47, 99-130, 164-181, 320-497, 563-682, 791-792, 1046-1053, and 1089-1203. In some embodiments, the tagRNA further comprises: 3′ mN*mN*mN*N and 5′ mN*mN*mN* modifications, where m indicates that the nucleotide contains a 2′-O-Me modification and a * indicates the presence of a phosphorothioate bond; and/or a structural motif at the 3′ terminus selected from the group consisting of: a prequeosine1-1 riboswitch aptamer (evopreQ1) and variants thereof, a frameshifting pseudoknot from Moloney murine leukemia virus (MMLV) (mpknot), G-quadruplexes, hairpin structures, xrRNA, and a P4-P6 domain of the group I intron; optionally, the structural motif is evopreQ1 or a variant thereof comprising a nucleotide sequence selected from SEQ ID NOs: 84-90. In some embodiments, a chemically modified 3′-terminus comprises inverted-dT. In some embodiments, the tagRNA further comprises: 3′ mN*mN*mN*N, 5′ mN*mN*mN*, and/or 3′ inverted-dT modifications.

Disclosed herein are reverse transcriptase (RT) editing systems. In some embodiments, the system comprises: a template armed guide RNA (tagRNA), or a nucleic acid encoding the tagRNA, wherein the tagRNA comprises: a spacer that is complementary to a search target sequence on a first strand of a nucleic acid molecule; an editing template that comprises a region of complementarity to an editing target sequence on a second strand of the nucleic acid molecule; and a scaffold sequence that associates with a reverse transcriptase (RT) editor comprising a DNA binding domain, a DNA endonuclease domain and a DNA polymerase domain; or a nucleic acid encoding the RT editor, wherein the DNA polymerase domain comprises a reverse transcriptase, optionally a reverse transcriptase comprising an amino acid sequence that is at least 80%, at least about 85%, at least 90%, at least 95%, or 100% identical to any one of SEQ ID NOs: 141, 250-269, 315-319, 899-909, and 1015-1018, optionally an amino acid sequence having at least one, at least two, at least three, at least four, or at least five mismatches relative to the sequence of any one of SEQ ID NOs: 141, 250-269, 315-319, 899-909, and 1015-1018, and wherein the DNA DNA endonuclease domain comprises a Cas9 nickase, optionally a Cas9 nickase comprising an amino acid sequence that is at least 80%, at least about 85%, at least 90%, at least 95%, or 100% identical to any one of SEQ ID NOs: 142-163 and 270-314, optionally an amino acid sequence having at least one, at least two, at least three, at least four, or at least five mismatches relative to the sequence of any one of SEQ ID NOs: 142-163 and 270-314.

Disclosed herein include reverse transcriptase (RT) editing systems. In some embodiments, the system comprises: any of the tagRNAs, or a nucleic acid encoding the tagRNA, described herein; and a reverse transcriptase (RT) editor comprising a DNA binding domain, a DNA endonuclease domain and a DNA polymerase domain, or a nucleic acid encoding the RT editor.

In some embodiments, RT editing system further comprises: an enhancer guide RNA (egRNA), or a nucleic acid encoding the egRNA, wherein the egRNA comprises a egRNA spacer that is complementary to a second search target sequence in the double stranded target DNA. In some embodiments, the second search target sequence is on the second strand of the double stranded target DNA. In some embodiments, the egRNA spacer is from 16 to 25 nucleotides in length, optionally 21-23 nucleotides in length, optionally 20 nucleotides in length. In some embodiments, the egRNA comprises a scaffold sequence. In some embodiments, the intended nucleotide edit incorporation rate of the RT editing system is greater than at least about 30%, about 40%, about 50%, about 60%, about 70%, or about 80%.

Disclosed herein include reverse transcriptase (RT) editing complexes. In some embodiments, the RT editing complex comprises: template armed guide RNA (tagRNA) comprising: a spacer that is complementary to a search target sequence on a first strand of a nucleic acid molecule; an editing template that comprises a region of complementarity to an editing target sequence on a second strand of the nucleic acid molecule; and a scaffold sequence that associates with a reverse transcriptase (RT) editor comprising a DNA binding domain, a DNA endonuclease domain and a DNA polymerase domain; or a nucleic acid encoding the RT editor, wherein the DNA polymerase domain comprises a reverse transcriptase, optionally a reverse transcriptase comprising an amino acid sequence that is at least 80%, at least about 85%, at least 90%, at least 95%, or 100% identical to any one of SEQ ID NOs: 141, 250-269, 315-319, 899-909, and 1015-1018, optionally an amino acid sequence having at least one, at least two, at least three, at least four, or at least five mismatches relative to the sequence of any one of SEQ ID NOs: 141, 250-269, 315-319, 899-909, and 1015-1018, and wherein the DNA endonuclease domain comprises a Cas9 nickase, optionally a Cas9 nickase comprising an amino acid sequence that is at least 80%, at least about 85%, at least 90%, at least 95%, or 100% identical to any one of SEQ ID NOs: 142-163 and 270-314, optionally an amino acid sequence having at least one, at least two, at least three, at least four, or at least five mismatches relative to the sequence of any one of SEQ ID NOs: 142-163 and 270-314.

In some embodiments, the RT editing complex comprises: (i) any of the tagRNAs disclosed herein and a reverse transcriptase (RT) editor comprising a DNA binding domain, a DNA endonuclease domain and a DNA polymerase domain; or (ii) any of the RT editing systems disclosed herein. In some embodiments, the intended nucleotide edit incorporation rate of the RT editing complex is greater than at least about 30%, about 40%, about 50%, about 60%, about 70%, or about 80%.

In some embodiments, the DNA binding domain, a DNA endonuclease domain is a CRISPR associated (Cas) protein domain. In some embodiments, the Cas protein domain has nickase activity. In some embodiments, the Cas protein domain is a Cas9. In some embodiments, the Cas9 comprises a mutation in an HNH domain. In some embodiments, the Cas9 comprises an H840A mutation in the HNH domain. In some embodiments, the Cas protein domain is a Cas12b. In some embodiments, the Cas protein domain is a Cas12a, Cas12b, Cas12c, Cas12d, Cas12e, Cas14a, Cas14b, Cas14c, Cas14d, Cas14e, Cas14f, Cas14g, Cas14h, Cas14u, Casϕ, Ssch1Cas9, Sro1Cas9, Sha4Cas9, SsuCas9, iSpyMacCas9, Ssi5Cas9, Ssi8Cas9, Ssci4Cas9, Shy1Cas9, Sag3Cas9, Slutr1Cas9, Ssch3Cas9, SpRYCas9, SpRYcCas9, Sma2Cas9, SsaCas9, EvoCjCas9, or iSpyMac. In some embodiments, the DNA polymerase domain is a reverse transcriptase, optionally the editing template is a reverse transcription template. In some embodiments, the reverse transcriptase is a retrovirus reverse transcriptase. In some embodiments, the reverse transcriptase is a Moloney murine leukemia virus (MMLV) reverse transcriptase. In some embodiments, the DNA polymerase domain and the DNA binding domain, a DNA endonuclease domain are fused or linked to form a fusion protein. In some embodiments, the DNA polymerase domain comprises a reverse transcriptase, optionally a reverse transcriptase comprising an amino acid sequence that is at least 80%, at least about 85%, at least 90%, at least 95%, or 100% identical to any one of SEQ ID NOs: 141, 250-269, 315-319, 899-909, and 1015-1018, optionally an amino acid sequence having at least one, at least two, at least three, at least four, or at least five mismatches relative to the sequence of any one of SEQ ID NOs: 141, 250-269, 315-319, 899-909, and 1015-1018; and/or the DNA binding domain, a DNA endonuclease domain comprises a Cas9 nickase, optionally a Cas9 nickase comprising an amino acid sequence that is at least 80%, at least about 85%, at least 90%, at least 95%, or 100% identical to any one of SEQ ID NOs: 142-163 and 270-314, optionally an amino acid sequence having at least one, at least two, at least three, at least four, or at least five mismatches relative to the sequence of any one of SEQ ID NOs: 142-163 and 270-314.

In some embodiments, the editing template comprises: (i) at least 1, 2, 3, 4, 5, 6, 7, 8, 9, 10, 11, 12, 13, or 14 2′ O-methyl RNA base(s); and/or (ii) at least 1, 2, 3, 4, 5, 6, 7, 8, 9, 10, 11, 12, 13, or 14 2′ Fluoro RNA base(s). In some embodiments, the FBS comprises: (i) at least 1, 2, 3, 4, 5, 6, 7, or 8 2′ O-methyl RNA base(s); and/or (ii) at least 1, 2, 3, 4, 5, 6, 7, or 8 2′ Fluoro RNA base(s).

In some embodiments, the scaffold sequence comprises a nucleotide sequence that is at least 80% identical (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) to any one of SEQ ID NOs: 683-718, 894-898, and 964-1014 (e.g., a nucleotide sequence having one, two, three, four, or five mismatches relative to the sequence of any one of SEQ ID NOs: 683-718, 894-898, and 964-1014). In some embodiments, the scaffold sequence comprises one or more nucleotide substitutions, insertions, and/or deletions at one or more nucleotide positions relative to the parental scaffold sequence of SEQ ID NO: 683, such as, for example: (i) dinucleotide substitutions at nucleotide positions 5-6 and 34-35, optionally configured such that the nucleotide sequence at positions 5-6 is complementary to the nucleotide sequence at positions 34-35; (ii) a deletion at nucleotide positions 55-87, 57-87, 59-87, 61-87, or 63-87; (iii) a substitution at one or more of nucleotide positions 17, 18, 19, and 20, optionally a UUCG substitution at nucleotide positions 17-20; (iv) a deletion at nucleotide positions 14-16 and 21-24; (v) a replacement of nucleotides at nucleotide positions 11-28 with GUUCGC; and/or (vi) a deletion at nucleotide positions 12-16 and 21-26.

In some embodiments, the scaffold sequence comprises one or more nucleotide substitutions, insertions, and/or deletions at one or more nucleotide positions relative to the parental scaffold sequence of SEQ ID NO: 700, such as, for example, a substitution at one or more of nucleotide positions 13, 18, 21, 40, 50, and 52-53 (e.g., a U-to-C substitution at nucleotide position 13; an A-to-G substitution at nucleotide position 18; an A-to-C or an A-to-U substitution at nucleotide position 21; a C-to-U or a C-to-A or a C-to-G substitution at nucleotide position 40; a U-to-C or a U-to-A or a U-to-G substitution at nucleotide position 50; an A-to-C or an A-to-U or an A-to-G substitution at nucleotide position 52; and/or an A-to-C or an A-to-U or an A-to-G substitution at nucleotide position 53). In some embodiments, the scaffold sequence comprises one or more modifications (e.g., nucleoside modification(s), sugar modification(s), modified internucleoside linkage(s), and/or backbone modification(s)) relative to the parental scaffold sequence of SEQ ID NO: 700, such as, for example, a 2′ O-methyl modification at one or more of nucleotide positions 5-9, 12-21, 29, 37-38, 40, 50, and 52-53. In some embodiments, the scaffold sequence comprises any one of the sequences of SEQ ID NOs: 894-898 and 964-1014, or a sequence that exhibits at least about 85% identity to any one of the sequences of SEQ ID NOs: 894-898 and 964-1014. In some embodiments, the scaffold sequence of the tagRNA, the scaffold sequence of the egRNA, or both, comprises the sequence of any one of SEQ ID NOs: 1210-1266 or a sequence that exhibits at least about 85% identity to any one of SEQ ID NOs: 1210-1266.

In some embodiments, the DNA polymerase domain comprises a reverse transcriptase. In some embodiments, the DNA polymerase domain comprises a reverse transcriptase comprising one or more mutations. In some embodiments, at least one of the one or more mutations is at an amino acid position functionally equivalent to V101, N200, A208, G248, P330, L435, K445, and/or A623 relative to SEQ ID NO: 8.

In some embodiments, the DNA polymerase domain comprises a reverse transcriptase comprising an amino acid sequence that is at least 80% identical to SEQ ID NO: 255. In some embodiments, the reverse transcriptase comprises one or more mutations. In some embodiments, at least one of the one or more mutations is at amino acid position V98, N197, A205, S245, P327, V432, R442, and/or A623 relative to SEQ ID NO: 255. In some embodiments, the reverse transcriptase comprises one or more transition mutations selected from the group consisting of V98R, N197C, N197D, N197G, A205T, S245C, P327E, P327Q, V432K, R442T, and A623F relative to SEQ ID NO: 255.

In some embodiments, the DNA polymerase domain comprises a reverse transcriptase comprising an amino acid sequence that is at least 80% identical to SEQ ID NO: 260. The reverse transcriptase can comprise one or more mutations. In some embodiments, at least one of the one or more mutations is at amino acid position V106, N204, E212, G252, P334, L440, E450, and/or A629 relative to SEQ ID NO: 260. In some embodiments, the reverse transcriptase comprises one or more transition mutations selected from the group consisting of V106R, N204C, N204D, N204G, E212T, G252C, P334E, P334Q, L440K, E450T, and/or A629F relative to SEQ ID NO: 260.

In some embodiments, the DNA polymerase domain comprises a reverse transcriptase comprising an amino acid sequence that is at least 80% identical to SEQ ID NO: 255. In some embodiments, the reverse transcriptase comprises an N197C or V98R transition mutation relative to SEQ ID NO: 255.

In some embodiments, the DNA polymerase domain comprises a reverse transcriptase comprising an amino acid sequence that is at least 80% identical to SEQ ID NO: 260. In some embodiments, the reverse transcriptase comprises an N204C or V106R transition mutation relative to SEQ ID NO: 260.

In some embodiments, the RT editor further comprises an accessory domain. In some embodiments, the accessory domain comprises a single-strand binding (SSB) protein domain or a stabilon. In some embodiments, the SSB protein domain is derived from RecA protein, Sso7d protein, or Sto7d protein. In some embodiments, the accessory domain is situated at the N-terminus, the C-terminus, or at an internal location of the RT editor. In some embodiments, the RT editor comprising an accessory domain comprises an amino acid sequence that is at least 85% identical to any one of SEQ ID NOs: 1276-1298. In some embodiments, the SSB protein domain is derived from Sso7d and comprises one or more mutations at an amino acid position functionally equivalent to K12 and/or E35 of a wild type Sso7d amino acid sequence. In some embodiments, the one or more mutations comprise K12L and/or E35L relative to a wild type Sso7d sequence. In some embodiments, the SSB protein domain comprises an amino acid sequence that is at least 85% identical to SEQ ID NO: 1205. In some embodiments, the RT editor comprising the SSB protein domain comprises from N-terminus to C-terminus: [N-terminus of nCas9]-[linker]-[SSB protein domain]-[linker]-[RT]-[linker]-[C-terminus of nCas9]. In some embodiments, the N-terminus of nCas9 comprises an amino acid sequence functionally equivalent to amino acids 1-1247 of SEQ ID NO: 6 and/or the C-terminus of nCas9 comprises an amino acid sequence functionally equivalent to amino acids 1248-1367 of SEQ ID NO: 6. In some embodiments, the RT editor comprising the SSB protein domain comprises an amino acid sequence that is at least 85% identical to any one of SEQ ID NOs: 1207-1209.

Disclosed herein include ribonucleoprotein (RNP) complexes. In some embodiments, the RNP complex comprises any of the RT editing complexes described herein, or a component thereof.

Disclosed herein include lipid nanoparticles (LNPs). In some embodiments, the LNP comprises any of the RT editing systems described herein, or a component thereof. In some embodiments, the LNP comprises the tagRNA and the nucleic acid encoding the RT editor. In some embodiments, the nucleic acid encoding the RT editor is mRNA. In some embodiments, the LNP comprises a egRNA.

Disclosed herein include polynucleotides. In some embodiments, the polynucleotide encodes any of the RT editors, the tagRNAs, the RT editing systems, or the RT editing complexes described herein. In some embodiments, the polynucleotide is an mRNA. In some embodiments, the polynucleotide is operably linked to a regulatory element, optionally the regulatory element is an inducible regulatory element.

Disclosed herein include vectors. In some embodiments, the vector comprises any of the polynucleotides described herein. In some embodiments, the vector is an AAV vector.

Also provided herein are isolated cells. In some embodiments, the isolated cell comprises an RT editor, a tagRNA, an RT editing system, an RT editing complex, an RNP, an LNP, a polynucleotide, or a vector of the disclosure. In some embodiments, the cell is a mammalian cell, optionally a human cell. In some embodiments, the cell is a primary cell. In some embodiments, the cell is a hepatocyte. In some embodiments, the cell is from a subject having a disease or disorder, optionally Wilson's disease, further optionally the subject is a human. In some embodiments, the cell is from a subject having a disease or disorder, optionally alpha1 antitrypsin deficiency disease, further optionally the subject is a human. The disease or disorder can be selected from the group comprising an autoimmune disease, a neurological disease or disorder, a cancer, an inflammatory disease, a cardiovascular disease, an infectious disease, a genetic disease, a trinucleotide repeat expansion disorder, a metabolic disease, or any combination thereof. In some embodiments, the cell is from a subject having a disease or disorder, optionally phenylketonuria or hyperphenylalaninemia, further optionally the subject is a human.

Disclosed herein include pharmaceutical compositions. In some embodiments, the pharmaceutical composition comprises: (i) a RT editor, a tagRNA, a RT editing system, a RT editing complex, a RNP, a LNP, a polynucleotide, a vector, or a cell of the disclosure; and (ii) a pharmaceutically acceptable carrier.

Disclosed herein include methods for editing a double stranded target DNA. In some embodiments, the method comprises contacting the double stranded target DNA with (i) any of the tagRNAs of the disclosure and a reverse transcriptase (RT) editor comprising a DNA binding domain, a DNA endonuclease domain and a DNA polymerase domain or (ii) any of the RT editing systems described herein, wherein the tagRNA directs the RT editor to incorporate the intended nucleotide edit in the double stranded target DNA, thereby editing the double stranded target DNA.

Disclosed herein include methods for editing a double stranded target DNA. In some embodiments, the method comprises contacting the double stranded target DNA with any of the RT editing complexes of the disclosure, wherein the tagRNA directs the RT editor to incorporate the intended nucleotide edit in the double stranded target DNA, thereby editing the double stranded target DNA.

In some embodiments, the RT editor synthesizes a single stranded DNA encoded by the editing template, wherein the single stranded DNA replaces the editing target sequence and results in incorporation of the intended nucleotide edit or edits into a region corresponding to the editing target in the double stranded target DNA. In some embodiments, the double stranded target DNA is in a cell. In some embodiments, the cell is a mammalian cell, optionally a human cell. In some embodiments, the cell is a primary cell. In some embodiments, the cell is a hepatocyte. In some embodiments, the cell is a stem cell; optionally, an embryonic stem cell, an induced pluripotent stem cell, or an adult stem cell. In some embodiments, the cell is in a subject, optionally the subject is a human. In some embodiments, the cell is from a subject having a disease or disorder, optionally Wilson's disease. In some embodiments, the cell is from a subject having a disease or disorder, optionally alpha1 antitrypsin deficiency disease. In some embodiments, the cell is from a subject having a disease or disorder, optionally phenylketonuria or hyperphenylalaninemia. The disease or disorder can selected from the group comprising an autoimmune disease, a neurological disease or disorder, a cancer, an inflammatory disease, a cardiovascular disease, an infectious disease, a genetic disease, a trinucleotide repeat expansion disorder, a metabolic disease, or any combination thereof. In some embodiments, the method further comprises administering the cell to the subject after incorporation of the intended nucleotide edit.

Disclosed herein include cells generated by any of the methods described herein. Disclosed herein include populations of cells generated by any of the methods disclosed herein.

Disclosed herein include methods for treating or preventing a disease or disorder in a subject in need thereof. In some embodiments, the method comprises administering to the subject any of the RT editing systems of the disclosure, wherein the editing template comprises an intended nucleotide edit compared to a double stranded target DNA of the subject, wherein the tagRNA directs the RT editor to incorporate the intended nucleotide edit in the double stranded target DNA, and wherein incorporation of the intended nucleotide edit corrects a mutation in the double stranded target DNA associated with the disease or disorder, thereby treating or preventing the disease or disorder in the subject. The disease or disorder can selected from the group comprising an autoimmune disease, a neurological disease or disorder, a cancer, an inflammatory disease, a cardiovascular disease, an infectious disease, a genetic disease, a trinucleotide repeat expansion disorder, a metabolic disease, or any combination thereof.

Disclosed herein include methods for treating or preventing a disease or disorder in a subject in need thereof. In some embodiments, the method comprises administering to the subject any of the RT editing complexes, the RNPs, the LNPs, or the pharmaceutical compositions of the disclosure, wherein the editing template comprises an intended nucleotide edit compared to a double stranded target DNA of the subject, wherein the tagRNA directs the RT editor to incorporate the intended nucleotide edit in the double stranded target DNA, and wherein incorporation of the intended nucleotide edit corrects a mutation in the double stranded target DNA associated with the disease or disorder, thereby treating or preventing the disease or disorder in the subject. The disease or disorder can selected from the group comprising an autoimmune disease, a neurological disease or disorder, a cancer, an inflammatory disease, a cardiovascular disease, an infectious disease, a genetic disease, a trinucleotide repeat expansion disorder, a metabolic disease, or any combination thereof.

Disclosed herein include messenger RNAs (mRNAs). In some embodiments, the mRNA encodes a reverse transcriptase (RT) editor. In some embodiments, the mRNA encodes any of the RT editors of the disclosure. In some embodiments, the mRNA comprises one or more of a 5′-cap structure, a 5′-UTR, a 3′-UTR, and a nuclear localization sequence (NLS). In some embodiments, the mRNA comprises (i) a 5′-cap, (ii) a 5′-untranslated region (UTR); (iii) an open reading frame (ORF) comprising a nucleotide sequence that encodes the RT editor; and (iv) a 3′ untranslated region (UTR). In some embodiments, the mRNA has a structure comprising or consisting of 5′-[5′UTR]-[NLS]-[nCas9]-[linker]-[RT]-[NLS]-[3′UTR and/or viral element]-[polyA sequence]-3′. In some embodiments, one or more nucleosides of the mRNA sequence are chemically modified. In some embodiments, the mRNA comprises one or more additional segmented polyA sequences configured to increase mRNA stability and/or half-life. In some embodiments, the mRNA comprises one or more viral element sequences, optionally 3′ of the 3′-UTR. In some embodiments, the viral element comprises a woodchuck hepatitis virus post-transcriptional regulatory element (WPRE) sequence, optionally comprising the sequence of SEQ ID NO: 189, or a sequence that exhibits at least about 80%, at least about 85%, at least 90%, at least 95%, or 100% identity to SEQ ID NO: 189. In some embodiments, the viral element comprises a eK5 sequence, optionally located 3′ of the 3′-UTR, optionally comprising the sequence of SEQ ID NO: 190, or a sequence that exhibits at least about 80%, at least about 85%, at least 90%, at least 95%, or 100% identity to SEQ ID NO: 190. In some embodiments, the mRNA comprises one or more secondary structure motifs. In some embodiments, a secondary structure motif comprises a triple helix sequence, optionally a synthetic triple helix (STH) sequence. In some embodiments, the STH sequence comprises the sequence derived from a sequence element of a long non-coding RNA, optionally MALAT1. In some embodiments, the STH sequence comprises any one of the sequences of SEQ ID NOs: 191-192 and 245-249, or a sequence that exhibits at least about 80%, at least about 85%, at least 90%, at least 95%, or 100% identity to any one of the sequences of SEQ ID NOs: 191-192 and 245-249.

In some embodiments, the mRNA comprises: a polyA sequence comprising any one of the sequences of SEQ ID NOs: 182-187, or a sequence that exhibits at least about 80%, at least about 85%, at least 90%, at least 95%, or 100% identity to any one of the sequences of SEQ ID NOs: 182-187; one or more 3′ UTR element sequences selected from the group comprising any one of the sequences of SEQ ID NOs: 188-190, or a sequence that exhibits at least about 80%, at least about 85%, at least 90%, at least 95%, or 100% identity to any one of the sequences of SEQ ID NOs: 188-190; one or more 3′ UTR structural motifs selected from the group comprising any one of the sequences of SEQ ID NOs: 191-192, or a sequence that exhibits at least about 80%, at least about 85%, at least 90%, at least 95%, or 100% identity to any one of the sequences of SEQ ID NOs: 191-192; a 5′UTR sequence comprising the sequence of any one of SEQ ID NOS: 193 and 910-933, or a sequence that exhibits at least about 80%, at least about 85%, at least 90%, at least 95%, or 100% identity to any one of SEQ ID NOS: 193 and 910-933; and/or an RT editor sequence comprising the sequence of SEQ ID NO: 194, or a sequence that exhibits at least about 80%, at least about 85%, at least 90%, at least 95%, or 100% identity to SEQ ID NO: 194. In some embodiments, the mRNA comprises a 3′ UTR comprising any one of the sequences of SEQ ID NOs: 195-219, or a sequence that exhibits at least about 80%, at least about 85%, at least 90%, at least 95%, or 100% identity to any one of the sequences of SEQ ID NOs: 195-219. The mRNA can comprise one or more 5′ UTR element sequences selected from the group comprising any one of the sequences of SEQ ID NOs: 940-950 and 1075, or a sequence that exhibits at least about 85% identity to any one of the sequences of SEQ ID NOs: 940-950 and 1075. The mRNA can comprise one or more 3′ UTR element sequences selected from the group comprising any one of the sequences of SEQ ID NOs: 951-961 and 1076-1080, or a sequence that exhibits at least about 85% identity to any one of the sequences of SEQ ID NOs: 951-961 and 1076-1080. The mRNA can comprise one or more additional elements selected from the group comprising any one of the sequences of SEQ ID NOs: 962-963, or a sequence that exhibits at least about 85% identity to any one of the sequences of SEQ ID NOs: 962-963, and in some embodiments said one or more additional elements are situated downstream of the 3′UTR and/or the polyA tail. In some embodiments, the mRNA comprises one or more 5′-UTR element sequences selected from the group comprising any one of the sequences of SEQ ID NOs: 1081-1082, wherein the second codon of the mRNA is a “gcc”. In some embodiments, the mRNA is codon optimized for expression in human cells. In some embodiments, the mRNA (e.g., codon-optimized) comprises the sequence of any one of SEQ ID NOs: 1086-1088. Disclosed herein include ribonucleoprotein (RNP) complexes, lipid nanoparticles (LNPs), vectors, isolated cells, and pharmaceutical compositions comprising any of the mRNA of the disclosure. Disclosed herein include polynucleotides encoding the mRNAs provided herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 displays ATP7B genomic and protein sequences near the location of amino acid position 1069. Also shown are exemplary locations of spacer sequences of the disclosure.

FIG. 2 displays non-limiting exemplary efficiencies for correction of the H1069Q mutation as assayed in HEK293T cells that contain the H1069Q mutation (H1069Q HEK293T). In this experiment, editing components were delivered by plasmid transfection.

FIG. 3A-FIG. 3C display editing efficiencies in H1069Q HEK293T cells using mRNA (encoding the RT editor) and synthetic gRNA.

FIG. 4A-FIG. 4C display editing efficiencies in H1069Q Huh7 cells using mRNA (encoding the RT editor) and synthetic gRNA.

FIG. 5 displays non-limiting exemplary results of a spacer screen in the indicated cell types containing the H1069Q mutation. Cells were transfected with Cas9 mRNA and a synthetic gRNA.

FIG. 6 depicts an exemplary 3D structure of an RNA comprising SEQ ID NO: 245 (Comp14 transcript). FIG. 6 is original FIG. 3A from Wilusz, Jeremy E., et al. “A triple helix stabilizes the 3′ ends of long noncoding RNAs that lack poly(A) tails.” GenesDev 26 (2012): 2392-2407.

FIG. 7 depicts exemplary 3D structure of an RNA comprising SEQ ID NO: 247, 248, or 249. FIG. 7 is original FIG. 1A from Brown, Jessica A., et al. “Formation of triple-helical structures by the 3′-end sequences of MALAT1 and MENβ noncoding RNAs.” PNAS 109 (2012): 19202-19207.

FIG. 8 depicts an exemplary structure of an RNA comprising SEQ ID NO: 245 (predicted secondary structure of the 3′ end of the mature Comp.14 transcript).

FIG. 9 depicts an exemplary structure of an RNA comprising SEQ ID NO: 246 (full-length segment from human MALAT1 (HTH)).

FIG. 10 depicts an exemplary structure of an RNA comprising SEQ ID NO: 191 (synthetic triple helix, trimmed, adaptation, based on synthetic mix MALAT1 (STH)).

FIG. 11 depicts exemplary modified nucleotides that can be used in the RNAs (e.g., tagRNAs) of the disclosure.

FIG. 12 depicts a non-limiting exemplary structure of a scaffold RNA of the disclosure. Shown are various regions of the scaffold that were modified (See, e.g., Table 14A-Table 14C and Table 23).

FIG. 13 depicts a non-limiting exemplary schematic of a tagRNA according to the methods and compositions of the disclosure.

FIG. 14 depicts a non-limiting exemplary flow diagram for improvement of RT editing methods of the disclosure.

FIG. 15 depicts a non-limiting exemplary secondary structure of a tagRNA scaffold, with regions for optimization outlined by a box.

FIG. 16 depicts non-limiting exemplary data related to sequence optimization of the tagRNA scaffold. Shown are editing efficiencies of tested tagRNA scaffold sequence variants.

FIG. 17A-FIG. 17B depict non-limiting exemplary data related to an ET-FBS screen with a SsaCas9-based RT editor in Huh7 cells. Shown are the % T>C correction data (FIG. 17A) and percent indel formation data (FIG. 17B) for flap binding sequence (FBS) and editing template (ET) length variants tested.

FIG. 18A-FIG. 18B depict non-limiting exemplary data related to an ET-FBS screen with a truncated scaffold and shorter lengths. Shown are editing data for the variants tested with a dose of 125 ng mRNA and either 0.25 pmol tagRNA (FIG. 18A) or 0.07 pmol tagRNA (FIG. 18B).

FIG. 19 depicts non-limiting exemplary data related to optimization of the tagRNA spacer length.

FIG. 20 depicts non-limiting exemplary data related to editing results derived chemically modified scaffolds.

FIG. 21 depicts a non-limiting exemplary phylogenetic tree of MMLV, bat, and avian reverse transcriptases.

FIG. 22 depicts non-limiting exemplary data of editing efficiencies of RTs of the disclosure in correction of ATP7B mutation in Huh7 cells.

FIG. 23 depicts non-limiting exemplary data of editing efficiencies of RTs of the disclosure in ATP7B surrogate mutation correction in primary human hepatocytes.

FIG. 24A-FIG. 24B depict non-limiting exemplary data related to editing efficiencies of M. brandtii variants. In FIG. 24B, lower concentrations of all RNA components were used as compared to FIG. 24A. The experimental data of FIG. 24B employed improved chemical modifications in the tagRNA as compared to FIG. 24A. P1_30 is also referred to herein as ChemMod_Set2_30.

FIG. 25A-FIG. 25B depict non-limiting exemplary data related to editing efficiencies of M. georgiana variants. In FIG. 25B, lower concentrations of all RNA components were used as compared to FIG. 25A. The tagRNA of FIG. 25B used improved chemical modifications in the tagRNA as compared to FIG. 25A.

FIG. 26A-FIG. 26B depict non-limiting exemplary data related to M. brandtii variants (FIG. 26A) and M. georgiana variants (FIG. 26B) in primary human hepatocytes.

FIG. 27 depicts non-limiting exemplary data related to improved editing exhibited by different RT variants.

FIG. 28 depicts non-limiting exemplary data related to editing efficiencies using different 5′ UTRs in the mRNA encoding the RT.

FIG. 29A-FIG. 29B depicts non-limiting exemplary data related to editing results in Groups 1, 2, and 4 (FIG. 29A) and Group 3 (FIG. 29B) of a first in vivo proof of concept study performed in a humanized AATD mouse model.

FIG. 30 depicts non-limiting exemplary data related to editing results in a second in vivo proof of concept study performed in a humanized AATD mouse model.

FIG. 31 depicts a non-limiting exemplary schematic of mouse exon 12 being replaced with the human exon 12 in the humanized PKU mouse model generated.

FIG. 32 depicts non-limiting exemplary data related to editing results in an in vivo proof of concept study performed in humanized PKU mice.

FIG. 33 depicts editing frequencies of codon-optimized RT editors for correction of ATP7B surrogate mutation in primary human hepatocytes.

FIG. 34 depicts editing frequencies of codon-optimized RT editors for correction of PAH surrogate mutation in primary human hepatocytes.

FIG. 35 displays an exemplary schematic of SSB insertion sites within an RT editor.

FIG. 36 depicts an exemplary schematic and 3D model for insertion of an SSB as a chimeric protein with nSsaCas9.

FIG. 37 displays a bar graph of exemplary editing data using the indicated RT editor variants.

FIG. 38 shows editing results from a stabilion variant screen.

FIG. 39 displays results of in vivo screening of indicated mRNAs encoding an RT editor.

FIG. 40 displays an exemplary schematic of an SSB (e.g., Sso7d) insertion sites within an RT editor.

FIG. 41 displays exemplary editing data using the indicated RT editors.

FIG. 42 displays editing data for correction of a Wilson's disease mutation from primary human hepatocytes using the indicated mRNA, for the purpose of testing different linkers, NLSs, UTRs, RTs, stabilizing elements, and polyA tails.

FIG. 43 displays editing data from tested in Huh7 cells containing the ATP7B H1069Q mutation using the indicated chemically modified tagRNA.

FIG. 44 displays editing data from tested in Huh7 cells containing the ATP7B H1069Q mutation using the indicated chemically modified tagRNA.

FIG. 45 displays editing data from tested in Huh7 cells containing the ATP7B H1069Q mutation using the indicated chemically modified tagRNA.

FIG. 46 displays exemplary in vivo data from mouse for correction of human ATP7B (H1069Q) using the indicated RT editor mRNA.

FIG. 47 displays exemplary in vivo data from mouse for correction of human ATP7B mutation using the indicated mRNA encoding RT editor and tagRNAs.

FIG. 48 displays in vivo data from mouse for correction of PAH-R408W mutation using the indicated mRNA encoding RT editor, tagRNA, and egRNA. Shown from left to right on the graph are data using: pTPRT-U-493, pTPRT-U-540, pTPRT-U-543. pTPRT-U-554, pTPRT-U-558, pVC313, pVC268, and pAM198.

FIG. 49 displays exemplary editing data using the indicated tagRNA scaffold.

FIG. 50 displays exemplary editing data using the indicated tagRNA scaffold.

FIG. 51 displays exemplary editing data using the indicated egRNA scaffold.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include compositions for reverse transcriptase (RT) editing. Compositions disclosed herein comprise an RT editing system, comprising an RT editor, template armed guide RNA (tagRNAs). In some embodiments, the RT editing system comprises an additional gRNA designated herein as ‘egRNA’.

Also provided herein are ribonucleoprotein (RNP) complexes comprising any of the RT editing complexes disclosed herein, or a component thereof. Disclosed herein include lipid nanoparticles (LNPs). In some embodiments, the LNP comprises any of the RT editing systems disclosed herein, or a component thereof. Provided herein include polynucleotides. In some embodiments the polynucleotide encodes any of the tagRNAs, the RT editor, the egRNA, or the RT editing complexes of the disclosure. Disclosed herein include isolated cells. In some embodiments, the isolated cell comprises any of the tagRNAs, the RT editor, the egRNA, the RT editing complexes, the RNPs, the LNPs, or the vectors provided herein.

Disclosed herein include compositions. In some embodiments, the composition is a pharmaceutical composition. In some embodiments, the pharmaceutical composition comprises: any of the tagRNAs, the RT editor, the egRNA, the RT editing systems, the RT editing RT editing complexes, the RNPs, the LNPs, the polynucleotides, the vectors, or the cells disclosed herein; and (ii) a pharmaceutically acceptable carrier.

Disclosed herein include methods for editing a mutated gene. In some embodiments, the method comprises contacting the gene at the site of mutation with (i) any of the tagRNAs of the disclosure and a RT editor. In some embodiments, the tagRNA directs the RT editor to incorporate the intended nucleotide edit or edits in the mutated gene, thereby editing the mutated gene and correcting the mutation.

Disclosed herein include methods for treating a disease or disorder in a subject in need thereof. In some embodiments, the method comprises administering to the subject (i) any of the tagRNAs of the disclosure and a RT editor. In some embodiments, the tagRNA directs the RT editor to incorporate the intended nucleotide edit or edits in the mutated gene in the subject, thereby treating the disease or disorder in the subject.

Disclosed herein include methods for editing an ATP7B gene. In some embodiments, the method comprises contacting the ATP7B gene with (i) any of the tagRNAs of the disclosure and a RT editor. In some embodiments, the tagRNA directs the RT editor to incorporate the intended nucleotide edit in the ATP7B gene, thereby editing the ATP7B gene.

Disclosed herein include methods for treating Wilson's disease in a subject in need thereof. In some embodiments, the method comprises administering to the subject (i) any of the tagRNAs of the disclosure and a RT editor. In some embodiments, the tagRNA directs the RT editor to incorporate the intended nucleotide edit in the ATP7B gene in the subject, thereby treating Wilson's disease in the subject.

Provided herein are methods and compositions of editing of the SERPINA1 gene that encodes A1AT serine protease inhibitor for treating alpha1 antitrypsin deficiency (AATD) disease. Editing of the S allele mutation or the Z allele mutation in SERPINA1 gene are contemplated herein. The Z allele mutation on exon 5 of SERPINA1 is an E342K mutation. E342K mutation leads to misfolding of the AAT protein leading to polymers and liver damage. Contemplated are methods for a single nucleotide (T->C) correction leading to a K342E correction at the SERPINA1 gene.

Provided herein are methods and compositions of editing of the PAH gene that encodes Phenylalanine hydroxylase enzyme for treating phenylketonuria or hyperphenylalaninemia. Editing of the R408W mutation is contemplated herein. The R408W mutation is located in exon 12 of the PAH gene, within the protein's catalytic domain, and causes the PAH protein to misfold. This structural alteration results in a significant reduction in enzyme activity. Contemplated are methods for a single nucleotide (T->C) correction leading to a W408R correction at the PAH gene.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” can mean plus or minus 5% of the provided value.

As used herein, the terms “DNA binding domain” and “nucleic acid programmable DNA binding protein” or “napDNAbp,” of which a Cas protein is an example, may be used interchangeably herein, and can refer to proteins that use RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence. As used herein, the term “nucleic acid programmable DNA binding protein” or “napDNAbp,” of which Cas9 is an example, refer to proteins that use RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

As used herein, the term “guide RNA” or “gRNA” can refer to a site-specific targeting RNA that can bind an RNA-guided endonuclease to form a complex, and direct the activities of the bound RNA-guided endonuclease (such as a Cas endonuclease) to a specific sequence within a target nucleic acid (e.g., a specific gene or region within a gene). The guide RNA can include one or more RNA molecules. In some embodiments, the gRNA is a template armed gRNA (tagRNA). In some embodiments, the gRNA is an enhancer gRNA (egRNA).

As used herein, the term “target DNA” can refer to the specific region on a double-stranded DNA within a subject's genome intended for editing by a gene editing system. In certain embodiments, the gene editing system is an RT editing system.

As used herein, the term “search target sequence” can refer to the sequence on target strand that is complementary or substantially complementary to the spacer sequence of the tagRNA.

As used herein, the term “editing target sequence” can refer to the sequence being edited on the non-target strand.

As used herein, the term “silent mutation” can refer to a nucleotide change or nucleotide changes (for e.g., substitution or substitutions) in a DNA sequence that does not result in a change to the amino acid sequence of the protein the DNA sequence encodes.

As used herein, the term “protospacer” refers to the sequence in DNA adjacent to the PAM (protospacer adjacent motif) sequence. In some embodiments, the protospacer is 20 nucleotides long. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). In order for Cas9 to function it also requires a specific protospacer adjacent motif (PAM) that varies depending on the bacterial species of the Cas9 gene. The skilled person will appreciate that the literature in the state of the art sometimes refers to the “protospacer” as the target-specific guide sequence on the guide RNA itself, rather than referring to it as a “spacer.” Thus, in some cases, the term “protospacer” as used herein may be used interchangeably with the term “spacer.” The context of the description surrounding the appearance of either “protospacer” or “spacer” will help inform the reader as to whether the term is in reference to the gRNA or the DNA target.

As used herein, the terms “upstream” and “downstream” define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

As used herein, the term “protospacer adjacent sequence” or “PAM” refers to a DNA sequence that is an important targeting component of a Cas9 nuclease. The PAM sequence can be on either strand, and is downstream in the 5′ to 3′ direction of the Cas9 cut site. Different PAM sequences can be associated with different Cas9 nucleases or equivalent proteins from different organisms.

As used herein, the term “spacer sequence” in connection with a guide RNA or a tagRNA refers to the portion of the guide RNA or tagRNA which contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

As used herein, a “secondary structure” of a nucleic acid molecule (e.g., an RNA fragment, or a gRNA) refers to the base pairing interactions within the nucleic acid molecule.

As used herein, the term “Cas endonuclease” or “Cas nuclease” refers to an RNA-guided DNA endonuclease associated with and/or derived from the CRISPR adaptive immunity system. The term “nickase” refers to a Cas9 or other endonuclease with one of the two nuclease domains inactivated. This enzyme is capable of cleaving only one strand of a target DNA.

Unless otherwise indicated “nuclease” and “endonuclease” are used interchangeably herein to refer to an enzyme which possesses endonucleolytic catalytic activity for polynucleotide cleavage.

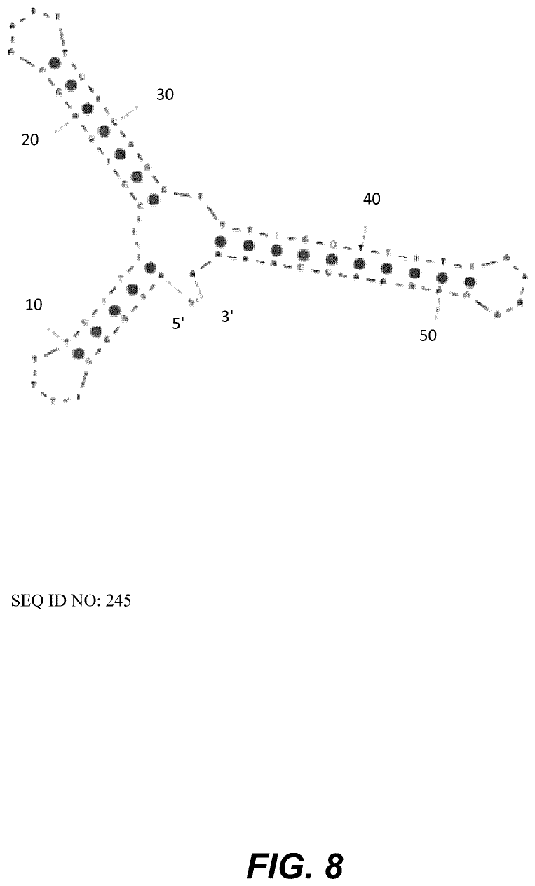

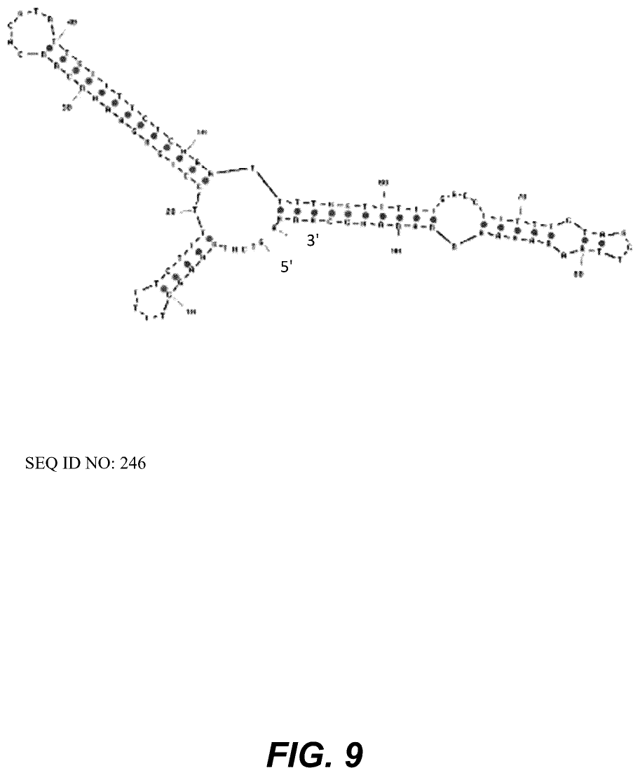

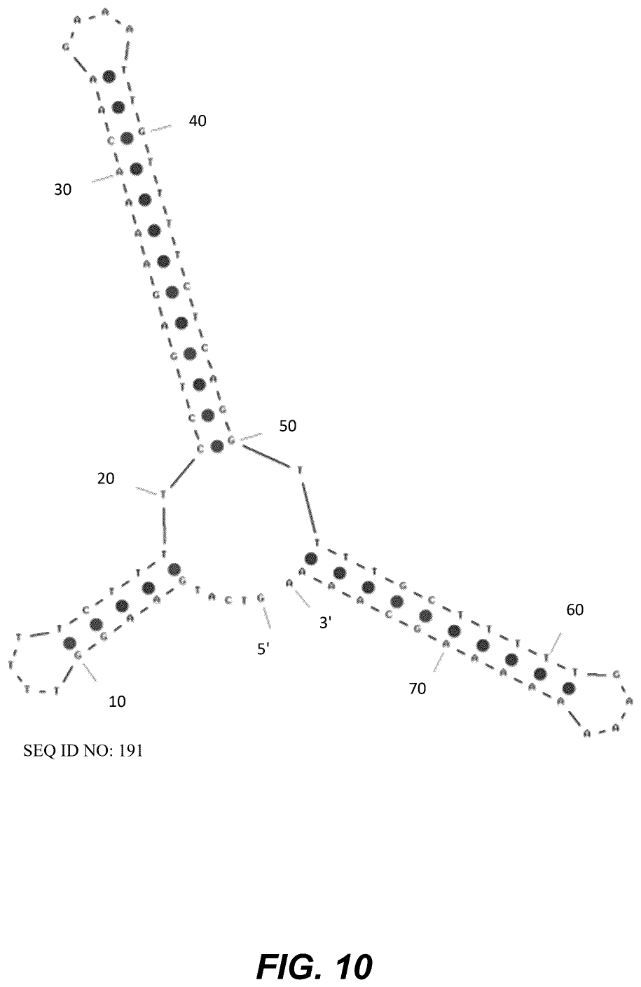

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases. Any of the RNA sequences disclosed herein may also be DNA (either single-stranded or double-stranded), e.g., wherein “U” is converted to “T.” Any of the DNA sequences disclosed herein (e.g., FIG. 8-FIG. 10) may also be RNA, e.g., wherein “T” is converted to “U.”

A “functional variant” or “functional mutant”, as used herein, refers to any variant or mutant of a reference protein (e.g., a wild-type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild-type reverse transcriptase but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (K_{d}), for example a K_{d} of, or a K_{d} less than, 10⁻⁶ M, 10⁻⁷ M, 10⁻⁸ M, 10⁻⁹M, 10⁻¹⁰ M, 10⁻¹¹ M, 10⁻¹² M, 10⁻¹³ M, 10⁻¹⁴ M, 10⁻¹ M, or a number or a range between any two of these values. K_{d} can be dependent on environmental conditions, e.g., pH and temperature. “Affinity” refers to the strength of binding, and increased binding affinity is correlated with a lower K_{d}.

As used herein, the term “hybridizing” or “hybridize” refers to the pairing of substantially complementary or complementary nucleic acid sequences within two different molecules. Pairing can be achieved by any process in which a nucleic acid sequence joins with a substantially or fully complementary sequence through base pairing to form a hybridization complex. “Hybridizing” or “hybridize” can comprise denaturing the molecules to disrupt the intramolecular structure(s) (e.g., secondary structure(s)) in the molecule. In some embodiments, denaturing the molecules comprises heating a solution comprising the molecules to a temperature sufficient to disrupt the intramolecular structures of the molecules. In some instances, denaturing the molecules comprises adjusting the pH of a solution comprising the molecules to a pH sufficient to disrupt the intramolecular structures of the molecules. For purposes of hybridization, two nucleic acid sequences or segments of sequences are “substantially complementary” if at least 80% of their individual bases are complementary to one another. The complementary portion of each sequence can be referred to herein as a “segment”, and the segments are substantially complementary if they have 80% or greater identity.

The terms “complementarity” and “complementary” mean that a nucleic acid can form hydrogen bond(s) with another nucleic acid based on traditional Watson-Crick base paring rule, that is, adenine (A) pairs with thymine (T, or uracil (U) in RNA) and guanine (G) pairs with cytosine (C). Complementarity can be perfect (e.g., complete complementarity) or imperfect (e.g. partial complementarity). Perfect or complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. In some embodiments, the complementarity can be at least 70%, 80%, 90%, 100% or a number or a range between any two of these values. In some embodiments, the complementarity is perfect, i.e., 100%. For example, the complementary candidate sequence segment is perfectly complementary to the candidate sequence segment, whose sequence can be deduced from the candidate sequence segment using the Watson-Crick base pairing rules.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The terms “DNA editing efficiency,” or “editing efficiency” may be used interchangeably herein and can refer to the number or proportion of intended target sequences that are edited. In some embodiments, the efficiency can be reported as % indel, e.g., the proportion of insertions and/or deletions detected in the target sequence. Indels (e.g., insertion-deletions) can result from repair of double-stranded DNA breaks caused by Cas9 cleavage by processes including, but not limited to, non-homologous end joining (NHEJ) repair. The terms “correction efficiency”, “precise correction efficiency”, or “precise edit efficiency” may be used interchangeably herein and can refer to the number or proportion of target sequences that contain the desired or intended edit. In some embodiments, the efficiency can be reported as % correction or % edit, e.g. the proportion of intended edits detected in the target sequence. In some embodiments, the intended edit or edits revert a pathogenic mutation to wildtype sequence, thereby correcting a disease-associated mutation.

The term “off-target editing frequency,” as used herein, refers to the number or proportion of unintended DNA sequences that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Since many of the Cas9-dependent off-target sites have high sequence identity to the target site of interest, nucleic acid primers with sufficient complementarity to regions upstream or downstream of the Cas9-dependent off-target site may be designed using techniques and kits known in the art. These kits make use of polymerase chain reaction (PCR) amplification, which produces amplicons as intermediate products. The target and off-target sequences may comprise genomic loci that further comprise protospacers and PAMs. Accordingly, the term “amplicons,” as used herein, may refer to nucleic acid molecules that constitute the aggregates of genomic loci, protospacers and PAMs. High-throughput sequencing techniques used herein may further include Sanger sequencing and/or whole genome sequencing (WGS).

As used herein, the terms “transfection” or “infection” refer to the introduction of a nucleic acid into a host cell, such as by contacting the cell with liposomes or nanoparticles (e.g., lipid nanoparticles) as described herein.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented.

As used herein, the terms “effective amount” or “pharmaceutically effective amount” or “therapeutically effective amount” refer to an amount sufficient to effect beneficial or desirable biological and/or clinical results.

The term “pharmaceutically acceptable excipient” as used herein refers to any suitable substance that provides a pharmaceutically acceptable carrier, additive or diluent for administration of a compound(s) of interest to a subject. Pharmaceutically acceptable excipients can encompass substances referred to as pharmaceutically acceptable diluents, pharmaceutically acceptable additives, and pharmaceutically acceptable carriers.

As used herein, a “subject” refers to an animal for whom a diagnosis, treatment, or therapy is desired. In some embodiments, the subject is a mammal. “Mammal,” as used herein, refers to an individual belonging to the class Mammalia and includes, but not limited to, humans. In some embodiments, the mammal is a primate. In some embodiments, the mammal is a human. In some embodiments, the mammal is not a human. In some embodiments, the subject has or is suspected of having a disease that can be corrected via gene editing. In some embodiments, the gene editing system is an RT editing system. In some embodiments, the subject has or is suspected of having Wilson's disease. In some embodiments, the subject has or is suspected of having alpha1 antitrypsin deficiency disease. In some embodiments, the subject has or is suspected of having phenylketonuria or hyperphenylalaninemia.

### Reverse Transcriptase (RT) Editing

RT editing methods and compositions disclosed herein are directed to correction of a disease mutation using reverse transcriptase (RT) editing. Compositions disclosed herein comprise and RT editor or an mRNA encoding a reverse transcriptase (RT) editor, a long guide RNA encoding the edit designated as ‘tagRNA’, and optionally, a second guide RNA designated as ‘egRNA’ (opposite strand gRNA). In some embodiments, the composition induces programmable editing of a target DNA using an RT editor complexed with a tagRNA to incorporate an intended nucleotide edit (also referred to herein as a nucleotide change) into the target DNA. In some embodiments, the composition comprises a second guide RNA (egRNA).

A target gene of the RT editing may comprise a double stranded DNA molecule having two complementary strands: a first strand that may be referred to as a “target strand” or a “non-edit strand”, and a second strand that may be referred to as a “non-target strand,” or an “edit strand” or an “opposite strand”. The RT editors provided herein can be employed to introduce more than one nucleotide change. In some embodiments, two (or more) RT editors provided herein can be employed to edit two (or more) different genes or two (or more) different sites of a gene. RT editors provided herein can correct more than one mutation. Two disclosed RT editors can be used to target two different genes or two different segments of a gene. In some embodiments, one RT editor (e.g., as an mRNA) can be delivered with two or more different tagRNAs (and optionally egRNAs)—either in the same or different LNP formulations. The mRNA of the RT editor can be the same for any two (or more) different sites. In some embodiments, one or more different editors can be employed. In some embodiments, the target is a non-coding element, e.g., a promoter, an enhancer, and the like.

### RT Editor

The term “RT editor” refers to the polypeptide or polypeptide components involved in RT editing, or any polynucleotide(s) encoding the polypeptide or polypeptide components. In various embodiments, a RT editor includes a polypeptide domain having DNA endonuclease activity and a polypeptide domain having DNA polymerase activity. In some embodiments, the RT editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the RT editor comprises a polypeptide domain that is an inactive nuclease, in some embodiments, the polypeptide domain having comprises a nucleic acid guided DNA endonuclease domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase, in some embodiments, the RT editor comprises additional polypeptides involved in RT editing, for example, a polypeptide domain having a 5′ endonuclease activity, e.g., a 5′ endogenous DNA flap endonucleases (e.g., FEN1), for helping to drive the RT editing process towards the edited product formation. In some embodiments, the RT editor further comprises an RNA-protein recruitment polypeptide, for example, a MS2 coat protein.

A RT editor may be engineered. In some embodiments, the polynucleotide or polypeptide components of a RT editor do not naturally occur in the same organism or cellular environment. In some embodiments, the polynucleotide or polypeptide components of a RT editor may be of different origins or from different organisms. In some embodiments, a RT editor comprises a DNA binding domain, a DNA endonuclease domain and a DNA polymerase domain that are derived from different species. In some embodiments, a RT editor comprises a Cas polypeptide (DNA endonuclease domain) and a reverse transcriptase polypeptide (DNA polymerase) that are derived from different species.

In some embodiments, polypeptide domains of a RT editor may be fused or linked by a peptide linker to form a fusion protein. In other embodiments, a RT editor comprises one or more polypeptide domains provided in trans as separate proteins, which are capable of being associated to each other through non-peptide linkages or through aptamers or recruitment sequences. For example, a RT editor may comprise a DNA binding domain and a reverse transcriptase domain associated with each other by an RNA-protein recruitment aptamer, e.g., an MS2 aptamer, which may be linked to a tagRNA. RT editor polypeptide components may be encoded by one or more polynucleotides in whole or in part, in some embodiments, a single polynucleotide, construct, or vector encodes the RT editor fusion protein. In some embodiments, multiple polynucleotides, constructs, or vectors each encode a polypeptide domain or portion of a domain of a RT editor, or a portion of a RT editor fusion protein. For example, a RT editor fusion protein may comprise an N-terminal portion fused to an intein-N and a C-terminal portion fused to an intein-C, each of which is individually encoded by an AAV vector

In some embodiments, the RT Editor is transcribed from an mRNA. In some embodiments, the mRNA comprises a 5′-cap structure. In some embodiments, the mRNA comprises a nuclear localization sequence (NLS). In some embodiments, the mRNA sequence comprises a dead Cas9 sequence. In some embodiments, the Cas9 is a Cas nickase sequence. In some embodiments, the RT Editor mRNA sequence comprises a 5′-UTR. In some embodiments, the RT Editor mRNA sequence comprises a 3′-UTR. In some embodiments, the RT Editor comprises a sequence of a viral element. In some embodiments, the viral element is a woodchuck hepatitis virus post-transcriptional regulatory element (WPRE) sequence. In some embodiments, the RT editor comprises secondary structure motifs. In some embodiments, the viral element sequence comprises or consists of any one of SEQ ID Nos: 189-190. In some embodiments, the RT editor comprises an ek5 sequence. The ek5 sequence can comprise the sequence of SEQ ID NO: 190, or a sequence that exhibits at least about 85% identity to SEQ ID NO: 190. The ek5 sequence can have one, two, three, four, or five mismatches relative to the sequence of SEQ ID NO: 190. The viral element sequence can be derived from Aichi virus 1 (AiV-1), such as, for example, the 3′ UTR K5 element (GenBank: NC_001918.1, 8,122-8,251). In some embodiments, the viral element sequence comprises the extended form of K5 (“ek5,” 8,067-8,251, 185 nt). Viral element sequences are described in Seo, Jenny J., et al. (“Functional viromic screens uncover regulatory RNA elements.” Cell 186.15 (2023): 3291-3306), the content of which is incorporated herein by reference in its entirety. In some embodiments, the secondary structure motif comprises a triple helix sequence. In some embodiments, the RT editor mRNA sequence has a structure comprising or consisting of a sequence comprising from 5′ to 3′ as [5′UTR]-[NLS]-[nCas9]-[linker]-[RT]-[NLS]-[3′UTR_and/or_viral_element]-[polyA_sequence]. In some embodiments, any nucleoside of the RT editor mRNA sequence may be chemically modified.

Compositions disclosed herein comprise a long guide RNA designated herein as “template armed guide RNA” or “tagRNA”. The tagRNA comprises a spacer sequence, a scaffold sequence, an editing template and a flap binding sequence. In some embodiments, the tagRNA comprises in 5′ to 3′ order: spacer sequence, scaffold sequence, editing template, and a flap binding sequence.

### Rt Editor Nucleotide Polymerase Domain and Endonuclease Domain

In some embodiments, a RT editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the RT editor comprises a DNA-dependent DNA polymerase. For example, a RT editor having a DNA-dependent DNA polymerase can synthesize a new single stranded DNA using a tagRNA editing template that comprises a DNA sequence as a template. In such cases, the tagRNA is a chimeric or hybrid tagRNA, and comprising an extension arm comprising a DNA strand. The chimeric or hybrid tagRNA may comprise an RNA portion (including the spacer and the gRNA core) and a DNA portion (the extension arm comprising the editing template that includes a strand of DNA).

In some embodiments, RT editor comprises a DNA endonuclease domain (e.g., a Cas9 nickase). In some embodiments, a Cas protein, e.g., Cas9, can be a wild type or a modified form of a Cas protein. In some embodiments, a Cas protein, e.g., Cas9, can be a nuclease active variant, nuclease inactive variant, a nickase, or a functional variant or functional fragment of a wild-type Cas protein. In some embodiments, a Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild-type Cas protein. A Cas protein, e.g., Cas9, can comprise an amino acid change such as a deletion, insertion, substitution, fusion, chimera, or any combination thereof relative to a corresponding wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, a DNA endonuclease domain, RNA binding domain, helicase domains, protein-protein interaction domains, and dimerization domains. In various embodiments, a Cas protein comprises a guide nucleic acid recognition and/or binding domain that can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a RT editor comprises a Cas protein, e.g., Cas9, wherein all nuclease domains of the Cas protein are active. In some embodiments, a RT editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a tagRNA.

In some embodiments, a RT editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a RT editor comprises a Cas nickase comprising two nuclease domains (e.g., Cas9), with one of the two nuclease domains modified to lack catalytic activity or deleted. In some embodiments, the Cas nickase of a RT editor comprises a nuclease inactive RuvC domain and a nuclease active HNH domain. In some embodiments, the Cas nickase of a RT editor comprises a nuclease inactive HNH domain and a nuclease active RuvC domain. In some embodiments, a RT editor comprises a Cas9 nickase having an amino acid substitution in the RuvC domain e.g., an amino acid substitution that reduces or abolishes nuclease activity of the RuvC domain. In some embodiments, the Cas9 nickase comprises a DOX amino acid substitution compared to a wild type S. pyogenes Cas9, wherein X is any amino acid other than D. In some embodiments, a RT editor comprises a Cas9 nickase having an amino acid substitution in the HNH domain, e.g., an amino acid substitution that reduces or abolishes nuclease activity of the HNH domain. In some embodiments, the Cas9 nickase comprises a H840X amino acid substitution compared to a wild type S. pyogenes Cas9, wherein X is any ammo acid other than H.

In some embodiments, a RT editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a RT editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some aspects, a dead Cas protein is a dead Cas9 protein. In some embodiments, a RT editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a RT editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a tagRNA. A Cas9 protein may refer to a wild-type Cas9 protein from any organism or a homolog, ortholog, or paralog from any organisms; any functional mutants or functional variants thereof, or any functional fragments or domains thereof. In some embodiments, a RT editor comprises a full-length Cas9 protein. In some embodiments, the Cas9 protein can generally comprises at least about 50%, 60%, 70%, 80%, 90%, 100% sequence identity to a wild-type reference Cas9 protein (e.g., Cas9 from S. pyogenes). In some embodiments, the Cas9 comprises an amino acid change such as a deletion, insertion, substitution, fusion, chimera, or any combination thereof as compared to a wild-type reference Cas9 protein.

In some embodiments the DNA endonuclease domain of the RT editor comprises a Cas9 protein (e.g., a Cas9 nickase). The Cas9 protein can comprise an amino acid sequence that is at least 80% identical to any one of SEQ ID NOS: 142-163 and 270-314. The Cas9 protein can comprise an amino acid sequence having one, two, three, four, or five mismatches relative to the sequence of any one of SEQ ID NOS: 142-163 and 270-314.

### Reverse Transcriptases

In some embodiments, a RT editor comprises an RNA-dependent DNA polymerase domain, for example, a reverse transcriptase (RT). An RT or an RT domain may be a wild-type RT domain, a full-length RT domain, or may be a functional mutant, a functional variant, or a functional fragment thereof. An RT or an RT domain of a RT editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a RT editor comprising the engineered RT has improved RT editing efficiency over a RT editor having a reference naturally occurring RT.

In some embodiments, a RT editor comprises a virus RT, for example, a retrovirus RT. Nonlimiting examples of virus RT include Moloney murine leukemia virus (M-MLV or MLVRT or M-MLV RT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (LJR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein. In some embodiments, an MMLV RT, e.g., reference MMLV RT, comprises a sequence as disclosed in SEQ ID NO: 15.

In some embodiments, the RT editor comprises a wild-type M-MLV RT, a functional mutant, a functional variant, or a functional fragment thereof. In some embodiments, the RT editor comprises a reference M-MLV RT, a functional mutant, a functional variant, or a functional fragment thereof.

In some embodiments, the RT is an RT (e.g., a retro-transposon RT) from an avian genome. The avian may be of the order Gallformes, Aseriformes, Passeriformes, Gruiformes, Struthioniformes, Rheiformes, Casuariformes, Apyerygiformes, Otidiformes, Columbiformes, Sphenisciformes, Cathartiformes, Accipitriformes, Strigiformes, Psittaciformes, Charadriiformes, or Falconiformes. Exemplary avians include those of Galiformes (e.g. chicken, quails, and turkey), Anseriformes (e.g. duck, and goose), Charadriiformes (e.g. gull, barred button quail, and plover), Columbiformes (e.g. pigeon), Struthioniformes (e.g. ostrich), Passeriformes (e.g. crow, finch, sparrow, starling, and swallow), Psittaciformes (e.g. parrot), Falconiformes (e.g. eagle, and falcon), Strigiformes (e.g. owl), Sphenisciformes (e.g. penguin), and Psittaciformes (e.g. parakeet, and parrot). The avian can belong to the order Passeriformes. The avian can belong to the family Passerellidae. The avian can belong to the genus Melospiza. In some embodiments, the RT is from the genome of M. georgiana. In some embodiments, the M. georgiana RT is an engineered RT. In some embodiments, the mutation sites on the M. georgiana RT may be one or more of G206N, M312K, W319F, E336P, and L611W. The DNA polymerase domain (e.g., RT) can comprise an amino acid sequence that is at least about 80% identical to SEQ ID NO: 141 (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 141). The DNA polymerase domain (e.g., RT) can comprise an amino acid sequence having one, two, three, four, five, six, seven, eight, nine, or ten mismatches relative to the sequence of SEQ ID NO: 141. There are provided, in some embodiments, RT editors. The RT editor can comprise a DNA endonuclease domain, a DNA binding domain and a DNA polymerase domain. The DNA polymerase domain can comprise an amino acid sequence that is at least 80% identical to SEQ ID NO: 141. The DNA polymerase domain can comprise an amino acid sequence having one, two, three, four, or five mismatches relative to the sequence of SEQ ID NO: 141. In some embodiments, the RT editor comprises a reverse transcriptase has a sequence of any one of SEQ ID NOs: 141, 250-269, 315-319, 899-909, and 1015-1018. In certain embodiments, the reverse transcriptase comprises an amino acid sequence that is at least 80% identical to SEQ ID NO: 141.

In some embodiments, the RT is an RT from a mammalian genome. In some embodiments, the mammal may be of the order Chiroptera (bat). That bat can be of the family Phyllostomidae (e.g., leaf-nosed bats), Noctilionidae (bulldog bats), Cistugidae, Thyropteridae (e.g., disk-winged bats), Molossidae (e.g., free-tailed bats), Miniopteridae (e.g., long winged bat), Mormoopidae, Mystacinidae (e.g., New Zealand short-tailed bats), Myzopodidae (e.g., sucker-footed bats), Natalidae (e.g., funnel-eared bats), Emballonuridae (e.g., sheath-tailed bats), Nycteridae (e.g., slit-faced bats), Furipteridae (e.g., smoky bats), Vespertilionidae (e.g., vesper bats), Craseonycteridae, Megadermatidae (e.g., false vampire bats), Rhinolophidae (e.g., horseshoe bats), Pteropodidae (e.g., Old World fruit bats), Hipposideridae (e.g., Old World leaf-nosed bats), or Rhinopomatidae. In some embodiments, the bat is of the genus Myotis, Pipistrelles, or Eptesicus. In some embodiments, the bat species is M. brandtii, M. daubentonii, P. kuhli, or E. nilssonii. In some embodiments, the RT editor comprises a reverse transcriptase having a sequence of any one of SEQ ID NOs: 250-255, 258-259, and 266-267. In some embodiments, the reverse transcriptase comprises an amino acid sequence that is at least 80% identical to any one of SEQ ID NOs: 250-255, 258-259, and 266-267.

In some embodiments, the DNA polymerase domain comprises a reverse transcriptase. In some embodiments, the DNA polymerase domain comprises a reverse transcriptase comprising one or more mutations. In some embodiments, at least one of the one or more mutations is at an amino acid position functionally equivalent to V101, N200, A208, G248, P330, L435, K445, and/or A623 relative to SEQ ID NO: 8.

In some embodiments, the DNA polymerase domain comprises a reverse transcriptase comprising an amino acid sequence that is at least 80% identical to SEQ ID NO: 255 (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 255). In some embodiments, the reverse transcriptase comprises one or more mutations. In some embodiments, at least one of the one or more mutations is at amino acid position V98, N197, A205, S245, P327, V432, R442, and/or A623 relative to SEQ ID NO: 255. In some embodiments, the reverse transcriptase comprises one or more transition mutations selected from the group consisting of V98R, N197C, N197D, N197G, A205T, S245C, P327E, P327Q, V432K, R442T, and A623F relative to SEQ ID NO: 255.

In some embodiments, the DNA polymerase domain comprises a reverse transcriptase comprising an amino acid sequence that is at least 80% identical to SEQ ID NO: 260 (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 260). The reverse transcriptase can comprise one or more mutations. In some embodiments, at least one of the one or more mutations is at amino acid position V106, N204, E212, G252, P334, L440, E450, and/or A629 relative to SEQ ID NO: 260. In some embodiments, the reverse transcriptase comprises one or more transition mutations selected from the group consisting of V106R, N204C, N204D, N204G, E212T, G252C, P334E, P334Q, L440K, E450T, and/or A629F relative to SEQ ID NO: 260.

In some embodiments, the DNA polymerase domain comprises a reverse transcriptase comprising an amino acid sequence that is at least 80% identical to SEQ ID NO: 255. In some embodiments, the reverse transcriptase comprises an N197C or V98R transition mutation relative to SEQ ID NO: 255 (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 255).

In some embodiments, the DNA polymerase domain comprises a reverse transcriptase comprising an amino acid sequence that is at least 80% identical to SEQ ID NO: 260 (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 260). In some embodiments, the reverse transcriptase comprises an N204C and/or V106R transition mutation relative to SEQ ID NO: 260.

### Accessory Domains

In some embodiments, the RT editor further comprises an accessory domain. In some embodiments, the accessory domain comprises a single-strand binding (SSB) protein domain or a stabilon. In some embodiments, the SSB protein domain is derived from RecA protein, Sso7d protein, or Sto7d protein. In some embodiments the accessory domain is situated at the N-terminus, the C-terminus, or at an internal location of the RT editor. In some embodiments, the RT editor comprising an accessory domain comprises an amino acid sequence that is at least 85% identical to any one of SEQ ID NOs: 1276-1298 (e.g., 85%, 86%, 87%, 88%, 89%, 90%, 91%, 92%, 93%, 94%, 95%, 96%, 97%, 98%, 99%, 100%, or a number or a range between any two of these values, identical to any one of SEQ ID NOs: 1276-1298). In some embodiments, the SSB protein domain is derived from Sso7d and comprises one or more mutations at an amino acid position functionally equivalent to K12 and/or E35 of a wild type Sso7d amino acid sequence. In some embodiments, the one or more mutations comprise K12L and/or E35L relative to a wild type Sso7d sequence. In some embodiments, the SSB protein domain comprises an amino acid sequence that is at least 85% identical to SEQ ID NO: 1205 (e.g., 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 1205). In some embodiments, the RT editor comprising the SSB protein domain comprises from N-terminus to C-terminus: [N-terminus of nCas9]-[linker]-[SSB protein domain]-[linker]-[RT]-[linker]-[C-terminus of nCas9]. In some embodiments, the N-terminus of nCas9 comprises an amino acid sequence functionally equivalent to amino acids 1-1247 of SEQ ID NO: 6 and/or the C-terminus of nCas9 comprises an amino acid sequence functionally equivalent to amino acids 1248-1367 of SEQ ID NO: 6. In some embodiments, the RT editor comprising the SSB protein domain comprises an amino acid sequence that is at least 85% identical to any one of SEQ ID NOs: 1207-1209 (e.g., 85%, 86%, 87%, 88%, 89%, 90%, 91%, 92%, 93%, 94%, 95%, 96%, 97%, 98%, 99%, 100%, or a number or a range between any two of these values, identical to any one of SEQ ID NOs: 1207-1209).

Additional Sequence Elements of the RT Editor mRNA

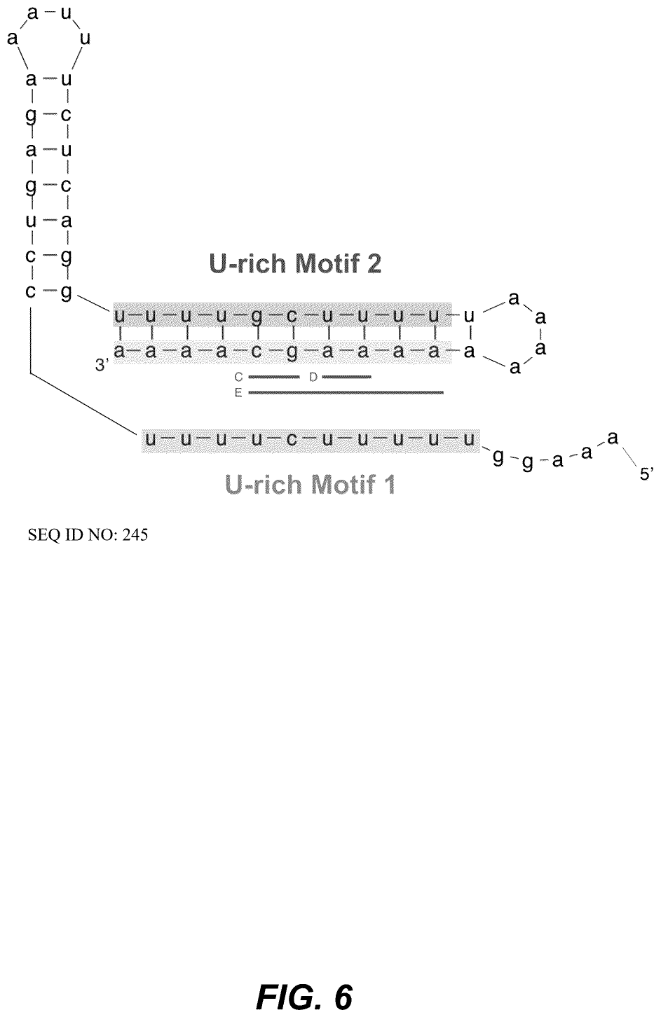

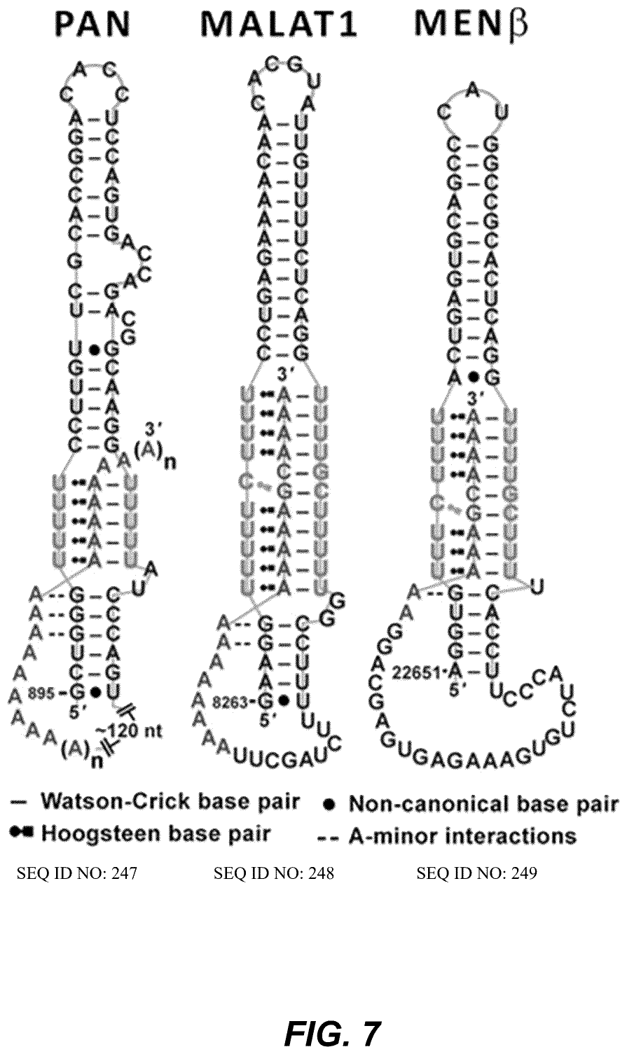

The present disclosure provides optimized mRNAs encoding a RT editor, that provide effective genome editing of a target cell population when administered with one or more gRNAs (e.g., tagRNA and egRNA). In some embodiments, additional segmented polyA sequences increase mRNA stability and half-life of the RT editor. In some embodiments, the disclosure provides an mRNA comprising (i) a 5′-cap, (ii) a 5′-untranslated region (UTR); (ii) an open reading frame (ORF) comprising a nucleotide sequence that encodes a RT editor; and (iv) a 3′ untranslated region (UTR). In some embodiments, the mRNA further comprises viral element sequences 3′ of the 3′-UTR. In some embodiments, the viral element sequence comprises the sequence of SEQ ID NO: 189. In some embodiments, the mRNA further comprises an eK5 sequence that is located 3′ of the 3′-UTR. eK5 is a regulatory RNA element derived from a virus that when placed in the 3′UTR of an mRNA can lead to increased protein expression (for instance, as in Seo et al Cell 2023, 186: 3291). In some embodiments, the ek5 sequence comprises the sequence of SEQ ID NO: 190. In some embodiments, the synthetic triple helix secondary structure sequence (STH) comprises the sequence derived from a sequence element of the long non-coding RNA, such as MALAT1. In some embodiments, the STH sequence comprises the sequence of SEQ ID NO: 246. FIG. 6-FIG. 10 provide exemplary secondary structure motifs employed in the methods and compositions provided herein. In some embodiments their presence in the 3′ UTR of mRNAs disclosed herein provides 3′ end stabilization and/or protection. Some embodiments provided herein employ the triple helix motif from MALAT1, or an engineered (minimal) version. In some embodiments, MALAT1 (Gene ID: 378938) based triple helix motif is situated at the 3′ end. MALAT1 (metastasis associated lung adenocarcinoma transcript 1) also known as NEAT2 (noncoding nuclear-enriched abundant transcript 2) is a large, infrequently spliced non-coding RNA, which is highly conserved amongst mammals and highly expressed in the nucleus.

tagRNAs

Disclosed herein include target priming RNAs (tagRNAs). The term “target priming RNA”, or “tagRNA”, refers to a guide polynucleotide that comprises one or more intended nucleotide edits for incorporation into the target DNA. In some embodiments, the tagRNA associates with and directs a RT editor to incorporate the one or more intended nucleotide edits into the target gene via RT editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene or may refer to the edit encoded by the editing template on the newly synthesized single stranded DNA that replaces the editing target sequence, as compared to the editing target sequence. In some embodiments, a tagRNA comprises a spacer sequence that is complementary or substantially complementary to a search target sequence on a target strand of the target gene, in some embodiments, the tagRNA comprises a gRNA core that associates with a DNA endonuclease domain, e.g., a CRISPR-Cas protein domain, of a RT editor. In some embodiments, the tagRNA further comprises an extended nucleotide sequence comprising one or more intended nucleotide edits compared to the endogenous sequence of the target gene, wherein the extended nucleotide sequence may be referred to as an extension arm.

In some embodiments, in a template armed gRNA (tagRNA), a spacer sequence is complementary or substantially complementary to a specific sequence on the target strand, which may be referred to as a “search target sequence.” In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand.” In some embodiments, the PAM strand comprises a protospacer sequence and optionally a protospacer adjacent motif (PAM) sequence. In RT editing using a Cas-protein-based RT editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease, in some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a tagRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is upstream of a PAM sequence recognized by a Cas9 nickase, wherein the Cas9 nickase comprises a nuclease active RuvC domain and a nuclease inactive HNH domain. In some embodiments, the nick site is 3 nucleotides upstream of the PAM sequence, and the PAM sequence is recognized by a Streptococcus pyogenes Cas9 nickase. In some embodiments, the Cas nickase comprises any one of the sequences recited in SEQ ID NO: 142-163.

In some embodiments, the nick site is 3 base pairs upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 nucleotides upstream of the PAM sequence, and the PAM sequence is recognized by a S. thermophilus Cas9 nickase that comprises a nuclease active RuvC domain and a nuclease inactive HNH domain. In some embodiments, the Cas nick site (e.g., a Cas9 nickase comprising an amino acid sequence that is at least 80% identical (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) to any one of SEQ ID NOs: 142-163 and 270-314, or an amino acid sequence having at least one, at least two, at least three, at least four, or at least five mismatches relative to the sequence of any one of SEQ ID NOs: 142-163 and 270-314), is 4, 5, 6 or more than 6 nucleotides base pairs upstream of the PAM sequence and the PAM sequence is recognized by a Cas9 nickase wherein the Cas9 nickase comprises a nuclease active HNH domain and a nuclease inactive RuvC domain.

An “editing template” of a tagRNA is a single-stranded portion of the tagRNA that is 5′ of the FB sequence and comprises a region of complementarity to the PAM strand (i.e., the non-target strand or the edit strand), and comprises one or more intended nucleotide edits compared to the endogenous sequence of the double stranded target DNA. In some embodiments, the editing template and the FB sequence are immediately adjacent to each other. Accordingly, in some embodiments, a tagRNA in RT editing comprises a single-stranded portion that comprises the editing template sequence and the FB sequence immediately adjacent to each other. In some embodiments, the single stranded portion of the tagRNA comprising both the editing template sequence and the flap binding sequence is complementary or substantially complementary to an endogenous sequence on the PAM strand (i.e., the non-target strand or the edit strand) of the double stranded target DNA except for one or more non-complementary nucleotides at the intended nucleotide edit positions. As used herein, regardless of relative 5-3′ positioning in other contexts, the relative positions as between the FB sequence and the editing template, and the relative positions as among elements of a tagRNA, are determined by the 5′ to 3′ order of the tagRNA as a single molecule regardless of the position of sequences in the double stranded target DNA that may have complementarity or identity to elements of the tagRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence.” In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits.

In some embodiments, the editing template comprises a nucleotide sequence selected from SEQ ID NOs: 95-98. In some embodiments, the editing template comprises a nucleotide sequence selected from SEQ ID NOs: 95-98 or a sequence having one, two, or three mismatches relative to a nucleotide sequence selected from SEQ ID NOs: 95-98. In some embodiments, the editing template comprises or consists of a nucleotide sequence selected from SEQ ID NOs: 95-98 or a sequence at least 85% identical (e.g., 85%, 86%, 87%, 88%, 89%, 90%, 91%, 92%, 93%, 94%, 95%, 96%, 97%, 98%, 99%, 100%, or a number or a range between any two of these values) to a nucleotide sequence selected from SEQ ID NOs: 95-98.

A “flap binding (FB) sequence” is a single-stranded portion of the tagRNA that comprises a region of complementarity to the PAM strand (i.e., the non-target strand or the edit strand). The FB sequence is complementary or substantially complementary to a sequence on the PAM strand of the double stranded target DNA that is immediately upstream of the nick site. In some embodiments, in the process of RT editing, the tagRNA complexes with and directs a RT editor to bind the search target sequence on the target strand of the double stranded target DNA and the RT editor generates a nick at the nick site on the non-target strand (e.g., the PAM strand) of the double stranded target DNA. In some embodiments, the FB sequence is complementary to or substantially complementary to, and can anneal to, a free 3′ end on the non-target strand of the double stranded target DNA at the nick site. In some embodiments, the FB sequence annealed to the free 3′ end on the non-target strand can initiate target-primed DNA synthesis. In some embodiments, the FB sequence is about 2 to 20 nucleotides in length. In some embodiments, the FB sequence is about 8 to 16 nucleotides in length. In some embodiments, the FBS is 6 nucleotides in length. In some embodiments, the FB site comprises a nucleotide sequence selected from GGCGTGGCAGTCA (SEQ ID NO: 91), GGCGTGGCAGTC (SEQ ID NO: 92), GGCGTGGCAGT (SEQ ID NO: 93), GGCGTGGCAG (SEQ ID NO: 94), GGCGTGGCA, GGCGTGGC, GGCGTGG, and GGCGTG. In some embodiments, the FB sequence comprises a nucleotide sequence selected from GGCGTGGCAGTCA (SEQ ID NO: 91), GGCGTGGCAGTC (SEQ ID NO: 92), GGCGTGGCAGT (SEQ ID NO: 93), GGCGTGGCAG (SEQ ID NO: 94), GGCGTGGCA, GGCGTGGC, GGCGTGG, and GGCGTG. In some embodiments, the FB sequence comprises a nucleotide sequence having one, two, or three mismatches relative to a sequence selected from GGCGTGGCAGTCA (SEQ ID NO: 91), GGCGTGGCAGTC (SEQ ID NO: 92), GGCGTGGCAGT (SEQ ID NO: 93), GGCGTGGCAG (SEQ ID NO: 94), GGCGTGGCA, GGCGTGGC, GGCGTGG, and GGCGTG. In some embodiments, the FB sequence comprises or consists of a sequence selected from GGCGTGGCAGTCA (SEQ ID NO: 91), GGCGTGGCAGTC (SEQ ID NO: 92), GGCGTGGCAGT (SEQ ID NO: 93), GGCGTGGCAG (SEQ ID NO: 94), GGCGTGGCA, GGCGTGGC, GGCGTGG, and GGCGTG or a sequence at least 85% identical (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from GGCGTGGCAGTCA (SEQ ID NO: 91), GGCGTGGCAGTC (SEQ ID NO: 92), GGCGTGGCAGT (SEQ ID NO: 93), GGCGTGGCAG (SEQ ID NO: 94), GGCGTGGCA, GGCGTGGC, GGCGTGG, and GGCGTG.

### Nucleic Acid Modifications

In some embodiments, any of the nucleic acids of the disclosure can comprise one or more modifications (e.g., gRNA, tagRNA, egRNA, or any nucleic acid encoding any component of the RT editor systems disclosed herein). In some embodiments, the gRNA (e.g., egRNA) or tagRNA is a chemically modified gRNA or tagRNA. Various types of RNA modifications can be introduced to the gRNAs or tagRNAs to enhance stability, reduce the likelihood or degree of innate immune response, and/or enhance other attributes as described in the art. The gRNAs or tagRNAs described herein can comprise one or more modifications including internucleoside linkages, purine or pyrimidine bases, or sugar. In some embodiments, a modification is introduced at the terminal of a gRNA or tagRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

In some embodiments, programmable editing of a target DNA comprises a template armed gRNA (tagRNA). In some embodiments, one or more nucleoside of the tagRNA is chemically modified. In some embodiments, the chemical modifications can be any one of an LNA, 2′-fluoro, DNA, 2′-OMe, and 2′ MethoxyEthoxy (2′-MOE)-chemical modification. In some embodiments, the tagRNA comprises one or more deoxyribonucleotides (e.g., DNA). In some embodiments, each nucleotide of the editing template comprises an LNA modification. In some embodiments, each nucleotide of the flap binding sequence comprises an LNA modification. In some embodiments, each nucleotide of both the editing template and the flap binding sequences comprises an LNA modification. In some embodiments, every second nucleotide of the editing template comprises an LNA modification. In some embodiments, every second nucleotide of the flap binding sequence comprises an LNA modification. In some embodiments, every second nucleotide of both the editing template and the flap binding sequences comprises an LNA modification. In some embodiments, every third nucleotide of the editing template comprises an LNA modification. In some embodiments, every third nucleotide of the flap binding sequence comprises an LNA modification. In some embodiments, every third nucleotide of both the editing template and the flap binding sequences comprises an LNA modification. In some embodiments, each nucleotide of the editing template comprises a 2′-fluoro modification. In some embodiments, each nucleotide of the flap binding sequence comprises a 2′-fluoro modification. In some embodiments, each nucleotide of both the editing template and the flap binding sequences comprises a 2′-fluoro modification. In some embodiments, every second nucleotide of the editing template comprises a 2′-fluoro modification. In some embodiments, every second nucleotide of the flap binding sequence comprises a 2′-fluoro modification. In some embodiments, every second nucleotide of both the editing template and the flap binding sequences comprises a 2′-fluoro modification. In some embodiments, every third nucleotide of the editing template comprises a 2′-fluoro modification. In some embodiments, every third nucleotide of the flap binding sequence comprises a 2′-fluoro modification. In some embodiments, every third nucleotide of both the editing template and the flap binding sequences comprises a 2′-fluoro modification. In some embodiments, each nucleotide of the editing template comprises a 2′-OMe modification. In some embodiments, each nucleotide of the flap binding sequence comprises a 2′-OMe modification. In some embodiments, each nucleotide of both the editing template and the flap binding sequences comprises a 2′-OMe modification. In some embodiments, every second nucleotide of the editing template comprises a 2′-OMe modification. In some embodiments, every second nucleotide of the flap binding sequence comprises a 2′-OMe modification. In some embodiments, every second nucleotide of both the editing template and the flap binding sequences comprises a 2′-OMe modification. In some embodiments, every third nucleotide of the editing template comprises a 2′-OMe modification. In some embodiments, every third nucleotide of the flap binding sequence comprises a 2′-OMe modification. In some embodiments, every third nucleotide of both the editing template and the flap binding sequences comprises a 2′-OMe modification. In some embodiments, each nucleotide of the editing template comprises a 2′-MOE modification. In some embodiments, each nucleotide of the flap binding sequence comprises a 2′-MOE modification. In some embodiments, each nucleotide of both the editing template and the flap binding sequences comprises a 2′-MOE modification. In some embodiments, every second nucleotide of the editing template comprises a 2′-MOE modification. In some embodiments, every second nucleotide of the flap binding sequence comprises a 2′-MOE modification. In some embodiments, every second nucleotide of both the editing template and the flap binding sequences comprises a 2′-MOE modification. In some embodiments, every third nucleotide of the editing template comprises a 2′-MOE modification. In some embodiments, every third nucleotide of the flap binding sequence comprises a 2′-MOE modification. In some embodiments, every third nucleotide of both the editing template and the flap binding sequences comprises a 2′-MOE modification. In some embodiments, the total number of tagRNA nucleotides comprising a chemical modification provided herein (e.g., an LNA, 2′-fluoro, DNA, 2′-OMe, and/or 2′-MOE chemical modification) can be at least, or can be at most, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, nucleotides. As understood by the skilled artisan, any modification pattern disclosed herein (e.g., according to any of SEQ ID NOs: 16-47, 99-130, 164-181, 320-497, 563-682, 791-792, 1046-1053, and 1089-1203) can be applied to a tagRNA comprising a desired spacer and editing template.

In some embodiments, a tagRNA complexes with and directs a RT editor to bind to the search target sequence of the target gene. In some embodiments, the bound RT editor generates a nick on the edit strand (PAM strand) of the target gene at the nick site. In some embodiments, a flap binding site (FB sequence) of the tagRNA anneals with a free 3′ end formed at the nick site, and the RT editor initiates DNA synthesis from the nick site, using the free 3′ end as a primer. Subsequently, a single-stranded DNA encoded by the editing template of the tagRNA is synthesized. In some embodiments, the newly synthesized single-stranded DNA comprises one or more intended nucleotide edits compared to an endogenous target gene sequence. Accordingly, in some embodiments, the editing template of a tagRNA is complementary to a sequence in the edit strand except for one or more mismatches at the intended nucleotide edit positions in the editing template. The endogenous, e.g., genomic, sequence that is partially complementary to the editing template may be referred to as an “editing target sequence.” Accordingly, in some embodiments, the newly synthesized single stranded DNA has identity or substantial identity to a sequence in the editing target sequence, except for one or more insertions, deletions, or substitutions at the intended nucleotide edit positions.

In some embodiments, the newly synthesized single-stranded DNA equilibrates with the editing target on the edit strand of the target gene for pairing with a target strand of a target gene. In some embodiments, an editing target sequence of a target gene is excised by a flap endonuclease (FEN), for example, FEN1. In some embodiments, the FEN is an endogenous FEN, for example, in a cell comprising a target gene. In some embodiments, the FEN is provided as part of the RT editor, either linked to other components of the RT editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

In some embodiments, a RT editor comprises a Cas9 functional variant that is of smaller molecular weight than a wild-type SPYCas9 protein. In some embodiments, a smaller-sized Cas9 functional variant may facilitate delivery to cells, e.g., by an expression vector, nanoparticle, or other means of delivery. In some embodiments, a smaller-sized Cas9 functional variant is a Class 2 Type II Cas protein. In some embodiments, a smaller-sized Cas9 functional variant is a Class 2 Type V Cas protein. In some embodiments, a smaller-sized Cas9 functional variant is a Class 2 Type VI Cas protein.

### Nuclear Localization Sequences and Linkers

In some embodiments, a RT editor further comprises one or more nuclear localization sequence (NLS). In some embodiments, the NLS helps promote translocation of a protein into the cell nucleus. In some embodiments, a RT editor comprises a fusion protein, e.g., a fusion protein comprising a DNA endonuclease domain and a DNA polymerase, that comprises one or more NLSs. In some embodiments, one or more polypeptides of the RT editor are fused to or linked to one or more NLSs. In some embodiments, the RT editor comprises a DNA endonuclease domain and a DNA polymerase domain that are provided in trans, wherein the DNA endonuclease domain and/or the DNA polymerase domain is fused or linked to one or more NLSs. In some embodiments the RT editor mRNA comprises or consists of the structure: [5′UTR]-[NLS]-[nCas9]-[linker]-[RT]-[NLS]-[3′UTR and/or viral element]-[polyA sequence]. The RT editor mRNA can comprises or consist of the structure [5′-cap]-[5′UTR]-[NLS]-[nCas9]-[linker and/or NLS]-[RT]-[NLS]-[3′UTR_and/or_viral_element]-[secondary structure motif and/or polyA sequence]. In some cases, the positions of nCas9 and RT are swapped to e.g. improve editing efficiency.

In some embodiments, a RT editor or RT editing complex comprises at least one NLS. In some embodiments, a RT editor or RT editing complex comprises at least two NLSs. In embodiments with at least two NLSs, the NLSs can be the same NLS, or they can be different NLSs.

In addition, the NLSs can be expressed as part of a RT editor complex. The location of the NLS fusion can be at the N-terminus, the C-terminus, or positioned anywhere within a sequence of a RT editor or a component thereof (e.g., inserted between the DNA endonuclease domain and the DNA polymerase domain of a RT editor fusion protein, between the DNA endonuclease domain and a linker sequence, between a DNA polymerase and a linker sequence, between two linker sequences of a RT editor fusion protein or a component thereof, in either N-terminus to C-terminus or C-terminus to N-terminus order).

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a RT editor comprise bipartite NLSs. In some embodiments, a nuclear localization signal (NLS) is predominantly basic. In some embodiments, the one or more NLSs of a RT editor are rich in lysine and arginine residues. In some embodiments, the one or more NLSs of a RT editor comprise proline residues.

In some embodiments, a nuclear localization signal (NLS) comprises the sequence of any one of: MDSLLMNRRKFLYQFKNVRWAKGRRETYLC (SEQ ID NO: 66), KRTADGSEFESPKKKRKV (SEQ ID NO: 11), KRTADGSEFEPKKKRKV (SEQ ID NO: 59), NLSKRPAAIKKAGQAKKKK (SEQ ID NO: 60), RQRRNELKRSF (SEQ ID NO: 61), and NQSSNFGPMKGGNFGGRSSGPYGGGGQYFAKPRNQGGY (SEQ ID NO: 62).

In some embodiments, an NLS is a monopartite NLS. For example, in some embodiments, a NLS is a SV40 large T antigen NLS PKKKRKV (SEQ ID NO: 65). In some embodiments, an NLS is a bipartite NLS. In some embodiments, a bipartite NLS comprises two basic domains separated by a spacer sequence comprising a variable number of amino acids. In some embodiments, an NLS is a bipartite NLS. In some embodiments, a bipartite NLS consists of two basic domains separated by a spacer sequence comprising a variable number of amino acids. In some embodiments, the spacer amino acid sequence comprises the sequence KRXXXXXXXXXXKKKL (Xenopus nucleoplasmin) (SEQ ID NO: 63), wherein X is any amino acid. In some embodiments, the NLS comprises a nucleoplasmin NLS sequence KRPAATKKAGQAKKKK (SEQ ID NO: 64). In some embodiments, an NLS is a noncanonical sequences such as M9 of the hnRNP A1 protein, the influenza virus nucleoprotein NLS, and the yeast Gal4 protein NLS. In some embodiments, a NLS is a noncanonical sequences such as M9 of the hnRNP A1 protein, the influenza virus nucleoprotein NLS, and the yeast Gal4 protein NLS.

In some embodiments, a bipartite NLS consists of two basic domains separated by a spacer sequence comprising a variable number of amino acids, in some embodiments, an NLS comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 5, 12, and 65-73. In some embodiments, an NLS comprises an amino acid sequence selected from the group consisting of SEQ ID NOs: 5, 12, and 65-73. in some embodiments, a RT editing composition comprises a polynucleotide that encodes an NLS that comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 12, and 65-73. In some embodiments, a RT editing composition comprises a polynucleotide that encodes an NLS that comprises an amino acid sequence selected from the group consisting of SEQ ID NOs: 5, 2, and 65-73.

Non-limiting examples of NLS sequences are provided in Table 1 below.

TABLE 1
EXEMPLARY NUCLEAR LOCALIZATION SIGNALS

| Name/Description | Sequence | SEQ ID NO: |
|---|---|---|
| NLS of SV40 Large T-AG | PKKKRKV | 65 |
| NLS | MKRTADGSEFESPKKKRKV | 5 |
| NLS | MDSLLMNRRKFLYQFKNVRWAKGRRETYLC | 66 |
| NLS of Nucleoplasmin | AVKRPAATKKAGQAKKKKLD | 67 |
| NLS of EGL-13 | MSRRRKANPTKLSENAKKLAKEVEN | 68 |
| NLS of C-Myc | PAAKRVKLD | 12 |
| NLS of Tus-protein | KLKIKRPVK | 69 |
| NLS of polyoma large T-AG | VSRKRPRP | 70 |
| NLS of Hepatitis D virus antigen | EGAPPAKRAR | 71 |
| NLS of murine p53 | PPQPKKKPLDGE | 72 |
| C-terminal linker and NLS of an | SGGSKRTADGSEFEPKKKRKV | 73 |
| exemplary RT editor fusion | | |
| protein | | |
| Hybrid NLS | PAAKKKKLD | 1054 |
| VirD NLS | PKRPRDRHDGELGGRKRARG | 1055 |

In some embodiments, components of a RT editor are directly fused to each other. In some embodiments, components of a RT editor are associated to each other via a linker. As used herein, a linker can be any chemical group or a molecule linking two molecules or moieties, e.g., a DNA binding domain, a DNA endonuclease domain and a polymerase domain of a RT editor. In some embodiments, a linker is an organic molecule, group, polymer, or chemical moiety. In some embodiments, the linker comprises a non-peptide moiety. The linker may be a covalent bond (e.g., a carbon-carbon bond, disulfide bond, carbon-heteroatom bond, etc.), or it may be a polymeric linker, for example, a polynucleotide sequence.

In some embodiments, two or more components of a RT editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 5, 6, 7, 8, 9, 10, 11, 12, 13, 14, 15, 16, 17, 18, 19, 20, 25, 35, 45, 50, 55, 60, 60, 65, 70, 70, 75, 80, 85, 90, 90, 95, 100, 101, 102, 103, 104, 105, 110, 120, 130, 140,150, 160, 175, 180, 190, or 200 amino acids in length. In some embodiments, the peptide linker is 5-100 amino acids in length. In some embodiments, the peptide linker is 10-80 amino acids in length. In some embodiments, the peptide linker is 15-70 amino acids in length. In some embodiments, the peptide linker is 16 amino acids in length, 24 amino acids in length, 64 amino acids in length, or 96 amino acids in length, in some embodiments, the peptide linker is at least 50 amino acids in length, in some embodiments, the peptide linker is at least 40 amino acids in length, in some embodiments, the peptide linker is at least 30 amino acids in length. In some embodiments, the peptide linker is 46 amino acids in length. In some embodiments, the peptide linker is 92 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 74), (G)n, (EAAAK)n (SEQ ID NO: 75), (GGS)n, (SGGS)n (SEQ ID NO: 76), (XP)n, or any combination thereof, wherein n is independently an integer between 1 and 30, and wherein X is any amino acid. In some embodiments, the linker comprises the amino acid sequence (GGS)n, wherein n is 1, 3, or 7. In some embodiments, the linker comprises the amino acid sequence SGSETPGTSESATPES (SEQ ID NO: 77). In some embodiments, the linker comprises the amino acid sequence SGGSSGGSSGSETPGTSESATPESSGGSSGGS (SEQ ID NO: 78). In some embodiments, the linker comprises the amino acid sequence SGGSGGSGGS (SEQ ID NO: 79). In some embodiments, the linker comprises the amino acid sequence SGGS (SEQ ID NO: 10). In some embodiments, the linker comprises the amino acid sequence SGGSSGGSSGSETPGTSESATPESAGSYPYDVPDYAGSAAPAAKKKKLDGSGSGGSSGG S (SEQ ID NO: 80). In some embodiments, the linker comprises the amino acid sequence GGSGGS (SEQ ID NO: 81), GGSGGSGGS (SEQ ID NO: 82), or SGGSSGGSSGSETPGTSESATPESSGGSSGGSS (SEQ ID NO: 83).

Components of a RT editor may be connected to each other in any order. In some embodiments, the DNA binding domain, a DNA endonuclease domain and the DNA polymerase domain of a RT editor may be fused to form a fusion protein or may be joined by a peptide or protein linker, in any order from the N-terminus to the C-terminus. In some embodiments, a RT editor comprises a DNA binding domain, a DNA endonuclease domain fused or linked to the C-terminal end of a DNA polymerase domain. In some embodiments, a RT editor comprises a DNA binding domain, a DNA endonuclease domain fused or linked to the N-terminal end of a DNA polymerase domain. In some embodiments, the RT editor comprises a fusion protein comprising the structure NH2-[DNA binding domain, a DNA endonuclease domain]-[polymerase]-COOH; or NH2-[polymerase]-[DNA binding domain, a DNA endonuclease domain]-COOH, wherein each instance of “]-[” indicates the presence of an optional linker sequence. In some embodiments, a RT editor comprises a fusion protein and a DNA polymerase domain provided in trans, wherein the fusion protein comprises the structure NH2-[DNA binding domain, a DNA endonuclease domain]-[RNA-protein recruitment polypeptide]-COOH. In some embodiments, a RT editor comprises a fusion protein and a DNA binding domain, a DNA endonuclease domain provided in trans, wherein the fusion protein comprises the structure NH2-[DNA polymerase domain]-[RNA-protein recruitment polypeptide]-COOH.

In some embodiments, a RT editor fusion protein, a polypeptide component of a RT editor, or a polynucleotide encoding the RT editor fusion protein or polypeptide component, may be split into an N-terminal half and a C-terminal half or polypeptides that encode the N-terminal half and the C-terminal half, and provided to a target DNA in a cell separately. For example, in some embodiments, a RT editor fusion protein may be split into a N-terminal and a C-terminal half for separate delivery in AAV vectors, and subsequently translated and colocalized in a target cell to reform the complete polypeptide or RT editor protein. In such cases, separate halves of a protein or a fusion protein may each comprise a split-intein to facilitate colocalization and reformation of the complete protein or fusion protein by the mechanism of intein facilitated trans splicing. In some embodiments, a RT editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete RT editor fusion protein in the target cell.

tagRNAs

Disclosed herein include template armed gRNAs (tagRNAs). In some embodiments, the tagRNA comprises: a spacer that is complementary to a search target sequence on a first strand of a target gene; a scaffold sequence, an editing template that comprises a region of complementarity to an editing target sequence on a second strand of the target gene; and a flap binding sequence, wherein the first strand and the second strand are complementary to each other. In some embodiments, the editing target sequence is within the mutation site of the target gene. In some embodiments, the editing target sequence is within exon 14 of the ATP7B gene. In some embodiments, the editing target sequence is within exon 5 of the SERPINA1 gene. In some embodiments, the editing target sequence is within exon 12 of the PAH gene.

The tagRNA can comprise from 5′ to 3′: the spacer, the scaffold sequence, the editing template, and the FB sequence. In some embodiments, spacer, the scaffold sequence, the editing template, and the FB sequence form a contiguous sequence in a single molecule. The editing template can comprise an intended nucleotide edit compared to the target gene. In some embodiments, the tagRNA guides the RT editor to incorporate the intended nucleotide edit into the target gene when the tagRNA is contacted with the target gene. In some embodiments, the RT editor synthesizes a single stranded DNA encoded by the editing template, wherein the single stranded DNA replaces the editing target sequence and results in incorporation of the intended nucleotide edit into a region corresponding to the editing target in the target gene. The search target sequence can be complementary to a protospacer sequence in the target gene. The protospacer sequence can be adjacent to a protospacer adjacent motif (PAM) in the target gene. In some embodiments, the tagRNA results in incorporation of the intended nucleotide edit in the PAM when contacted with the target gene. In some embodiments, the target gene is the ATP7B gene. In some embodiments, the target gene is the SERPINA1 gene. In some embodiments, the target gene is the ATP7B gene. In some embodiments, the target gene is the PAH gene.

In some embodiments, the extension arm comprises a flap binding site sequence (FB sequence) that can initiate target-primed DNA synthesis. In some embodiments, the FB sequence is complementary or substantially complementary to a free 3′ end on the edit strand of the target gene at a nick site generated by the RT editor. In some embodiments, the extension arm further comprises an editing template that comprises one or more intended nucleotide edits to be incorporated in the target gene by RT editing. In some embodiments, the editing template is a template for an RNA-dependent DNA polymerase domain or polypeptide of the RT editor, for example, a reverse transcriptase domain. The reverse transcriptase editing template may also be referred to herein as an editing template. In some embodiments, the editing template comprises partial complementarity to an editing target sequence in the target gene, e.g., an ATP7B gene, a PAH gene, or a SERPINA1 gene. In some embodiments, the editing template comprises substantial or partial complementarity to the editing target sequence except at the position of the intended nucleotide edits to be incorporated into the target gene.

In some embodiments, a tagRNA includes only RNA nucleotides and forms an RNA polynucleotide. In some embodiments, a tagRNA is a chimeric polynucleotide that includes both RNA and DNA nucleotides. For example, a tagRNA can include DNA in the spacer sequence, the scaffold sequence, or the extension arm. In some embodiments, a tagRNA comprises DNA in the spacer sequence. In some embodiments, the entire spacer sequence of a tagRNA is a DNA sequence. In some embodiments, the tagRNA comprises DNA in the scaffold sequence, for example, in a stem region of the scaffold sequence. In some embodiments, the tagRNA comprises DNA in the extension arm, for example, in the editing template. An editing template that comprises a DNA sequence may serve as a DNA synthesis template for a DNA polymerase in a RT editor, for example, a DNA-dependent DNA polymerase. Accordingly, the tagRNA may be a chimeric polynucleotide that comprises RNA in the spacer, scaffold sequence, and/or the FB sequence sequences and DNA in the editing template.

In some embodiments, a spacer sequence comprises a region that has substantial complementarity to a search target sequence on the target strand of a double stranded target DNA, e.g., an AT7B gene, a PAH gene, or a SERPINA1 gene. In some embodiments, the spacer sequence of a tagRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

The spacer of the tagRNA can be from 16 to 25 nucleotides in length. The spacer can be 20 nucleotides in length. The spacer can be 21-23 nucleotides in length. In some embodiments, the length of the spacer varies from about 10 to about 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, or 20 to 30 nucleotides in length. In some embodiments, the spacer is 16 to 22 nucleotides in length. In some embodiments, the spacer is 16 to 20 nucleotides in length. In some embodiments, the spacer is 17 to 18 nucleotides in length.

As used herein in a gRNA (e.g., a tagRNA or a enhancer guide egRNA sequence), or fragments thereof such as a spacer, FB sequence, or editing template sequence, unless indicated otherwise, it should be appreciated that the letter “T” or “thymine” indicates a nucleobase in a DNA sequence that encodes the tagRNA or guide RNA sequence, and is intended to refer to a uracil (U) nucleobase of the tagRNA or guide RNA or any chemically modified uracil nucleobase known in the art, such as 5-methoxyuracil.

The extension arm of a tagRNA may comprise a flap binding sequence (FB sequence; FBS) and an editing template (e.g., an ET). The extension arm may be partially complementary to the spacer. In some embodiments, the editing template (e.g., ET) is partially complementary to the spacer. In some embodiments, the editing template (e.g., ET) and the flap binding sequence (FB sequence; FBS) are each partially complementary to the spacer. An extension arm of a tagRNA may comprise a flap binding sequence (FB sequence, or FBS) that comprises complementarity to and can hybridize with a free 3′ end of a single stranded DNA in the target gene (e.g., the ATP7B gene, PAH gene, or SERPINA1 gene) generated by nicking with a RT editor at the nick site on the PAM strand.

The length of the FB sequence may vary depending on, e.g., the RT editor components, the search target sequence and other components of the tagRNA. The FB sequence can be about 2 to 20 nucleotides in length. The FB sequence can be about 8 to 16 nucleotides in length. In some embodiments, the FB sequence is 6 nucleotides in length In some embodiments, the FB sequence is about 3 to 19 nucleotides in length, or about 3 to 17 nucleotides in length. In some embodiments, the FB sequence is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the FB sequence is 8 to 17 nucleotides in length. In some embodiments, the FB sequence is 8 to 16 nucleotides in length. In some embodiments, the FB sequence is 8 to 15 nucleotides in length. In some embodiments, the FB sequence is 8 to 14 nucleotides in length. In some embodiments, the FB sequence is 8 to 13 nucleotides in length. In some embodiments, the FB sequence is 8 to 12 nucleotides in length. In some embodiments, the FB sequence is 8 to 11 nucleotides in length. In some embodiments, the FB sequence is 8 to 10 nucleotides in length. In some embodiments, the FB sequence is 8 or 9 nucleotides in length. In some embodiments, the FB sequence is 16 or 17 nucleotides in length, in some embodiments, the FB sequence is 15 to 17 nucleotides in length. In some embodiments, the FB sequence is 14 to 17 nucleotides in length. In some embodiments, the FB sequence is 13 to 17 nucleotides in length. In some embodiments, the FB sequence is 12 to 17 nucleotides in length. In some embodiments, the FB sequence is 11 to 17 nucleotides in length. In some embodiments, the FB sequence is 10 to 17 nucleotides in length. In some embodiments, the FB sequence is 9 to 17 nucleotides in length. In some embodiments, the FB sequence is about 7 to 15 nucleotides in length. In some embodiments, the FB sequence is 6, 7, 8, 9, 10, 11, 12, 13, 14, 15, 16, 17, 18, or 19 nucleotides in length. In some embodiments, the FB sequence is 8, 9, 10, 11, 12, 13, or 14 nucleotides in length.

The FB sequence may be complementary or substantially complementary to a DNA sequence in the edit strand of the target gene. By annealing with the edit strand at a free hydroxy group, e.g., a free 3′ end generated by RT editor nicking activity, the FB sequence may initiate synthesis of a new single stranded DNA encoded by the editing template at the nick site. In some embodiments, the FB sequence is at least about 70%, 75%, 80%, 85%, 90%, 95%, or 100% complementary to a region of the edit strand of the target gene (e.g., the ATP7B gene, PAH gene, or SERPINA1 gene). In some embodiments, the FB sequence is perfectly complementary, or 100% complementary, to a region of the edit strand of the target gene (e.g., the ATP7B gene, PAH gene, or SERPINA1 gene).

In some embodiments, the FB sequence comprises a nucleotide sequence selected from GGCGTGGCAGTCA (SEQ ID NO: 91), GGCGTGGCAGTC (SEQ ID NO: 92), GGCGTGGCAGT (SEQ ID NO: 93), GGCGTGGCAG (SEQ ID NO: 94), GGCGTGGCA, GGCGTGGC, GGCGTGG, and GGCGTG. In some embodiments, the FB sequence comprises a nucleotide sequence having one, two, or three mismatches relative to a sequence selected from GGCGTGGCAGTCA (SEQ ID NO: 91), GGCGTGGCAGTC (SEQ ID NO: 92), GGCGTGGCAGT (SEQ ID NO: 93), GGCGTGGCAG (SEQ ID NO: 94), GGCGTGGCA, GGCGTGGC, GGCGTGG, and GGCGTG. In some embodiments, the FB sequence comprises or consists of a sequence selected from GGCGTGGCAGTCA (SEQ ID NO: 91), GGCGTGGCAGTC (SEQ ID NO: 92), GGCGTGGCAGT (SEQ ID NO: 93), GGCGTGGCAG (SEQ ID NO: 94), GGCGTGGCA, GGCGTGGC, GGCGTGG, and GGCGTG or a sequence at least 85% identical (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from GGCGTGGCAGTCA (SEQ ID NO: 91), GGCGTGGCAGTC (SEQ ID NO: 92), GGCGTGGCAGT (SEQ ID NO: 93), GGCGTGGCAG (SEQ ID NO: 94), GGCGTGGCA, GGCGTGGC, GGCGTGG, and GGCGTG.

An extension arm of a tagRNA may comprise an editing template that serves as a DNA synthesis template for the DNA polymerase in a RT editor during RT editing. The length of an editing template may vary depending on, e.g., the RT editor components, the search target sequence and other components of the tagRNA. In some embodiments, the editing template serves as a DNA synthesis template for a reverse transcriptase. The editing template can be about 4 to 30 nucleotides in length. The editing template can be about 10 to 30 nucleotides in length. The editing template can be 6 to 9 nucleotides in length. In some embodiments, the editing template is 2, 3, 4, 5, 6, 7, 8, 9, 10, 11, 12, 13, 14, 15, 16, 17, 18, 19, 20, 21, 22, 23, 24, or 25 nucleotides in length.

In some embodiments, the editing template sequence is about 70%, 75%, 80%, 85%, 90%, 95%, or 99% complementary to the editing target sequence on the edit strand of the target gene. In some embodiments, the editing template sequence is complementary to the editing target sequence except at positions of the intended nucleotide edits to be incorporated int the target gene. In some embodiments, the editing template comprises a nucleotide sequence comprising about 85% to about 95% complementarity to (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, complementarity to) an editing target sequence in the edit strand in the target gene. In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene (e.g., the ATP7B gene, PAH gene, or SERPINA1 gene).

In some embodiments, the editing template comprises a nucleotide sequence selected from SEQ ID NOs: 95-98. In some embodiments, the editing template comprises a nucleotide sequence selected from SEQ ID NOs: 95-98 or a sequence having one, two, or three mismatches relative to a nucleotide sequence selected from SEQ ID NOs: 95-98. In some embodiments, the editing template comprises or consists of a nucleotide sequence selected from SEQ ID NOs: 95-98 or a sequence at least 85% identical (e.g., 85%, 86%, 87%, 88%, 89%, 90%, 91%, 92%, 93%, 94%, 95%, 96%, 97%, 98%, 99%, 100%, or a number or a range between any two of these values) to a nucleotide sequence selected from SEQ ID NOs: 95-98.

An intended nucleotide edit or intended nucleotide edits in an editing template of a tagRNA may comprise various types of alterations as compared to the target gene sequence, in some embodiments, the nucleotide edit or edits is nucleotide substitution(s) as compared to the target gene sequence. In some embodiments, the nucleotide edit comprise deletion(s) as compared to the target gene sequence. In some embodiments, the nucleotide edit comprise an insertion as compared to the target gene sequence. In some embodiments, the editing template comprises one to ten intended nucleotide edits as compared to the target gene sequence, in some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two or more single nucleotide substitutions, insertions, deletions, or any combination thereof, as compared to the target gene sequence. In some embodiments, the editing template comprises three single nucleotide substitutions, insertions, deletions, or any combination thereof as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution, in some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution, in some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 22 nucleotides, at least 24 nucleotides, at least 26 nucleotides, at least 28 nucleotides, at least 30 nucleotides, at least 32 nucleotides, at least 34 nucleotides, at least 36 nucleotides, at least 38 nucleotides, at least 40 nucleotides, at least 42 nucleotides, at least 44 nucleotides, at least 46 nucleotides, at least 48 nucleotides, or at least 50 nucleotides, in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a tagRNA may comprise one or more intended nucleotide edits, compared to the target gene to be edited. Position of the intended nucleotide edit(s) relevant to other components of the tagRNA, or to particular nucleotides (e.g., mutations) in the target gene may vary. In some embodiments, the nucleotide edit is in a region of the tagRNA corresponding to or homologous to the protospacer sequence. In some embodiments, the nucleotide edit is in a region of the tagRNA corresponding to a region of the target gene outside of the protospacer sequence.

In some embodiments, the position of a nucleotide edit incorporation in the target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence, in some embodiments, position of an intended nucleotide edit in the editing template may be referred to by aligning the editing template with the partially complementary edit strand of the target gene, and referring to nucleotide positions on the editing strand where the intended nucleotide edit is incorporated, in some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream of the 5′ most nucleotide of the PAM sequence in the edit strand of the target gene. By 0 base pair upstream or downstream of a reference position, it is meant that the intended nucleotide is immediately upstream or downstream of the reference position. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream of the 5′ most nucleotide of the PAM sequence. In some embodiments, the nucleotide edit is incorporated at a position corresponding to 3 nucleotides upstream of the 5′ most nucleotide of the PAM sequence. In some embodiments, the nucleotide edit in is incorporated at a position corresponding to 4 nucleotides upstream of the 5′ most nucleotide of the PAM sequence. In some embodiments, the nucleotide edit is incorporated at a position corresponding to 5 nucleotides upstream of the 5′ most nucleotide of the PAM sequence. In some embodiments, the nucleotide edit in the editing template is at a position corresponding to 6 nucleotides upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides downstream of the 5′ most nucleotide of the PAM sequence in the edit strand of the target gene. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides downstream of the 5′ most nucleotide of the PAM sequence. In some embodiments, a nucleotide edit is incorporated at a position corresponding to 3 nucleotides downstream of the 5′ most nucleotide of the PAM sequence. In some embodiments, a nucleotide edit is incorporated at a position corresponding to 4 nucleotides downstream of the 5′ most nucleotide of the PAM sequence. In some embodiments, a nucleotide edit is incorporated at a position corresponding to 5 nucleotides downstream of the 5′ most nucleotide of the PAM sequence. In some embodiments, a nucleotide edit is incorporated at a position corresponding to 6 nucleotides downstream of the 5′ most nucleotide of the PAM sequence.

In some embodiments, the position of a nucleotide edit incorporation in the target gene can be determined based on position of the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides downstream of the nick site on the PAM strand (or the non-target strand, or the edit strand) of the double stranded target DNA. In some embodiments, position of the intended nucleotide edit in the editing template can be referred to by aligning the editing template with the partially complementary editing target sequence on the edit strand and referring to nucleotide positions on the editing strand where the intended nucleotide edit is incorporated. Accordingly, in some embodiments, a nucleotide edit in an editing template is at a position corresponding to a position about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 tol6 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides apart from the nick site. In some embodiments, when referred to in the context of the PAM strand (or the non-target strand, or the edit strand), a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 tol6 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides downstream from the nick site. The relative positions of the intended nucleotide edit(s) and nick site may be referred to by numbers. For example, in some embodiments, the nucleotide immediately downstream of the nick site on a PAM strand (or the non-target strand, or the edit strand) may be referred to as at position 0. The nucleotide immediately upstream of the nick site on the PAM strand (or the non-target strand, or the edit strand) may be referred to as at position −1. The nucleotides downstream of position 0 on the PAM strand can be referred to as at positions +1, +2, +3, +4, . . . +n, and the nucleotides upstream of position −1 on the PAM strand may be referred to as at positions −2, −3, −4, . . . −n. Accordingly, in some embodiments, the nucleotide in the editing template that corresponds to position 0 when the editing template is aligned with the partially complementary editing target sequence by complementarity can also be referred to as position 0 in the editing template, the nucleotides in the editing template corresponding to the nucleotides at positions +1, +2, +3, +4, . . . , +n on the PAM strand of the double stranded target DNA can also be referred to as at positions +1, +2, +3, +4, . . . , −in in the editing template, and the nucleotides in the editing template corresponding to the nucleotides at positions −1, −2, −3, −4, . . . , −n on the PAM strand on the double stranded target DNA may also be referred to as at positions −1, −2, −3, −4 −n on the editing template, even though when the tagRNA is viewed as a standalone nucleic acid, positions +1, +2, +3, +4, . . . , +n are 5′ of position 0 and positions −1, −2, −3, −4, . . . −n are 3′ of position 0 in the editing template. In some embodiments, an intended nucleotide edit is at position +n of the editing template relative to position 0. Accordingly, the intended nucleotide edit may be incorporated at position +n of the PAM strand of the double stranded target DNA (and subsequently, the target strand of the double stranded target DNA) by RT editing. The number n may be referred to as the nick to edit distance.

When referred to within the tagRNA, positions of the one or more intended nucleotide edits may be referred to relevant to components of the tagRNA. For example, an intended nucleotide edit may be 5′ or 3′ to the FB sequence. In some embodiments, a tagRNA comprises the structure, from 5′ to 3′: a spacer, a gRNA core, an editing template, and a FB sequence. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream to the 5′ most nucleotide of the FB sequence. in some embodiments, the intended nucleotide edit is 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream to the 5′ most nucleotide of the FB sequence.

The corresponding positions of the intended nucleotide edit incorporated in the target gene may also be referred to based on the nicking position (i.e., the nick site) generated by a RT editor based on sequence homology and complementarity. For example, in some embodiments, the distance between the intended nucleotide edit to be incorporated into the target gene and the nick site (also referred to as the “nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In some embodiments, the position of the nucleotide edit can be in any position downstream of the nick site on the edit strand (or the PAM strand) generated by the RT editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pair from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

In some embodiments, the editing template extends beyond a nucleotide edit to be incorporated into the target gene sequence. For example, in some embodiments, the editing template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides 3′ to the nucleotide edit to be incorporated into the target gene sequence. In some embodiments, the editing template comprises 1 to 80 nucleotides 3′ to the nucleotide edit to be incorporated into the target gene sequence.

In some embodiments, the editing template can comprise a second editing sequence comprising a second mutation relative to a target sequence. The second mutation can be designed to mutate or otherwise silence a PAM sequence such that a corresponding nucleic acid guided nuclease or CRISPR nuclease is no longer able to cleave the target sequence. In some embodiments, this mutation or silencing of a PAM can serve as a method for selecting transformants in which the first editing sequence has been incorporated. In some embodiments, the mutation is in at least 1, 2, 3, 4, 5, 6, 7, 8, 9, 10 or more nucleic acids in a PAM motif.

The editing template of a tagRNA may encode a new single stranded DNA (e.g., by reverse transcription) to replace a editing target sequence in the target gene. In some embodiments, the editing target sequence in the edit strand of the target gene is replaced by the newly synthesized strand, and the nucleotide edit(s) are incorporated into the region of the target gene.

In some embodiments, the target gene is ATP7B gene. In some embodiments, the editing template of the tagRNA encodes a newly synthesized single stranded DNA that comprises a wild type ATP7B gene sequence. In some embodiments, the newly synthesized DNA strand replaces the editing target sequence in the target ATP7B gene, wherein the editing target sequence (or the endogenous sequence complementary to the editing target sequence on the target strand of the ATP7B gene) comprises a mutation compared to a wild-type ATP7B gene. In some embodiments, the mutation is associated with Wilson's disease.

In some embodiments, the target gene is SERPINA1 gene. In some embodiments, the editing template of the tagRNA encodes a newly synthesized single stranded DNA that comprises a wild type SERPINA1 gene sequence. In some embodiments, the newly synthesized DNA strand replaces the editing target sequence in the target SERPINA1 gene, wherein the editing target sequence (or the endogenous sequence complementary to the editing target sequence on the target strand of the SERPINA1 gene) comprises a mutation compared to a wild-type SERPINA1 gene. In some embodiments, the mutation is associated with A1ATD disease or disorder.

In some embodiments, the target gene is the PAH gene. In some embodiments, the editing template of the tagRNA encodes a newly synthesized single stranded DNA that comprises a wild type PAH gene sequence. In some embodiments, the newly synthesized DNA strand replaces the editing target sequence in the target PAH gene, wherein the editing target sequence (or the endogenous sequence complementary to the editing target sequence on the target strand of the PAH gene) comprises a mutation compared to a wild-type PAH gene. In some embodiments, the mutation is associated with phenylketonuria or hyperphenylalaninemia.

In some embodiments, the editing target sequence comprises a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, or exon 21 of the target gene as compared to a wild type target gene, in some embodiments, the editing target sequence comprises a mutation in exon 8, exon 13, exon 14, exon 15, or exon 17 of the ATP7B gene as compared to a wild type ATP7B gene. In some embodiments, the editing target sequence comprises a mutation in exon 14 of the ATP7B gene as compared to a wild type ATP7B gene. In some embodiments, the editing target sequence comprises a mutation that encodes an amino acid substitution H1069Q relative to a wild type ATP7B polypeptide. In some embodiments, the ATP7B gene sequence comprises a C>A (e.g., C-A, C to A) mutation. In some embodiments, the mutation is the S allele mutation or the Z allele mutation in SERPINA1 gene associated with alpha-1 antitrypsin deficiency disease (A1ATD). In some embodiments, the mutation is the Z allele mutation on exon 5 of SERPINA1. In some embodiment, the mutation is an E342K mutation commonly associated with A1ATD. E342K mutation leads to misfolding of the alpha-1-antitrypsin (AAT) protein leading to polymers and liver damage. In some embodiments, the mutation is the R408W mutation in PAH gene associated with phenylketonuria or hyperphenylalaninemia.

The compositions and methods described herein can be used to correct a disease-causing mutation or otherwise edit the PAH gene at any position. Exemplary mutations in PAH that can be edited with the disclosed compositions and methods include, but are not limited To: p.M1L; p.M1V; p.M1R; p.M1T; p.M1I; p.V5Sfs*3; p.N8Ifs*30; p.G10G; p.R13Qfs*5; p.L11*; p.L15Qfs*24; p.S16P; p.D17Lfs*22; p.S16*; p.S16Y; p.D17*; p.Q20Kfs*17; p.Q20*; p.Q20L; p.Q20P; p.Q20Pfs*7; p.Q20H; p.T22K; p.E26Lfs*13; p.I25Mfs*13; p.N28=; p.C29*; p.I35M; p.S36*; p.L37P; p.I38Dfs*19; p.F39del; p.F39L; p.S40L; p.L41F; p.L41P; p.K42del; p.K42E; p.K42I; p.E43*; p.E44del; p.V45A; p.G46R; p.G46S; p.G46Vfs*15; p.A47E; p.A47V; p.L48=; p.L48V; p.L48S; p.L48W; pA49T; p.V51L; p.L52Cfs*9; p.L52S; p.L52F; p.R53C; p.R53H; p.L54S; p.F55del; p.F55S; p.F55L; p.F55Lfs*6; p.E56=; p.E56D; p.E57Rfs*3; p.E57*; p.E57Cfs*77; p.E57del; p.E57K; p.N58*; p.N58*; p.D59Y; p.D59G; p.D59V; p.N61D; p.N61S; p.N61K; p.L62*; p.L62Pfs*7; p.L62V; p.H64Dfs*4; p.L62P; p.L62Pfs*3; p.T63_H64delinsPN; p.T63P; p.H64Pfs*10; p.H64*; p.H64N; p.H64Tfs*9; p.I65V; p.I65Kfs*9; p.I65N; p.I65S; p.I65T; p.I65M; p.E66*; p.E66K; p.E66Afs*8; p.S67A; p.S67P; p.R68G; p.R68S; p.P69_S70dup; p.P69S; p.P69T; p.S70del; p.S70Ffs*7; p.S70del; p.S70P; p.S70F; p.R71C; p.R71H; p.R71P; p.D75N; p.N75H; p.D75G; p.D75V; p.E76*; p.E76K; p.E76A; p.E76G; p.E76D; p.Y77H; p.Y77*; p.E78K; p.E78Q; p.E78V; p.E78Ffs*13; p.F79Ifs*7; p.T81P; p.T81Vfs*6; p.T81N; p.L83Wfs*9; p.D84Y; p.D84G; p.K85*; p.R86P; p.S87R; p.P89S; p.P89T; p.A90Cfs*12; p.T92I; p.Q93Sfs*5; p.N93Kfs*5; p.I95F; p.I95del; p.I95T; p.I95I; p.I97L; p.L98V; p.L98S; p.H100R; p.D101N; p.I102T; p.G103C; p.G103S; p.G103D; p.A104_V106del; p.A104S; p.A104D; p.A104V; p.V106A; p.H107P; p.H107R; p.E108*; p.E108Dfs*4; p.S110L; p.R111*; p.R111Q; p.D112H; p.D112Efs*2; p.K113_K114fs*81; p.K115Rfs*81; p.K115Tfs*79; p.D116Hfs*29; p.T117I; p.T117Kfs*78; p.P119S; p.P119L; p.W120Gfs*75; p.W120*; p.F121L; p.F121V; p.F121S; p.P122S; p.P122Q; p.R123I; p.T124I; p.E127K; p.E127G; p.L128P; p.D129Y; p.D129G; p.D129V; p.A132V; p.N133Rfs*61; p.Q134*; p.S137S; p.L142P; p.D143G; p.D143V; p.D145N; p.D145V; p.H146Y; p.P147S; p.P147L; p.P147=; p.G148Lfs*105; p.G148R; p.G148S; p.G148Wfs*29; p.G148D; p.G148V; p.F149S; p.D151RfsTer13; p.D151H; p.D151G; p.D151E; p.P152Lfs*43; p.P152T; p.R155Lfs*43; p.Y154H; p.Y154N; p.Y154C; p.Y154F; p.Y154*; p.R155C; p.R155Vfs*40; p.R155H; p.R155P; p.A156P; p.R157I; p.R157K; p.R157N; p.R157T; p.R157S; p.R158G; p.R158W; p.R158P; p.R158Q; p.K159*; p.Q160*; p.Q160P; p.F161S; p.D163N; p.I164V; p.I164T; p.A165P; p.A165T; p.A165Vfs*35; p.A165D; p.Y166*; p.N167Y; p.N167I; p.N167S; p.Y168H; p.Y168N; p.Y168Sfs*27; p.Y168*; p.R169C; p.R169G; p.R169S; p.R169H; p.R169Pfs*26; p.H170D; p.H170P; p.H170R; p.H170Q; p.G171R; p.G171W; p.G171A; p.G171E; p.Q172*; p.Q172R; p.Q172H; p.P173T; p.I174V; p.I174N; p.I174T; p.P175A; p.P175S; p.P175R; p.R176*; p.R176L; p.R176P; p.R176Q; p.V177L; p.V177M; p.V177A; p.E178K; p.E178G; p.E178V; p.Y179H; p.Y179N; p.M180V; p.M180Ifs*18; p.E181Kfs*13; p.E183del; p.E182K; p.E182G; p.E183L; p.E183Q; p.E183G; p.K184Rfs*11; p.K184N; p.T186Hfs*9; p.T186I; p.W187Gfs*12; p.W187Gfs*8; p.W187R; p.W187*; p.W187C; p.G188Afs*7; p.G188D; p.G188V; p.V190M; p.V190A; p.V190G; p.K192*; p. T193P; p.T193I; p.L194Dfs*6; p.L194Efs*5; p.L194P; p.L194R; p.S196Vfs*4; p.S196Lfs*2; p.S196T; p.S196Y; p.L197*; p.L197W; p.Y198Sfs*136; p.L197F; p.Y198Vfs*9; p.Y198D; p.Y198Sfs*136; p.Y198*; p.T200Nfs*6; p.T200N; p.H201Y; p.H201P; p.H201R; p.H201Q; p.A202T; p.A202V; p.C203G; p.C203Lfs*3; p.C203S; p.C203Y; p.C203*; p.C203C; p.C203W; p.Y204*; p.Y204Y; p.E205K; p.E205A; p.E205D; p.Y206D; p.Y206C; p.Y206*; p.N207D; p.N207S; p.P211T; p.P211Hfs*130; p.P211L; p.L212P; p.E214Kfs*127; p.L213P; p.E214G; p.Y216*; p.C217G; p.C217R; p.C217F; p.C217Y; p.G218Afs*123; p.G218V; p.F219S; p.H220P; p.E221G; p.D222*; p.D222G; p.D222V; p.N223Y; p.Q226Tfs*118; p.N223I; p.N223Kfs*118; p.I224T; p.I224M; p.P225A; p.P225T; p.P225L; p.P225R; p.Q226*; p.Q226K; p.Q226H; p.L227M; p.L227V; p.L227Q; p.E228*; p.E228K; p.E228G; p.E228D; p.D229Efs*54; p.D229G; p.V230I; p.V230A; p.V230G; p.S231Vfs*52; p.S231P; p.S231F; p.Q232*; p.Q232E; p.Q232P; p.Q232R; p.Q232Q; p.F233I; p.F233L; p.Q235*; p.Q235P; p.T236Mfs*60; p.C237F; p.T238A; p.T238P; p.G239S; p.G239A; p.G239D; p.G239V; p.F240V; p.F240S; p.F240=; p.R241C; p.R241H; p.R241L; p.R241P; p.R241Pfs*100; p.L242F; p.L242=; p.R243*; p.R243L; p.R243Q; p.P244S; p.P244L; p.V245I; p.V245L; p.V245M; p.V245A; p.V245E; p.V245V; p.A246D; p.A246V; p.A246Vfs*95; p.G247R; p.G247S; p.G247Afs*94; p.G247D; p.G247V; p.L248P; p.L248R; p.L248Rfs*93; p.L249F; p.L249Ffs*92; p.L249H; p.L249P; p.S250F; p.R252Gfs*30; p.R252Gfs*89; p.R252G; p.R252W; p.R252L; p.R252P; p.R252Q; p.F254I; p.L255=; p.L255V; p.L255S; p.L255W; p.G257C; p.G257S; p.G257D; p.G257V; p.L258=; p.L258P; p.L258R; p.A259T; p.A259V; p.F260I; p.F260L; p.R261*; p.R261G; p.R261L; p.R261P; p.R261Q; p.V262G; p.F263S; p.F263L; p.H264L; p.C265G; p.C265R; p.C265Y; p.C265*; p.T266A; p.T266E; p.T266P; p.T266I; p.Q267*; p.Q267E; p.Q267L; p.Q267R; p.Q267H; p.Y268H; p.Y268C; p.Y268*; p.I269L; p.I269N; p.I269Tfs*72; p.R270G; p.R270I; p.R270K; p.H271Ifs*10; p.R270S; p.H271Y; p.H271L; p.H271R; p.H271Q; p.G272*; p.S273P; p.S273F; p.K274E; p.K274Nfs*5; p.P275A; p.P275S; p.P275L; p.P275R; p.P275=; p.M276V; p.M276K; p.M276R; p.M276T; p.M276I; p.Y277D; p.Y277C; p.T278A; p.E280Nfs*61; p. T278I; p.T278N; p.T278S; p.P279T; p.P279L; p.E280Nfs*61; p.P279P; p.E280_IVS7+3fs; p.E280*; p.E280K; p.E280Q; p.E280A; p.E280Dfs*3; p.E280G; p.P281A; p.P281fs; p.P281S; p.P281L; p.P281R; p.D282N; p.D282G; p.I283F; p.I283V; p.I283N; p.C284R; p.C284Y; p.C284*; p.H285Y; p.E286K; p.E286=; p.L287V; p.L288F; p.G289R; p.G289R; p.H290Y; p.H290L; p.H290R; p.H290Q; p.V291L; p.V291M; p.P292S; p.P292H; p.P292L; p.292=; p.L293M; p.L293S; p. S295*; p.D296H; p.D296G; p.R297C; p.R297H; p.R297L; p.F299del; p.F299C; p.F299S; p.A300S; p.A300D; p.A300V; p.Q301*; p.Q301P; p.Q301H; p.F302fs*39; p.F302V; p.S303A; p.S303P; p.S303Pfs*38; p.Q304*; p.Q304K; p.Q304P; p.Q304R; p.Q304Q; p.I306Lfs*35; p.I306V; p.G307Afs*34; p.G307D; p.L308F; p.L308V; p.A309T; p.A309D; p.A309V; p.S310_L311del; p.S310P; p.L311*; p.S310C; p.S310F; p.S310Y; p.L311*; p.L311Gfs*4; p.L311P; p.L311R; p.G312C; p.G312R; p.G312S; p.G312D; p.G312V; p.G312Vfs*29; p.A313T; p.A313V; p.P314A; p.P314S; p.P314T; p.P314H; p.P314Lfs*27; p.D315Y; p.Y317H; p.I318T; p.K320N; p.L321F; p.L321L; p.A322T; p.A322D; p.A322G; p.A322V; p.T323del; p.T323I; p.T323T; p.I324V; p.I324N; p.Y325C; p.Y325S; p.Y325*; p.W326Gfs*15; p.W326*; p.W326S; p.F327L; p.T328A; p.T328P; p.T328I; p.T328N; p.E330D; p.F331L; p.F331C; p.F331S; p.G332R; p.G332E; p.G332V; p.L333F; p.L333P; p.C334R; p.C334S; p.C334*; p.K335E; p.K335T; p.Q336*; p.Q336R; p.G337V; p.D338Y; p.S339F; p.S339Y; p.I340T; p.K341*; p.K341R; p.K341T; p.A342Hfs*59; p.K341K; p.K341N; p.A342Hfs*58; p.A342P; p.A342S; p.A342T; p.A342E; p.Y343D; p.Y343N; p.Y343C; p.Y343F; p.Y343*; p.G344R; p.G344S; p.G344D; p.G344V; p.A345S; p.A345T; p.G346R; p.G346E; p.L347Sfs*53; p.L347F; p.L347=; p.L348Cfs*52; p.L348V; p.L348P; p.L348Rfs*2; p.S350Vfs*5; p.S349A; p.S349P; p.S349*; p.S349L; p.S350T; p.S350Y; p.G352C; p.G352Cfs*48; p.G352R; p.G352Vfs*48; p.E353Nfs*47; p.E353*; p.E353Vfs*42; p.L354Ffs*40; p.Q355*; p.Y356D; p.Y356H; p.Y356*; p.C357G; p.C357R; p.C357Y; p.C357*; p.L358V; p.L358F; p.S359*; p.S359L; p.E360*; p.K361Q; p.P362S; p.P362T; p.P362L; p.K363Afs*30; p.K363N; p.K363Nfs*37; p.L364F; p.L365_L369del; p.L365del; p.P366A; p.P366H; p.L367Pfs*27; p.L367V; p.L367Wfs*33; p.L367P; p.L367R; p.L367L; p.E368K; p.E368G; p.L369V; p.E370Afs*25; p.L369L; p.E370G; p.E370D; p.K371R; p.T372S; p.T372R; p.A373Hfs*20; p.A373T; p.A373D; p.A373=; p.I374Yfs*20; p.Q375E; p.Q375R; p.Q375H; p.N376Ifs*24; p.N376T; p.Y377D; p.Y377Tfs*23; p.Y377C; p.T378S; p.V379A; p.T380Gfs*13; p.T380M; p.F382I; p.F382L; p.F382L; p.Q383*; p.Q383E; p.P384S; p.L385P; p.L385=; p.Y386D; p.Y386H; p.Y386C; p.Y386Ffs*14; p.Y387D; p.Y387H; p.Y387*; p.Y387=; p.V388Gfs*5; p.V388L; p.V388M; p.V388A; p.V388Gfs*5; p.A389E; p.A389Efs*11; p.A389G; p.E390_S391delin; sGG; p.E390Dfs*4; p.E390G; p.S391Ffs*2; p.S391G; p.S391I; p.S391T; p.F392I; p.F392S; p.N393Ifs*2; p.D394H; p.D394Y; p.D394A; p.A395P; p.A395S; p.A395D; p.A395G; p.K396R; p.K398E; p.K398K; p.K398N; p.V399A; p.V399Gfs*52; p.V399V; p.R400Gfs*52; p.R400R; p.R400K; p.R400T; p.N401_S439del; p.N401Tfs*51; p.R400S; p.F402I; p.F402L; p.F402V; p.F402C; p.A403V; p.I406Sfs*17; p.I406Sfs*15; p.I406V; p.I406T; p.I406M; p.P407S; p.P407T; p.P407L; p.P407Lfs*45; p.R408Gfs*44; p.R408W; p.R408L; p.R408Q; p.P409S; p.P409L; p.F410I; p.F410L; p.F410C; p.F410S; p.S411*; p.V412D; p.V412G; p.R413C; p.R413G; p.R413S; p.R413H; p.R413P; p.Y414H; p.Y414C; p.Y414*; p.Y414Y; p.D415N; p.D415Y; p.D415A; p.D415V; p.P416Hfs*36; p.P416T; p.P416Q; p.Y417D; p.Y417H; p.Y417N; p.Y417C; p.Y417*; p.T418P; p.T418N; p.Q419P; p.Q419R; p.R420M; p.I421S; p.I421T; p.E422K; p.L424Wfs*28; p.L424*; p.L424S; p.N426N; p.Q428Sfs*24; p.Q429K; p.Q429P; p.L430P; p.L430R; p.L433Ffs*3; p.A434D; p.A434V; p.D435N; p.D435V; p.S436Pfs*16; p.N438D; p.N438fs; p.G442Wfs*47; p.L444F; p.L444P; p.A447P; p.A447D; p.L448Rfs*3; p.*453Vext*35; p.*453Pext*33; 1378G>T; 1503A>G; 1546G>A; 4171_-405del; −4173_-407del; −626G>A; −480delACT; −224G>A; −147C>T; −81C>T; −71A>C; −52G>C; −1C>T; c.60+4A>T; c.60+5_60+6del; c.60+5G>A; c.60+5G>C; c.60+5G>T; c.60+40T>G; c.60+62C>T; c.61-13del9; c.61-3T>C; c.168+1G>A; c.168+1G>T; c.168+2T>C; c.168+5G>A; c.168+5G>C; c.168+5G>T; c.168+6T>G; c.168+19T>C; c.169-13T>G; c.169-2A>G; c.169-1G>A; c.352+1G>A; c.352+7757C>G; c.353-22C>A; c.353-22C>T; c.353-6T>A; c.353-2A>G; c.353-2A>T; c.353-1G>A; c.353-1G>C; c.353-1G>T; c.441+1delGT; c.441+1G>A; c.441+1G>C; c.441+1G>T; c.441+2T>C; c.441+2_441+3del; c.441+2T>A; c.441+2T>G; c.441+3G>C; c.441+4A>G; c.441+5G>A; c.441+5G>T; c.441+6T>A; c.441+6T>C; c.441+47C>T; c.442-14C>T; c.442-5C>G; c.442-2A>C; c.442-1G>A; c.509+1delG; c.509+1G>A; c.509+5delG; c.509+54C>G; c.509+101A>C; c.510-21_665del177; c.510-54G>A; c.510-6T>A; c.510-6T>G; c.510-2A>G; c.510-1G>A; c.706+1G>T; c.706+4A>T; c.706+5G>A; c.706+17G>T; c.706+36T>G; c.706+44T>G; c.707-96A>G; c.707-59C>G; c.707-7A>T; c.707-2A>G; c.707-1G>A; c.707-1G>C; c.842+1G>A; c.842+1G>T; c.842+2T>A; c.842+3G>C; c.842+4A>G; c.842+4A>T; c.842+5G>A; c.842+6T>A; c.842+25G>T; c.843-14_11del; c.843-6T>C; c.843-5T>C; c.843-4del; c.843-2A>T; c.912+1G>A; c.912+1G>C; c.912+1G>T; c.912+2T>C; c.912+3A>C; c.912+16T>A; c.913-8A>G; c.913-7A>G; c.913-5T>C; c.913-5T>G; c.913-3C>G; c.913-2A>G; c.969+1G>A; c.969+2insT; c.969+4A>T; c.969+5G>A; c.969+5G>C; c.969+6T>A; c.969+6T>C; c.969+34G>A; c.969+43G>T; c.970-7A>G; c.970-6T>G; c.970-5T>A; c.970-3C>T; c.970-2A>C; c.970-2A>G; c.970-1G>A; c.970-1G>C; c.970-1G>T; c.1065+1G>A; c.1065+1G>T; c.1065+3A>C; c.1065+3A>G; c.1065+7C>A; c.1065+32T>A; c.1065+39G>T; c.1065+97G>A; c.1066-193G>C; c.1066-31G>A; c.1066-15A>C; c.1066-14C>G; c.1066-13del; c.1066-13T>G; c.1066-12del; c.1066-11G>A; c.1066-10G>A; c.1066-7C>A; c.1066-3C>G; c.1066-3C>T; c.1066-2A>G; c.1066-2A>T; c.1066-1G>A; c.1066-1G>C; c.1066-1G>T; c.1199+1G>A; c.1199+1G>C; c.1199+1G>T; c.1199+2T>C; c.1199+2T>G; c.1199+4A>G; c.1199+5G>A; c.1199+5G>A; c.1199+17G>A; c.1199+19T>C; c.1199+20G>C; c.1199+50G>A; c.1199+88del; c.1200-35C>T; c.1200-12del; c.1200-8G>A; c.1200-3T>G; c.1200-2A>G; c.1200-2A>C; c.1200-1del; c.1200-1G>A; c.1200-1G>C; c.1200-1G>T; c.1315+1G>A; c.1315+1G>T; c.1315+2T>C; c.1315+4A>G; c.1315+5G>A; c.1315+5G>C; c.1315+5G>T; c.1315+6T>A; c.1316-35C>T; c.1316-16T>G; c.1316-15T>C; c.1316-5T>C; c.1316-2A>C; and c.1316-1G>A. Mutations in the PAH gene are also described in Chen, Ting, et al. (“Mutational and phenotypic spectrum of phenylalanine hydroxylase deficiency in Zhejiang Province, China.” Scientific Reports 8.1 (2018): 17137) and Hillert, Alicia, et al. (“The genetic landscape and epidemiology of phenylketonuria.” The American Journal of Human Genetics 107.2 (2020): 234-250), the contents of which are hereby incorporated by reference in their entireties.

In some embodiments, the tagRNA results in incorporation of the intended nucleotide edit about 0 to 27 base pairs downstream (e.g., 0, 1, 2, 3, 4, 5, 6, 7, 8, 9, 10, 11, 12, 13, 14, 15, 16, 17, 18, 19, 20, 21, 22, 23, 24, 25, 26, 27 base pairs downstream) of the 5′ end of the PAM when contacted with the target gene. The intended nucleotide edit can comprise a single nucleotide substitution compared to the region corresponding to the editing target in the target gene. The single nucleotide substitution can be a T>G substitution. The intended nucleotide edit can comprise an insertion compared to the region corresponding to the editing target in the target gene. The intended nucleotide edit can comprise a deletion compared to the region corresponding to the editing target in the target gene. The editing target sequence can comprise a mutation associated with a disease or disorder. In some embodiments, the mutation encodes an amino acid substitution. In some embodiments, the amino acid substitution is H1069Q. The editing template can comprise a wild-type ATP7B gene sequence. In some embodiments, the tagRNA results in correction of the mutation when contacted with the ATP7B gene. In some embodiments, the mutation is the S allele mutation or the Z allele mutation in SERPINA1 gene associated with alpha-1 antitrypsin deficiency disease (A1ATD). The Z allele mutation on exon 5 of SERPINA1 is an E342K mutation commonly associated with A1ATD. E342K mutation leads to misfolding of the alpha-1-antitrypsin (AAT) protein leading to polymers and liver damage. The editing template can comprise a wild-type SERPINA1 gene sequence. In some embodiments, the tagRNA results in correction of the mutation when contacted with the SERPINA1 gene.

In some embodiments, the tagRNA comprises a nucleotide sequence selected from SEQ ID NOs: 16-47, 99-130, 164-181, 320-497, 563-682, 791-792, 1046-1053, and 1089-1203. In some embodiments, the tagRNA comprises a nucleotide sequence selected from SEQ ID NOs: 16-47, 99-130, 164-181, 320-497, 563-682, 791-792, 1046-1053, and 1089-1203 or a sequence have one, two, or three mismatches relative to a nucleotide sequence selected from SEQ ID NOs: 16-47, 99-130, 164-181, 320-497, 563-682, 791-792, 1046-1053, and 1089-1203. In some embodiments, the tagRNA comprises or consists of a nucleotide sequence selected from SEQ ID NOs: 16-47, 99-130, 164-181, 320-497, 563-682, 791-792, 1046-1053, and 1089-1203 or a sequence at least 85% identical to a nucleotide sequence selected from SEQ ID NOs: 16-47, 99-130, 164-181, 320-497, 563-682, 791-792, 1046-1053, and 1089-1203 (e.g., 85%, 86%, 87%, 88%, 89%, 90%, 91%, 92%, 93%, 94%, 95%, 96%, 97%, 98%, 99%, 100%, or a number or a range between any two of these values, identical to a nucleotide sequence selected from SEQ ID NOs: 16-47, 99-130, 164-181, 320-497, 563-682, 791-792, 1046-1053, and 1089-1203).

A scaffold sequence (also referred to herein as the gRNA core, gRNA scaffold, or gRNA backbone sequence) of a tagRNA may contain a polynucleotide sequence that binds to a DNA binding domain, a DNA endonuclease domain (e.g., Cas9) of a RT editor. The scaffold sequence may interact with a RT editor as described herein, for example, by association with a DNA binding domain, a DNA endonuclease domain, such as a DNA nickase of the RT editor.

One of skill in the art will recognize that different RT editors having different DNA binding domain and a DNA endonuclease domains from different DNA binding domain and a DNA endonuclease proteins may require different gRNA core sequences specific to the DNA binding domain and a DNA endonuclease protein. In some embodiments, the scaffold sequence is capable of binding to a Cas9-based RT editor. In some embodiments, the scaffold sequence is capable of binding to a Cpf1-based RT editor. In some embodiments, the scaffold sequence is capable of binding to a Cas12b-based RT editor. In some embodiments, the scaffold sequence is capable of binding to a Cas9 nickase of any one of the sequences selected from SEQ ID NOs: 6, 142-163 and 270-314.

In some embodiments, the scaffold sequence comprises regions and secondary structures involved in binding with specific CRISPR Cas proteins. For example, in a Cas9 based RT editing system, the scaffold sequence of a tagRNA may comprise one or more regions of a base paired “lower stem” adjacent to the spacer sequence and a base paired “upper stem” following the lower stem, where the lower stem and upper stem may be connected by a “bulge” comprising unpaired RNAs. The scaffold sequence may further comprise a “nexus” distal from the spacer sequence, followed by a hairpin structure, e.g., at the 3′ end. In some embodiments, the scaffold sequence comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, and/or the hairpin regions may be replaced with one or more DNA sequences. In some embodiments, the scaffold sequence comprises unmodified or wild type RNA sequences in the nexus and/or the bulge regions. In some embodiments, the scaffold sequence does not include long stretches of A-T pairs, for example, a GUUUU-AAAAC (SEQ ID NO: 889) pairing element.

A tagRNA may also comprise optional modifiers, e.g., 3′ end modifier region and/or an 5′ end modifier region. In some embodiments, a tagRNA comprises at least one nucleotide that is not part of a spacer, a scaffold sequence, or an extension arm. The optional sequence modifiers can be positioned within or between any of the other regions of the tagRNA, and not limited to being located at the 3′ and 5′ ends. In some embodiments, the tagRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a tagRNA comprises a short stretch of uracil at the 5′ end or the 3′ end. For example, in some embodiments, a tagRNA comprising a 3′ extension arm comprises a “UUU” sequence at the 3′ end of the extension arm. In some embodiments, a tagRNA comprises a toeloop sequence at the 3′ end. In some embodiments, the tagRNA comprises a 3′ extension arm and a toeloop sequence at the 3′ end of the extension arm. In some embodiments, the tagRNA comprises a 5′ extension arm and a toeloop sequence at the 5′ end of the extension arm. In some embodiments, the tagRNA comprises a toeloop element having the sequence S′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the FB sequence and the editing template. In some embodiments the secondary structure is positioned at the 3′ end or at the 5′ end of the tagRNA. In some embodiments, the tagRNA comprises a transcriptional lamination signal at the 3′ end of the tagRNA. In addition to secondary RNA structures, the tagRNA may comprise a chemical linker or a poly(N) linker or tail, where “N” can be any nucleobase. In some embodiments, the chemical linker may function to prevent reverse transcription of the gRNA core.

The tagRNAs may be modified in one or more ways to improve their overall stability and/or performance in RT editing. The tagRNA can comprise 3′ mN*mN*mN*N and 5′ mN*mN*mN* modifications, where m indicates that the nucleotide contains a 2′-O-Me modification and a * indicates the presence of a phosphorothioate bond. The tagRNA can comprise a structural motif at the 3′ terminus selected from the group consisting of: an inverted-dT, a prequeosine1-1 riboswitch aptamer (evopreQ1) and variants thereof, a frameshifting pseudoknot from Moloney murine leukemia virus (MMLV) (mpknot), G-quadruplexes, hairpin structures, xrRNA, and a P4-P6 domain of the group I intron. In some embodiments, the structural motif is evopreQ1 or a variant thereof comprising a nucleotide sequence selected from SEQ ID NOs: 84-90. In some embodiments, the structural motif is evopreQ1 or a variant thereof comprising a nucleotide sequence selected from SEQ ID NOs: 84-90 or a sequence having one, two or three mismatches relative a sequence selected from SEQ ID NOs: 84-90. In some embodiments, the structural motif is evopreQ1 or a variant thereof comprising a nucleotide sequence selected from SEQ ID NOs: 84-90 or a sequence at least 85% identical (85%, 86%, 87%, 88%, 89%, 90%, 91%, 92%, 93%, 94%, 95%, 96%, 97%, 98%, 99%, 100%, or a number or a range between any two of these values) to a nucleotide sequence selected from SEQ ID NOs: 84-90. In some embodiments, the evopreQ1 is trimmed (TevopreQ1).

In some embodiments, appending one or more RNA structural motifs to a tagRNA can protect against degradation of the tagRNA. Such RNA structural motifs can include, but are not limited to (i) a prequeosine1-1 riboswitch aptamer (evopreQ1) and variants thereof, (ii) a frameshifting pseudoknot from Moloney murine leukemia virus (MMLV), hereafter referred to as “mpknot,” and variants thereof (iii) G-quadruplexes, (iv) hairpin structures (e.g., 15-bp hairpins), (v) xrRNA, and (vi) a P4-P6 domain of the group I intron.

The present disclosure provides modified tagRNAs with improved properties, including but not limited to, increased stability and cellular lifespan, and improved binding affinity for a RT editor. These modified tagRNAs result in improved genome editing as demonstrated by increase editing efficiency at a wide variety of genomic sites. In some embodiments, by appending certain nucleic acid structural motifs to terminus of the extension arm of a tagRNA, including but limited to, a prequeosin₁-1 riboswitch aptamer (“evopreQ₁-1”) or variant thereof, a pseudoknot from the MMLV viral genome (“evopreQ₁-1”) or variant thereof, a modified tRNA used by MMLV RT as a primer for reverse transcription or variant thereof, and a G quadruplex or variant thereof, a consistent increase in editing activity can be achieved. In some embodiments, the 3′ terminus of the tagRNA comprises a evopreQ1 aptamer or variant thereof, comprising or consisting of a sequence of any one of: TTGACGCGGTTCTATCTAGTTACGCGTTAAACCAACTAGAAA (SEQ ID NO: 84), CGCGAGTCTAGGGGATAACGCGTTAAACTTCCTAGAAGGCGGTT (SEQ ID NO: 85), CGCGGATCTAGATTGTAACGCGTTAAACCATCTAGAAGGCGGTT (SEQ ID NO: 86), CGCGTCGCTACCGCCCGGCGCGTTAAACACACTAGAAGGCGGTT (SEQ ID NO: 87), CGCGGTTCTATCTAGTTACGCGTTAAACCAACTAGAA (SEQ ID NO: 88), TTGACGCGCTTCTATCTAGTTACGCGTTAAACCAACTAGAAA (SEQ ID NO: 89), and TTGACGCGGTTCTATCTACTTACGCGTTAAACCAACTAGAAA (SEQ ID NO: 90). For RNA sequences, T can be U.

In one embodiment, the modified tagRNAs include a nucleic acid moiety at the 3′ end of the tagRNA. Optionally, the 3′ end of the tagRNA is fused to the nucleic acid moiety through a nucleotide linker. In various embodiments, it will be appreciated that a wide variety of nucleotide sequences will work reasonably well for each genomic target site. Linker length can also be variable. In some cases, linkers ranging in length from 3-18 nucleotides can be used. In other cases, the linker may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides.

In general, the nucleic acid moieties that may be used to modify a tagRNA, for example, by attaching it to the 3′ end of a tagRNA, may include any nucleic acid moiety, including, for instance, a nucleic acid molecule comprising or which forms a double-helix moiety, toeloop moiety, hairpin moiety, stem-loop moiety, pseudoknot moiety, aptamer moiety, G quadraplex moiety, tRNA moiety, or a ribozyme moiety. The nucleic acid moiety may be characterized as forming a secondary nucleic acid structure, a tertiary nucleic acid structure, or a quadruple nucleic acid structure. In other words, the nucleic acid moiety may form any two dimensional or three dimensional structure known to be formed by such structures. The nucleic acid moiety may be DNA or RNA.

In some embodiments, a tagRNA or a nick guide RNA (egRNA) can be chemically synthesized, or can be assembled or cloned and transcribed from a DNA sequence, e.g., a plasmid DNA sequence, or by any RNA oligonucleotide synthesis method known in the art. In some embodiments, DNA sequence that encodes a tagRNA (or egRNA) can be designed to append one or more nucleotides at the 5′ end or the 3′ end of the tagRNA (or nick guide RNA) encoding sequence to enhance tagRNA transcription. For example, in some embodiments, a DNA sequence that encodes a tagRNA (or an egRNA) can be designed to append a nucleotide G at the 5′ end. Accordingly, in some embodiments, the tagRNA (or nick guide RNA) can comprise an appended nucleotide G at the 5′ end. In some embodiments, a DNA sequence that encodes a tagRNA (or nick guide RNA) can be designed to append a sequence that enhances transcription, e.g., a Kozak sequence, at the 5′ end. In some embodiments, a DNA sequence that encodes a tagRNA (or nick guide RNA) can be designed to append the sequence CACC or CCACC at the 5′ end. Accordingly, in some embodiments, the tagRNA (or nick guide RNA) can comprise an appended sequence CACC or CCACC at the 5′ end. In some embodiments, a DNA sequence that encodes a tagRNA (or nick guide RNA) can be designed to append the sequence TTT, TTTT, TTTTT, TTTTTT, TTTTTTT at the 3′ end. Accordingly, in some embodiments, the tagRNA (or nick guide RNA) can comprise an appended sequence UUU, UUIUU, UUUUU, UUU UUU, or UUTJUUUUU at the 3′ end.

Disclosed herein include RT editing systems. In some embodiments, the RT editing system comprises: any of the tagRNAs disclosed herein, or a nucleic acid encoding the tagRNA; and a RT editor comprising a DNA binding domain, a DNA endonuclease domain and a DNA polymerase domain, or a nucleic acid encoding the RT editor. In some embodiments, the intended nucleotide edit incorporation rate of the RT editing system is greater than at least about 30%, about 40%, about 50%, about 60%, about 70%, or about 80% (e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values).

Disclosed herein include RT editing complexes. In some embodiments, the RT editing complex comprises: (i) any of the tagRNA disclosed herein and a RT editor comprising a DNA endonuclease domain and a DNA polymerase domain; or (ii) any of the RT editing systems of the disclosure. In some embodiments, the intended nucleotide edit incorporation rate of the RT editing complex is greater than at least about 30%, about 40%, about 50%, about 60%, about 70%, or about 80% (e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values).

Enhancer gRNA (egRNA)

Disclosed herein include RT editing systems. In some embodiments, the RT editing system comprises: any of the tagRNAs disclosed herein, or a nucleic acid encoding the tagRNA; and a RT editor comprising a DNA binding domain, a DNA endonuclease domain and a DNA polymerase domain, or a nucleic acid encoding the RT editor. The RT editing system can comprise: an enhancer guide RNA (egRNA), or a nucleic acid encoding the egRNA, wherein the egRNA comprises a egRNA spacer that is complementary to a second search target sequence in the target gene.

In some embodiments, a RT editing system or composition further comprises an enhancer guide polynucleotide, such as an enhancer guide RNA (egRNA). Without wishing to be bound by any particular theory, the non-edit strand of a double stranded target DNA in the target gene may be nicked by a CRISPR-Cas nickase directed by an egRNA. In some embodiments, the nick on the non-edit strand directs endogenous DNA repair machinery to use the edit strand as a template for repair of the non-edit strand, which may increase efficiency of RT editing. In some embodiments, the non-edit strand is nicked by a RT editor localized to the non-edit strand by the egRNA. Accordingly, also provided herein are tagRNA systems comprising at least one tagRNA and at least one egRNA.

In some embodiments, the egRNA is a guide RNA which contains a variable spacer sequence and a guide RNA scaffold or core region that interacts with the DNA binding domain, a DNA endonuclease domain, e.g., Cas9 of the RT editor, in some embodiments, the egRNA comprises a spacer sequence (referred to herein as an ng spacer, or a second spacer) that is substantially complementary to a second search target sequence (or ng search target sequence), which is located on the edit strand, or the non-target strand. Thus, in some embodiments, the egRNA search target sequence recognized by the egRNA spacer and the search target sequence recognized by the spacer sequence of the tagRNA are on opposite strands of the double stranded target DNA of target gene, e.g., the ATP7B gene, PAH gene, or SERPINA1 gene. In some embodiments, an egRNA spacer sequence is complementary to, and may hybridize with the second search target sequence only after an intended nucleotide edit has been incorporated on the edit strand, by the editing template of a tagRNA.

In some embodiments, the egRNA search target sequence is located on the non-target strand, within 10 base pairs to 100 base pairs of an intended nucleotide edit incorporated by the tagRNA on the edit strand, in some embodiments, the egRNA target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the tagRNA on the edit strand. In some embodiments, the 5′ ends of the egRNA search target sequence and the tagRNA search target sequence are within 1, 2, 3, 4, 5, 6, 7, 8, 9, or 10 bp apart from each other. In some embodiments, the 5′ ends of the egRNA search target sequence and the tagRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an egRNA spacer sequence is complementary to, and may hybridize with the second search target sequence only after an intended nucleotide edit has been incorporated on the edit strand, by the editing template of a tagRNA. In some embodiments, the egRNA comprises a spacer sequence that matches only the edit strand after incorporation of the nucleotide edits, but not the endogenous target gene sequence on the edit strand. Accordingly, in some embodiments, an intended nucleotide edit is incorporated within the egRNA search target sequence. In some embodiments, the intended nucleotide edit is incorporated within about 1-10 nucleotides of the position corresponding to the PAM of the egRNA search target sequence.

The RT editing system can comprise: a nick guide RNA (egRNA), or a nucleic acid encoding the egRNA, wherein the egRNA comprises an egRNA spacer that is complementary to a second search target sequence in the target gene. The second search target sequence can be on the second strand of the target gene. The egRNA spacer can be from 16 to 22 nucleotides in length. In some embodiments, the length of the spacer varies from about 10 to about 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, or 20 to 30 nucleotides in length. In some embodiments, the spacer is 16 to 22 nucleotides in length. In some embodiments, the spacer is 16 to 20 nucleotides in length. In some embodiments, the spacer is 17 to 18 nucleotides in length. In some embodiments, the egRNA spacer is 20 nucleotides in length. In some embodiments, the egRNA comprises a scaffold sequence. The scaffold sequence can comprise a nucleotide sequence selected from SEQ ID NOs: 1210-1266.

In some embodiments, the egRNA comprises a nucleotide sequence selected from SEQ ID NOs: 48-49, 498-555, and 1048-1049. In some embodiments, the egRNA comprises a nucleotide sequence selected from SEQ ID NOs: 48-49, 498-555, and 1048-1049 or a sequence have one, two, or three mismatches relative to a nucleotide sequence selected from SEQ ID NOs: 48-49, 498-555, and 1048-1049. In some embodiments, the egRNA comprises or consists of a nucleotide sequence selected from SEQ ID NOs: 48-49, 498-555, and 1048-1049 or a sequence at least 85% identical to a nucleotide sequence selected from SEQ ID NOs: 48-49, 498-555, and 1048-1049 (e.g., 85%, 86%, 87%, 88%, 89%, 90%, 91%, 92%, 93%, 94%, 95%, 96%, 97%, 98%, 99%, 100%, or a number or a range between any two of these values, identical to a nucleotide sequence selected from SEQ ID NOs: 48-49, 498-555, and 1048-1049).

Different tagRNAs can be used with different egRNAs. In some embodiments, the tagRNA comprises or consists of any one of SEQ ID NOs: 16-47 and the egRNA comprises or consists of SEQ ID NO: 48. In some embodiments, the tagRNA comprises or consists of any one of SEQ ID NOs: 16-47 and the egRNA comprises or consists of SEQ ID NO: 49.

In some embodiments, the egRNA spacer comprises a nucleotide sequence selected from SEQ ID NOs: 51-52. In some embodiments, the egRNA spacer comprises a nucleotide sequence selected from SEQ ID NOs: 51-52 or a sequence have one, two, or three mismatches relative to a nucleotide sequence selected from SEQ ID NOs: 51-52. In some embodiments, the egRNA spacer comprises or consists of a nucleotide sequence selected from SEQ ID NOs: 51-52 or a sequence at least 85% identical to a nucleotide sequence selected from SEQ ID NOs: 51-52 (e.g., 85%, 86%, 87%, 88%, 89%, 90%, 91%, 92%, 93%, 94%, 95%, 96%, 97%, 98%, 99%, 100%, or a number or a range between any two of these values, identical to a nucleotide sequence selected from SEQ ID NOs: 51-52).

In some embodiments, the intended nucleotide edit incorporation rate of the RT editing system is greater than at least about 30%, about 40%, about 50%, about 60%, about 70%, or about 80% (e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values).

Provided in Table 2A-Table 2B below are exemplary protein (Table 2A) and mRNA (Table 2B) sequences of RT Editors and components thereof. Table 2C provides exemplary RT Editor linker sequences.

TABLE 2A
RT EDITING PROTEIN AND COMPONENTS
NAME and SEQ ID NO

RT editor fusion (SEQ ID NO: 4)
bipartite SV40 NLS (SEQ ID NO: 5)
SPYCas9 R221K N394K H840A (SEQ ID NO: 6)
Linker (SEQ ID NO: 7)
codon optimized MMLV RT pentamutant (SEQ ID NO: 8)
Linker - SGGS* (SEQ ID NO: 9 or 10)
*may be repeated one or more times
bipartite SV40 NLS (SEQ ID NO: 11)
c-Myc NLS (SEQ ID NO: 12)
RT editor fusion cDNA (SEQ ID NO: 13)
Reference/wild-type SPYCas9 (SEQ ID NO: 14)
wild type MMLV RT (SEQ ID NO: 15)
M. georgiana RT (SEQ ID NO: 141)

TABLE 2B
EXEMPLARY mRNA SEQUENCES ENCODING RT EDITORS
NAME and SEQ ID NO

pPG275 mRNA (SEQ ID NO: 137)
pPG276 mRNA (SEQ ID NO: 138)
pPG277 mRNA (SEQ ID NO: 139)
pPG278 mRNA (SEQ ID NO: 140)
*For RNA, U are T in the sequence listing.

TABLE 2C
EXEMPLARY RT EDITOR LINKER SEQUENCES

| | Shorthand | | SEQ |
|---|---|---|---|
| Name | Sequence | AA Residues | ID NO |
| GS3 | (GGGGS)3 | GGGGSGGGGSGGGGS | 1026 |
| GS4 | (GGGGS)4 | GGGGSGGGGSGGGGSGGGGS | 1027 |
| GS5 | (GGGGS)5 | GGGGSGGGGSGGGGSGGGGSGGGGS | 1056 |
| GS6 | (GGGGS)6 | GGGGSGGGGSGGGGSGGGGSGGGGSGGGGS | 1057 |
| GS7 | (GGGGS)7 | GGGGSGGGGSGGGGSGGGGSGGGGSGGGGSGGGGS | 1028 |
| EAK3 | (EAAAK)3 | EAAAKEAAAKEAAAK | 1022 |
| EAK4 | (EAAAK)4 | EAAAKEAAAKEAAAKEAAAK | 1023 |
| EAK5 | (EAAAK)5 | EAAAKEAAAKEAAAKEAAAKEAAAK | 1058 |
| EAK6 | (EAAAK)6 | EAAAKEAAAKEAAAKEAAAKEAAAKEAAAK | 1024 |
| EAK7 | (EAAAK)7 | EAAAKEAAAKEAAAKEAAAKEAAAKEAAAKEAAAK | 1059 |
| AEAK4- | A(EAAAK)4ALEA | AEAAAKEAAAKEAAAKEAAAKALEAEAAAKEAAAKEA | 1020 |
| ALEA | (EAAAK)4A | AAKEAAAKA | |
| GS- | GSA(EAAAK)4AL | GSAEAAAKEAAAKEAAAKEAAAKALEAEAAAKEAAAKE | 1060 |
| AEAK4- | EA(EAAAK)4ASG | AAAKEAAAKASG | |
| ALEA | | | |
| GGS- | GGGGS(EAAAK)4 | GGGGSEAAAKEAAAKEAAAKEAAAKALEAEAAAKEAA | 1021 |
| AEAK4- | ALEA | AKEAAAKEAAAKSGGGG | |
| ALEA | (EAAAAK)4SGGG | | |
| | G | | |
| AEAK | AEAAAKEAAAK | AEAAAKEAAAKA | 1061 |
| | A | | |
| AEAK2 | (AEAAAKEAAAK | AEAAAKEAAAKAAEAAAKEAAAKA | 1019 |
| | A)2 | | |
| AEAK3 | (AEAAAKEAAAK | AEAAAKEAAAKAAEAAAKEAAAKAAEAAAKEAAAKA | 1062 |
| | A)3 | | |
| GGS- | GGGGS(EAAAK)3 | GGGGSEAAAKEAAAKEAAAKGGGGS | 1025 |
| EAK3- | GGGGS | | |
| SGG | | | |
| GGS- | GGGGS(EAAAK)4 | GGGGSEAAAKEAAAKEAAAKEAAAKGGGGS | 1063 |
| EAK4- | GGGGS | | |
| SGG | | | |
| GGS- | GGGGS(EAAAK)5 | GGGGSEAAAKEAAAKEAAAKEAAAKEAAAKGGGGS | 1064 |
| EAK5- | GGGGS | | |
| SGG | | | |
| GGS- | GGGGS(EAAAK)3 | GGGGSEAAAKEAAAKEAAAKGGSGGSGEAAAKEAAAKE | 1065 |
| EAK3- | GGSGGSG(EAAA | AAAKGGGGS | |
| GGS | K)3GGGGS | | |
| GSA-F | SGAGSAAGSGEF | GSAGSAAGSGEF | 1066 |
| | GS | | |
| GSA-F- | SGAGSAAGSGEF | GSAGSAAGSGEFEGSGSAASGASG | 1067 |
| flip | EGSGSAASGAGS | | |
| K19D | KESGSVSSEQLA | KESGSVSSEQLAQFRSLD | 1033 |
| | QFRSLD | | |
| E14T | EGKSSGSGSESKS | EGKSSGSGSESKST | 1032 |
| | T | | |
| PAPAP | PAPAP | PAPAP | 1068 |
| PAPAP-2 | (PAPAP)2 | PAPAPPAPAP | 1029 |
| PAPAP-3 | (PAPAP)3 | PAPAPPAPAPPAPAP | 1069 |
| PAPAP-4 | (PAPAP)4 | PAPAPPAPAPPAPAPPAPAP | 1030 |
| PAPAP-5 | (PAPAP)5 | PAPAPPAPAPPAPAPPAPAPPAPAP | 1070 |
| PAPAP-6 | (PAPAP)6 | PAPAPPAPAPPAPAPPAPAPPAPAPPAPAP | 1071 |
| PAPAP-7 | (PAPAP)7 | PAPAPPAPAPPAPAPPAPAPPAPAPPAPAPPAPAP | 1072 |
| PAPAP-8 | (PAPAP)8 | PAPAPPAPAPPAPAPPAPAPPAPAPPAPAPPAPAPPAPAP | 1031 |
| GS- | | SGGGGSSGSETPGTSESATPESSGGGGS | 1034 |
| XTEN16- | | | |
| GS | | | |
| SGGSx2- | | SGGSSGGSSGSETPGTSESATPESSGGSSGGS | 1035 |
| XTEN16- | | | |
| SGGSx2 | | | |
| GGS- | | SGGGGSAEAAAKEAAAKEAAAKEAAAKALEAEAAAKEA | 1073 |
| AEAK- | | AAKEAAAKEAAAKASGGGGS | |
| ALEA_ | | | |
| mod1-gs | | | |
| GS-NLS- | | SGGSSGGSKRTADGSEFESPKKKRKVSGGSSGGS | 1074 |
| GS_4-gs | | | |

Provided in Table 3 below are exemplary spacer and edit target sequences for editing ATP7B3 gene.

TABLE 3
EXEMPLARY SPACER AND PROTOSPACER SEQUENCES

| SEQ | | SEQ | |
|---|---|---|---|
| ID | | ID | |
| NO: | SPACER SEQUENCE¹ | NO: | PROTOSPACER SEQUENCE |
| 50 | UUGGUGACUGCCACGCCCAA | 53 | TTGGTGACTGCCACGCCCAA |
| 51 | GGCCAGCAGUGAACACCCCU | 54 | GGCCAGCAGTGAACACCCCT |
| 52 | GCCAGCAGUGAACACCCCUU | 55 | GCCAGCAGTGAACACCCCTT |
| 133 | UUUGGUGACUGCCACGCCCA | 135 | TTTGGTGACTGCCACGCCCA |
| 134 | CAGUGAACACCCCUUGGGCG | 136 | CAGTGAACACCCCTTGGGCG |

¹For RNA, U are T in the sequence listing.

The tables below provide sequences related to the gRNAs of the disclosure (e.g., tagRNA or egRNA). For guide RNA sequences, the spacer sequences are underlined, and the extension arms are bold. Descriptions provide information regarding, e.g., the length of the FBS and the homology or editing template (ET) region of the extension arm. As would be understood by a person of ordinary skill in the art, the extension arm can comprise, from 5′ to 3′, an editing template (comprising a homology region) and flap binding sequence (FB sequence; FBS). As also understood by the skilled person, the nucleotide thymine (T) is used in DNA sequences and uracil (U) in RNA sequences. In some embodiments, a “T” can represent “U” when the molecule is an RNA molecule. Provided in Table 4 below are exemplary tagRNA (e.g., long gRNA) and nicking guide RNA (egRNA) sequences of the disclosure.

TABLE 4
EXEMPLARY tagRNA AND egRNA SEQUENCES

| | | SEQ ID | |
|---|---|---|---|
| NAME | DESCRIPTION | NO: | SEQUENCE¹ |
| BK g050 | Mut1.Hom14. | 16 | UUGGUGACUGCCACGCCCAAgUUUUAGAGCUAGAAAUAGC |
| | FBS13 | | AAGUUAAAAUAAGGCUAGUCCGUUAUCAACUUGAAAAAGU |
| | | | GGCACCGAGUCGGUGCGCCAGCAGUGAACAcCCCUUGGG |
| | | | CGUGGCAGUCAUUUU |
| BK g051 | Mut1.Hom14. | 17 | UUGGUGACUGCCACGCCCAAgUUUUAGAGCUAGAAAUAGC |
| | FBS12 | | AAGUUAAAAUAAGGCUAGUCCGUUAUCAACUUGAAAAAGU |
| | | | GGCACCGAGUCGGUGCGCCAGCAGUGAACAcCCCUUGGG |
| | | | CGUGGCAGUCUUUU |
| BK g052 | Mut1.Hom14. | 18 | UUGGUGACUGCCACGCCCAAgUUUUAGAGCUAGAAAUAGC |
| | FBS11 | | AAGUUAAAAUAAGGCUAGUCCGUUAUCAACUUGAAAAAGU |
| | | | GGCACCGAGUCGGUGCGCCAGCAGUGAACAcCCCUUGGG |
| | | | CGUGGCAGUUUUU |
| BK g053 | Mut1.Hom14. | 19 | UUGGUGACUGCCACGCCCAAgUUUUAGAGCUAGAAAUAGC |
| | FBS10 | | AAGUUAAAAUAAGGCUAGUCCGUUAUCAACUUGAAAAAGU |
| | | | GGCACCGAGUCGGUGCGCCAGCAGUGAACAcCCCUUGGG |
| | | | CGUGGCAGUUUU |
| BK g054 | Mut1.Hom14. | 20 | UUGGUGACUGCCACGCCCAAgUUUUAGAGCUAGAAAUAGC |
| | FBS9 | | AAGUUAAAAUAAGGCUAGUCCGUUAUCAACUUGAAAAAGU |
| | | | GGCACCGAGUCGGUGCGCCAGCAGUGAACAcCCCUUGGG |
| | | | CGUGGCAUUUU |
| BK g055 | Mut1.Hom14. | 21 | UUGGUGACUGCCACGCCCAAgUUUUAGAGCUAGAAAUAGC |
| | FBS8 | | AAGUUAAAAUAAGGCUAGUCCGUUAUCAACUUGAAAAAGU |
| | | | GGCACCGAGUCGGUGCGCCAGCAGUGAACAcCCCUUGGG |
| | | | CGUGGCUUUU |
| BK g056 | Mut1.Hom14. | 22 | UUGGUGACUGCCACGCCCAAgUUUUAGAGCUAGAAAUAGC |
| | FBS7 | | AAGUUAAAAUAAGGCUAGUCCGUUAUCAACUUGAAAAAGU |
| | | | GGCACCGAGUCGGUGCGCCAGCAGUGAACAcCCCUUGGG |
| | | | CGUGGUUUU |
| BK g057 | Mut1.Hom14. | 23 | UUGGUGACUGCCACGCCCAAgUUUUAGAGCUAGAAAUAGC |
| | FBS6 | | AAGUUAAAAUAAGGCUAGUCCGUUAUCAACUUGAAAAAGU |
| | | | GGCACCGAGUCGGUGCGCCAGCAGUGAACAcCCCUUGGG |
| | | | CGUGUUUU |
| BK g058 | Mut1.Hom10. | 24 | UUGGUGACUGCCACGCCCAAgUUUUAGAGCUAGAAAUAGC |
| | FBS13 | | AAGUUAAAAUAAGGCUAGUCCGUUAUCAACUUGAAAAAGU |
| | | | GGCACCGAGUCGGUGCGCAGUGAACAcCCCUUGGGCGUG |
| | | | GCAGUCAUUUU |
| BK g059 | Mut1.Hom10. | 25 | UUGGUGACUGCCACGCCCAAgUUUUAGAGCUAGAAAUAGC |
| | FBS12 | | AAGUUAAAAUAAGGCUAGUCCGUUAUCAACUUGAAAAAGU |
| | | | GGCACCGAGUCGGUGCGCAGUGAACAcCCCUUGGGCGUG |
| | | | GCAGUCUUUU |
| BK g060 | Mut1.Hom10. | 26 | UUGGUGACUGCCACGCCCAAgUUUUAGAGCUAGAAAUAGC |
| | FBS11 | | AAGUUAAAAUAAGGCUAGUCCGUUAUCAACUUGAAAAAGU |
| | | | GGCACCGAGUCGGUGCGCAGUGAACAcCCCUUGGGCGUG |
| | | | GCAGUUUUU |
| BK g061 | Mut1.Hom10. | 27 | UUGGUGACUGCCACGCCCAAgUUUUAGAGCUAGAAAUAGC |
| | FBS10 | | AAGUUAAAAUAAGGCUAGUCCGUUAUCAACUUGAAAAAGU |
| | | | GGCACCGAGUCGGUGCGCAGUGAACAcCCCUUGGGCGUG |
| | | | GCAGUUUU |
| BK g062 | Mut1.Hom10. | 28 | UUGGUGACUGCCACGCCCAAgUUUUAGAGCUAGAAAUAGC |
| | FBS9 | | AAGUUAAAAUAAGGCUAGUCCGUUAUCAACUUGAAAAAGU |
| | | | GGCACCGAGUCGGUGCGCAGUGAACAcCCCUUGGGCGUG |
| | | | GCAUUUU |
| BK g063 | Mut1.Hom10. | 29 | UUGGUGACUGCCACGCCCAAgUUUUAGAGCUAGAAAUAGC |
| | FBS8 | | AAGUUAAAAUAAGGCUAGUCCGUUAUCAACUUGAAAAAGU |
| | | | GGCACCGAGUCGGUGCGCAGUGAACAcCCCUUGGGCGUG |
| | | | GCUUUU |
| BK g064 | Mut1.Hom10. | 30 | UUGGUGACUGCCACGCCCAAgUUUUAGAGCUAGAAAUAGC |
| | FBS7 | | AAGUUAAAAUAAGGCUAGUCCGUUAUCAACUUGAAAAAGU |
| | | | GGCACCGAGUCGGUGCGCAGUGAACAcCCCUUGGGCGUG |
| | | | GUUUU |
| BK g065 | Mut1.Hom10. | 31 | UUGGUGACUGCCACGCCCAAgUUUUAGAGCUAGAAAUAGC |
| | FBS6 | | AAGUUAAAAUAAGGCUAGUCCGUUAUCAACUUGAAAAAGU |
| | | | GGCACCGAGUCGGUGCGCAGUGAACAcCCCUUGGGCGUG |
| | | | UUUU |
| BK g066 | Mut1.Hom7. | 32 | UUGGUGACUGCCACGCCCAAgUUUUAGAGCUAGAAAUAGC |
| | FBS13 | | AAGUUAAAAUAAGGCUAGUCCGUUAUCAACUUGAAAAAGU |
| | | | GGCACCGAGUCGGUGCGUGAACAcCCCUUGGGCGUGGCA |
| | | | GUCAUUUU |
| BK g067 | Mut1.Hom7. | 33 | UUGGUGACUGCCACGCCCAAgUUUUAGAGCUAGAAAUAGC |
| | FBS12 | | AAGUUAAAAUAAGGCUAGUCCGUUAUCAACUUGAAAAAGU |
| | | | GGCACCGAGUCGGUGCGUGAACAcCCCUUGGGCGUGGCA |
| | | | GUCUUUU |
| BK g068 | Mut1.Hom7. | 34 | UUGGUGACUGCCACGCCCAAgUUUUAGAGCUAGAAAUAGC |
| | FBS11 | | AAGUUAAAAUAAGGCUAGUCCGUUAUCAACUUGAAAAAGU |
| | | | GGCACCGAGUCGGUGCGUGAACAcCCCUUGGGCGUGGCA |
| | | | GUUUUU |
| BK g069 | Mut1.Hom7. | 35 | UUGGUGACUGCCACGCCCAAgUUUUAGAGCUAGAAAUAGC |
| | FBS10 | | AAGUUAAAAUAAGGCUAGUCCGUUAUCAACUUGAAAAAGU |
| | | | GGCACCGAGUCGGUGCGUGAACAcCCCUUGGGCGUGGCA |
| | | | GUUUU |
| BK g070 | Mut1.Hom7.FBS9 | 36 | UUGGUGACUGCCACGCCCAAgUUUUAGAGCUAGAAAUAGC |
| | | | AAGUUAAAAUAAGGCUAGUCCGUUAUCAACUUGAAAAAGU |
| | | | GGCACCGAGUCGGUGCGUGAACAcCCCUUGGGCGUGGCA |
| | | | UUUU |
| BK g071 | Mut1.Hom7.FBS8 | 37 | UUGGUGACUGCCACGCCCAAgUUUUAGAGCUAGAAAUAGC |
| | | | AAGUUAAAAUAAGGCUAGUCCGUUAUCAACUUGAAAAAGU |
| | | | GGCACCGAGUCGGUGCGUGAACAcCCCUUGGGCGUGGCU |
| | | | UUU |
| BK g072 | Mut1.Hom7.FBS7 | 38 | UUGGUGACUGCCACGCCCAAgUUUUAGAGCUAGAAAUAGC |
| | | | AAGUUAAAAUAAGGCUAGUCCGUUAUCAACUUGAAAAAGU |
| | | | GGCACCGAGUCGGUGCGUGAACAcCCCUUGGGCGUGGUU |
| | | | UU |
| BK g073 | Mut1.Hom7.FBS6 | 39 | UUGGUGACUGCCACGCCCAAgUUUUAGAGCUAGAAAUAGC |
| | | | AAGUUAAAAUAAGGCUAGUCCGUUAUCAACUUGAAAAAGU |
| | | | GGCACCGAGUCGGUGCGUGAACAcCCCUUGGGCGUGUUU |
| | | | U |
| BK g074 | Mut1.Hom5. | 40 | UUGGUGACUGCCACGCCCAAgUUUUAGAGCUAGAAAUAGC |
| | FBS13 | | AAGUUAAAAUAAGGCUAGUCCGUUAUCAACUUGAAAAAGU |
| | | | GGCACCGAGUCGGUGCGAACAcCCCUUGGGCGUGGCAGU |
| | | | CAUUUU |
| BK g075 | Mut1.Hom5. | 41 | UUGGUGACUGCCACGCCCAAgUUUUAGAGCUAGAAAUAGC |
| | FBS12 | | AAGUUAAAAUAAGGCUAGUCCGUUAUCAACUUGAAAAAGU |
| | | | GGCACCGAGUCGGUGCGAACAcCCCUUGGGCGUGGCAGU |
| | | | CUUUU |
| BK g076 | Mut1.Hom5. | 42 | UUGGUGACUGCCACGCCCAAgUUUUAGAGCUAGAAAUAGC |
| | FBS11 | | AAGUUAAAAUAAGGCUAGUCCGUUAUCAACUUGAAAAAGU |
| | | | GGCACCGAGUCGGUGCGAACAcCCCUUGGGCGUGGCAGU |
| | | | UUUU |
| BK g077 | Mut1.Hom5. | 43 | UUGGUGACUGCCACGCCCAAgUUUUAGAGCUAGAAAUAGC |
| | FBS10 | | AAGUUAAAAUAAGGCUAGUCCGUUAUCAACUUGAAAAAGU |
| | | | GGCACCGAGUCGGUGCGAACAcCCCUUGGGCGUGGCAGU |
| | | | UUU |
| BK g078 | Mut1.Hom5.FBS9 | 44 | UUGGUGACUGCCACGCCCAAgUUUUAGAGCUAGAAAUAGC |
| | | | AAGUUAAAAUAAGGCUAGUCCGUUAUCAACUUGAAAAAGU |
| | | | GGCACCGAGUCGGUGCGAACAcCCCUUGGGCGUGGCAUU |
| | | | UU |
| BK g079 | Mut1.Hom5.FBS8 | 45 | UUGGUGACUGCCACGCCCAAgUUUUAGAGCUAGAAAUAGC |
| | | | AAGUUAAAAUAAGGCUAGUCCGUUAUCAACUUGAAAAAGU |
| | | | GGCACCGAGUCGGUGCGAACAcCCCUUGGGCGUGGCUUU |
| | | | U |
| BK g080 | Mut1.Hom5.FBS7 | 46 | UUGGUGACUGCCACGCCCAAgUUUUAGAGCUAGAAAUAGC |
| | | | AAGUUAAAAUAAGGCUAGUCCGUUAUCAACUUGAAAAAGU |
| | | | GGCACCGAGUCGGUGCGAACAcCCCUUGGGCGUGGUUUU |
| BK g081 | Mut1.Hom5.FBS6 | 47 | UUGGUGACUGCCACGCCCAAgUUUUAGAGCUAGAAAUAGC |
| | | | AAGUUAAAAUAAGGCUAGUCCGUUAUCAACUUGAAAAAGU |
| | | | GGCACCGAGUCGGUGCGAACAcCCCUUGGGCGUGUUUU |
| BK g040² | | 48 | GGCCAGCAGUGAACACCCCUGUUUUAGAGCUAGAAAUAGC |
| | | | AAGUUAAAAUAAGGCUAGUCCGUUAUCAACUUGAAAAAGU |
| | | | GGCACCGAGUCGGUGCUUUU |
| BK g041² | | 49 | GCCAGCAGUGAACACCCCUUGUUUUAGAGCUAGAAAUAGC |
| | | | AAGUUAAAAUAAGGCUAGUCCGUUAUCAACUUGAAAAAGU |
| | | | GGCACCGAGUCGGUGCUUUU |

¹For RNA, U are T in the sequence listing. For each entry, the spacer sequence is underlined and the extension arm is bold. The description indicates the length of the FBS and ET or homology region (Hom) of the extension arm.
²egRNA

TABLE 5
EXEMPLARY FLAP BINDING SEQUENCE (FBS) AND EDITING TEMPLATE
SEQUENCES

| | | | | | SEQ ID |
|---|---|---|---|---|---|
| NAME | DESCRIPTION | FBS¹ | SEQ ID NO: | EDITING TEMPLATE¹ | NO: |
| BK g050 | Mut1.Hom14.FBS13 | GGCGTGGCAGTCA | 91 | GCCAGCAGTGAACA | 95 |
| | | | | CCCCTTG | |
| BK g051 | Mut1.Hom14.FBS12 | GGCGTGGCAGTC | 92 | GCCAGCAGTGAACA | 95 |
| | | | | CCCCTTG | |
| BK g052 | Mut1.Hom14.FBS11 | GGCGTGGCAGT | 93 | GCCAGCAGTGAACA | 95 |
| | | | | CCCCTTG | |
| BK g053 | Mut1.Hom14.FBS10 | GGCGTGGCAG | 94 | GCCAGCAGTGAACA | 95 |
| | | | | CCCCTTG | |
| BK g054 | Mut1.Hom14.FBS9 | GGCGTGGCA | | GCCAGCAGTGAACA | 95 |
| | | | | CCCCTTG | |
| BK g055 | Mut1.Hom14.FBS8 | GGCGTGGC | | GCCAGCAGTGAACA | 95 |
| | | | | CCCCTTG | |
| BK g056 | Mut1.Hom14.FBS7 | GGCGTGG | | GCCAGCAGTGAACA | 95 |
| | | | | CCCCTTG | |
| BK g057 | Mut1.Hom14.FBS6 | GGCGTG | | GCCAGCAGTGAACA | 95 |
| | | | | CCCCTTG | |
| BK g058 | Mut1.Hom10.FBS13 | GGCGTGGCAGTCA | 91 | GCAGTGAACACCCC | \|96 |
| | | | | TTG | |
| BK g059 | Mut1.Hom10.FBS12 | GGCGTGGCAGTC | 92 | GCAGTGAACACCCC | 96 |
| | | | | TTG | |
| BK g060 | Mut1.Hom10.FBS11 | GGCGTGGCAGT | 93 | GCAGTGAACACCCC | 96 |
| | | | | TTG | |
| BK g061 | Mut1.Hom10.FBS10 | GGCGTGGCAG | 94 | GCAGTGAACACCCC | 96 |
| | | | | TTG | |
| BK g062 | Mut1.Hom10.FBS9 | GGCGTGGCA | | GCAGTGAACACCCC | 196 |
| | | | | TTG | |
| BK g063 | Mut1.Hom10.FBS8 | GGCGTGGC | | GCAGTGAACACCCC | 96 |
| | | | | TTG | |
| BK g064 | Mut1.Hom10.FBS7 | GGCGTGG | | GCAGTGAACACCCC | 96 |
| | | | | TTG | |
| BK g065 | Mut1.Hom10.FBS6 | GGCGTG | | GCAGTGAACACCCC | 96 |
| | | | | TTG | |
| BK g066 | Mut1.Hom7.FBS13 | GGCGTGGCAGTCA | 91 | GTGAACACCCCTTG | 97 |
| BK g067 | Mut1.Hom7.FBS12 | GGCGTGGCAGTC | 92 | GTGAACACCCCTTG | 97 |
| BK g068 | Mut1.Hom7.FBS11 | GGCGTGGCAGT | 93 | GTGAACACCCCTTG | 97 |
| BK g069 | Mut1.Hom7.FBS10 | GGCGTGGCAG | 94 | GTGAACAcCCCTTG | 97 |
| BK g070 | Mut1.Hom7.FBS9 | GGCGTGGCA | | GTGAACACCCCTTG | 97 |
| BK g071 | Mut1.Hom7.FBS8 | GGCGTGGC | | GTGAACACCCCTTG | 97 |
| BK g072 | Mut1.Hom7.FBS7 | GGCGTGG | | GTGAACAcCCCTTG | 97 |
| BK g073 | Mut1.Hom7.FBS6 | GGCGTG | | GTGAACAcCCCTTG | 97 |
| BK g074 | Mut1.Hom5.FBS13 | GGCGTGGCAGTCA | 91 | GAACACCCCTTG | 98 |
| BK g075 | Mut1.Hom5.FBS12 | GGCGTGGCAGTC | 92 | GAACACCCCTTG | 98 |
| BK g076 | Mut1.Hom5.FBS11 | GGCGTGGCAGT | 93 | GAACACCCCTTG | 98 |
| BK g077 | Mut1.Hom5.FBS10 | GGCGTGGCAG | 94 | GAACACCCCTTG | 98 |
| BK g078 | Mut1.Hom5.FBS9 | GGCGTGGCA | | GAACACCCCTTG | 98 |
| BK g079 | Mut1.Hom5.FBS8 | GGCGTGGC | | GAACACCCCTTG | 98 |
| BK g080 | Mut1.Hom5.FBS7 | GGCGTGG | | GAACACCCCTTG | 98 |
| BK g081 | Mut1.Hom5.FBS6 | GGCGTG | | GAACACCCCTTG | 98 |

¹For RNA, T is U; U are T in the sequence listing. The description indicates the length of the FBS and ET or homology region (Hom) of the extension arm.

TABLE 6
EXEMPLARY gRNAs

| NAME | SEQUENCE^{1,2} | SEQ ID NO: | DESCRIPTION |
|---|---|---|---|
| BK g050³ | mU*mU*mG*rGrUrGrArCrUrGrCrCrArCrGrCr | 99 | Human.ATP7B.NGG1.Mut1. |
| | CrCrArArGrUrUrUrUrArGrAmGmCmUmAmG | | Hom14.FBS13.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrCrArGrCrArGrUrGrArArCrArC | | |
| | rCrCrCrUrUrGrGrGrCrGrUrGrGrCrArGrU | | |
| | rCrAmU*mU*mU*mU | | |
| BK g051³ | mU*mU*mG*rGrUrGrArCrUrGrCrCrArCrGrCr | 100 | Human.ATP7B.NGG1.Mut1. |
| | CrCrArArGrUrUrUrUrArGrAmGmCmUmAmG | | Hom14.FBS12.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrCrArGrCrArGrUrGrArArCrArC | | |
| | rCrCrCrUrUrGrGrGrCrGrUrGrGrCrArGrU | | |
| | rCmU*mU*mU*mU | | |
| BK g052³ | mU*mU*mG*rGrUrGrArCrUrGrCrCrArCrGrCr | 101 | Human.ATP7B.NGG1.Mut1. |
| | CrCrArArGrUrUrUrUrArGrAmGmCmUmAmG | | Hom14.FBS11.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrCrArGrCrArGrUrGrArArCrArC | | |
| | rCrCrCrUrUrGrGrGrCrGrUrGrGrCrArGrU | | |
| | mU*mU*mU*mU | | |
| BK g053³ | mU*mU*mG*rGrUrGrArCrUrGrCrCrArCrGrCr | 102 | Human.ATP7B.NGG1.Mut1. |
| | CrCrArArGrUrUrUrUrArGrAmGmCmUmAmG | | Hom14.FBS10.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrCrArGrCrArGrUrGrArArCrArC | | |
| | rCrCrCrUrUrGrGrGrCrGrUrGrGrCrArGm | | |
| | U*mU*mU*mU | | |
| BK g054³ | mU*mU*mG*rGrUrGrArCrUrGrCrCrArCrGrCr | 103 | Human.ATP7B.NGG1.Mut1. |
| | CrCrArArGrUrUrUrUrArGrAmGmCmUmAmG | | Hom14.FBS9.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrCrArGrCrArGrUrGrArArCrArC | | |
| | rCrCrCrUrUrGrGrGrCrGrUrGrGrCrAmU* | | |
| | mU*mU*mU | | |
| BK g055³ | mU*mU*mG*rGrUrGrArCrUrGrCrCrArCrGrCr | 104 | Human.ATP7B.NGG1.Mut1. |
| | CrCrArArGrUrUrUrUrArGrAmGmCmUmAmG | | Hom14.FBS8.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrCrArGrCrArGrUrGrArArCrArC | | |
| | rCrCrCrUrUrGrGrGrCrGrUrGrGrCmU*mU | | |
| | *mU*mU | | |
| BK g056³ | mU*mU*mG*rGrUrGrArCrUrGrCrCrArCrGrCr | 105 | Human.ATP7B.NGG1.Mut1. |
| | CrCrArArGrUrUrUrUrArGrAmGmCmUmAmG | | Hom14.FBS7.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrCrArGrCrArGrUrGrArArCrArC | | |
| | rCrCrCrUrUrGrGrGrCrGrUrGrGmU*mU* | | |
| | mU*mU | | |
| BK g057³ | mU*mU*mG*rGrUrGrArCrUrGrCrCrArCrGrCr | 106 | Human. ATP7B.NGG1.Mut1. |
| | CrCrArArGrUrUrUrUrArGrAmGmCmUmAmG | | Hom14.FBS6.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrCrArGrCrArGrUrGrArArCrArC | | |
| | rCrCrCrUrUrGrGrGrCrGrUrGmU*mU*mU | | |
| | *mU | | |
| BK g058³ | mU*mU*mG*rGrUrGrArCrUrGrCrCrArCrGrCr | 107 | Human. ATP7B.NGG1.Mut1. |
| | CrCrArArGrUrUrUrUrArGrAmGmCmUmAmG | | Hom10.FBS13.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrArGrUrGrArArCrArCrCrCrCrU | | |
| | rUrGrGrGrCrGrUrGrGrCrArGrUrCrAmU* | | |
| | mU*mU*mU | | |
| BK g059³ | mU*mU*mG*rGrUrGrArCrUrGrCrCrArCrGrCr | 108 | Human.ATP7B.NGG1.Mut1. |
| | CrCrArArGrUrUrUrUrArGrAmGmCmUmAmG | | Hom10.FBS12.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrArGrUrGrArArCrArCrCrCrCrU | | |
| | rUrGrGrGrCrGrUrGrGrCrArGrUrCmU*mU | | |
| | *mU*mU | | |
| BK g060³ | mU*mU*mG*rGrUrGrArCrUrGrCrCrArCrGrCr | 109 | Human. ATP7B.NGG1.Mut1. |
| | CrCrArArGrUrUrUrUrArGrAmGmCmUmAmG | | Hom10.FBS11.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrArGrUrGrArArCrArCrCrCrCrU | | |
| | rUrGrGrGrCrGrUrGrGrCrArGrUmU*mU* | | |
| | mU*mU | | |
| BK g061³ | mU*mU*mG*rGrUrGrArCrUrGrCrCrArCrGrCr | 110 | Human. ATP7B.NGG1.Mut1. |
| | CrCrArArGrUrUrUrUrArGrAmGmCmUmAmG | | Hom10.FBS10.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrArGrUrGrArArCrArCrCrCrCrU | | |
| | rUrGrGrGrCrGrUrGrGrCrArGmU*mU*mU | | |
| | *mU | | |
| BK g062³ | mU*mU*mG*rGrUrGrArCrUrGrCrCrArCrGrCr | 111 | Human.ATP7B.NGG1.Mut1. |
| | CrCrArArGrUrUrUrUrArGrAmGmCmUmAmG | | Hom10.FBS9.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrArGrUrGrArArCrArCrCrCrCrU | | |
| | rUrGrGrGrCrGrUrGrGrCrAmU*mU*mU*m | | |
| | U | | |
| BK g063³ | mU*mU*mG*rGrUrGrArCrUrGrCrCrArCrGrCr | 112 | Human.ATP7B.NGG1.Mut1. |
| | CrCrArArGrUrUrUrUrArGrAmGmCmUmAmG | | Hom10.FBS8.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrArGrUrGrArArCrArCrCrCrCrU | | |
| | rUrGrGrGrCrGrUrGrGrCmU*mU*mU*mU | | |
| BK g064³ | mU*mU*mG*rGrUrGrArCrUrGrCrCrArCrGrCr | 113 | Human.ATP7B.NGG1.Mut1. |
| | CrCrArArGrUrUrUrUrArGrAmGmCmUmAmG | | Hom10.FBS7.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrArGrUrGrArArCrArCrCrCrCrU | | |
| | rUrGrGrGrCrGrUrGrGmU*mU*mU*mU | | |
| BK g065³ | mU*mU*mG*rGrUrGrArCrUrGrCrCrArCrGrCr | 114 | Human.ATP7B.NGG1.Mut1. |
| | CrCrArArGrUrUrUrUrArGrAmGmCmUmAmG | | Hom10.FBS6.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrCrArGrUrGrArArCrArCrCrCrCrU | | |
| | rUrGrGrGrCrGrUrGmU*mU*mU*mU | | |
| BK g066³ | mU*mU*mG*rGrUrGrArCrUrGrCrCrArCrGrCr | 115 | Human.ATP7B.NGG1.Mut1. |
| | CrCrArArGrUrUrUrUrArGrAmGmCmUmAmG | | Hom7.FBS13.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrUrGrArArCrArCrCrCrCrUrUrGrG | | |
| | rGrCrGrUrGrGrCrArGrUrCrAmU*mU*mU | | |
| | *mU | | |
| BK g067³ | mU*mU*mG*rGrUrGrArCrUrGrCrCrArCrGrCr | 116 | Human.ATP7B.NGG1.Mut1. |
| | CrCrArArGrUrUrUrUrArGrAmGmCmUmAmG | | Hom7.FBS12.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrUrGrArArCrArCrCrCrCrUrUrGrG | | |
| | rGrCrGrUrGrGrCrArGrUrCmU*mU*mU*m | | |
| | U | | |
| BK g068³ | mU*mU*mG*rGrUrGrArCrUrGrCrCrArCrGrCr | 117 | Human.ATP7B.NGG1.Mut1. |
| | CrCrArArGrUrUrUrUrArGrAmGmCmUmAmG | | Hom7.FBS11.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrUrGrArArCrArCrCrCrCrUrUrGrG | | |
| | rGrCrGrUrGrGrCrArGrUmU*mU*mU*mU | | |
| BK g069³ | mU*mU*mG*rGrUrGrArCrUrGrCrCrArCrGrCr | 118 | Human.ATP7B.NGG1.Mut1. |
| | CrCrArArGrUrUrUrUrArGrAmGmCmUmAmG | | Hom7.FBS10.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrUrGrArArCrArCrCrCrCrUrUrGrG | | |
| | rGrCrGrUrGrGrCrArGmU*mU*mU*mU | | |
| BK g070³ | mU*mU*mG*rGrUrGrArCrUrGrCrCrArCrGrCr | 119 | Human.ATP7B.NGG1.Mut1. |
| | CrCrArArGrUrUrUrUrArGrAmGmCmUmAmG | | Hom7.FBS9.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrUrGrArArCrArCrCrCrCrUrUrGrG | | |
| | rGrCrGrUrGrGrCrAmU*mU*mU*mU | | |
| BK g071³ | mU*mU*mG*rGrUrGrArCrUrGrCrCrArCrGrCr | 120 | Human.ATP7B.NGG1.Mut1. |
| | CrCrArArGrUrUrUrUrArGrAmGmCmUmAmG | | Hom7.FBS8.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrUrGrArArCrArCrCrCrCrUrUrGrG | | |
| | rGrCrGrUrGrGrCmU*mU*mU*mU | | |
| BK g072³ | mU*mU*mG*rGrUrGrArCrUrGrCrCrArCrGrCr | 121 | Human. ATP7B.NGG1.Mut1. |
| | CrCrArArGrUrUrUrUrArGrAmGmCmUmAmG | | Hom7.FBS7.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrUrGrArArCrArCrCrCrCrUrUrGrG | | |
| | rGrCrGrUrGrGmU*mU*mU*mU | | |
| BK g073³ | mU*mU*mG*rGrUrGrArCrUrGrCrCrArCrGrCr | 122 | Human.ATP7B.NGG1.Mut1. |
| | CrCrArArGrUrUrUrUrArGrAmGmCmUmAmG | | Hom7.FBS6.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrUrGrArArCrArCrCrCrCrUrUrGrG | | |
| | rGrCrGrUrGmU*mU*mU*mU | | |
| BK g074³ | mU*mU*mG*rGrUrGrArCrUrGrCrCrArCrGrCr | 123 | Human. ATP7B.NGG1.Mut1. |
| | CrCrArArGrUrUrUrUrArGrAmGmCmUmAmG | | Hom5.FBS13.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrArArCrArCrCrCrCrUrUrGrGrGrC | | |
| | rGrUrGrGrCrArGrUrCrAmU*mU*mU*mU | | |
| BK g075³ | mU*mU*mG*rGrUrGrArCrUrGrCrCrArCrGrCr | 124 | Human.ATP7B.NGG1.Mut1. |
| | CrCrArArGrUrUrUrUrArGrAmGmCmUmAmG | | Hom5.FBS12.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrArArCrArCrCrCrCrUrUrGrGrGrC | | |
| | rGrUrGrGrCrArGrUrCmU*mU*mU*mU | | |
| BK g076³ | mU*mU*mG*rGrUrGrArCrUrGrCrCrArCrGrCr | 125 | Human.ATP7B.NGG1.Mut1. |
| | CrCrArArGrUrUrUrUrArGrAmGmCmUmAmG | | Hom5.FBS11.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrArArCrArCrCrCrCrUrUrGrGrGrC | | |
| | rGrUrGrGrCrArGrUmU*mU*mU*mU | | |
| BK g077³ | mU*mU*mG*rGrUrGrArCrUrGrCrCrArCrGrCr | 126 | Human.ATP7B.NGG1.Mut1. |
| | CrCrArArGrUrUrUrUrArGrAmGmCmUmAmG | | Hom5.FBS10.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrArArCrArCrCrCrCrUrUrGrGrGrC | | |
| | rGrUrGrGrCrArGmU*mU*mU*mU | | |
| BK g078³ | mU*mU*mG*rGrUrGrArCrUrGrCrCrArCrGrCr | 127 | Human.ATP7B.NGG1.Mut1. |
| | CrCrArArGrUrUrUrUrArGrAmGmCmUmAmG | | Hom5.FBS9.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrArArCrArCrCrCrCrUrUrGrGrGrC | | |
| | rGrUrGrGrCrAmU*mU*mU*mU | | |
| BK g079³ | mU*mU*mG*rGrUrGrArCrUrGrCrCrArCrGrCr | 128 | Human.ATP7B.NGG1.Mut1. |
| | CrCrArArGrUrUrUrUrArGrAmGmCmUmAmG | | Hom5.FBS8.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrArArCrArCrCrCrCrUrUrGrGrGrC | | |
| | rGrUrGrGrCmU*mU*mU*mU | | |
| BK g080³ | mU*mU*mG*rGrUrGrArCrUrGrCrCrArCrGrCr | 129 | Human.ATP7B.NGG1.Mut1. |
| | CrCrArArGrUrUrUrUrArGrAmGmCmUmAmG | | Hom5.FBS7.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrArArCrArCrCrCrCrUrUrGrGrGrC | | |
| | rGrUrGrGmU*mU*mU*mU | | |
| BK g081³ | mU*mU*mG*rGrUrGrArCrUrGrCrCrArCrGrCr | 130 | Human.ATP7B.NGG1.Mut1. |
| | CrCrArArGrUrUrUrUrArGrAmGmCmUmAmG | | Hom5.FBS6.mU*mU*mU*mU |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCrGrArArCrArCrCrCrCrUrUrGrGrGrC | | |
| | rGrUrGmU*mU*mU*mU | | |
| BK g040⁴ | mG*mG*mC*rCrArGrCrArGrUrGrArArCrArCr | 131 | Human.ATP7B.Mut1.3b.1.mU* |
| | CrCrCrUrGrUrUrUrUrArGrAmGmCmUmAmG | | mU*mU*mU.NGG2 |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCmU*mU*mU*mU | | |
| BK g041⁴ | mG*mC*mC*rArGrCrArGrUrGrArArCrArCrCr | 132 | Human.ATP7B.Mut1.3b.2.mU* |
| | CrCrUrUrGrUrUrUrUrArGrAmGmCmUmAmG | | mU*mU*mU.NGG2 |
| | mAmAmAmUmAmGmCrArArGrUrUrArArArA | | |
| | rUrArArGrGrCrUrArGrUrCrCrGrUrUrArUrCm | | |
| | AmAmCmUmUmGmAmAmAmAmAmGmUmG | | |
| | mGmCmAmCmCmGmAmGmUmCmGmGmUm | | |
| | GmCmU*mU*mU*mU | | |

¹For RNA, U are T in the sequence listing. For each entry, the spacer sequence is underlined and the extension arm is bold. The description indicates the length of the FBS and ET or homology region (Hom) of the extension arm.
²For the sequences: * , phosphorothioate modification; mN, 2′O-methyl modification; rN, unmodified ribonucleotide. N = A, C, T/U, G.
³tagRNA
⁴egRNA

TABLE 7
EXEMPLARY tagRNAs

| | | | SEQ ID |
|---|---|---|---|
| NAME | DESCRIPTION | SEQUENCE^{1,2} | NO: |
| PG g060 | Human.ATP7B.NGG1. | mU*mU*mG*rGrUrGrArCrUrGrCrCrArCrGrCrCrCrA | 164 |
| | Mut1.Hom7.FBS8.mU*mU* | rArGrUrUrUrUrArGrAmGmCmUmAmGmAmAmAm | |
| | mU*mU.ET14OMe | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCmGmUmGmAmAmCmAmC | |
| | | mCmCmCmUmUmGrGrGrCrGrUrGrGrCmU*mU | |
| | | *mU*mU | |
| PG g061 | Human.ATP7B.NGG1. | mU*mU*mG*rGrUrGrArCrUrGrCrCrArCrGrCrCrCrA | 165 |
| | Mut1.Hom7.FBS8.mU*mU* | rArGrUrUrUrUrArGrAmGmCmUmAmGmAmAmAm | |
| | mU*mU.FBS8OMe | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrUrGrArArCrArCrCrCrC | |
| | | rUrUrGmGmGmCmGmUmGmGmCmU*mU*mU* | |
| | | mU | |
| PG g062 | Human.ATP7B.NGG1. | mU*mU*mG*rGrUrGrArCrUrGrCrCrArCrGrCrCrCrA | 166 |
| | Mut1.Hom7.FBS8.mU*mU* | rArGrUrUrUrUrArGrAmGmCmUmAmGmAmAmAm | |
| | mU*mU.ET14OMe.FBS8OMe | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCmGmUmGmAmAmCmAmC | |
| | | mCmCmCmUmUmGmGmGmCmGmUmGmGmC | |
| | | mU*mU*mU*mU | |
| PG g063 | Human.ATP7B.NGG1. | mU*mU*mG*rGrUrGrArCrUrGrCrCrArCrGrCrCrCrA | 167 |
| | Mut1.Hom7.FBS8.mU*mU* | rArGrUrUrUrUrArGrAmGmCmUmAmGmAmAmAm | |
| | mU*mU.ET7OMe | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGmUrGmArAmCrAmCrC | |
| | | mCrCmUrUmGrGrGrCrGrUrGrGrCmU*mU*mU | |
| | | *mU | |
| PG g064 | Human.ATP7B.NGG1. | mU*mU*mG*rGrUrGrArCrUrGrCrCrArCrGrCrCrCrA | 168 |
| | Mut1.Hom7.FBS8.mU*mU* | rArGrUrUrUrUrArGrAmGmCmUmAmGmAmAmAm | |
| | mU*mU.FBS4OMe | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrUrGrArArCrArCrCrCrC | |
| | | rUrUrGrGmGrCmGrUmGrGmCmU*mU*mU*mU | |
| PG g065 | Human.ATP7B.NGG1. | mU*mU*mG*rGrUrGrArCrUrGrCrCrArCrGrCrCrCrA | 169 |
| | Mut1.Hom7.FBS8.mU*mU* | rArGrUrUrUrUrArGrAmGmCmUmAmGmAmAmAm | |
| | mU*mU.ET7OMe.FBS4OMe | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGm UrGmArAmCrAmCrC | |
| | | mCrCmUrUmGrGmGrCmGrUmGrGmCmU*mU* | |
| | | mU*mU | |
| PG g066 | Human.ATP7B.NGG1. | mU*mU*mG*rGrUrGrArCrUrGrCrCrArCrGrCrCrCrA | 170 |
| | Mut1.Hom7.FBS8.mU*mU* | rArGrUrUrUrUrArGrAmGmCmUmAmGmAmAmAm | |
| | mU*mU.ET4OMe | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrUmGrArAmCrArCmCrC | |
| | | rCmUrUrGrGrGrCrGrUrGrGrCmU*mU*mU*mU | |
| PG g067 | Human.ATP7B.NGG1. | mU*mU*mG*rGrUrGrArCrUrGrCrCrArCrGrCrCrCrA | 171 |
| | Mut1.Hom7.FBS8.mU*mU* | rArGrUrUrUrUrArGrAmGmCmUmAmGmAmAmAm | |
| | mU*mU.FBS3OMe | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrUrGrArArCrArCrCrCrC | |
| | | rUrUrGmGrGrCmGrUrGmGrCmU*mU*mU*mU | |
| PG g068 | Human.ATP7B.NGG1. | mU*mU*mG*rGrUrGrArCrUrGrCrCrArCrGrCrCrCrA | 172 |
| | Mut1.Hom7.FBS8.mU*mU* | rArGrUrUrUrUrArGrAmGmCmUmAmGmAmAmAm | |
| | mU*mU.ET4OMe.FBS3OMe | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrUmGrArAmCrArCmCrC | |
| | | rCmUrUrGmGrGrCmGrUrGmGrCmU*mU*mU* | |
| | | mU | |
| PG g069 | Human.ATP7B.NGG1. | mU*mU*mG*rGrUrGrArCrUrGrCrCrArCrGrCrCrCrA | 173 |
| | Mut1.Hom7.FBS8.mU*mU* | rArGrUrUrUrUrArGrAmGmCmUmAmGmAmAmAm | |
| | mU*mU.ET14F | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmC/i2FG//i2FU//i2FG//i2FA//i2FA// | |
| | | i2FC//i2FA//i2FC//i2FC//i2FC//i2FC//i2FU//i2FU// | |
| | | i2FG/rGrGrCrGrUrGrGrCmU*mU*mU*mU | |
| PG g070 | Human.ATP7B.NGG1. | mU*mU*mG*rGrUrGrArCrUrGrCrCrArCrGrCrCrCrA | 174 |
| | Mut1.Hom7.FBS8.mU*mU* | rArGrUrUrUrUrArGrAmGmCmUmAmGmAmAmAm | |
| | mU*mU.FBS8F | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrUrGrArArCrArCrCrCrC | |
| | | rUrUrG/i2FG//i2FG//i2FC//i2FG//i2FU//i2FG//i2FG// | |
| | | i2FC/mU*mU*mU*mU | |
| PG g071 | Human.ATP7B.NGG1. | mU*mU*mG*rGrUrGrArCrUrGrCrCrArCrGrCrCrCrA | 175 |
| | Mut1.Hom7.FBS8.mU*mU* | rArGrUrUrUrUrArGrAmGmCmUmAmGmAmAmAm | |
| | mU*mU.ET14F.FBS8F | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmC/i2FG//i2FU//i2FG//i2FA//i2FA// | |
| | | i2FC//i2FA//i2FC//i2FC//i2FC//i2FC//i2FU//i2FU// | |
| | | i2FG//i2FG//i2FG//i2FC//i2FG//i2FU//i2FG//i2FG// | |
| | | i2FC/mU*mU*mU*mU | |
| PG g072 | Human.ATP7B.NGG1. | mU*mU*mG*rGrUrGrArCrUrGrCrCrArCrGrCrCrCrA | 176 |
| | Mut1.Hom7.FBS8.mU*mU* | rArGrUrUrUrUrArGrAmGmCmUmAmGmAmAmAm | |
| | mU*mU.ET7F | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrG/i2FU/rG/i2FA/rA/i2FC/rA/ | |
| | | i2FC/rC/i2FC/rC/i2FU/rU/i2FG/rGrGrCrGrUrGr | |
| | | GrCmU*mU*mU*mU | |
| PG g073 | Human.ATP7B.NGG1. | mU*mU*mG*rGrUrGrArCrUrGrCrCrArCrGrCrCrCrA | 177 |
| | Mut1.Hom7.FBS8.mU*mU* | rArGrUrUrUrUrArGrAmGmCmUmAmGmAmAmAm | |
| | mU*mU.FBS4F | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrUrGrArArCrArCrCrCrC | |
| | | rUrUrGrG/i2FG/rC/i2FG/rU/i2FG/rG/i2FC/mU*m | |
| | | U*mU*mU | |
| PG g074 | Human.ATP7B.NGG1. | mU*mU*mG*rGrUrGrArCrUrGrCrCrArCrGrCrCrCrA | 178 |
| | Mut1.Hom7.FBS8.mU*mU* | rArGrUrUrUrUrArGrAmGmCmUmAmGmAmAmAm | |
| | mU*mU.ET7F.FBS4F | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrG/i2FU/rG/i2FA/rA/i2FC/rA/ | |
| | | i2FC/rC/i2FC/rC/i2FU/rU/i2FG/rG/i2FG/rC/i2FG/rU/ | |
| | | i2FG/rG/i2FC/mU*mU*mU*mU | |
| PG g075 | Human.ATP7B.NGG1. | mU*mU*mG*rGrUrGrArCrUrGrCrCrArCrGrCrCrCrA | 179 |
| | Mut1.Hom7.FBS8.mU*mU* | rArGrUrUrUrUrArGrAmGmCmUmAmGmAmAmAm | |
| | mU*mU.ET4F | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrU/i2FG/rArA/i2FC/rArC/ | |
| | | i2FC/rCrC/i2FU/rUrGrGrGrCrGrUrGrGrCmU*m | |
| | | U*mU*mU | |
| PG g076 | Human.ATP7B.NGG1. | mU*mU*mG*rGrUrGrArCrUrGrCrCrArCrGrCrCrCrA | 180 |
| | Mut1.Hom7.FBS8.mU*mU* | rArGrUrUrUrUrArGrAmGmCmUmAmGmAmAmAm | |
| | mU*mU.FBS3F | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrUrGrArArCrArCrCrCrC | |
| | | rUrUrG/i2FG/rGrC/i2FG/rUrG/i2FG/rCmU*mU*m | |
| | | U*mU | |
| PG g077 | Human.ATP7B.NGG1. | mU*mU*mG*rGrUrGrArCrUrGrCrCrArCrGrCrCrCrA | 181 |
| | Mut1.Hom7.FBS8.mU*mU* | rArGrUrUrUrUrArGrAmGmCmUmAmGmAmAmAm | |
| | mU*mU.ET4F.FBS3F | UmAmGmCrArArGrUrUrArArArArUrArArGrGrCrUr | |
| | | ArGrUrCrCrGrUrUrArUrCmAmAmCmUmUmGmAm | |
| | | AmAmAmAmGmUmGmGmCmAmCmCmGmAmGm | |
| | | UmCmGmGmUmGmCrGrU/i2FG/rArA/i2FC/rArC/ | |
| | | i2FC/rCrC/i2FU/rUrG/i2FG/rGrC/i2FG/rUrG/i2FG/ | |
| | | rCmU*mU*mU*mU | |

¹For RNA, T is U; U are T in the sequence listing. For each entry, the spacer sequence is underlined and the extension arm is bold. The description indicates the length of the FBS and ET or homology region of the extension arm.
²For the sequences: * , phosphorothioate modification; mN, 2′O-methyl modification; rN, unmodified ribonucleotide; i2FN, Int Fluoro modification. N = A, C, T/U, G.

TABLE 8A
EXEMPLARY CAS9 SEQUENCES

| DESCRIPTION | SEQ ID NO: |
|---|---|
| Ssch1Cas9_N583A | 142 |
| Sro1Cas9_N584A | 143 |
| Sha4Cas9_N583A | 144 |
| SsuCas9_N622A | 145 |
| evoCjCas9_H559A | 146 |
| iSpyMac_H840A | 147 |
| Ssi5Cas9_N580A | 148 |
| Ssi8Cas9_N580A | 149 |
| Ssci4Cas9_N580A | 150 |
| Shy1Cas9_N582A | 151 |
| Sag3Cas9_N582A | 152 |
| Slutr1Cas9_N583A | 153 |
| Ssch3Cas9_N583A | 154 |
| SpRYCas9_H840A | 155 |
| SpRYcCas9_H849A | 156 |
| Sma2Cas9_N622A | 157 |
| SsaCas9_N628A | 158 |
| EvoSluCas9_N582A | 159 |
| SluCas9_N582A | 160 |
| EvosRGN3.1_N585A | 161 |
| EvoNme2Cas9_H588A | 162 |
| EvoAnaCas9_H582A | 163 |

TABLE 8B
EXEMPLARY CAS9 SEQUENCES

| | | | SEQ ID |
|---|---|---|---|
| NAME | SPECIES (native or derived) | Length | NO: |
| St1Cas9- | Methanobacterium sp | 1097 | 270 |
| sim3_MetCas9_MBW4258562.1_Methanobacterium-sp- | | | |
| YSL_01 | | | |
| SsaCas9-sim1_ARC34060.1_Streptococcus-equinus | Streptococcus equinus | 1128 | 271 |
| SsaCas9-sim1_SsaCas9_AR00_CCB95040.1_Streptococcus- | Streptococcus salivarius | 1128 | 272 |
| salivarius-JIM8777_01 | | | |
| SsaCas9-sim1_SsaCas9_AR01_ancestral-seq- | Streptococcus salivarius | 1128 | 273 |
| recon_Streptococcus-salivarius_01 | | | |
| SsaCas9-sim1_SsaCas9_AR02_ancestral-seq- | Streptococcus salivarius | 1128 | 274 |
| recon_Streptococcus-salivarius_01 | | | |
| SsaCas9-sim1_WP_003097512.1_Streptococcus-vestibularis | Streptococcus vestibularis | 1128 | 275 |
| SsaCas9-sim1_WP_014634195.1_Streptococcus-salivarius | Streptococcus salivarius | 1127 | 276 |
| SsaCas9-sim1_WP_037611471.1_Streptococcus-sp-SR4 | Streptococcus sp SR4 | 1128 | 277 |
| SsaCas9-sim1_WP_048787825.1_Streptococcus | Streptococcus | 1122 | 278 |
| SsaCas9-sim1_WP_048790568.1_Streptococcus-salivarius | Streptococcus salivarius | 1139 | 279 |
| SsaCas9-sim1_WP_064519118.1_Streptococcus-vestibularis | Streptococcus vestibularis | 1128 | 280 |
| SsaCas9-sim1_WP_145516278.1_Streptococcus-salivarius | Streptococcus salivarius | 1121 | 281 |
| SsaCas9-sim1_WP_227278337.1_Streptococcus-vestibularis | Streptococcus vestibularis | 1122 | 282 |
| SsaCas9-sim2_clustered_ARC34060.1_Streptococcus- | Streptococcus equinus | 1128 | 283 |
| equinus | | | |
| SsaCas9-sim2_clustered_HEM3702072.1_Streptococcus-suis | Streptococcus suis | 1122 | 284 |
| SsaCas9-sim2_clustered_HEM4744366.1_Streptococcus-suis | Streptococcus suis | 1122 | 285 |
| SsaCas9-sim2_clustered_MDU6911040.1_Streptococcus- | Streptococcus salivarius | 1127 | 286 |
| salivarius | | | |
| SsaCas9-sim2_clustered_WP_002299171.1_Streptococcus- | Streptococcus mutans | 1125 | 287 |
| mutans | | | |
| SsaCas9-sim2_clustered_WP_021002704.1_Streptococcus- | Streptococcus intermedius | 1125 | 288 |
| intermedius | | | |
| SsaCas9-sim2_clustered_WP_084910940.1_Streptococcus- | Streptococcus oralis | 1129 | 289 |
| oralis | | | |
| SsaCas9-sim2_clustered_WP_115265868.1_Streptococcus- | Streptococcus macedonicus | 1130 | 290 |
| macedonicus | | | |
| SsaCas9-sim2_clustered_WP_126440388.1_Streptococcus- | Streptococcus equinus | 1130 | 291 |
| equinus | | | |
| SsaCas9-sim2_clustered_WP_155974233.1_Streptococcus- | Streptococcus ruminantium | 1125 | 292 |
| ruminantium | | | |
| SsaCas9-sim2_clustered_WP_198459777.1_Streptococcus- | Streptococcus oralis | 1122 | 293 |
| oralis | | | |
| SsaCas9-sim2_clustered_WP_219967735.1_Streptococcus- | Streptococcus gordonii | 1136 | 294 |
| gordonii | | | |
| SsaCas9-sim2_clustered_WP_223322622.1_Streptococcus- | Streptococcus sanguinis | 1121 | 295 |
| sanguinis | | | |
| SsaCas9-sim2_clustered_WP_257737268.1_Streptococcus- | Streptococcus uberis | 1122 | 296 |
| uberis | | | |
| SsaCas9-sim2_clustered_WP_268734931.1_Streptococcus- | Streptococcus cristatus | 1127 | 297 |
| cristatus | | | |
| SsaCas9-sim2_clustered_WP_270615522.1_Streptococcus- | Streptococcus koreensis | 1123 | 298 |
| koreensis | | | |
| SsaCas9-sim2_clustered_WP_281734300.1_Streptococcus- | Streptococcus lutetiensis | 1123 | 299 |
| lutetiensis | | | |
| SsaCas9-sim2_clustered_WP_311154762.1_uncultured- | Streptococcus sp | 1123 | 300 |
| Streptococcus-sp | | | |
| SsaCas9-sim3_Structure-Ssa-Streptococcus- | Streptococcus cristatus | 1130 | 301 |
| cristatus_A0A3R9LSK4_STRCR_Streptococcus-cristatus_01 | | | |
| SsaCas9-sim3_structure-Ssa-Streptococcus- | Streptococcus massiliensis | 1126 | 302 |
| massiliensis_A0A380KY87_9STRE_Streptococcus- | | | |
| massiliensis_01 | | | |
| SsaCas9-sim3_structure-Ssa-Streptococcus- | Streptococcus vestibularis | 1128 | 303 |
| vestibularis_A0A3S4QG93_Streptococcus-vestibularis_01 | | | |
| SsaCas9-sim3_structure-St1_A0A7X2QGI6_Streptococcus- | Streptococcus uberis | 1122 | 304 |
| uberis_01 | | | |
| SsaCas9-sim3_structure-St1-Enterococcus- | Enterococcus asini | 1123 | 305 |
| asini_A0A413AJC9_9ENTE_Enterococcus-asini_01 | | | |
| SsaCas9-sim3_structure-St1-Enterococcus- | Enterococcus asini | 1123 | 306 |
| asini_MCD5030292.1 | | | |
| SsaCas9-sim3_structure-St1-Enterococcus- | Enterococcus asini | 1123 | 307 |
| asini_RGW10367.1 | | | |
| SsaCas9-sim3_structure-St1-Enterococcus- | Enterococcus asini | 1130 | 308 |
| asini_WP_231453593.1 | | | |
| SsaCas9-sim3_structure-St1-Enterococcus- | Enterococcus asini | 1130 | 309 |
| asini_WP_259305198.1 | | | |
| SsaCas9-sim3_structure-St1-Enterococcus- | Enterococcus asini | 1131 | 310 |
| asini_WP_303220258.1 | | | |
| SsaCas9-sim3_structure-St1-Enterococcus- | Enterococcus asini | 1130 | 311 |
| asini_WP_311832475.1 | | | |
| SsaCas9-sim3_structure-St1-Enterococcus- | Enterococcus asini | 1130 | 312 |
| asini_WP_311835806.1 | | | |
| SsaCas9-sim3_structure-St1-Enterococcus- | Enterococcus asini | 1130 | 313 |
| asini_WP_311877876.1 | | | |
| SsaCas9-sim3_structure-St1-Enterococcus- | Enterococcus asini | 1130 | 314 |
| asini_WP_311896923.1 | | | |

TABLE 9A
EXEMPLARY mRNA SEQUENCES
RELATED TO RT EDITORS

| DESCRIPTION | SEQ ID NO:¹ |
|---|---|
| SegPolyA_1 | 182 |
| SegPolyA_2 | 183 |
| SegPolyA_3 | 184 |
| SegPolyA_4 | 185 |
| 120_PolyA | 186 |
| 130_PolyA | 187 |
| 3′UTR | 188 |
| WPRE | 189 |
| ek5 | 190 |
| STH | 191 |
| STH-2 | 192 |
| 5′UTR | 193 |
| RT editor mRNA sequence | 194 |
| 3′UTR-WPRE-SegPolyA_1 | 195 |
| 3′UTR-WPRE-SegPolyA_2 | 196 |
| 3′UTR-WPRE-SegPolyA_3 | 197 |
| 3′UTR-WPRE-SegPolyA_4 | 198 |
| 3′UTR-WPRE-120_PolyA | 199 |
| 3′UTR-WPRE-130_PolyA | 200 |
| 3′UTR-STH | 201 |
| 3′UTR-eK5-120_PolyA | 202 |
| 3′UTR-eK5-130_PolyA | 203 |
| 3′UTR-eK5-SegPolyA_1 | 204 |
| 3′UTR-eK5-SegPolyA_2 | 205 |
| 3′UTR-eK5-SegPolyA_3 | 206 |
| 3′UTR-eK5-SegPolyA_4 | 207 |
| eK5-120_PolyA | 208 |
| eK5-130_PolyA | 209 |
| eK5-SegPolyA_1 | 210 |
| eK5-SegPolyA_2 | 211 |
| eK5-SegPolyA_3 | 212 |
| eK5-SegPolyA_4 | 213 |
| 3′UTR-STH-120_PolyA | 214 |
| 3′UTR-STH-130_PolyA | 215 |
| 3′UTR-STH-SegPolyA_1 | 216 |
| 3′UTR-STH-SegPolyA_2 | 217 |
| 3′UTR-STH-SegPolyA_3 | 218 |
| 3′UTR-STH-SegPolyA_4 | 219 |
| RT_editor-3′UTR-WPRE-SegPolyA_1 | 220 |
| RT_editor-3′UTR-WPRE-SegPolyA_2 | 221 |
| RT_editor-3′UTR-WPRE-SegPolyA_3 | 222 |
| RT_editor-3′UTR-WPRE-SegPolyA_4 | 223 |
| RT_editor-3′UTR-WPRE-120_PolyA | 224 |
| RT_editor-3′UTR-WPRE-130_PolyA | 225 |
| RT_editor-3′UTR-STH | 226 |
| RT_editor-3′UTR-eK5-120_PolyA | 227 |
| RT_editor-3′UTR-eK5-130_PolyA | 228 |
| RT_editor-3′UTR-eK5-SegPolyA_1 | 229 |
| RT_editor-3′UTR-eK5-SegPolyA_2 | 230 |
| RT_editor-3′UTR-eK5-SegPolyA_3 | 231 |
| RT_editor-3′UTR-eK5-SegPolyA_4 | 232 |
| RT_editor-eK5-120_PolyA | 233 |
| RT_editor-eK5-130_PolyA | 234 |
| RT_editor-eK5-SegPolyA_1 | 235 |
| RT_editor-eK5-SegPolyA_2 | 236 |
| RT_editor-eK5-SegPolyA_3 | 237 |
| RT_editor-eK5-SegPolyA_4 | 238 |
| RT_editor-3′UTR-STH-120_PolyA | 239 |
| RT_editor-3′UTR-STH-130_PolyA | 240 |
| RT_editor-3′UTR-STH-SegPolyA_1 | 241 |
| RT_editor-3′UTR-STH-SegPolyA_2 | 242 |
| RT_editor-3′UTR-STH-SegPolyA_3 | 243 |
| RT_editor-3′UTR-STH-SegPolyA_4 | 244 |
| Comp14 transcript | 245 |
| Full-length segment from human | 246 |
| MALAT1 (HTH) | |

¹For RNA, T is U; U are T in the sequence listing.

TABLE 9B
EXEMPLARY mRNA Elements

| | | | SEQ |
|---|---|---|---|
| | Element | | ID |
| Name | Position | Sequence | NO: |
| SAA1 | 5UTR | AGGGACCCGCAGCTCAGCTACAGCACAGATCAGCACC | 940 |
| APOC3 | 5UTR | CTGCTCAGTTCATCCCTAGAGGCAGCTGCTCCAGGAACAGAGGT | 941 |
| | | GCC | |
| ALB | 5UTR | CTAGCTTTTCTCTTCTGTCAACCCCACACGCCTTTGGCACA | 942 |
| HP | 5UTR | ACTGGAAAAGATAGTGACCTTACCAGGGCCAAAGTTTGTAGAC | 943 |
| | | ACAGGAATTACGAAATGGAGAAGGGGGAGAAGTGAGCTAGTGG | |
| | | CAGCATAAAAAGACCAGCAGATGCCCCACAGCACTGCTCTTCCA | |
| | | GAGGCAAGACCAACCAAG | |
| ORM1 | 5UTR | AGCACTGCCTGGCTCCACGTGCCTCCTGGTCTCAGT | 944 |
| APOA2 | 5UTR | AGGCACAGACACCAAGGACAGAGACGCTGGCTAGGCCGCCCTC | 945 |
| | | CCCACTGTTACCAAC | |
| SAA2 | 5UTR | ACTATAAATAGCAGCCACCTCTCCCTGGCAGACAGGGACCCGCA | 946 |
| | | GCTCAGCTACAGCACAGATCAGCACC | |
| SERPINA1 | 5UTR | CTCCTCAGCTTCAGGCACCACCACTGACCTGGGACAGTGAATCG | 947 |
| | | ACA | |
| APOC1 | 5UTR | AGGCGGTCAGGGGAAGGCTCAGGAGGAGGGAGATCAACATCAA | 948 |
| | | CCTGCCCCGCCCCCTCCCCAGCCTGATAAAGGTCCTGCGGGCAG | |
| | | GACAGGACCTCCCAACCAAGCCCTCCAGCAAGGATTCAGAGTG | |
| | | CCCCTCCGGCCTCGCC | |
| Apt17_custom | 5UTR | ACTCACTATTTGTTTTCGCGCCCAGTTGCAAAAAGTGTCG | 949 |
| custom | 5UTR | AGGATAATATACTTACATACTTACTAATTAATACTAAACTCAAC | 950 |
| | | GCCACC | |
| Syn_5′UTR | 5UTR | AGGAAATAAGAGAGAAAAGAAGAGTAAGAAGAAATATAAGAG | 1075 |
| | | CCACC | |
| SAA1 | 3UTR | GCTTCCTCTTCACTCTGCTCTCAGGAGATCTGGCTGTGAGGCCCT | 951 |
| | | CAGGGCAGGGATACAAAGCGGGGAGAGGGTACACAATGGGTAT | |
| | | CTAATAAATACTTAAGAGGTGGAA | |
| APOC3 | 3UTR | GACCTCAATACCCCAAGTCCACCTGCCTATCCATCCTGCGAGCT | 952 |
| | | CCTTGGGTCCTGCAATCTCCAGGGCTGCCCCTGTAGGTTGCTTA | |
| | | AAAGGGACAGTATTCTCAGTGCTCTCCTACCCCACCTCATGCCT | |
| | | GGCCCCCCTCCAGGCATGCTGGCCTCCCAATAAAGCTGGACAAG | |
| | | AAGCTGCTATGA | |
| ALB | 3UTR | CATCACATTTAAAAGCATCTCAGCCTACCATGAGAATAAGAGAA | 953 |
| | | AGAAAATGAAGATCAAAAGCTTATTCATCTGTTTTTCTTTTTCGT | |
| | | TGGTGTAAAGCCAACACCCTGTCTAAAAAACATAAATTTCTTTA | |
| | | ATCATTTTGCCTCTTTTCTCTGTGCTTCAATTAATAAAAAATGGA | |
| | | AAGAATCTAATAGAGTGGTACAGCACTGTTATTTTTCAAAGATG | |
| | | TGTTGCTATCCTGAAAATTCTGTAGGTTCTGTGGAAGTTCCAGTG | |
| | | TTCTCTCTTATTCCACTTCGGTAGAGGATTTCTAGTTTCTTGTGG | |
| | | GCTAATTAAATAAATCATTAATACTCTTCTAAGTTATGGATTATA | |
| | | AACATTCAAAATAATATTTTGACATTATGATAATTCTGAATAAA | |
| | | AGAACAAAAACCA | |
| HP | 3UTR | TGCAAGGCTGGCCGGAAGCCCTTGCCTGAAAGCAAGATTTCAGC | 954 |
| | | CTGGAAGAGGGCAAAGTGGACGGGAGTGGACAGGAGTGGATGC | |
| | | GATAAGATGTGGTTTGAAGCTGATGGGTGCCAGCCCTGCATTGC | |
| | | TGAGTCAATCAATAAAGAGCTTTCTTTTGACCCA | |
| ORM1 | 3UTR | CAGGACACAGCCTTGGATCAGGACAGAGACTTGGGGGCCATCC | 955 |
| | | TGCCCCTCCAACCCGACATGTGTACCTCAGCTTTTTCCCTCACTT | |
| | | GCATCAATAAAGCTTCTGTGTTTGGAACAGCTAA | |
| APOA2 | 3UTR | AGTGTCCAGACCATTGTCTTCCAACCCCAGCTGGCCTCTAGAAC | 956 |
| | | ACCCACTGGCCAGTCCTAGAGCTCCTGTCCCTACCCACTCTTTGC | |
| | | TACAATAAATGCTGAATGAATCCA | |
| SAA2 | 3UTR | GCTTCCTCTTCACTCTGCTCTCAGGAGACCTGGCTATGAGGCCCT | 957 |
| | | CGGGGCAGGGATACAAAGTTAGTGAGGTCTATGTCCAGAGAAG | |
| | | CTGAGATATGGCATATAATAGGCATCTAATAAATGCTTAAGAGG | |
| | | TGGAA | |
| SERPINA1 | 3UTR | CTGCCTCTCGCTCCTCAACCCCTCCCCTCCATCCCTGGCCCCCTC | 958 |
| | | CCTGGATGACATTAAAGAAGGGTTGAGCTGGTCCCTGCCTGCAT | |
| | | GTGACTGTAAATCCCTCCCATGTTTTCTCTGAGTCTCCCTTTGCC | |
| | | TGCTGAGGCTGTATGTGGGCTCCAGGTAACAGTGCTGTCTTCGG | |
| | | GCCCCCTGAACTGTGTTCATGGAGCATCTGGCTGGGTAGGCACA | |
| | | TGCTGGGCTTGAATCCAGGGGGGACTGAATCCTCAGCTTACGGA | |
| | | CCTGGGCCCATCTGTTTCTGGAGGGCTCCAGTCTTCCTTGTCCTG | |
| | | TCTTGGAGTCCCCAAGAAGGAATCACAGGGGAGGAACCAGATA | |
| | | CCAGCCATGACCCCAGGCTCCACCAAGCATCTTCATGTCCCCCT | |
| | | GCTCATCCCCCACTCCCCCCCACCCAGAGTTGCTCATCCTGCCA | |
| | | GGGCTGGCTGTGCCCACCCCAAGGCTGCCCTCCTGGGGGCCCCA | |
| | | GAACTGCCTGATCGTGCCGTGGCCCAGTTTTGTGGCATCTGCAG | |
| | | CAACACAAGAGAGAGGACAATGTCCTCCTCTTGACCCGCTGTCA | |
| | | CCTAACCAGACTCGGGCCCTGCACCTCTCAGGCACTTCTGGAAA | |
| | | ATGACTGAGGCAGATTCTTCCTGAAGCCCATTCTCCATGGGGCA | |
| | | ACAAGGACACCTATTCTGTCCTTGTCCTTCCATCGCTGCCCCAGA | |
| | | AAGCCTCACATATCTCCGTTTAGAATCAGGTCCCTTCTCCCCAG | |
| | | ATGAAGAGGAGGGTCTCTGCTTTGTTTTCTCTATCTCCTCCTCAG | |
| | | ACTTGACCAGGCCCAGCAGGCCCCAGAAGACCATTACCCTATAT | |
| | | CCCTTCTCCTCCCTAGTCACATGGCCATAGGCCTGCTGATGGCTC | |
| | | AGGAAGGCCATTGCAAGGACTCCTCAGCTATGGGAGAGGAAGC | |
| | | ACATCACCCATTGACCCCCGCAACCCCTCCCTTTCCTCCTCTGAG | |
| | | TCCCGACTGGGGCCACATGCAGCCTGACTTCTTTGTGCCTGTTGC | |
| | | TGTCCCTGCAGTCTTCAGAGGGCCACCGCAGCTCCAGTGCCACG | |
| | | GCAGGAGGCTGTTCCTGAATAGCCCCTGTGGTAAGGGCCAGGA | |
| | | GAGTCCTTCCATCCTCCAAGGCCCTGCTAAAGGACACAGCAGCC | |
| | | AGGAAGTCCCCTGGGCCCCTAGCTGAAGGACAGCCTGCTCCCTC | |
| | | CGTCTCTACCAGGAATGGCCTTGTCCTATGGAAGGCACTGCCCC | |
| | | ATCCCAAACTAATCTAGGAATCACTGTCTAACCACTCACTGTCA | |
| | | TGAATGTGTACTTAAAGGATGAGGTTGAGTCATACCAAATAGTG | |
| | | ATTTCGATAGTTCAAAATGGTGAAATTAGCAATTCTACATGATT | |
| | | CAGTCTAATCAATGGATACCGACTGTTTCCCACACAAGTCTCCT | |
| | | GTTCTCTTAAGCTTACTCACTGACAGCCTTTCACTCTCCACAAAT | |
| | | ACATTAAAGATATGGCCATCACCAAGCCCCCTAGGATGACACCA | |
| | | GACCTGAGAGTCTGAAGACCTGGATCCAAGTTCTGACTTTTCCC | |
| | | CCTGACAGCTGTGTGACCTTCGTGAAGTCGCCAAACCTCTCTGA | |
| | | GCCCCAGTCATTGCTAGTAAGACCTGCCTTTGAGTTGGTATGAT | |
| | | GTTCAAGTTAGATAACAAAATGTTTATACCCATTAGAACAGAGA | |
| | | ATAAATAGAACTACATTTCTTGCA | |
| APOC1 | 3UTR | GGACCTGAAGGGTGACATCCCAGGAGGGGCCTCTGAAATTTCCC | 959 |
| | | ACACCCCAGCGCCTGTGCTGAGGACTCCCTCCATGTGGCCCCAG | |
| | | GTGCCACCAATAAAAATCCTACAGAAAA | |
| 3WJ1_custom | 3UTR | TTGCCATGTGTATGTGGGTTTTTTTTTTCCCACATACTCTGATGA | 960 |
| | | TCCTTTTTTTTTTGGATCATTCATGGCAA | |
| 3WJ2_custom | 3UTR | TTGCCATGTGTATGTGGGAAAAAAAAAACCCACATACTCTGATG | 961 |
| | | ATCCAAAAAAAAAAGGATCATTCATGGCAA | |
| HBB | 3UTR | gtgctggtctgtgtgctggcccatcactttggcaaagaattcaccccaccagtgcaggctgcctatcagaaa | 1076 |
| Extended | | gtggtggctggtgtggctaatgccctggcccacaagtatcactaagctcgctttcttgctgtccaatttctatta | |
| | | aaggttcctttgttccctaagtccaactactaaactgggggatattatgaagggccttgagcatctggattctg | |
| | | cctaataaaaaacatttattttcattgca | |
| hHBA | 3UTR | GCTGGAGCCTCGGTGGCCATGCTTCTTGCCCCTTGGGCCTCCCCC | 1077 |
| | | CAGCCCCTCCTCCCCTTCCTGCACCCGTACCCCCGTGGTCTTTGA | |
| | | ATAAAGTCTGAGTGGGCGGC | |
| mHBA | 3UTR | GCGGCCGCTTAATTAAGCTGCCTTCTGCGGGGCTTGCCTTCTGG | 1078 |
| | | CCATGCCCTTCTTCTCTCCCTTGCACCTGTACCTCTTGGTCTTTGA | |
| | | ATAAAGCCTGAGTAGGAAGTCTAG | |
| HBB_extended | 3UTR | GTGCTGGTCTGTGTGCTGGCCCATCACTTTGGCAAAGAATTCAC | 1079 |
| (HBBext) | | CCCACCAGTGCAGGCTGCCTATCAGAAAGTGGTGGCTGGTGTGG | |
| | | CTAATGCCCTGGCCCACAAGTATCACTAAGCTCGCTTTCTTGCTG | |
| | | TCCAATTTCTATTAAAGGTTCCTTTGTTCCCTAAGTCCAACTACT | |
| | | AAACTGGGGGATATTATGAAGGGCCTTGAGCATCTGGATTCTGC | |
| | | CTAATAAAAAACATTTATTTTCATTGCA | |
| AES-mtRNR1 | 3UTR | CTGGTACTGCATGCACGCAATGCTAGCTGCCCCTTTCCCGTCCTG | 1080 |
| | | GGTACCCCGAGTCTCCCCCGACCTCGGGTCCCAGGTATGCTCCC | |
| | | ACCTCCACCTGCCCCACTCACCACCTCTGCTAGTTCCAGACACCT | |
| | | CCCAAGCACGCAGCAATGCAGCTCAAAACGCTTAGCCTAGCCA | |
| | | CACCCCCACGGGAAACAGCAGTGATTAACCTTTAGCAATAAAC | |
| | | GAAAGTTTAACTAAGCTATACTAACCCCAGGGTTGGTCAATTTC | |
| | | GTGCCAGCCACACC | |
| EK5 | AE¹ | cactcctccatggtgatataaagaccacccacttccttcgggtgagcccctaagccatggttgtactgcacta | 962 |
| | | tcatcctaagacggtccttcttcggatcgcaatctcaccctggtgccgcgcttccttegggaactgcacccg | |
| | | cggaccagggccgtctttgaacttttctaactgttcttac | |
| STH | AE¹ | GTCATGAAGGTTTTTCTTTTCCTGAGAAAACAAgaaaTTGTTTTCT | 963 |
| | | CAGGTTTTGCTTTTTGAAAAAAAAGCAAAA | |

¹AE (additional elements) can be attached either downstream of the 3′-UTR or the poly A tail

Table 9C below additionally discloses 5′-UTR sequence variants wherein the difference in sequences are not within the UTR itself but in the second codon, as shown in the fourth column. For CESvar1, CESvar2, and CESvar3, ‘gcc’ was inserted in the second codon position. Column 4 shows the sequence of the first three codons.

TABLE 9C
EXEMPLARY mRNA Elements

| | Element | | First 3 | SEQ ID |
|---|---|---|---|---|
| Name | Position | Sequence of UTR | codons | NO |
| CES1 | 5UTR | agACAGAGACCTCGCAGGCCCCGAGAACTGTCGCCCT | ATGgccAAA | 1081 |
| var1 | | TCCACG | | |
| CES1 | 5UTR | agacagagacctcgcaggccccgagaactgtcgcccttcc | ATGgccAAA | 1082 |
| var2 | | acc | | |
| CES1 | 5UTR | agacagagacctcgcaggccccgagaactgtcgccctgcc | ATGgccAAA | 1083 |
| var3 | | acc | | |
| CES1 | 5UTR | AGACAGAGACCTCGCAGGCCCCGAGAACTGTCGCCC | ATG---AAA | 1084 |
| var4 | | TTCCACG | | |

Presented in Table 10 below are mRNA sequences of the components of exemplary RT editors comprising variant 3′UTR sequences. The components are listed from left-to-right in the 5′ to 3′ direction, using SEQ TD NOs to denote the sequence.

TABLE 10
EXEMPLARY 3′UTR VARIANTS

| | | | 3′UTR | 3′UTR | Structure | |
|---|---|---|---|---|---|---|
| | 5′ UTR | RT EDITOR | element#1 | element#2 | motif | Poly A |
| DESCRIPTION | SEQ ID NO: | SEQ ID NO: | SEQ ID NO: | SEQ ID NO: | SEQ ID NO: | SEQ ID NO: |
| RT_editor-3′UTR- | 193 | 194 | 188 | 189 | | 182 |
| WPRE- | | | | | | |
| SegPolyA_1 | | | | | | |
| (SEQ ID NO: 220) | | | | | | |
| RT_editor-3′UTR- | 193 | 194 | 188 | 189 | | 183 |
| WPRE- | | | | | | |
| SegPolyA_2 | | | | | | |
| (SEQ ID NO: 221) | | | | | | |
| RT editor-3′UTR- | 193 | 194 | 188 | 189 | | 184 |
| WPRE- | | | | | | |
| SegPolyA_3 | | | | | | |
| (SEQ ID NO: 222) | | | | | | |
| RT_editor-3′UTR- | 193 | 194 | 188 | 189 | | 185 |
| WPRE- | | | | | | |
| SegPolyA_4 | | | | | | |
| (SEQ ID NO: 223) | | | | | | |
| RT editor-3′UTR- | 193 | 194 | 188 | 189 | | 186 |
| WPRE- | | | | | | |
| 120_PolyA | | | | | | |
| (SEQ ID NO: 224) | | | | | | |
| RT editor-3′UTR- | 193 | 194 | 188 | 189 | | 187 |
| WPRE- | | | | | | |
| 130_Poly A | | | | | | |
| (SEQ ID NO: 225) | | | | | | |
| RT editor-3′UTR- | 193 | 194 | 188 | | 191 | |
| STH | | | | | | |
| (SEQ ID NO: 226) | | | | | | |
| RT_editor-3′UTR- | 193 | 194 | 188 | 190 | | 186 |
| eK5-120_PolyA | | | | | | |
| (SEQ ID NO: 227) | | | | | | |
| RT editor-3′UTR- | 193 | 194 | 188 | 190 | | 187 |
| eK5-130_PolyA | | | | | | |
| (SEQ ID NO: 228) | | | | | | |
| RT editor-3′UTR- | 193 | 194 | 188 | 190 | | 182 |
| eK5-SegPolyA_1 | | | | | | |
| (SEQ ID NO: 229) | | | | | | |
| RT editor-3′UTR- | 193 | 194 | 188 | 190 | | 183 |
| eK5-SegPolyA_2 | | | | | | |
| (SEQ ID NO: 230) | | | | | | |
| RT editor-3′UTR- | 193 | 194 | 188 | 190 | | 184 |
| eK5-SegPolyA_3 | | | | | | |
| (SEQ ID NO: 231) | | | | | | |
| RT editor-3′UTR- | 193 | 194 | 188 | 190 | | 185 |
| eK5-SegPolyA_4 | | | | | | |
| (SEQ ID NO: 232) | | | | | | |
| RT editor-eK5- | 193 | 194 | 190 | | | 186 |
| 120_Poly A | | | | | | |
| (SEQ ID NO: 233) | | | | | | |
| RT editor-eK5- | 193 | 194 | 190 | | | 187 |
| 130_Poly A | | | | | | |
| (SEQ ID NO: 234) | | | | | | |
| RT editor-eK5- | 193 | 194 | 190 | | | 182 |
| SegPoly A_1 | | | | | | |
| (SEQ ID NO: 235) | | | | | | |
| RT editor-eK5- | 193 | 194 | 190 | | | 183 |
| SegPolyA_2 | | | | | | |
| (SEQ ID NO: 236) | | | | | | |
| RT editor-eK5- | 193 | 194 | 190 | | | 184 |
| SegPolyA_3 | | | | | | |
| (SEQ ID NO: 237) | | | | | | |
| RT editor-eK5- | 193 | 194 | 190 | | | 185 |
| SegPolyA_4 | | | | | | |
| (SEQ ID NO: 238) | | | | | | |
| RT_editor-3′UTR- | 193 | 194 | 188 | | 192 | 186 |
| STH-120_PolyA | | | | | | |
| (SEQ ID NO: 239) | | | | | | |
| RT_editor-3′UTR- | 193 | 194 | 188 | | 192 | 187 |
| STH-130_PolyA | | | | | | |
| (SEQ ID NO: 240) | | | | | | |
| RT editor-3′UTR- | 193 | 194 | 188 | | 192 | 182 |
| STH-SegPolyA_1 | | | | | | |
| (SEQ ID NO: 241) | | | | | | |
| RT editor-3′UTR- | 193 | 194 | 188 | | 192 | 183 |
| STH-SegPolyA_2 | | | | | | |
| (SEQ ID NO: 242) | | | | | | |
| RT_editor-3′UTR- | 193 | 194 | 188 | | 192 | 184 |
| STH-SegPolyA_3 | | | | | | |
| (SEQ ID NO: 243) | | | | | | |
| RT editor-3′UTR- | 193 | 194 | 188 | | 192 | 185 |
| STH-SegPolyA_4 | | | | | | |
| (SEQ ID NO: 244) | | | | | | |

TABLE 11
EXEMPLARY REVERSE TRANSCRIPTASE VARIANTS

| NAME | SPECIES (native or derived) | Length | SEQ ID NO: |
|---|---|---|---|
| RT_RTBat-A_ARS01_RTEni-V-derived | Eptesicus nilssonii, bat | 672 | 250 |
| RT_RTBat-A_ARS01_With-RT5m_Prot-neg2 | Eptesicus nilssonii, bat | 668 | 251 |
| RT_RTEni_With-RT5m_Eptesicus- | Eptesicus nilssonii, bat | 679 | 252 |
| nilssonii | | | |
| RT_RTEni_With-RT5m_Eptesicus- | Eptesicus nilssonii, bat | 672 | 253 |
| nilssonii_within-prot | | | |
| RT_RTEni_With-RT5m_Eptesicus- | Eptesicus nilssonii, bat | 668 | 315 |
| nilssonii_Prot-neg2 | | | |
| RT_RTMbr-A_Myotis-brandtii | Myotis brandtii, bat | 673 | 254 |
| RT_RTMbr-A_Myotis-brandtii_With- | Myotis brandtii, bat | 669 | 255 |
| RT5m_Prot-neg2 | | | |
| RT_RTMcr-A | Melozone crissalis, bird | 741 | 256 |
| RT_RTMcr-A_Prot-neg2 | Melozone crissalis, bird | 675 | 257 |
| RT_RTMcr-A_Melozone-crissalis_Prot- | Melozone crissalis, bird | 675 | 316 |
| neg2_With-RT5m | | | |
| RT_RTMda-A | Myotis daubertonii, bat | 673 | 258 |
| RT_RTMda-A_Myotis-daubentonii_With- | Myotis daubentonii, bat | 669 | 259 |
| RT5m_Prot-neg2 | | | |
| RT_RTMge_With-RT5m | Melospiza georgiana, bird | 685 | 141 |
| RT_RTMge_Melospiza-georgiana_With- | Melospiza georgiana, bird | 675 | 260 |
| RT5m_Prot-neg2 | | | |
| RT_RTMge-A | Melospiza georgiana, bird | 713 | 261 |
| RT_RTMge-A_Prot-neg2 | Melospiza georgiana, bird | 671 | 262 |
| RT_RTMge-A_Prot-neg2_With-RT5m | Melospiza georgiana, bird | 671 | 317 |
| RT_RTMge-B | Melospiza georgiana, bird | 741 | 263 |
| RT_RTMge-C_var-of-B_Prot-neg2 | Melospiza georgiana, bird | 675 | 264 |
| RT_RTMge-C_var-of-B_With- | Melospiza georgiana, bird | 675 | 318 |
| RT5m_Prot-neg2 | | | |
| RT_RTMge-C_var-of-B_Within-prot- | Melospiza georgiana, bird | 679 | 265 |
| cleavage | | | |
| RT_RTPku-A_Pipistrellus-kuhlii | Pipistrellus kuhlii, bat | 673 | 266 |
| RT_RTPku-A_Pipistrellus-kuhlii_With- | Pipistrellus kuhlii, bat | 669 | 267 |
| RT5m_Prot-neg2 | | | |
| RT_RTZal-A | Zonotrichia albicollis, bird | 741 | 268 |
| RT_RTZal-A_Prot-neg2 | Zonotrichia albicollis, bird | 675 | 269 |
| RT_RTZal-A_Zonotrichia-albicollis_Prot- | Zonotrichia albicollis, bird | 675 | 319 |
| neg2_With-RT5m | | | |
| Klebsiella pneumonia mut¹ | Klebsiella pneumonia (added | 425 | 899 |
| | mutations E48K, S339K, | | |
| | Y395E) | | |
| Burkholderia cepacia mut¹ | Burkholderia cepacia (added | 423 | 900 |
| | mutations T47K, S87E, S338K, | | |
| | Y394E) | | |
| Caldibacillus thermoamylovorans | Caldibacillus | 442 | 901 |
| | thermoamylovorans | | |
| Caldibacillus thermoamylovorans mut¹ | Caldibacillus | 442 | 902 |
| | thermoamylovorans (added | | |
| | mutations T53K, S357K, | | |
| | Y408E) | | |
| Lysinibacillus sphaericus | Lysinibacillus sphaericus | 459 | 903 |
| Lysinibacillus sphaericus mut¹ | Lysinibacillus sphaericus | 459 | 904 |
| | (added mutations E63K, | | |
| | S374K, Y425E) | | |
| Bacillus sp. | Bacillus sp. | 438 | 905 |
| Bacillus sp. mut¹ | Bacillus sp. (added mutations | 1438 | 906 |
| | S339I, S351K, Y402E) | | |
| Truncated RT from Myotis brandtii | Myotis brandtii | 492 | 907 |
| Truncated RT from Pipistrellus kuhlii | Pipistrellus kuhlii | 492 | 908 |
| Truncated RT from MMLV | MMLV | 492 | 909 |
| pPG273_MMLV_protneg_RT | | 667 | 1015 |
| PG270_Woolly_monkey_RT | | 672 | 1016 |
| PG273_Melospiza_consensus_protneg_RT | | 675 | 1017 |
| PG273_Avian_protneg_RT | | 694 | 1018 |

TABLE 12A
EXEMPLARY tagRNA SEQUENCES FOR EDITING PAH

| NAME | SEQUENCE¹ | SEQ ID NO: |
|---|---|---|
| hPAH_NGG1_F15E6_ | UAGCGAACUGAGAAGGGCCAGUUUUAGAGCUAGAAAUAGCAAG | 320 |
| TtoC | UUAAAAUAAGGCUAGUCCGUUAUCAACUUGAAAAAGUGGCACCG | |
| | AGUCGGUGCCCUCGGCCCUUCUCAGUUCGC | |
| hPAH_NGG1_F14E6_ | UAGCGAACUGAGAAGGGCCAGUUUUAGAGCUAGAAAUAGCAAG | 321 |
| TtoC | UUAAAAUAAGGCUAGUCCGUUAUCAACUUGAAAAAGUGGCACCG | |
| | AGUCGGUGCCCUCGGCCCUUCUCAGUUCG | |
| hPAH_NGG1_F13E6_ | UAGCGAACUGAGAAGGGCCAGUUUUAGAGCUAGAAAUAGCAAG | 322 |
| TtoC | UUAAAAUAAGGCUAGUCCGUUAUCAACUUGAAAAAGUGGCACCG | |
| | AGUCGGUGCCCUCGGCCCUUCUCAGUUC | |
| hPAH_NGG1_F12E6_ | UAGCGAACUGAGAAGGGCCAGUUUUAGAGCUAGAAAUAGCAAG | 323 |
| TtoC | UUAAAAUAAGGCUAGUCCGUUAUCAACUUGAAAAAGUGGCACCG | |
| | AGUCGGUGCCCUCGGCCCUUCUCAGUU | |
| hPAH_NGG1_F11E6_ | UAGCGAACUGAGAAGGGCCAGUUUUAGAGCUAGAAAUAGCAAG | 324 |
| TtoC | UUAAAAUAAGGCUAGUCCGUUAUCAACUUGAAAAAGUGGCACCG | |
| | AGUCGGUGCCCUCGGCCCUUCUCAGU | |
| hPAH_NGG1_F10E6_ | UAGCGAACUGAGAAGGGCCAGUUUUAGAGCUAGAAAUAGCAAG | 325 |
| TtoC | UUAAAAUAAGGCUAGUCCGUUAUCAACUUGAAAAAGUGGCACCG | |
| | AGUCGGUGCCCUCGGCCCUUCUCAG | |
| hPAH_NGG1_F9E6_ | UAGCGAACUGAGAAGGGCCAGUUUUAGAGCUAGAAAUAGCAAG | 326 |
| TtoC | UUAAAAUAAGGCUAGUCCGUUAUCAACUUGAAAAAGUGGCACCG | |
| | AGUCGGUGCCCUCGGCCCUUCUCA | |
| hPAH_NGG1_F8E6_ | UAGCGAACUGAGAAGGGCCAGUUUUAGAGCUAGAAAUAGCAAG | 327 |
| TtoC | UUAAAAUAAGGCUAGUCCGUUAUCAACUUGAAAAAGUGGCACCG | |
| | AGUCGGUGCCCUCGGCCCUUCUC | |
| hPAH_NGG1_F7E6_ | UAGCGAACUGAGAAGGGCCAGUUUUAGAGCUAGAAAUAGCAAG | 328 |
| TtoC | UUAAAAUAAGGCUAGUCCGUUAUCAACUUGAAAAAGUGGCACCG | |
| | AGUCGGUGCCCUCGGCCCUUCU | |
| hPAH_NGG1_F15E8_ | UAGCGAACUGAGAAGGGCCAGUUUUAGAGCUAGAAAUAGCAAG | 329 |
| TtoC | UUAAAAUAAGGCUAGUCCGUUAUCAACUUGAAAAAGUGGCACCG | |
| | AGUCGGUGCUACCUCGGCCCUUCUCAGUUCGC | |
| hPAH_NGG1_F14E8_ | UAGCGAACUGAGAAGGGCCAGUUUUAGAGCUAGAAAUAGCAAG | 330 |
| TtoC | UUAAAAUAAGGCUAGUCCGUUAUCAACUUGAAAAAGUGGCACCG | |
| | AGUCGGUGCUACCUCGGCCCUUCUCAGUUCG | |
| hPAH_NGG1_F13E8_ | UAGCGAACUGAGAAGGGCCAGUUUUAGAGCUAGAAAUAGCAAG | 331 |
| TtoC | UUAAAAUAAGGCUAGUCCGUUAUCAACUUGAAAAAGUGGCACCG | |
| | AGUCGGUGCUACCUCGGCCCUUCUCAGUUC | |
| hPAH_NGG1_F12E8_ | UAGCGAACUGAGAAGGGCCAGUUUUAGAGCUAGAAAUAGCAAG | 332 |
| TtoC | UUAAAAUAAGGCUAGUCCGUUAUCAACUUGAAAAAGUGGCACCG | |
| | AGUCGGUGCUACCUCGGCCCUUCUCAGUU | |
| hPAH_NGG1_F11E8_ | UAGCGAACUGAGAAGGGCCAGUUUUAGAGCUAGAAAUAGCAAG | 333 |
| TtoC | UUAAAAUAAGGCUAGUCCGUUAUCAACUUGAAAAAGUGGCACCG | |
| | AGUCGGUGCUACCUCGGCCCUUCUCAGU | |
| hPAH_NGG1_F10E8_ | UAGCGAACUGAGAAGGGCCAGUUUUAGAGCUAGAAAUAGCAAG | 334 |
| TtoC | UUAAAAUAAGGCUAGUCCGUUAUCAACUUGAAAAAGUGGCACCG | |
| | AGUCGGUGCUACCUCGGCCCUUCUCAG | |
| hPAH_NGG1_F9E8_ | UAGCGAACUGAGAAGGGCCAGUUUUAGAGCUAGAAAUAGCAAG | 335 |
| TtoC | UUAAAAUAAGGCUAGUCCGUUAUCAACUUGAAAAAGUGGCACCG | |
| | AGUCGGUGCUACCUCGGCCCUUCUCA | |
| hPAH_NGG1_F8E8_ | UAGCGAACUGAGAAGGGCCAGUUUUAGAGCUAGAAAUAGCAAG | 336 |
| TtoC | UUAAAAUAAGGCUAGUCCGUUAUCAACUUGAAAAAGUGGCACCG | |
| | AGUCGGUGCUACCUCGGCCCUUCUC | |
| hPAH_NGG1_F7E8_ | UAGCGAACUGAGAAGGGCCAGUUUUAGAGCUAGAAAUAGCAAG | 337 |
| TtoC | UUAAAAUAAGGCUAGUCCGUUAUCAACUUGAAAAAGUGGCACCG | |
| | AGUCGGUGCUACCUCGGCCCUUCU | |
| hPAH_NGG1_F15E10_ | UAGCGAACUGAGAAGGGCCAGUUUUAGAGCUAGAAAUAGCAAG | 338 |
| TtoC | UUAAAAUAAGGCUAGUCCGUUAUCAACUUGAAAAAGUGGCACCG | |
| | AGUCGGUGCAAUACCUCGGCCCUUCUCAGUUCGC | |
| hPAH_NGG1_F14E10_ | UAGCGAACUGAGAAGGGCCAGUUUUAGAGCUAGAAAUAGCAAG | 339 |
| TtoC | UUAAAAUAAGGCUAGUCCGUUAUCAACUUGAAAAAGUGGCACCG | |
| | AGUCGGUGCAAUACCUCGGCCCUUCUCAGUUCG | |
| hPAH_NGG1_F13E10_ | UAGCGAACUGAGAAGGGCCAGUUUUAGAGCUAGAAAUAGCAAG | 340 |
| TtoC | UUAAAAUAAGGCUAGUCCGUUAUCAACUUGAAAAAGUGGCACCG | |
| | AGUCGGUGCAAUACCUCGGCCCUUCUCAGUUC | |
| hPAH_NGG1_F12E10_ | UAGCGAACUGAGAAGGGCCAGUUUUAGAGCUAGAAAUAGCAAG | 341 |
| TtoC | UUAAAAUAAGGCUAGUCCGUUAUCAACUUGAAAAAGUGGCACCG | |
| | AGUCGGUGCAAUACCUCGGCCCUUCUCAGUU | |
| hPAH_NGG1_F11E10_ | UAGCGAACUGAGAAGGGCCAGUUUUAGAGCUAGAAAUAGCAAG | 342 |
| TtoC | UUAAAAUAAGGCUAGUCCGUUAUCAACUUGAAAAAGUGGCACCG | |
| | AGUCGGUGCAAUACCUCGGCCCUUCUCAGU | |
| hPAH_NGG1_F10E10_ | UAGCGAACUGAGAAGGGCCAGUUUUAGAGCUAGAAAUAGCAAG | 343 |
| TtoC | UUAAAAUAAGGCUAGUCCGUUAUCAACUUGAAAAAGUGGCACCG | |
| | AGUCGGUGCAAUACCUCGGCCCUUCUCAG | |
| hPAH_NGG1_F9E10 | UAGCGAACUGAGAAGGGCCAGUUUUAGAGCUAGAAAUAGCAAG | 344 |
| TtoC | UUAAAAUAAGGCUAGUCCGUUAUCAACUUGAAAAAGUGGCACCG | |
| | AGUCGGUGCAAUACCUCGGCCCUUCUCA | |
| hPAH_NGG1_F8E10_ | UAGCGAACUGAGAAGGGCCAGUUUUAGAGCUAGAAAUAGCAAG | 345 |
| TtoC | UUAAAAUAAGGCUAGUCCGUUAUCAACUUGAAAAAGUGGCACCG | |
| | AGUCGGUGCAAUACCUCGGCCCUUCUC | |
| hPAH_NGG1_F7E10_ | UAGCGAACUGAGAAGGGCCAGUUUUAGAGCUAGAAAUAGCAAG | 346 |
| TtoC | UUAAAAUAAGGCUAGUCCGUUAUCAACUUGAAAAAGUGGCACCG | |
| | AGUCGGUGCAAUACCUCGGCCCUUCU | |
| hPAH_NGG1_F14E12_ | UAGCGAACUGAGAAGGGCCAGUUUUAGAGCUAGAAAUAGCAAG | 347 |
| TtoC | UUAAAAUAAGGCUAGUCCGUUAUCAACUUGAAAAAGUGGCACCG | |
| | AGUCGGUGCACAAUACCUCGGCCCUUCUCAGUUCG | |
| hPAH_NGG1_F13E12_ | UAGCGAACUGAGAAGGGCCAGUUUUAGAGCUAGAAAUAGCAAG | 348 |
| TtoC | UUAAAAUAAGGCUAGUCCGUUAUCAACUUGAAAAAGUGGCACCG | |
| | AGUCGGUGCACAAUACCUCGGCCCUUCUCAGUUC | |
| hPAH_NGG1_F12E12_ | UAGCGAACUGAGAAGGGCCAGUUUUAGAGCUAGAAAUAGCAAG | 349 |
| TtoC | UUAAAAUAAGGCUAGUCCGUUAUCAACUUGAAAAAGUGGCACCG | |
| | AGUCGGUGCACAAUACCUCGGCCCUUCUCAGUU | |
| hPAH_NGG1_F11E12_ | UAGCGAACUGAGAAGGGCCAGUUUUAGAGCUAGAAAUAGCAAG | 350 |
| TtoC | UUAAAAUAAGGCUAGUCCGUUAUCAACUUGAAAAAGUGGCACCG | |
| | AGUCGGUGCACAAUACCUCGGCCCUUCUCAGU | |
| hPAH_NGG1_F10E12_ | UAGCGAACUGAGAAGGGCCAGUUUUAGAGCUAGAAAUAGCAAG | 351 |
| TtoC | UUAAAAUAAGGCUAGUCCGUUAUCAACUUGAAAAAGUGGCACCG | |
| | AGUCGGUGCACAAUACCUCGGCCCUUCUCAG | |
| hPAH_NGG1_F9E12_ | UAGCGAACUGAGAAGGGCCAGUUUUAGAGCUAGAAAUAGCAAG | 352 |
| TtoC | UUAAAAUAAGGCUAGUCCGUUAUCAACUUGAAAAAGUGGCACCG | |
| | AGUCGGUGCACAAUACCUCGGCCCUUCUCA | |
| hPAH_NGG1_F8E12_ | UAGCGAACUGAGAAGGGCCAGUUUUAGAGCUAGAAAUAGCAAG | 353 |
| TtoC | UUAAAAUAAGGCUAGUCCGUUAUCAACUUGAAAAAGUGGCACCG | |
| | AGUCGGUGCACAAUACCUCGGCCCUUCUC | |
| hPAH_NGG1_F7E12_ | UAGCGAACUGAGAAGGGCCAGUUUUAGAGCUAGAAAUAGCAAG | 354 |
| TtoC | UUAAAAUAAGGCUAGUCCGUUAUCAACUUGAAAAAGUGGCACCG | |
| | AGUCGGUGCACAAUACCUCGGCCCUUCU | |
| hPAH_NGG1_F15E14_ | UAGCGAACUGAGAAGGGCCAGUUUUAGAGCUAGAAAUAGCAAG | 355 |
| TtoC | UUAAAAUAAGGCUAGUCCGUUAUCAACUUGAAAAAGUGGCACCG | |
| | AGUCGGUGCCCACAAUACCUCGGCCCUUCUCAGUUCGC | |
| hPAH_NGG1_F14E14_ | UAGCGAACUGAGAAGGGCCAGUUUUAGAGCUAGAAAUAGCAAG | 356 |
| TtoC | UUAAAAUAAGGCUAGUCCGUUAUCAACUUGAAAAAGUGGCACCG | |
| | AGUCGGUGCCCACAAUACCUCGGCCCUUCUCAGUUCG | |
| hPAH_NGG1_F13E14_ | UAGCGAACUGAGAAGGGCCAGUUUUAGAGCUAGAAAUAGCAAG | 357 |
| TtoC | UUAAAAUAAGGCUAGUCCGUUAUCAACUUGAAAAAGUGGCACCG | |
| | AGUCGGUGCCCACAAUACCUCGGCCCUUCUCAGUUC | |
| hPAH_NGG1_F12E14_ | UAGCGAACUGAGAAGGGCCAGUUUUAGAGCUAGAAAUAGCAAG | 358 |
| TtoC | UUAAAAUAAGGCUAGUCCGUUAUCAACUUGAAAAAGUGGCACCG | |
| | AGUCGGUGCCCACAAUACCUCGGCCCUUCUCAGUU | |
| hPAH_NGG1_F11E14_ | UAGCGAACUGAGAAGGGCCAGUUUUAGAGCUAGAAAUAGCAAG | 359 |
| TtoC | UUAAAAUAAGGCUAGUCCGUUAUCAACUUGAAAAAGUGGCACCG | |
| | AGUCGGUGCCCACAAUACCUCGGCCCUUCUCAGU | |
| hPAH_NGG1_F10E14_ | UAGCGAACUGAGAAGGGCCAGUUUUAGAGCUAGAAAUAGCAAG | 360 |
| TtoC | UUAAAAUAAGGCUAGUCCGUUAUCAACUUGAAAAAGUGGCACCG | |
| | AGUCGGUGCCCACAAUACCUCGGCCCUUCUCAG | |
| hPAH_NGG1_F9E14_ | UAGCGAACUGAGAAGGGCCAGUUUUAGAGCUAGAAAUAGCAAG | 361 |
| TtoC | UUAAAAUAAGGCUAGUCCGUUAUCAACUUGAAAAAGUGGCACCG | |
| | AGUCGGUGCCCACAAUACCUCGGCCCUUCUCA | |
| hPAH_NGG1_F8E14_ | UAGCGAACUGAGAAGGGCCAGUUUUAGAGCUAGAAAUAGCAAG | 362 |
| TtoC | UUAAAAUAAGGCUAGUCCGUUAUCAACUUGAAAAAGUGGCACCG | |
| | AGUCGGUGCCCACAAUACCUCGGCCCUUCUC | |
| hPAH_NGG1_F7E14_ | UAGCGAACUGAGAAGGGCCAGUUUUAGAGCUAGAAAUAGCAAG | 363 |
| TtoC | UUAAAAUAAGGCUAGUCCGUUAUCAACUUGAAAAAGUGGCACCG | |
| | AGUCGGUGCCCACAAUACCUCGGCCCUUCU | |
| hPAH_NGG2_F15E7_ | ACUUUGCUGCCACAAUACCUGUUUUAGAGCUAGAAAUAGCAAGU | 364 |
| AtoG | UAAAAUAAGGCUAGUCCGUUAUCAACUUGAAAAAGUGGCACCGA | |
| | GUCGGUGCGCCGAGGUAUUGUGGCAGCAAA | |
| hPAH_NGG2_F14E7_ | ACUUUGCUGCCACAAUACCUGUUUUAGAGCUAGAAAUAGCAAGU | 365 |
| AtoG | UAAAAUAAGGCUAGUCCGUUAUCAACUUGAAAAAGUGGCACCGA | |
| | GUCGGUGCGCCGAGGUAUUGUGGCAGCAA | |
| hPAH_NGG2_F13E7_ | ACUUUGCUGCCACAAUACCUGUUUUAGAGCUAGAAAUAGCAAGU | 366 |
| AtoG | UAAAAUAAGGCUAGUCCGUUAUCAACUUGAAAAAGUGGCACCGA | |
| | GUCGGUGCGCCGAGGUAUUGUGGCAGCA | |
| hPAH_NGG2_F12E7_ | ACUUUGCUGCCACAAUACCUGUUUUAGAGCUAGAAAUAGCAAGU | 367 |
| AtoG | UAAAAUAAGGCUAGUCCGUUAUCAACUUGAAAAAGUGGCACCGA | |
| | GUCGGUGCGCCGAGGUAUUGUGGCAGC | |
| hPAH_NGG2_F11E7_ | ACUUUGCUGCCACAAUACCUGUUUUAGAGCUAGAAAUAGCAAGU | 368 |
| AtoG | UAAAAUAAGGCUAGUCCGUUAUCAACUUGAAAAAGUGGCACCGA | |
| | GUCGGUGCGCCGAGGUAUUGUGGCAG | |
| hPAH_NGG2_F10E7_ | ACUUUGCUGCCACAAUACCUGUUUUAGAGCUAGAAAUAGCAAGU | 369 |
| AtoG | UAAAAUAAGGCUAGUCCGUUAUCAACUUGAAAAAGUGGCACCGA | |
| | GUCGGUGCGCCGAGGUAUUGUGGCA | |
| hPAH_NGG2_F9E7_ | ACUUUGCUGCCACAAUACCUGUUUUAGAGCUAGAAAUAGCAAGU | 370 |
| AtoG | UAAAAUAAGGCUAGUCCGUUAUCAACUUGAAAAAGUGGCACCGA | |
| | GUCGGUGCGCCGAGGUAUUGUGGC | |
| hPAH_NGG2_F8E7_ | ACUUUGCUGCCACAAUACCUGUUUUAGAGCUAGAAAUAGCAAGU | 371 |
| AtoG | UAAAAUAAGGCUAGUCCGUUAUCAACUUGAAAAAGUGGCACCGA | |
| | GUCGGUGCGCCGAGGUAUUGUGG | |
| hPAH_NGG2_F7E7_ | ACUUUGCUGCCACAAUACCUGUUUUAGAGCUAGAAAUAGCAAGU | 372 |
| AtoG | UAAAAUAAGGCUAGUCCGUUAUCAACUUGAAAAAGUGGCACCGA | |
| | GUCGGUGCGCCGAGGUAUUGUG | |
| hPAH_NGG2_F15E9_ | ACUUUGCUGCCACAAUACCUGUUUUAGAGCUAGAAAUAGCAAGU | 373 |
| AtoG | UAAAAUAAGGCUAGUCCGUUAUCAACUUGAAAAAGUGGCACCGA | |
| | GUCGGUGCGGGCCGAGGUAUUGUGGCAGCAAA | |
| hPAH_NGG2_F14E9_ | ACUUUGCUGCCACAAUACCUGUUUUAGAGCUAGAAAUAGCAAGU | 374 |
| AtoG | UAAAAUAAGGCUAGUCCGUUAUCAACUUGAAAAAGUGGCACCGA | |
| | GUCGGUGCGGGCCGAGGUAUUGUGGCAGCAA | |
| hPAH_NGG2_F13E9_ | ACUUUGCUGCCACAAUACCUGUUUUAGAGCUAGAAAUAGCAAGU | 375 |
| AtoG | UAAAAUAAGGCUAGUCCGUUAUCAACUUGAAAAAGUGGCACCGA | |
| | GUCGGUGCGGGCCGAGGUAUUGUGGCAGCA | |
| hPAH_NGG2_F12E9_ | ACUUUGCUGCCACAAUACCUGUUUUAGAGCUAGAAAUAGCAAGU | 376 |
| AtoG | UAAAAUAAGGCUAGUCCGUUAUCAACUUGAAAAAGUGGCACCGA | |
| | GUCGGUGCGGGCCGAGGUAUUGUGGCAGC | |
| hPAH_NGG2_F11E9_ | ACUUUGCUGCCACAAUACCUGUUUUAGAGCUAGAAAUAGCAAGU | 377 |
| AtoG | UAAAAUAAGGCUAGUCCGUUAUCAACUUGAAAAAGUGGCACCGA | |
| | GUCGGUGCGGGCCGAGGUAUUGUGGCAG | |
| hPAH_NGG2_F10E9_ | ACUUUGCUGCCACAAUACCUGUUUUAGAGCUAGAAAUAGCAAGU | 378 |
| AtoG | UAAAAUAAGGCUAGUCCGUUAUCAACUUGAAAAAGUGGCACCGA | |
| | GUCGGUGCGGGCCGAGGUAUUGUGGCA | |
| hPAH_NGG2_F9E9_ | ACUUUGCUGCCACAAUACCUGUUUUAGAGCUAGAAAUAGCAAGU | 379 |
| AtoG | UAAAAUAAGGCUAGUCCGUUAUCAACUUGAAAAAGUGGCACCGA | |
| | GUCGGUGCGGGCCGAGGUAUUGUGGC | |
| hPAH_NGG2_F8E9_ | ACUUUGCUGCCACAAUACCUGUUUUAGAGCUAGAAAUAGCAAGU | 380 |
| AtoG | UAAAAUAAGGCUAGUCCGUUAUCAACUUGAAAAAGUGGCACCGA | |
| | GUCGGUGCGGGCCGAGGUAUUGUGG | |
| hPAH_NGG2_F7E9_ | ACUUUGCUGCCACAAUACCUGUUUUAGAGCUAGAAAUAGCAAGU | 381 |
| AtoG | UAAAAUAAGGCUAGUCCGUUAUCAACUUGAAAAAGUGGCACCGA | |
| | GUCGGUGCGGGCCGAGGUAUUGUG | |
| hPAH_NGG2_F15E11_ | ACUUUGCUGCCACAAUACCUGUUUUAGAGCUAGAAAUAGCAAGU | 382 |
| AtoG | UAAAAUAAGGCUAGUCCGUUAUCAACUUGAAAAAGUGGCACCGA | |
| | GUCGGUGCAAGGGCCGAGGUAUUGUGGCAGCAAA | |
| hPAH_NGG2_F14E11_ | ACUUUGCUGCCACAAUACCUGUUUUAGAGCUAGAAAUAGCAAGU | 383 |
| AtoG | UAAAAUAAGGCUAGUCCGUUAUCAACUUGAAAAAGUGGCACCGA | |
| | GUCGGUGCAAGGGCCGAGGUAUUGUGGCAGCAA | |
| hPAH_NGG2_F13E11_ | ACUUUGCUGCCACAAUACCUGUUUUAGAGCUAGAAAUAGCAAGU | 384 |
| AtoG | UAAAAUAAGGCUAGUCCGUUAUCAACUUGAAAAAGUGGCACCGA | |
| | GUCGGUGCAAGGGCCGAGGUAUUGUGGCAGCA | |
| hPAH_NGG2_F12E11_ | ACUUUGCUGCCACAAUACCUGUUUUAGAGCUAGAAAUAGCAAGU | 385 |
| AtoG | UAAAAUAAGGCUAGUCCGUUAUCAACUUGAAAAAGUGGCACCGA | |
| | GUCGGUGCAAGGGCCGAGGUAUUGUGGCAGC | |
| hPAH_NGG2_F11E11_ | ACUUUGCUGCCACAAUACCUGUUUUAGAGCUAGAAAUAGCAAGU | 386 |
| AtoG | UAAAAUAAGGCUAGUCCGUUAUCAACUUGAAAAAGUGGCACCGA | |
| | GUCGGUGCAAGGGCCGAGGUAUUGUGGCAG | |
| hPAH_NGG2_F10E11_ | ACUUUGCUGCCACAAUACCUGUUUUAGAGCUAGAAAUAGCAAGU | 387 |
| AtoG | UAAAAUAAGGCUAGUCCGUUAUCAACUUGAAAAAGUGGCACCGA | |
| | GUCGGUGCAAGGGCCGAGGUAUUGUGGCA | |
| hPAH_NGG2_F9E11_ | ACUUUGCUGCCACAAUACCUGUUUUAGAGCUAGAAAUAGCAAGU | 388 |
| AtoG | UAAAAUAAGGCUAGUCCGUUAUCAACUUGAAAAAGUGGCACCGA | |
| | GUCGGUGCAAGGGCCGAGGUAUUGUGGC | |
| hPAH_NGG2_F8E11_ | ACUUUGCUGCCACAAUACCUGUUUUAGAGCUAGAAAUAGCAAGU | 389 |
| AtoG | UAAAAUAAGGCUAGUCCGUUAUCAACUUGAAAAAGUGGCACCGA | |
| | GUCGGUGCAAGGGCCGAGGUAUUGUGG | |
| hPAH_NGG2_F7E11_ | ACUUUGCUGCCACAAUACCUGUUUUAGAGCUAGAAAUAGCAAGU | 390 |
| AtoG | UAAAAUAAGGCUAGUCCGUUAUCAACUUGAAAAAGUGGCACCGA | |
| | GUCGGUGCAAGGGCCGAGGUAUUGUG | |
| hPAH_NGG2_F15E13_ | ACUUUGCUGCCACAAUACCUGUUUUAGAGCUAGAAAUAGCAAGU | 391 |
| AtoG | UAAAAUAAGGCUAGUCCGUUAUCAACUUGAAAAAGUGGCACCGA | |
| | GUCGGUGCAGAAGGGCCGAGGUAUUGUGGCAGCAAA | |
| hPAH_NGG2_F14E13_ | ACUUUGCUGCCACAAUACCUGUUUUAGAGCUAGAAAUAGCAAGU | 392 |
| AtoG | UAAAAUAAGGCUAGUCCGUUAUCAACUUGAAAAAGUGGCACCGA | |
| | GUCGGUGCAGAAGGGCCGAGGUAUUGUGGCAGCAA | |
| hPAH_NGG2_F13E13_ | ACUUUGCUGCCACAAUACCUGUUUUAGAGCUAGAAAUAGCAAGU | 393 |
| AtoG | UAAAAUAAGGCUAGUCCGUUAUCAACUUGAAAAAGUGGCACCGA | |
| | GUCGGUGCAGAAGGGCCGAGGUAUUGUGGCAGCA | |
| hPAH_NGG2_F12E13_ | ACUUUGCUGCCACAAUACCUGUUUUAGAGCUAGAAAUAGCAAGU | 394 |
| AtoG | UAAAAUAAGGCUAGUCCGUUAUCAACUUGAAAAAGUGGCACCGA | |
| | GUCGGUGCAGAAGGGCCGAGGUAUUGUGGCAGC | |
| hPAH_NGG2_F11E13_ | ACUUUGCUGCCACAAUACCUGUUUUAGAGCUAGAAAUAGCAAGU | 395 |
| AtoG | UAAAAUAAGGCUAGUCCGUUAUCAACUUGAAAAAGUGGCACCGA | |
| | GUCGGUGCAGAAGGGCCGAGGUAUUGUGGCAG | |
| hPAH_NGG2_F10E13_ | ACUUUGCUGCCACAAUACCUGUUUUAGAGCUAGAAAUAGCAAGU | 396 |
| AtoG | UAAAAUAAGGCUAGUCCGUUAUCAACUUGAAAAAGUGGCACCGA | |
| | GUCGGUGCAGAAGGGCCGAGGUAUUGUGGCA | |
| hPAH_NGG2_F9E13_ | ACUUUGCUGCCACAAUACCUGUUUUAGAGCUAGAAAUAGCAAGU | 397 |
| AtoG | UAAAAUAAGGCUAGUCCGUUAUCAACUUGAAAAAGUGGCACCGA | |
| | GUCGGUGCAGAAGGGCCGAGGUAUUGUGGC | |
| hPAH_NGG2_F8E13_ | ACUUUGCUGCCACAAUACCUGUUUUAGAGCUAGAAAUAGCAAGU | 398 |
| AtoG | UAAAAUAAGGCUAGUCCGUUAUCAACUUGAAAAAGUGGCACCGA | |
| | GUCGGUGCAGAAGGGCCGAGGUAUUGUGG | |
| hPAH_NGG2_F7E13_ | ACUUUGCUGCCACAAUACCUGUUUUAGAGCUAGAAAUAGCAAGU | 399 |
| AtoG | UAAAAUAAGGCUAGUCCGUUAUCAACUUGAAAAAGUGGCACCGA | |
| | GUCGGUGCAGAAGGGCCGAGGUAUUGUG | |
| hPAH_NGG2_F15E15_ | ACUUUGCUGCCACAAUACCUGUUUUAGAGCUAGAAAUAGCAAGU | 400 |
| AtoG | UAAAAUAAGGCUAGUCCGUUAUCAACUUGAAAAAGUGGCACCGA | |
| | GUCGGUGCUGAGAAGGGCCGAGGUAUUGUGGCAGCAAA | |
| hPAH_NGG2_F14E15_ | ACUUUGCUGCCACAAUACCUGUUUUAGAGCUAGAAAUAGCAAGU | 401 |
| AtoG | UAAAAUAAGGCUAGUCCGUUAUCAACUUGAAAAAGUGGCACCGA | |
| | GUCGGUGCUGAGAAGGGCCGAGGUAUUGUGGCAGCAA | |
| hPAH_NGG2_F13E15_ | ACUUUGCUGCCACAAUACCUGUUUUAGAGCUAGAAAUAGCAAGU | 402 |
| AtoG | UAAAAUAAGGCUAGUCCGUUAUCAACUUGAAAAAGUGGCACCGA | |
| | GUCGGUGCUGAGAAGGGCCGAGGUAUUGUGGCAGCA | |
| hPAH_NGG2_F12E15_ | ACUUUGCUGCCACAAUACCUGUUUUAGAGCUAGAAAUAGCAAGU | 403 |
| AtoG | UAAAAUAAGGCUAGUCCGUUAUCAACUUGAAAAAGUGGCACCGA | |
| | GUCGGUGCUGAGAAGGGCCGAGGUAUUGUGGCAGC | |
| hPAH_NGG2_F11E15_ | ACUUUGCUGCCACAAUACCUGUUUUAGAGCUAGAAAUAGCAAGU | 404 |
| AtoG | UAAAAUAAGGCUAGUCCGUUAUCAACUUGAAAAAGUGGCACCGA | |
| | GUCGGUGCUGAGAAGGGCCGAGGUAUUGUGGCAG | |
| hPAH_NGG2_F10E15_ | ACUUUGCUGCCACAAUACCUGUUUUAGAGCUAGAAAUAGCAAGU | 405 |
| AtoG | UAAAAUAAGGCUAGUCCGUUAUCAACUUGAAAAAGUGGCACCGA | |
| | GUCGGUGCUGAGAAGGGCCGAGGUAUUGUGGCA | |
| hPAH_NGG2_F9E15_ | ACUUUGCUGCCACAAUACCUGUUUUAGAGCUAGAAAUAGCAAGU | 406 |
| AtoG | UAAAAUAAGGCUAGUCCGUUAUCAACUUGAAAAAGUGGCACCGA | |
| | GUCGGUGCUGAGAAGGGCCGAGGUAUUGUGGC | |
| hPAH_NGG2_F8E15_ | ACUUUGCUGCCACAAUACCUGUUUUAGAGCUAGAAAUAGCAAGU | 407 |
| AtoG | UAAAAUAAGGCUAGUCCGUUAUCAACUUGAAAAAGUGGCACCGA | |
| | GUCGGUGCUGAGAAGGGCCGAGGUAUUGUGG | |
| hPAH_NGG2_F7E15_ | ACUUUGCUGCCACAAUACCUGUUUUAGAGCUAGAAAUAGCAAGU | 408 |
| AtoG | UAAAAUAAGGCUAGUCCGUUAUCAACUUGAAAAAGUGGCACCGA | |
| | GUCGGUGCUGAGAAGGGCCGAGGUAUUGUG | |

¹For RNA, T is U; U are T in the sequence listing. For each entry, the spacer sequence is underlined and the extension arm is bold. The description indicates the length of the FBS (F) and ET (E) or homology region of the extension arm.

TABLE 12B
EXEMPLARY MODIFIED tagRNA SEQUENCES FOR EDITING PAH

| NAME | SEQUENCE^{1,2} | SEQ ID NO: |
|---|---|---|
| hPAH_NGG1_F15E6_ | mU*mA*mG*rCrGrArArCrUrGrArGrArArGrGrGrCrCrArGrUrUrUrUrAr | 409 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrUrCrGrGrCrCrCrUrUrCrUrCrArGrUrU*mC*mG*mC | |
| hPAH_NGG1_F14E6_ | mU*mA*mG*rCrGrArArCrUrGrArGrArArGrGrGrCrCrArGrUrUrUrUrAr | 410 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrUrCrGrGrCrCrCrUrUrCrUrCrArGrU*mU*mC*mG | |
| hPAH_NGG1_F13E6_ | mU*mA*mG*rCrGrArArCrUrGrArGrArArGrGrGrCrCrArGrUrUrUrUrAr | 411 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrUrCrGrGrCrCrCrUrUrCrUrCrArG*mU*mU*mC | |
| hPAH_NGG1_F12E6_ | mU*mA*mG*rCrGrArArCrUrGrArGrArArGrGrGrCrCrArGrUrUrUrUrAr | 412 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrUrCrGrGrCrCrCrUrUrCrUrCrA*mG*mU*mU | |
| hPAH_NGG1_F11E6_ | mU*mA*mG*rCrGrArArCrUrGrArGrArArGrGrGrCrCrArGrUrUrUrUrAr | 413 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrUrCrGrGrCrCrCrUrUrCrUrC*mA*mG*mU | |
| hPAH_NGG1_F10R6_ | mU*mA*mG*rCrGrArArCrUrGrArGrArArGrGrGrCrCrArGrUrUrUrUrAr | 414 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrUrCrGrGrCrCrCrUrUrCrU*mC*mA*mG | |
| hPAH_NGG1_F9E6_ | mU*mA*mG*rCrGrArArCrUrGrArGrArArGrGrGrCrCrArGrUrUrUrUrAr | 415 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrUrCrGrGrCrCrCrUrUrC*mU*mC*mA | |
| hPAH_NGG1_F8E6_ | mU*mA*mG*rCrGrArArCrUrGrArGrArArGrGrGrCrCrArGrUrUrUrUrAr | 416 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrUrCrGrGrCrCrCrUrU*mC*mU*mC | |
| hPAH_NGG1_F7R6_ | mU*mA*mG*rCrGrArArCrUrGrArGrArArGrGrGrCrCrArGrUrUrUrUrAr | 417 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrUrCrGrGrCrCrCrU*mU*mC*mU | |
| hPAH_NGG1_F15E8_ | mU*mA*mG*rCrGrArArCrUrGrArGrArArGrGrGrCrCrArGrUrUrUrUrAr | 418 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrU | |
| | rArCrCrUrCrGrGrCrCrCrUrUrCrUrCrArGrUrU*mC*mG*mC | |
| hPAH_NGG1_F14E8_ | mU*mA*mG*rCrGrArArCrUrGrArGrArArGrGrGrCrCrArGrUrUrUrUrAr | 419 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrU | |
| | rArCrCrUrCrGrGrCrCrCrUrUrCrUrCrArGrU*mU*mC*mG | |
| hPAH_NGG1_F13E8_ | mU*mA*mG*rCrGrArArCrUrGrArGrArArGrGrGrCrCrArGrUrUrUrUrAr | 420 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrU | |
| | rArCrCrUrCrGrGrCrCrCrUrUrCrUrCrArG*mU*mU*mC | |
| hPAH_NGG1_F12E8_ | mU*mA*mG*rCrGrArArCrUrGrArGrArArGrGrGrCrCrArGrUrUrUrUrAr | 421 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrU | |
| | rArCrCrUrCrGrGrCrCrCrUrUrCrUrCrA*mG*mU*mU | |
| hPAH_NGG1_F11E8_ | mU*mA*mG*rCrGrArArCrUrGrArGrArArGrGrGrCrCrArGrUrUrUrUrAr | 422 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrU | |
| | rArCrCrUrCrGrGrCrCrCrUrUrCrUrC*mA*mG*mU | |
| hPAH_NGG1_F10E8_ | mU*mA*mG*rCrGrArArCrUrGrArGrArArGrGrGrCrCrArGrUrUrUrUrAr | 423 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrU | |
| | rArCrCrUrCrGrGrCrCrCrUrUrCrU*mC*mA*mG | |
| hPAH_NGG1_F9E8_ | mU*mA*mG*rCrGrArArCrUrGrArGrArArGrGrGrCrCrArGrUrUrUrUrAr | 424 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrU | |
| | rArCrCrUrCrGrGrCrCrCrUrUrC*mU*mC*mA | |
| hPAH_NGG1_F8E8_ | mU*mA*mG*rCrGrArArCrUrGrArGrArArGrGrGrCrCrArGrUrUrUrUrAr | 425 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrU | |
| | rArCrCrUrCrGrGrCrCrCrUrU*mC*mU*mC | |
| hPAH_NGG1_F7E8_ | mU*mA*mG*rCrGrArArCrUrGrArGrArArGrGrGrCrCrArGrUrUrUrUrAr | 426 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrU | |
| | rArCrCrUrCrGrGrCrCrCrU*mU*mC*mU | |
| hPAH_NGG1_F15E10_ | mU*mA*mG*rCrGrArArCrUrGrArGrArArGrGrGrCrCrArGrUrUrUrUrAr | 427 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rArUrArCrCrUrCrGrGrCrCrCrUrUrCrUrCrArGrUrU*mC*mG*mC | |
| hPAH_NGG1_F14E10_ | mU*mA*mG*rCrGrArArCrUrGrArGrArArGrGrGrCrCrArGrUrUrUrUrAr | 428 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rArUrArCrCrUrCrGrGrCrCrCrUrUrCrUrCrArGrU*mU*mC*mG | |
| hPAH_NGG1_F13E10_ | mU*mA*mG*rCrGrArArCrUrGrArGrArArGrGrGrCrCrArGrUrUrUrUrAr | 429 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rArUrArCrCrUrCrGrGrCrCrCrUrUrCrUrCrArG*mU*mU*mC | |
| hPAH_NGG1_F12E10_ | mU*mA*mG*rCrGrArArCrUrGrArGrArArGrGrGrCrCrArGrUrUrUrUrAr | 430 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rArUrArCrCrUrCrGrGrCrCrCrUrUrCrUrCrA*mG*mU*mU | |
| hPAH_NGG1_F11E10_ | mU*mA*mG*rCrGrArArCrUrGrArGrArArGrGrGrCrCrArGrUrUrUrUrAr | 431 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rArUrArCrCrUrCrGrGrCrCrCrUrUrCrUrC*mA*mG*mU | |
| hPAH_NGG1_F10E10_ | mU*mA*mG*rCrGrArArCrUrGrArGrArArGrGrGrCrCrArGrUrUrUrUrAr | 432 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rArUrArCrCrUrCrGrGrCrCrCrUrUrCrU*mC*mA*mG | |
| hPAH_NGG1_F9E10_ | mU*mA*mG*rCrGrArArCrUrGrArGrArArGrGrGrCrCrArGrUrUrUrUrAr | 433 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rArUrArCrCrUrCrGrGrCrCrCrUrUrC*mU*mC*mA | |
| hPAH_NGG1_F8E10_ | mU*mA*mG*rCrGrArArCrUrGrArGrArArGrGrGrCrCrArGrUrUrUrUrAr | 434 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rArUrArCrCrUrCrGrGrCrCrCrUrU*mC*mU*mC | |
| hPAH_NGG1_F7E10_ | mU*mA*mG*rCrGrArArCrUrGrArGrArArGrGrGrCrCrArGrUrUrUrUrAr | 435 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rArUrArCrCrUrCrGrGrCrCrCrU*mU*mC*mU | |
| hPAH_NGG1_F14E12_ | mU*mA*mG*rCrGrArArCrUrGrArGrArArGrGrGrCrCrArGrUrUrUrUrAr | 436 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rCrArArUrArCrCrUrCrGrGrCrCrCrUrUrCrUrCrArGrU*mU*mC* | |
| | mG | |
| hPAH_NGG1_F13E12_ | mU*mA*mG*rCrGrArArCrUrGrArGrArArGrGrGrCrCrArGrUrUrUrUrAr | 437 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rCrArArUrArCrCrUrCrGrGrCrCrCrUrUrCrUrCrArG*mU*mU*mC | |
| hPAH_NGG1_F12E12_ | mU*mA*mG*rCrGrArArCrUrGrArGrArArGrGrGrCrCrArGrUrUrUrUrAr | 438 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rCrArArUrArCrCrUrCrGrGrCrCrCrUrUrCrUrCrA*mG*mU*mU | |
| hPAH_NGG1_F11E12_ | mU*mA*mG*rCrGrArArCrUrGrArGrArArGrGrGrCrCrArGrUrUrUrUrAr | 439 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rCrArArUrArCrCrUrCrGrGrCrCrCrUrUrCrUrC*mA*mG*mU | |
| hPAH_NGG1_F10E12_ | mU*mA*mG*rCrGrArArCrUrGrArGrArArGrGrGrCrCrArGrUrUrUrUrAr | 440 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rCrArArUrArCrCrUrCrGrGrCrCrCrUrUrCrU*mC*mA*mG | |
| hPAH_NGG1_F9R12_ | mU*mA*mG*rCrGrArArCrUrGrArGrArArGrGrGrCrCrArGrUrUrUrUrAr | 441 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rCrArArUrArCrCrUrCrGrGrCrCrCrUrUrC*mU*mC*mA | |
| hPAH_NGG1_F8E12_ | mU*mA*mG*rCrGrArArCrUrGrArGrArArGrGrGrCrCrArGrUrUrUrUrAr | 442 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rCrArArUrArCrCrUrCrGrGrCrCrCrUrU*mC*mU*mC | |
| hPAH_NGG1_F7E12_ | mU*mA*mG*rCrGrArArCrUrGrArGrArArGrGrGrCrCrArGrUrUrUrUrAr | 443 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rCrArArUrArCrCrUrCrGrGrCrCrCrU*mU*mC*mU | |
| hPAH_NGG1_F15E14_ | mU*mA*mG*rCrGrArArCrUrGrArGrArArGrGrGrCrCrArGrUrUrUrUrAr | 444 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrArCrArArUrArCrCrUrCrGrGrCrCrCrUrUrCrUrCrArGrUrU*m | |
| | C*mG*mC | |
| hPAH_NGG1_F14E14_ | mU*mA*mG*rCrGrArArCrUrGrArGrArArGrGrGrCrCrArGrUrUrUrUrAr | 445 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrArCrArArUrArCrCrUrCrGrGrCrCrCrUrUrCrUrCrArGrU*mU* | |
| | mC*mG | |
| hPAH_NGG1_F13R14_ | mU*mA*mG*rCrGrArArCrUrGrArGrArArGrGrGrCrCrArGrUrUrUrUrAr | 446 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrArCrArArUrArCrCrUrCrGrGrCrCrCrUrUrCrUrCrArG*mU*m | |
| | U*mC | |
| hPAH_NGG1_F12E14_ | mU*mA*mG*rCrGrArArCrUrGrArGrArArGrGrGrCrCrArGrUrUrUrUrAr | 447 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrArCrArArUrArCrCrUrCrGrGrCrCrCrUrUrCrUrCrA*mG*mU* | |
| | mU | |
| hPAH_NGG1_F11E14_ | mU*mA*mG*rCrGrArArCrUrGrArGrArArGrGrGrCrCrArGrUrUrUrUrAr | 448 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrArCrArArUrArCrCrUrCrGrGrCrCrCrUrUrCrUrC*mA*mG*mU | |
| hPAH_NGG1_F10E14_ | mU*mA*mG*rCrGrArArCrUrGrArGrArArGrGrGrCrCrArGrUrUrUrUrAr | 449 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrArCrArArUrArCrCrUrCrGrGrCrCrCrUrUrCrU*mC*mA*mG | |
| hPAH_NGG1_F9E14_ | mU*mA*mG*rCrGrArArCrUrGrArGrArArGrGrGrCrCrArGrUrUrUrUrAr | 450 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrArCrArArUrArCrCrUrCrGrGrCrCrCrUrUrC*mU*mC*mA | |
| hPAH_NGG1_F8R14_ | mU*mA*mG*rCrGrArArCrUrGrArGrArArGrGrGrCrCrArGrUrUrUrUrAr | 451 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrArCrArArUrArCrCrUrCrGrGrCrCrCrUrU*mC*mU*mC | |
| hPAH_NGG1_F7E14_ | mU*mA*mG*rCrGrArArCrUrGrArGrArArGrGrGrCrCrArGrUrUrUrUrAr | 452 |
| TtoC | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrC | |
| | rCrArCrArArUrArCrCrUrCrGrGrCrCrCrU*mU*mC*mU | |
| hPAH_NGG2_F15E7_ | mA*mC*mU*rUrUrGrCrUrGrCrCrArCrArArUrArCrCrUrGrUrUrUrUrArG | 453 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | CrCrGrArGrGrUrArUrUrGrUrGrGrCrArGrC*mA*mA*mA | |
| hPAH_NGG2_F14E7_ | mA*mC*mU*rUrUrGrCrUrGrCrCrArCrArArUrArCrCrUrGrUrUrUrUrArG | 454 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | CrCrGrArGrGrUrArUrUrGrUrGrGrCrArG*mC*mA*mA | |
| hPAH_NGG2_F13E7_ | mA*mC*mU*rUrUrGrCrUrGrCrCrArCrArArUrArCrCrUrGrUrUrUrUrArG | 455 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | CrCrGrArGrGrUrArUrUrGrUrGrGrCrA*mG*mC*mA | |
| hPAH_NGG2_F12E7_ | mA*mC*mU*rUrUrGrCrUrGrCrCrArCrArArUrArCrCrUrGrUrUrUrUrArG | 456 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | CrCrGrArGrGrUrArUrUrGrUrGrGrC*mA*mG*mC | |
| hPAH_NGG2_F11E7_ | mA*mC*mU*rUrUrGrCrUrGrCrCrArCrArArUrArCrCrUrGrUrUrUrUrArG | 457 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | CrCrGrArGrGrUrArUrUrGrUrGrG*mC*mA*mG | |
| hPAH_NGG2_F10E7_ | mA*mC*mU*rUrUrGrCrUrGrCrCrArCrArArUrArCrCrUrGrUrUrUrUrArG | 458 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | CrCrGrArGrGrUrArUrUrGrUrG*mG*mC*mA | |
| hPAH_NGG2_F9E7_ | mA*mC*mU*rUrUrGrCrUrGrCrCrArCrArArUrArCrCrUrGrUrUrUrUrArG | 459 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | CrCrGrArGrGrUrArUrUrGrU*mG*mG*mC | |
| hPAH_NGG2_F8E7_ | mA*mC*mU*rUrUrGrCrUrGrCrCrArCrArArUrArCrCrUrGrUrUrUrUrArG | 460 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | CrCrGrArGrGrUrArUrUrG*mU*mG*mG | |
| hPAH_NGG2_F7E7_ | mA*mC*mU*rUrUrGrCrUrGrCrCrArCrArArUrArCrCrUrGrUrUrUrUrArG | 461 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | CrCrGrArGrGrUrArUrU*mG*mU*mG | |
| hPAH_NGG2_F15E9_ | mA*mC*mU*rUrUrGrCrUrGrCrCrArCrArArUrArCrCrUrGrUrUrUrUrArG | 462 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | GrGrCrCrGrArGrGrUrArUrUrGrUrGrGrCrArGrC* mA*mA*mA | |
| hPAH_NGG2_F14E9_ | mA*mC*mU*rUrUrGrCrUrGrCrCrArCrArArUrArCrCrUrGrUrUrUrUrArG | 463 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | GrGrCrCrGrArGrGrUrArUrUrGrUrGrGrCrArG*mC*mA*mA | |
| hPAH_NGG2_F13E9_ | mA*mC*mU*rUrUrGrCrUrGrCrCrArCrArArUrArCrCrUrGrUrUrUrUrArG | 464 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | GrGrCrCrGrArGrGrUrArUrUrGrUrGrGrCrA*mG*mC*mA | |
| hPAH_NGG2_F12E9_ | mA*mC*mU*rUrUrGrCrUrGrCrCrArCrArArUrArCrCrUrGrUrUrUrUrArG | 465 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | GrGrCrCrGrArGrGrUrArUrUrGrUrGrGrC*mA*mG*mC | |
| hPAH_NGG2_F11E9_ | mA*mC*mU*rUrUrGrCrUrGrCrCrArCrArArUrArCrCrUrGrUrUrUrUrArG | 466 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | GrGrCrCrGrArGrGrUrArUrUrGrUrGrG*mC*mA*mG | |
| hPAH_NGG2_F10E9_ | mA*mC*mU*rUrUrGrCrUrGrCrCrArCrArArUrArCrCrUrGrUrUrUrUrArG | 467 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | GrGrCrCrGrArGrGrUrArUrUrGrUrG*mG*mC*mA | |
| hPAH_NGG2_F9E9_ | mA*mC*mU*rUrUrGrCrUrGrCrCrArCrArArUrArCrCrUrGrUrUrUrUrArG | 468 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | GrGrCrCrGrArGrGrUrArUrUrGrU*mG*mG*mC | |
| hPAH_NGG2_F8E9_ | mA*mC*mU*rUrUrGrCrUrGrCrCrArCrArArUrArCrCrUrGrUrUrUrUrArG | 469 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | GrGrCrCrGrArGrGrUrArUrUrG*mU*mG*mG | |
| hPAH_NGG2_F7E9_ | mA*mC*U*rUrUrGrCrUrGrCrCrArCrArArUrArCrCrUrGrUrUrUrUrArG | 470 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | GrGrCrCrGrArGrGrUrArUrU*mG*mU*mG | |
| hPAH_NGG2_F15E11_ | mA*mC*mU*rUrUrGrCrUrGrCrCrArCrArArUrArCrCrUrGrUrUrUrUrArG | 471 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | ArGrGrGrCrCrGrArGrGrUrArUrUrGrUrGrGrCrArGrC*mA*mA* | |
| | mA | |
| hPAH_NGG2_F14E11_ | mA*mC*mU*rUrUrGrCrUrGrCrCrArCrArArUrArCrCrUrGrUrUrUrUrArG | 472 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | ArGrGrGrCrCrGrArGrGrUrArUrUrGrUrGrGrCrArG*mC*mA*mA | |
| hPAH_NGG2_F13E11_ | mA*mC*mU*rUrUrGrCrUrGrCrCrArCrArArUrArCrCrUrGrUrUrUrUrArG | 473 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | ArGrGrGrCrCrGrArGrGrUrArUrUrGrUrGrGrCrA*mG*mC*mA | |
| hPAH_NGG2_F12E11_ | mA*mC*mU*rUrUrGrCrUrGrCrCrArCrArArUrArCrCrUrGrUrUrUrUrArG | 474 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | ArGrGrGrCrCrGrArGrGrUrArUrUrGrUrGrGrC*mA*mG*mC | |
| hPAH_NGG2_F11E11_ | mA*mC*mU*rUrUrGrCrUrGrCrCrArCrArArUrArCrCrUrGrUrUrUrUrArG | 475 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | ArGrGrGrCrCrGrArGrGrUrArUrUrGrUrGrG*mC*mA*mG | |
| hPAH_NGG2_F10E11_ | mA*mC*mU*rUrUrGrCrUrGrCrCrArCrArArUrArCrCrUrGrUrUrUrUrArG | 476 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | ArGrGrGrCrCrGrArGrGrUrArUrUrGrUrG*mG*mC*mA | |
| hPAH_NGG2_F9E11_ | mA*mC*mU*rUrUrGrCrUrGrCrCrArCrArArUrArCrCrUrGrUrUrUrUrArG | 477 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | ArGrGrGrCrCrGrArGrGrUrArUrUrGrU*mG*mG*mC | |
| hPAH_NGG2_F8E11_ | mA*mC*mU*rUrUrGrCrUrGrCrCrArCrArArUrArCrCrUrGrUrUrUrUrArG | 478 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | ArGrGrGrCrCrGrArGrGrUrArUrUrG*mU*mG*mG | |
| hPAH_NGG2_F7E11_ | mA*mC*mU*rUrUrGrCrUrGrCrCrArCrArArUrArCrCrUrGrUrUrUrUrArG | 479 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | ArGrGrGrCrCrGrArGrGrUrArUrU*mG*mU*mG | |
| hPAH_NGG2_F15E13_ | mA*mC*mU*rUrUrGrCrUrGrCrCrArCrArArUrArCrCrUrGrUrUrUrUrArG | 480 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | GrArArGrGrGrCrCrGrArGrGrUrArUrUrGrUrGrGrCrArGrC*mA* | |
| | mA*mA | |
| hPAH_NGG2_F14E13_ | mA*mC*mU*rUrUrGrCrUrGrCrCrArCrArArUrArCrCrUrGrUrUrUrUrAr | G481 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | GrArArGrGrGrCrCrGrArGrGrUrArUrUrGrUrGrGrCrArG*mC*m | |
| | A*mA | |
| hPAH_NGG2_F13E13_ | mA*mC*mU*rUrUrGrCrUrGrCrCrArCrArArUrArCrCrUrGrUrUrUrUrArG | 482 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | GrArArGrGrGrCrCrGrArGrGrUrArUrUrGrUrGrGrCrA*mG*mC* | |
| | mA | |
| hPAH_NGG2_F12E13_ | mA*mC*mU*rUrUrGrCrUrGrCrCrArCrArArUrArCrCrUrGrUrUrUrUrArG | 483 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | GrArArGrGrGrCrCrGrArGrGrUrArUrUrGrUrGrGrC*mA*mG*mC | |
| hPAH_NGG2_F11E13_ | mA*mC*mU*rUrUrGrCrUrGrCrCrArCrArArUrArCrCrUrGrUrUrUrUrArG | 484 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | GrArArGrGrGrCrCrGrArGrGrUrArUrUrGrUrGrG*mC*mA*mG | |
| hPAH_NGG2_F10E13_ | mA*mC*mU*rUrUrGrCrUrGrCrCrArCrArArUrArCrCrUrGrUrUrUrUrArG | 485 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | GrArArGrGrGrCrCrGrArGrGrUrArUrUrGrUrG*mG*mC*mA | |
| hPAH_NGG2_F9E13_ | mA*mC*mU*rUrUrGrCrUrGrCrCrArCrArArUrArCrCrUrGrUrUrUrUrArG | 486 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | GrArArGrGrGrCrCrGrArGrGrUrArUrUrGrU*mG*mG*mC | |
| hPAH_NGG2_F8E13_ | mA*mC*mU*rUrUrGrCrUrGrCrCrArCrArArUrArCrCrUrGrUrUrUrUrArG | 487 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | GrArArGrGrGrCrCrGrArGrGrUrArUrUrG*mU*mG*mG | |
| hPAH_NGG2_F7E13_ | mA*mC*mU*rUrUrGrCrUrGrCrCrArCrArArUrArCrCrUrGrUrUrUrUrArG | 488 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrAr | |
| | GrArArGrGrGrCrCrGrArGrGrUrArUrU*mG*mU*mG | |
| hPAH_NGG2_F15E15_ | mA*mC*mU*rUrUrGrCrUrGrCrCrArCrArArUrArCrCrUrGrUrUrUrUrArG | 489 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrUr | |
| | GrArGrArArGrGrGrCrCrGrArGrGrUrArUrUrGrUrGrGrCrArGrC | |
| | *mA*mA*mA | |
| hPAH_NGG2_F14E15_ | mA*mC*mU*rUrUrGrCrUrGrCrCrArCrArArUrArCrCrUrGrUrUrUrUrArG | 490 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrUr | |
| | GrArGrArArGrGrGrCrCrGrArGrGrUrArUrUrGrUrGrGrCrArG*m | |
| | C*mA*mA | |
| hPAH_NGG2_F13E15_ | mA*mC*mU*rUrUrGrCrUrGrCrCrArCrArArUrArCrCrUrGrUrUrUrUrArG | 491 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrUr | |
| | GrArGrArArGrGrGrCrCrGrArGrGrUrArUrUrGrUrGrGrCrA*mG* | |
| | mC*mA | |
| hPAH_NGG2_F12E15_ | mA*mC*mU*rUrUrGrCrUrGrCrCrArCrArArUrArCrCrUrGrUrUrUrUrArG | 492 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrUr | |
| | GrArGrArArGrGrGrCrCrGrArGrGrUrArUrUrGrUrGrGrC*mA*m | |
| | G*mC | |
| hPAH_NGG2_F11E15_ | mA*mC*mU*rUrUrGrCrUrGrCrCrArCrArArUrArCrCrUrGrUrUrUrUrArG | 493 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrUr | |
| | GrArGrArArGrGrGrCrCrGrArGrGrUrArUrUrGrUrGrG*mC*mA* | |
| | mG | |
| hPAH_NGG2_F10E15_ | mA*mC*mU*rUrUrGrCrUrGrCrCrArCrArArUrArCrCrUrGrUrUrUrUrArG | 494 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrUr | |
| | GrArGrArArGrGrGrCrCrGrArGrGrUrArUrUrGrUrG*mG*mC*mA | |
| hPAH_NGG2_F9E15_ | mA*mC*mU*rUrUrGrCrUrGrCrCrArCrArArUrArCrCrUrGrUrUrUrUrArG | 495 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrUr | |
| | GrArGrArArGrGrGrCrCrGrArGrGrUrArUrUrGrU*mG*mG*mC | |
| hPAH_NGG2_F8E15_ | mA*mC*mU*rUrUrGrCrUrGrCrCrArCrArArUrArCrCrUrGrUrUrUrUrArG | 496 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrUr | |
| | GrArGrArArGrGrGrCrCrGrArGrGrUrArUrUrG*mU*mG*mG | |
| hPAH_NGG2_F7E15_ | mA*mC*mU*rUrUrGrCrUrGrCrCrArCrArArUrArCrCrUrGrUrUrUrUrArG | 497 |
| AtoG | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrUr | |
| | GrArGrArArGrGrGrCrCrGrArGrGrUrArUrU*mG*mU*mG | |

¹For RNA, T is U; U are T in the sequence listing. For each entry, the spacer sequence is underlined and the extension arm is bold. The description indicates the length of the FBS (F) and ET (E) or homology region of the extension arm.
²For the sequences: *, phosphorothioate modification; mN, 2′O-methyl modification; rN, unmodified ribonucleotide; i2FN, Int Fluoro modification. N = A, C, T/U, G.

TABLE 12C
EXEMPLARY SPACER AND PROTOSPACER SEQUENCES FOR EDITING
PAH

| SEQ ID | | SEQ ID | |
|---|---|---|---|
| NO: | SPACER SEQUENCE¹ | NO: | PROTOSPACER SEQUENCE |
| 793 | UAGCGAACUGAGAAGGGCCA | 795 | TAGCGAACTGAGAAGGGCCA |
| 794 | ACUUUGCUGCCACAAUACCU | 796 | ACTTTGCTGCCACAATACCT |

¹For RNA, U are T in the sequence listing.

TABLE 12D
EXEMPLARY FLAP BINDING SEQUENCES AND TEMPLATES FOR
EDITING PAH

| DESCRIPTION | FBS¹ | SEQ ID NO: | EDITING TEMPLATE¹ | SEQ ID NO: |
|---|---|---|---|---|
| hPAH_NGG1_F15E6_TtoC | CCCUUCUCAGUUCGC | 803 | CCUCGG | |
| hPAH_NGG1_F14E6_TtoC | CCCUUCUCAGUUCG | 804 | CCUCGG | |
| hPAH_NGG1_F13E6_TtoC | CCCUUCUCAGUUC | 805 | CCUCGG | |
| hPAH_NGG1_F12E6_TtoC | CCCUUCUCAGUU | 806 | CCUCGG | |
| hPAH_NGG1_F11E6_TtoC | CCCUUCUCAGU | 807 | CCUCGG | |
| hPAH_NGG1_F10E6_TtoC | CCCUUCUCAG | 808 | CCUCGG | |
| hPAH_NGG1_F9E6_TtoC | CCCUUCUCA | | CCUCGG | |
| hPAH_NGG1_F8E6_TtoC | CCCUUCUC | | CCUCGG | |
| hPAH_NGG1_F7E6_TtoC | CCCUUCU | | CCUCGG | |
| hPAH_NGG1_F15E8_TtoC | CCCUUCUCAGUUCGC | 803 | UACCUCGG | |
| hPAH_NGG1_F14E8_TtoC | CCCUUCUCAGUUCG | 804 | UACCUCGG | |
| hPAH_NGG1_F13E8_TtoC | CCCUUCUCAGUUC | 805 | UACCUCGG | |
| hPAH_NGG1_F12E8_TtoC | CCCUUCUCAGUU | 806 | UACCUCGG | |
| hPAH_NGG1_F11E8_TtoC | CCCUUCUCAGU | 807 | UACCUCGG | |
| hPAH_NGG1_F10E8_TtoC | CCCUUCUCAG | 808 | UACCUCGG | |
| hPAH_NGG1_F9E8_TtoC | CCCUUCUCA | | UACCUCGG | |
| hPAH_NGG1_F8E8_TtoC | CCCUUCUC | | UACCUCGG | |
| hPAH_NGG1_F7E8_TtoC | CCCUUCU | | UACCUCGG | |
| hPAH_NGG1_F15E10_TtoC | CCCUUCUCAGUUCGC | 803 | AAUACCUCGG | 797 |
| hPAH_NGG1_F14E10_TtoC | CCCUUCUCAGUUCG | 804 | AAUACCUCGG | 797 |
| hPAH_NGG1_F13E10_TtoC | CCCUUCUCAGUUC | 805 | AAUACCUCGG | 797 |
| hPAH_NGG1_F12E10_TtoC | CCCUUCUCAGUU | 806 | AAUACCUCGG | 797 |
| hPAH_NGG1_F11E10_TtoC | CCCUUCUCAGU | 807 | AAUACCUCGG | 797 |
| hPAH_NGG1_F10E10_TtoC | CCCUUCUCAG | 808 | AAUACCUCGG | 797 |
| hPAH_NGG1_F9E10_TtoC | CCCUUCUCA | | AAUACCUCGG | 797 |
| hPAH_NGG1_F8E10_TtoC | CCCUUCUC | | AAUACCUCGG | 797 |
| hPAH_NGG1_F7E10_TtoC | CCCUUCU | | AAUACCUCGG | 797 |
| hPAH_NGG1_F14E12_TtoC | CCCUUCUCAGUUCG | 804 | ACAAUACCUCGG | 798 |
| hPAH_NGG1_F13E12_TtoC | CCCUUCUCAGUUC | 805 | ACAAUACCUCGG | 798 |
| hPAH_NGG1_F12E12_TtoC | CCCUUCUCAGUU | 806 | ACAAUACCUCGG | 798 |
| hPAH_NGG1_F11E12_TtoC | CCCUUCUCAGU | 807 | ACAAUACCUCGG | 798 |
| hPAH_NGG1_F10E12_TtoC | CCCUUCUCAG | 808 | ACAAUACCUCGG | 798 |
| hPAH_NGG1_F9E12_TtoC | CCCUUCUCA | | ACAAUACCUCGG | 798 |
| hPAH_NGG1_F8E12_TtoC | CCCUUCUC | | ACAAUACCUCGG | 798 |
| hPAH_NGG1_F7E12_TtoC | CCCUUCU | | ACAAUACCUCGG | 798 |
| hPAH_NGG1_F15E14_TtoC | CCCUUCUCAGUUCGC | 803 | CCACAAUACCUCGG | 799 |
| hPAH_NGG1_F14E14_TtoC | CCCUUCUCAGUUCG | 804 | CCACAAUACCUCGG | 799 |
| hPAH_NGG1_F13E14_TtoC | CCCUUCUCAGUUC | 805 | CCACAAUACCUCGG | 799 |
| hPAH_NGG1_F12E14_TtoC | CCCUUCUCAGUU | 806 | CCACAAUACCUCGG | 799 |
| hPAH_NGG1_F11E14_TtoC | CCCUUCUCAGU | 807 | CCACAAUACCUCGG | 799 |
| hPAH_NGG1_F10E14_TtoC | CCCUUCUCAG | 808 | CCACAAUACCUCGG | 799 |
| hPAH_NGG1_F9E14_TtoC | CCCUUCUCA | | CCACAAUACCUCGG | 799 |
| hPAH_NGG1_F8E14_TtoC | CCCUUCUC | | CCACAAUACCUCGG | 799 |
| hPAH_NGG1_F7E14_TtoC | CCCUUCU | | CCACAAUACCUCGG | 799 |
| hPAH_NGG2_F15E7_AtoG | UAUUGUGGCAGCAAA | 809 | GCCGAGG | |
| hPAH_NGG2_F14E7_AtoG | UAUUGUGGCAGCAA | 810 | GCCGAGG | |
| hPAH_NGG2_F13E7_AtoG | UAUUGUGGCAGCA | 811 | GCCGAGG | |
| hPAH_NGG2_F12E7_AtoG | UAUUGUGGCAGC | 812 | GCCGAGG | |
| hPAH_NGG2_F11E7_AtoG | UAUUGUGGCAG | 813 | GCCGAGG | |
| hPAH_NGG2_F10E7_AtoG | UAUUGUGGCA | 814 | GCCGAGG | |
| hPAH_NGG2_F9E7_AtoG | UAUUGUGGC | | GCCGAGG | |
| hPAH_NGG2_F8E7_AtoG | UAUUGUGG | | GCCGAGG | |
| hPAH_NGG2_F7E7_AtoG | UAUUGUG | | GCCGAGG | |
| hPAH_NGG2_F15E9_AtoG | UAUUGUGGCAGCAAA | 809 | GGGCCGAGG | |
| hPAH_NGG2_F14E9_AtoG | UAUUGUGGCAGCAA | 810 | GGGCCGAGG | |
| hPAH_NGG2_F13E9_AtoG | UAUUGUGGCAGCA | 811 | GGGCCGAGG | |
| hPAH_NGG2_F12E9_AtoG | UAUUGUGGCAGC | 812 | GGGCCGAGG | |
| hPAH_NGG2_F11E9_AtoG | UAUUGUGGCAG | 813 | GGGCCGAGG | |
| hPAH_NGG2_F10E9_AtoG | UAUUGUGGCA | 814 | GGGCCGAGG | |
| hPAH_NGG2_F9E9_AtoG | UAUUGUGGC | | GGGCCGAGG | |
| hPAH_NGG2_F8E9_AtoG | UAUUGUGG | | GGGCCGAGG | |
| hPAH_NGG2_F7E9_AtoG | UAUUGUG | | GGGCCGAGG | |
| hPAH_NGG2_F15E11_AtoG | UAUUGUGGCAGCAAA | 809 | AAGGGCCGAGG | 800 |
| hPAH_NGG2_F14E11_AtoG | UAUUGUGGCAGCAA | 810 | AAGGGCCGAGG | 800 |
| hPAH_NGG2_F13E11_AtoG | UAUUGUGGCAGCA | 811 | AAGGGCCGAGG | 800 |
| hPAH_NGG2_F12E11_AtoG | UAUUGUGGCAGC | 812 | AAGGGCCGAGG | 800 |
| hPAH_NGG2_F11E11_AtoG | UAUUGUGGCAG | 813 | AAGGGCCGAGG | 800 |
| hPAH_NGG2_F10E11_AtoG | UAUUGUGGCA | 814 | AAGGGCCGAGG | 800 |
| hPAH_NGG2_F9E11_AtoG | UAUUGUGGC | | AAGGGCCGAGG | 800 |
| hPAH_NGG2_F8E11_AtoG | UAUUGUGG | | AAGGGCCGAGG | 800 |
| hPAH_NGG2_F7E11_AtoG | UAUUGUG | | AAGGGCCGAGG | 800 |
| hPAH_NGG2_F15E13_AtoG | UAUUGUGGCAGCAAA | 809 | AGAAGGGCCGAGG | 801 |
| hPAH_NGG2_F14E13_AtoG | UAUUGUGGCAGCAA | 810 | AGAAGGGCCGAGG | 801 |
| hPAH_NGG2_F13E13_AtoG | UAUUGUGGCAGCA | 811 | AGAAGGGCCGAGG | 801 |
| hPAH_NGG2_F12E13_AtoG | UAUUGUGGCAGC | 812 | AGAAGGGCCGAGG | 801 |
| hPAH_NGG2_F11E13_AtoG | UAUUGUGGCAG | 813 | AGAAGGGCCGAGG | 801 |
| hPAH_NGG2_F10E13_AtoG | UAUUGUGGCA | 814 | AGAAGGGCCGAGG | 801 |
| hPAH_NGG2_F9E13_AtoG | UAUUGUGGC | | AGAAGGGCCGAGG | 801 |
| hPAH_NGG2_F8E13_AtoG | UAUUGUGG | | AGAAGGGCCGAGG | 801 |
| hPAH_NGG2_F7E13_AtoG | UAUUGUG | | AGAAGGGCCGAGG | 801 |
| hPAH_NGG2_F15E15_AtoG | UAUUGUGGCAGCAAA | 809 | UGAGAAGGGCCGAGG | 802 |
| hPAH_NGG2_F14E15_AtoG | UAUUGUGGCAGCAA | 810 | UGAGAAGGGCCGAGG | 802 |
| hPAH_NGG2_F13E15_AtoG | UAUUGUGGCAGCA | 811 | UGAGAAGGGCCGAGG | 802 |
| hPAH_NGG2_F12E15_AtoG | UAUUGUGGCAGC | 812 | UGAGAAGGGCCGAGG | 802 |
| hPAH_NGG2_F11E15_AtoG | UAUUGUGGCAG | 813 | UGAGAAGGGCCGAGG | 802 |
| hPAH_NGG2_F10E15_AtoG | UAUUGUGGCA | 814 | UGAGAAGGGCCGAGG | 802 |
| hPAH_NGG2_F9E15_AtoG | UAUUGUGGC | | UGAGAAGGGCCGAGG | 802 |
| hPAH_NGG2_F8E15_AtoG | UAUUGUGG | | UGAGAAGGGCCGAGG | 802 |
| hPAH_NGG2_F7E15_AtoG | UAUUGUG | | UGAGAAGGGCCGAGG | 802 |

¹For RNA, T is U; U are T in the sequence listing. The description indicates the length of the FBS (F) and ET (E) or homology region of the extension arm.

TABLE 12E
EXEMPLARY egRNA SEQUENCES FOR EDITING PAH

| NAME | SEQUENCE¹ | SEQ ID NO: |
|---|---|---|
| hPAH_egRNA_118038 | UUACACCUUACGAGGCCACUGUUUUAGAGCUAGAAAUAGCAAG | 498 |
| | UUAAAAUAAGGCUAGUCCGUUAUCAACUUGAAAAAGUGGCACC | |
| | GAGUCGGUGCUUUU | |
| hPAH_egRNA_118030 | UAAGUAAUUUACACCUUACGGUUUUAGAGCUAGAAAUAGCAAG | 499 |
| | UUAAAAUAAGGCUAGUCCGUUAUCAACUUGAAAAAGUGGCACC | |
| | GAGUCGGUGCUUUU | |
| hPAH_egRNA_117991 | UACCCAGCAGCUUAAGAUUUGUUUUAGAGCUAGAAAUAGCAAG | 500 |
| | UUAAAAUAAGGCUAGUCCGUUAUCAACUUGAAAAAGUGGCACC | |
| | GAGUCGGUGCUUUU | |
| hPAH_egRNA_117964 | CACCCAAAGGAUUGAGGUCUGUUUUAGAGCUAGAAAUAGCAAG | 501 |
| | UUAAAAUAAGGCUAGUCCGUUAUCAACUUGAAAAAGUGGCACC | |
| | GAGUCGGUGCUUUU | |
| hPAH_egRNA_117958 | CCCAUACACCCAAAGGAUUGGUUUUAGAGCUAGAAAUAGCAAG | 502 |
| | UUAAAAUAAGGCUAGUCCGUUAUCAACUUGAAAAAGUGGCACC | |
| | GAGUCGGUGCUUUU | |
| hPAH_egRNA_117951 | GCUACGACCCAUACACCCAAGUUUUAGAGCUAGAAAUAGCAAG | 503 |
| | UUAAAAUAAGGCUAGUCCGUUAUCAACUUGAAAAAGUGGCACC | |
| | GAGUCGGUGCUUUU | |
| hPAH_egRNA_117891 | AAGCCUGUGGUUUUGGUCUUGUUUUAGAGCUAGAAAUAGCAAG | 504 |
| | UUAAAAUAAGGCUAGUCCGUUAUCAACUUGAAAAAGUGGCACC | |
| | GAGUCGGUGCUUUU | |
| hPAH_egRNA_117884 | UUCACUCAAGCCUGUGGUUUGUUUUAGAGCUAGAAAUAGCAAG | 505 |
| | UUAAAAUAAGGCUAGUCCGUUAUCAACUUGAAAAAGUGGCACC | |
| | GAGUCGGUGCUUUU | |
| hPAH_egRNA_117878 | GUGCCCUUCACUCAAGCCUGGUUUUAGAGCUAGAAAUAGCAAG | 506 |
| | UUAAAAUAAGGCUAGUCCGUUAUCAACUUGAAAAAGUGGCACC | |
| | GAGUCGGUGCUUUU | |
| hPAH_egRNA_117856 | UAAGACUACCUUUCUCCAAAGUUUUAGAGCUAGAAAUAGCAAG | 507 |
| | UUAAAAUAAGGCUAGUCCGUUAUCAACUUGAAAAAGUGGCACC | |
| | GAGUCGGUGCUUUU | |
| hPAH_egRNA_117802 | UUACUUCUUUUUUAGGAACAGUUUUAGAGCUAGAAAUAGCAAG | 508 |
| | UUAAAAUAAGGCUAGUCCGUUAUCAACUUGAAAAAGUGGCACC | |
| | GAGUCGGUGCUUUU | |
| hPAH_egRNA_117927 | GGGUCGUAGCGAACUGAGAAGUUUUAGAGCUAGAAAUAGCAAG | 509 |
| | UUAAAAUAAGGCUAGUCCGUUAUCAACUUGAAAAAGUGGCACC | |
| | GAGUCGGUGCUUUU | |
| hPAH_egRNA_118009 | UGUAAAUUACUUACUGUUAAGUUUUAGAGCUAGAAAUAGCAAG | 510 |
| | UUAAAAUAAGGCUAGUCCGUUAUCAACUUGAAAAAGUGGCACC | |
| | GAGUCGGUGCUUUU | |
| hPAH_egRNA_117809 | UGGCAUUUUACUUCUUUUUUGUUUUAGAGCUAGAAAUAGCAAG | 511 |
| | UUAAAAUAAGGCUAGUCCGUUAUCAACUUGAAAAAGUGGCACC | |
| | GAGUCGGUGCUUUU | |
| hPAH_egRNA_117928 | UGGGUCGUAGCGAACUGAGAGUUUUAGAGCUAGAAAUAGCAAG | 512 |
| | UUAAAAUAAGGCUAGUCCGUUAUCAACUUGAAAAAGUGGCACC | |
| | GAGUCGGUGCUUUU | |
| hPAH_egRNA_118032 | AGAAACCGAGUGGCCUCGUAGUUUUAGAGCUAGAAAUAGCAAG | 513 |
| | UUAAAAUAAGGCUAGUCCGUUAUCAACUUGAAAAAGUGGCACC | |
| | GAGUCGGUGCUUUU | |
| hPAH_egRNA_117829 | AGUCUUAAGAGAGUUCUCAGGUUUUAGAGCUAGAAAUAGCAAG | 514 |
| | UUAAAAUAAGGCUAGUCCGUUAUCAACUUGAAAAAGUGGCACC | |
| | GAGUCGGUGCUUUU | |
| hPAH_egRNA_117947 | CCUCAAUCCUUUGGGUGUAUGUUUUAGAGCUAGAAAUAGCAAG | 515 |
| | UUAAAAUAAGGCUAGUCCGUUAUCAACUUGAAAAAGUGGCACC | |
| | GAGUCGGUGCUUUU | |
| hPAH_egRNA_118042 | UCGAUUACUGAGAAACCGAGGUUUUAGAGCUAGAAAUAGCAAG | 516 |
| | UUAAAAUAAGGCUAGUCCGUUAUCAACUUGAAAAAGUGGCACC | |
| | GAGUCGGUGCUUUU | |
| hPAH_egRNA_117853 | GAAGGGCACCAUUUGGAGAAGUUUUAGAGCUAGAAAUAGCAAG | 517 |
| | UUAAAAUAAGGCUAGUCCGUUAUCAACUUGAAAAAGUGGCACC | |
| | GAGUCGGUGCUUUU | |
| hPAH_egRNA_117948 | ACCUCAAUCCUUUGGGUGUAGUUUUAGAGCUAGAAAUAGCAAG | 518 |
| | UUAAAAUAAGGCUAGUCCGUUAUCAACUUGAAAAAGUGGCACC | |
| | GAGUCGGUGCUUUU | |
| hPAH_egRNA_117860 | CUUGAGUGAAGGGCACCAUUGUUUUAGAGCUAGAAAUAGCAAG | 519 |
| | UUAAAAUAAGGCUAGUCCGUUAUCAACUUGAAAAAGUGGCACC | |
| | GAGUCGGUGCUUUU | |
| hPAH_egRNA_117955 | GUCCAAGACCUCAAUCCUUUGUUUUAGAGCUAGAAAUAGCAAG | 520 |
| | UUAAAAUAAGGCUAGUCCGUUAUCAACUUGAAAAAGUGGCACC | |
| | GAGUCGGUGCUUUU | |
| hPAH_egRNA_117870 | AAACCACAGGCUUGAGUGAAGUUUUAGAGCUAGAAAUAGCAAG | 521 |
| | UUAAAAUAAGGCUAGUCCGUUAUCAACUUGAAAAAGUGGCACC | |
| | GAGUCGGUGCUUUU | |
| hPAH_egRNA_117956 | UGUCCAAGACCUCAAUCCUUGUUUUAGAGCUAGAAAUAGCAAG | 522 |
| | UUAAAAUAAGGCUAGUCCGUUAUCAACUUGAAAAAGUGGCACC | |
| | GAGUCGGUGCUUUU | |
| hPAH_egRNA_117871 | AAAACCACAGGCUUGAGUGAGUUUUAGAGCUAGAAAUAGCAAG | 523 |
| | UUAAAAUAAGGCUAGUCCGUUAUCAACUUGAAAAAGUGGCACC | |
| | GAGUCGGUGCUUUU | |
| hPAH_egRNA_117982 | AGCCAAAAUCUUAAGCUGCUGUUUUAGAGCUAGAAAUAGCAAG | 524 |
| | UUAAAAUAAGGCUAGUCCGUUAUCAACUUGAAAAAGUGGCACC | |
| | GAGUCGGUGCUUUU | |
| hPAH_egRNA_117883 | GUUCCUAAGACCAAAACCACGUUUUAGAGCUAGAAAUAGCAAG | 525 |
| | UUAAAAUAAGGCUAGUCCGUUAUCAACUUGAAAAAGUGGCACC | |
| | GAGUCGGUGCUUUU | |
| hPAH_egRNA_117983 | CAGCCAAAAUCUUAAGCUGCGUUUUAGAGCUAGAAAUAGCAAG | 526 |
| | UUAAAAUAAGGCUAGUCCGUUAUCAACUUGAAAAAGUGGCACC | |
| | GAGUCGGUGCUUUU | |

¹For RNA, Tis U; U are T in the sequence listing. For each entry, the spacer sequence is underlined.

TABLE 12F
EXEMPLARY MODIFIED egRNA SEQUENCES FOR EDITING PAH

| NAME | SEQUENCE^{1,2} | SEQ ID NO: |
|---|---|---|
| hPAH_egRNA_118038 | mU*mU*mA*rCrArCrCrUrUrArCrGrArGrGrCrCrArCrUrGrUrUrUrUrArG | 527 |
| | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCmU* | |
| | mU*mU*mU | |
| hPAH_egRNA_118030 | mU*mA*mA*rGrUrArArUrUrUrArCrArCrCrUrUrArCrGrGrUrUrUrUrAr | 528 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117991 | mU*mA*mC*rCrCrArGrCrArGrCrUrUrArArGrArUrUrUrGrUrUrUrUrAr | 529 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117964 | mC*mA*mC*rCrCrArArArGrGrArUrUrGrArGrGrUrCrUrGrUrUrUrUrAr | 530 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117958 | mC*mC*mC*rArUrArCrArCrCrCrArArArGrGrArUrUrGrGrUrUrUrUrArG | 531 |
| | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCmU* | |
| | mU*mU*mU | |
| hPAH_egRNA_117951 | mG*mC*mU*rArCrGrArCrCrCrArUrArCrArCrCrCrArArGrUrUrUrUrArG | 532 |
| | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCmU* | |
| | mU*mU*mU | |
| hPAH_egRNA_117891 | mA*mA*mG*rCrCrUrGrUrGrGrUrUrUrUrGrGrUrCrUrUrGrUrUrUrUrAr | 533 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117884 | mU*mU*mC*rArCrUrCrArArGrCrCrUrGrUrGrGrUrUrUrGrUrUrUrUrAr | 534 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117878 | mG*mU*mG*rCrCrCrUrUrCrArCrUrCrArArGrCrCrUrGrGrUrUrUrUrArG | 535 |
| | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCmU* | |
| | mU*mU*mU | |
| hPAH_egRNA_117856 | mU*mA*mA*rGrArCrUrArCrCrUrUrUrCrUrCrCrArArArGrUrUrUrUrAr | 536 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117802 | mU*mU*mA*rCrUrUrCrUrUrUrUrUrUrArGrGrArArCrArGrUrUrUrUrAr | 537 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117927 | mG*mG*mG*rUrCrGrUrArGrCrGrArArCrUrGrArGrArArGrUrUrUrUrAr | 538 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_118009 | mU*mG*mU*rArArArUrUrArCrUrUrArCrUrGrUrUrArArGrUrUrUrUrAr | 539 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117809 | mU*mG*mG*rCrArUrUrUrUrArCrUrUrCrUrUrUrUrUrUrGrUrUrUrUrAr | 540 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117928 | mU*mG*mG*rGrUrCrGrUrArGrCrGrArArCrUrGrArGrArGrUrUrUrUrAr | 541 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_118032 | mA*mG*mA*rArArCrCrGrArGrUrGrGrCrCrUrCrGrUrArGrUrUrUrUrAr | 542 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117829 | mA*mG*mU*rCrUrUrArArGrArGrArGrUrUrCrUrCrArGrGrUrUrUrUrAr | 543 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117947 | mC*mC*mU*rCrArArUrCrCrUrUrUrGrGrGrUrGrUrArUrGrUrUrUrUrAr | 544 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_118042 | mU*mC*mG*rArUrUrArCrUrGrArGrArArArCrCrGrArGrGrUrUrUrUrAr | 545 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117853 | mG*mA*mA*rGrGrGrCrArCrCrArUrUrUrGrGrArGrArArGrUrUrUrUrAr | 546 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117948 | mA*mC*mC*rUrCrArArUrCrCrUrUrUrGrGrGrUrGrUrArGrUrUrUrUrAr | 547 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117850 | mC*mU*mU*rGrArGrUrGrArArGrGrGrCrArCrCrArUrUrGrUrUrUrUrAr | 548 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117955 | mG*mU*mC*rCrArArGrArCrCrUrCrArArUrCrCrUrUrUrGrUrUrUrUrArG | 549 |
| | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCmU* | |
| | mU*mU*mU | |
| hPAH_egRNA_117870 | mA*mA*mA*rCrCrArCrArGrGrCrUrUrGrArGrUrGrArArGrUrUrUrUrAr | 550 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117956 | mU*mG*mU*rCrCrArArGrArCrCrUrCrArArUrCrCrUrUrGrUrUrUrUrArG | 551 |
| | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCmU* | |
| | mU*mU*mU | |
| hPAH_egRNA_117871 | mA*mA*mA*rArCrCrArCrArGrGrCrUrUrGrArGrUrGrArGrUrUrUrUrAr | 552 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117982 | mA*mG*mC*rCrArArArArUrCrUrUrArArGrCrUrGrCrUrGrUrUrUrUrAr | 553 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |
| hPAH_egRNA_117883 | mG*mU*mU*rCrCrUrArArGrArCrCrArArArArCrCrArCrGrUrUrUrUrArG | 554 |
| | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCmU* | |
| | mU*mU*mU | |
| hPAH_egRNA_117983 | mC*mA*mG*rCrCrArArArArUrCrUrUrArArGrCrUrGrCrGrUrUrUrUrAr | 555 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCm | |
| | U*mU*mU*mU | |

¹For RNA, T is U; U are T in the sequence listing. For each entry, the spacer sequence is underlined.
²For the sequences: *, phosphorothioate modification; mN, 2′O-methyl modification; rN, unmodified ribonucleotide; i2FN, Int Fluoro modification. N = A, C, T/U, G.

TABLE 12G
EXEMPLARY egRNA SPACER AND PROTOSPACER SEQUENCES FOR
EDITING PAH

| SEQ ID NO: | SPACER SEQUENCE¹ | SEQ ID NO: | PROTOSPACER SEQUENCE |
|---|---|---|---|
| 831 | UUACACCUUACGAGGCCACU | 860 | TTACACCTTACGAGGCCACT |
| 832 | UAAGUAAUUUACACCUUACG | 861 | TAAGTAATTTACACCTTACG |
| 833 | UACCCAGCAGCUUAAGAUUU | 862 | TACCCAGCAGCTTAAGATTT |
| 834 | CACCCAAAGGAUUGAGGUCU | 863 | CACCCAAAGGATTGAGGTCT |
| 835 | CCCAUACACCCAAAGGAUUG | 864 | CCCATACACCCAAAGGATTG |
| 836 | GCUACGACCCAUACACCCAA | 865 | GCTACGACCCATACACCCAA |
| 837 | AAGCCUGUGGUUUUGGUCUU | 866 | AAGCCTGTGGTTTTGGTCTT |
| 838 | UUCACUCAAGCCUGUGGUUU | 867 | TTCACTCAAGCCTGTGGTTT |
| 839 | GUGCCCUUCACUCAAGCCUG | 868 | GTGCCCTTCACTCAAGCCTG |
| 840 | UAAGACUACCUUUCUCCAAA | 869 | TAAGACTACCTTTCTCCAAA |
| 841 | UUACUUCUUUUUUAGGAACA | 870 | TTACTTCTTTTTTAGGAACA |
| 842 | GGGUCGUAGCGAACUGAGAA | 871 | GGGTCGTAGCGAACTGAGAA |
| 843 | UGUAAAUUACUUACUGUUAA | 872 | TGTAAATTACTTACTGTTAA |
| 844 | UGGCAUUUUACUUCUUUUUU | 873 | TGGCATTTTACTTCTTTTTT |
| 845 | UGGGUCGUAGCGAACUGAGA | 874 | TGGGTCGTAGCGAACTGAGA |
| 846 | AGAAACCGAGUGGCCUCGUA | 875 | AGAAACCGAGTGGCCTCGTA |
| 847 | AGUCUUAAGAGAGUUCUCAG | 876 | AGTCTTAAGAGAGTTCTCAG |
| 848 | CCUCAAUCCUUUGGGUGUAU | 877 | CCTCAATCCTTTGGGTGTAT |
| 849 | UCGAUUACUGAGAAACCGAG | 878 | TCGATTACTGAGAAACCGAG |
| 850 | GAAGGGCACCAUUUGGAGAA | 879 | GAAGGGCACCATTTGGAGAA |
| 851 | ACCUCAAUCCUUUGGGUGUA | 880 | ACCTCAATCCTTTGGGTGTA |
| 852 | CUUGAGUGAAGGGCACCAUU | 881 | CTTGAGTGAAGGGCACCATT |
| 853 | GUCCAAGACCUCAAUCCUUU | 882 | GTCCAAGACCTCAATCCTTT |
| 854 | AAACCACAGGCUUGAGUGAA | 883 | AAACCACAGGCTTGAGTGAA |
| 855 | UGUCCAAGACCUCAAUCCUU | 884 | TGTCCAAGACCTCAATCCTT |
| 856 | AAAACCACAGGCUUGAGUGA | 885 | AAAACCACAGGCTTGAGTGA |
| 857 | AGCCAAAAUCUUAAGCUGCU | 886 | AGCCAAAATCTTAAGCTGCT |
| 858 | GUUCCUAAGACCAAAACCAC | 887 | GTTCCTAAGACCAAAACCAC |
| 859 | CAGCCAAAAUCUUAAGCUGC | 888 | CAGCCAAAATCTTAAGCTGC |

¹For RNA, U are T in the sequence listing.

TABLE 13A
EXEMPLARY tagRNA SEQUENCES FOR EDITING SERPINA1

| NAME | SEQUENCE¹ | SEQ ID NO: |
|---|---|---|
| N20_NNAGAAA_+5_ | AAGGCUGUGCUGACCAUCGAGUUUUUGUACUCUCAAGAAAUUG | 563 |
| S_F12E8_TtoC | CAGAAGCUACAAAGAUAAGGCUUCAUGCCGAAAUCAACACCCU | |
| | GUCAUUUUAUGGCAGGGUGUUUCUCGUCGAUGGUCAGCACA | |
| N20_NNAGAAA_+5_ | AAGGCUGUGCUGACCAUCGAGUUUUUGUACUCUCAAGAAAUUG | 564 |
| S_F11E8_TtoC | CAGAAGCUACAAAGAUAAGGCUUCAUGCCGAAAUCAACACCCU | |
| | GUCAUUUUAUGGCAGGGUGUUUCUCGUCGAUGGUCAGCAC | |
| N20_NNAGAAA_+5_ | AAGGCUGUGCUGACCAUCGAGUUUUUGUACUCUCAAGAAAUUG | 565 |
| S_F10E8_TtoC | CAGAAGCUACAAAGAUAAGGCUUCAUGCCGAAAUCAACACCCU | |
| | GUCAUUUUAUGGCAGGGUGUUUCUCGUCGAUGGUCAGCA | |
| N20_NNAGAAA_+5_ | AAGGCUGUGCUGACCAUCGAGUUUUUGUACUCUCAAGAAAUUG | 566 |
| S_F9E8_TtoC | CAGAAGCUACAAAGAUAAGGCUUCAUGCCGAAAUCAACACCCU | |
| | GUCAUUUUAUGGCAGGGUGUUUCUCGUCGAUGGUCAGC | |
| N20_NNAGAAA_+5_ | AAGGCUGUGCUGACCAUCGAGUUUUUGUACUCUCAAGAAAUUG | 567 |
| S_F8E8_TtoC | CAGAAGCUACAAAGAUAAGGCUUCAUGCCGAAAUCAACACCCU | |
| | GUCAUUUUAUGGCAGGGUGUUUCUCGUCGAUGGUCAG | |
| N20_NNAGAAA_+5_ | AAGGCUGUGCUGACCAUCGAGUUUUUGUACUCUCAAGAAAUUG | 568 |
| S_F7E8_TtoC | CAGAAGCUACAAAGAUAAGGCUUCAUGCCGAAAUCAACACCCU | |
| | GUCAUUUUAUGGCAGGGUGUUUCUCGUCGAUGGUCA | |
| N20_NNAGAAA_+5_ | AAGGCUGUGCUGACCAUCGAGUUUUUGUACUCUCAAGAAAUUG | 569 |
| S_F12E10_TtoC | CAGAAGCUACAAAGAUAAGGCUUCAUGCCGAAAUCAACACCCU | |
| | GUCAUUUUAUGGCAGGGUGUUUUUCUCGUCGAUGGUCAGCAC | |
| | A | |
| N20_NNAGAAA_+5_ | AAGGCUGUGCUGACCAUCGAGUUUUUGUACUCUCAAGAAAUUG | 570 |
| S_F11E10_TtoC | CAGAAGCUACAAAGAUAAGGCUUCAUGCCGAAAUCAACACCCU | |
| | GUCAUUUUAUGGCAGGGUGUUUUUCUCGUCGAUGGUCAGCAC | |
| N20_NNAGAAA_+5_ | AAGGCUGUGCUGACCAUCGAGUUUUUGUACUCUCAAGAAAUUG | 571 |
| S_F10E10_TtoC | CAGAAGCUACAAAGAUAAGGCUUCAUGCCGAAAUCAACACCCU | |
| | GUCAUUUUAUGGCAGGGUGUUUUUCUCGUCGAUGGUCAGCA | |
| N20_NNAGAAA_+5_ | AAGGCUGUGCUGACCAUCGAGUUUUUGUACUCUCAAGAAAUUG | 572 |
| S_F9E10_TtoC | CAGAAGCUACAAAGAUAAGGCUUCAUGCCGAAAUCAACACCCU | |
| | GUCAUUUUAUGGCAGGGUGUUUUUCUCGUCGAUGGUCAGC | |
| N20_NNAGAAA_+5_ | AAGGCUGUGCUGACCAUCGAGUUUUUGUACUCUCAAGAAAUUG | 573 |
| S_F8E10_TtoC | CAGAAGCUACAAAGAUAAGGCUUCAUGCCGAAAUCAACACCCU | |
| | GUCAUUUUAUGGCAGGGUGUUUUUCUCGUCGAUGGUCAG | |
| N20_NNAGAAA_+5_ | AAGGCUGUGCUGACCAUCGAGUUUUUGUACUCUCAAGAAAUUG | 574 |
| S_F7E10_TtoC | CAGAAGCUACAAAGAUAAGGCUUCAUGCCGAAAUCAACACCCU | |
| | GUCAUUUUAUGGCAGGGUGUUUUUCUCGUCGAUGGUCA | |
| N20_NNAGAAA_+5_ | AAGGCUGUGCUGACCAUCGAGUUUUUGUACUCUCAAGAAAUUG | 575 |
| S_F12E12_TtoC | CAGAAGCUACAAAGAUAAGGCUUCAUGCCGAAAUCAACACCCU | |
| | GUCAUUUUAUGGCAGGGUGUUCCUUUCUCGUCGAUGGUCAGC | |
| | ACA | |
| N20_NNAGAAA_+5_ | AAGGCUGUGCUGACCAUCGAGUUUUUGUACUCUCAAGAAAUUG | 576 |
| S_F11E12_TtoC | CAGAAGCUACAAAGAUAAGGCUUCAUGCCGAAAUCAACACCCU | |
| | GUCAUUUUAUGGCAGGGUGUUCCUUUCUCGUCGAUGGUCAGC | |
| | AC | |
| N20_NNAGAAA_+5_ | AAGGCUGUGCUGACCAUCGAGUUUUUGUACUCUCAAGAAAUUG | 577 |
| S_F10E12_TtoC | CAGAAGCUACAAAGAUAAGGCUUCAUGCCGAAAUCAACACCCU | |
| | GUCAUUUUAUGGCAGGGUGUUCCUUUCUCGUCGAUGGUCAGC | |
| | A | |
| N20_NNAGAAA_+5_ | AAGGCUGUGCUGACCAUCGAGUUUUUGUACUCUCAAGAAAUUG | 578 |
| S_F9E12_TtoC | CAGAAGCUACAAAGAUAAGGCUUCAUGCCGAAAUCAACACCCU | |
| | GUCAUUUUAUGGCAGGGUGUUCCUUUCUCGUCGAUGGUCAGC | |
| N20_NNAGAAA_+5_ | AAGGCUGUGCUGACCAUCGAGUUUUUGUACUCUCAAGAAAUUG | 579 |
| S_F8E12_TtoC | CAGAAGCUACAAAGAUAAGGCUUCAUGCCGAAAUCAACACCCU | |
| | GUCAUUUUAUGGCAGGGUGUUCCUUUCUCGUCGAUGGUCAG | |
| N20_NNAGAAA_+5_ | AAGGCUGUGCUGACCAUCGAGUUUUUGUACUCUCAAGAAAUUG | 580 |
| S_F7E12_TtoC | CAGAAGCUACAAAGAUAAGGCUUCAUGCCGAAAUCAACACCCU | |
| | GUCAUUUUAUGGCAGGGUGUUCCUUUCUCGUCGAUGGUCA | |
| N20_NNAGAAA_+5_ | AAGGCUGUGCUGACCAUCGAGUUUUUGUACUCUCAAGAAAUUG | 581 |
| S_F12E14_TtoC | CAGAAGCUACAAAGAUAAGGCUUCAUGCCGAAAUCAACACCCU | |
| | GUCAUUUUAUGGCAGGGUGUUUCCCUUUCUCGUCGAUGGUCA | |
| | GCACA | |
| N20_NNAGAAA_+5_ | AAGGCUGUGCUGACCAUCGAGUUUUUGUACUCUCAAGAAAUUG | 582 |
| S_F11E14_TtoC | CAGAAGCUACAAAGAUAAGGCUUCAUGCCGAAAUCAACACCCU | |
| | GUCAUUUUAUGGCAGGGUGUUUCCCUUUCUCGUCGAUGGUCA | |
| | GCAC | |
| N20_NNAGAAA_+5_ | AAGGCUGUGCUGACCAUCGAGUUUUUGUACUCUCAAGAAAUUG | 583 |
| S_F10E14_TtoC | CAGAAGCUACAAAGAUAAGGCUUCAUGCCGAAAUCAACACCCU | |
| | GUCAUUUUAUGGCAGGGUGUUUCCCUUUCUCGUCGAUGGUCA | |
| | GCA | |
| N20_NNAGAAA_+5_ | AAGGCUGUGCUGACCAUCGAGUUUUUGUACUCUCAAGAAAUUG | 584 |
| S_F9E14_TtoC | CAGAAGCUACAAAGAUAAGGCUUCAUGCCGAAAUCAACACCCU | |
| | GUCAUUUUAUGGCAGGGUGUUUCCCUUUCUCGUCGAUGGUCA | |
| | GC | |
| N20_NNAGAAA_+5_ | AAGGCUGUGCUGACCAUCGAGUUUUUGUACUCUCAAGAAAUUG | 585 |
| S_F8E14_TtoC | CAGAAGCUACAAAGAUAAGGCUUCAUGCCGAAAUCAACACCCU | |
| | GUCAUUUUAUGGCAGGGUGUUUCCCUUUCUCGUCGAUGGUCA | |
| | G | |
| N20_NNAGAAA_+5_ | AAGGCUGUGCUGACCAUCGAGUUUUUGUACUCUCAAGAAAUUG | 586 |
| S_F7E14_TtoC | CAGAAGCUACAAAGAUAAGGCUUCAUGCCGAAAUCAACACCCU | |
| | GUCAUUUUAUGGCAGGGUGUUUCCCUUUCUCGUCGAUGGUCA | |
| N20_NNAGAAA_+5_ | AAGGCUGUGCUGACCAUCGAGUUUUUGUACUCUCAAGAAAUUG | 587 |
| S_F12E16_TtoC | CAGAAGCUACAAAGAUAAGGCUUCAUGCCGAAAUCAACACCCU | |
| | GUCAUUUUAUGGCAGGGUGUUAGUCCCUUUCUCGUCGAUGGU | |
| | CAGCACA | |
| N20_NNAGAAA_+5_ | AAGGCUGUGCUGACCAUCGAGUUUUUGUACUCUCAAGAAAUUG | 588 |
| S_F11E16_TtoC | CAGAAGCUACAAAGAUAAGGCUUCAUGCCGAAAUCAACACCCU | |
| | GUCAUUUUAUGGCAGGGUGUUAGUCCCUUUCUCGUCGAUGGU | |
| | CAGCAC | |
| N20_NNAGAAA_+5_ | AAGGCUGUGCUGACCAUCGAGUUUUUGUACUCUCAAGAAAUUG | 589 |
| S_F10E16_TtoC | CAGAAGCUACAAAGAUAAGGCUUCAUGCCGAAAUCAACACCCU | |
| | GUCAUUUUAUGGCAGGGUGUUAGUCCCUUUCUCGUCGAUGGU | |
| | CAGCA | |
| N20_NNAGAAA_+5_ | AAGGCUGUGCUGACCAUCGAGUUUUUGUACUCUCAAGAAAUUG | 590 |
| S_F9E16_TtoC | CAGAAGCUACAAAGAUAAGGCUUCAUGCCGAAAUCAACACCCU | |
| | GUCAUUUUAUGGCAGGGUGUUAGUCCCUUUCUCGUCGAUGGU | |
| | CAGC | |
| N20_NNAGAAA_+5_ | AAGGCUGUGCUGACCAUCGAGUUUUUGUACUCUCAAGAAAUUG | 591 |
| S_F8E16_TtoC | CAGAAGCUACAAAGAUAAGGCUUCAUGCCGAAAUCAACACCCU | |
| | GUCAUUUUAUGGCAGGGUGUUAGUCCCUUUCUCGUCGAUGGU | |
| | CAG | |
| N20_NNAGAAA_+5_ | AAGGCUGUGCUGACCAUCGAGUUUUUGUACUCUCAAGAAAUUG | 592 |
| S_F7E16_TtoC | CAGAAGCUACAAAGAUAAGGCUUCAUGCCGAAAUCAACACCCU | |
| | GUCAUUUUAUGGCAGGGUGUUAGUCCCUUUCUCGUCGAUGGU | |
| | CA | |
| N20_NAA_+1_S_ | CUGUGCUGACCAUCGACAAGGUUUUAGAGCUAGAAAUAGCAAG | 593 |
| F12E4_TtoC | UUAAAAUAAGGCUAGUCCGUUAUCAACUUGAAAAAGUGGCACC | |
| | GAGUCGGUGCUCUCGUCGAUGGUCAG | |
| N20_NAA_+1_S_ | CUGUGCUGACCAUCGACAAGGUUUUAGAGCUAGAAAUAGCAAG | 594 |
| F11E4_TtoC | UUAAAAUAAGGCUAGUCCGUUAUCAACUUGAAAAAGUGGCACC | |
| | GAGUCGGUGCUCUCGUCGAUGGUCA | |
| N20_NAA_+1_S_ | CUGUGCUGACCAUCGACAAGGUUUUAGAGCUAGAAAUAGCAAG | 595 |
| F10E4_TtoC | UUAAAAUAAGGCUAGUCCGUUAUCAACUUGAAAAAGUGGCACC | |
| | GAGUCGGUGCUCUCGUCGAUGGUC | |
| N20_NAA_+1_S_ | CUGUGCUGACCAUCGACAAGGUUUUAGAGCUAGAAAUAGCAAG | 596 |
| F9E4_TtoC | UUAAAAUAAGGCUAGUCCGUUAUCAACUUGAAAAAGUGGCACC | |
| | GAGUCGGUGCUCUCGUCGAUGGU | |
| N20_NAA_+1_S_ | CUGUGCUGACCAUCGACAAGGUUUUAGAGCUAGAAAUAGCAAG | 597 |
| F8E4_TtoC | UUAAAAUAAGGCUAGUCCGUUAUCAACUUGAAAAAGUGGCACC | |
| | GAGUCGGUGCUCUCGUCGAUGG | |
| N20_NAA_+1_S_ | CUGUGCUGACCAUCGACAAGGUUUUAGAGCUAGAAAUAGCAAG | 598 |
| F7E4_TtoC | UUAAAAUAAGGCUAGUCCGUUAUCAACUUGAAAAAGUGGCACC | |
| | GAGUCGGUGCUCUCGUCGAUG | |
| N20_NAA_+1_S_ | CUGUGCUGACCAUCGACAAGGUUUUAGAGCUAGAAAUAGCAAG | 599 |
| F12E6_TtoC | UUAAAAUAAGGCUAGUCCGUUAUCAACUUGAAAAAGUGGCACC | |
| | GAGUCGGUGCUUUCUCGUCGAUGGUCAG | |
| N20_NAA_+1_S_ | CUGUGCUGACCAUCGACAAGGUUUUAGAGCUAGAAAUAGCAAG | 600 |
| F11E6_Tto | UUAAAAUAAGGCUAGUCCGUUAUCAACUUGAAAAAGUGGCACC | |
| | GAGUCGGUGCUUUCUCGUCGAUGGUCA | |
| N20_NAA_+1_S_ | CUGUGCUGACCAUCGACAAGGUUUUAGAGCUAGAAAUAGCAAG | 601 |
| F10E6_TtoC | UUAAAAUAAGGCUAGUCCGUUAUCAACUUGAAAAAGUGGCACC | |
| | GAGUCGGUGCUUUCUCGUCGAUGGUC | |
| N20_NAA_+1_S_ | CUGUGCUGACCAUCGACAAGGUUUUAGAGCUAGAAAUAGCAAG | 602 |
| F9E6_TtoC | UUAAAAUAAGGCUAGUCCGUUAUCAACUUGAAAAAGUGGCACC | |
| | GAGUCGGUGCUUUCUCGUCGAUGGU | |
| N20_NAA_+1_S_ | CUGUGCUGACCAUCGACAAGGUUUUAGAGCUAGAAAUAGCAAG | 603 |
| F8E6_TtoC | UUAAAAUAAGGCUAGUCCGUUAUCAACUUGAAAAAGUGGCACC | |
| | GAGUCGGUGCUUUCUCGUCGAUGG | |
| N20_NAA_+1_S_ | CUGUGCUGACCAUCGACAAGGUUUUAGAGCUAGAAAUAGCAAG | 604 |
| F7E6_TtoC | UUAAAAUAAGGCUAGUCCGUUAUCAACUUGAAAAAGUGGCACC | |
| | GAGUCGGUGCUUUCUCGUCGAUG | |
| N20_NAA_+1_S_ | CUGUGCUGACCAUCGACAAGGUUUUAGAGCUAGAAAUAGCAAG | 605 |
| F12E8_TtoC | UUAAAAUAAGGCUAGUCCGUUAUCAACUUGAAAAAGUGGCACC | |
| | GAGUCGGUGCCCUUUCUCGUCGAUGGUCAG | |
| N20_NAA_+1_S_ | CUGUGCUGACCAUCGACAAGGUUUUAGAGCUAGAAAUAGCAAG | 606 |
| F11E8_TtoC | UUAAAAUAAGGCUAGUCCGUUAUCAACUUGAAAAAGUGGCACC | |
| | GAGUCGGUGCCCUUUCUCGUCGAUGGUCA | |
| N20_NAA_+1_S_ | CUGUGCUGACCAUCGACAAGGUUUUAGAGCUAGAAAUAGCAAG | 607 |
| F10E8_TtoC | UUAAAAUAAGGCUAGUCCGUUAUCAACUUGAAAAAGUGGCACC | |
| | GAGUCGGUGCCCUUUCUCGUCGAUGGUC | |
| N20_NAA_+1_S_ | CUGUGCUGACCAUCGACAAGGUUUUAGAGCUAGAAAUAGCAAG | 608 |
| F9E8_TtoC | UUAAAAUAAGGCUAGUCCGUUAUCAACUUGAAAAAGUGGCACC | |
| | GAGUCGGUGCCCUUUCUCGUCGAUGGU | |
| N20_NAA_+1_S_ | CUGUGCUGACCAUCGACAAGGUUUUAGAGCUAGAAAUAGCAAG | 609 |
| F8E8_TtoC | UUAAAAUAAGGCUAGUCCGUUAUCAACUUGAAAAAGUGGCACC | |
| | GAGUCGGUGCCCUUUCUCGUCGAUGG | |
| N20_NAA_+1_S_ | CUGUGCUGACCAUCGACAAGGUUUUAGAGCUAGAAAUAGCAAG | 610 |
| F7E8_TtoC | UUAAAAUAAGGCUAGUCCGUUAUCAACUUGAAAAAGUGGCACC | |
| | GAGUCGGUGCCCUUUCUCGUCGAUG | |
| N20_NAA_+1_S_ | CUGUGCUGACCAUCGACAAGGUUUUAGAGCUAGAAAUAGCAAG | 611 |
| F12E10_TtoC | UUAAAAUAAGGCUAGUCCGUUAUCAACUUGAAAAAGUGGCACC | |
| | GAGUCGGUGCUCCCUUUCUCGUCGAUGGUCAG | |
| N20_NAA_+1_S_ | CUGUGCUGACCAUCGACAAGGUUUUAGAGCUAGAAAUAGCAAG | 612 |
| F11E10_TtoC | UUAAAAUAAGGCUAGUCCGUUAUCAACUUGAAAAAGUGGCACC | |
| | GAGUCGGUGCUCCCUUUCUCGUCGAUGGUCA | |
| N20_NAA_+1_S_ | CUGUGCUGACCAUCGACAAGGUUUUAGAGCUAGAAAUAGCAAG | 613 |
| F10E10_TtoC | UUAAAAUAAGGCUAGUCCGUUAUCAACUUGAAAAAGUGGCACC | |
| | GAGUCGGUGCUCCCUUUCUCGUCGAUGGUC | |
| N20_NAA_+1_S_ | CUGUGCUGACCAUCGACAAGGUUUUAGAGCUAGAAAUAGCAAG | 614 |
| F9E10_TtoC | UUAAAAUAAGGCUAGUCCGUUAUCAACUUGAAAAAGUGGCACC | |
| | GAGUCGGUGCUCCCUUUCUCGUCGAUGGU | |
| N20_NAA_+1_S_ | CUGUGCUGACCAUCGACAAGGUUUUAGAGCUAGAAAUAGCAAG | 615 |
| F8E10_TtoC | UUAAAAUAAGGCUAGUCCGUUAUCAACUUGAAAAAGUGGCACC | |
| | GAGUCGGUGCUCCCUUUCUCGUCGAUGG | |
| N20_NAA_+1_S_ | CUGUGCUGACCAUCGACAAGGUUUUAGAGCUAGAAAUAGCAAG | 616 |
| F7E10_TtoC | UUAAAAUAAGGCUAGUCCGUUAUCAACUUGAAAAAGUGGCACC | |
| | GAGUCGGUGCUCCCUUUCUCGUCGAUG | |
| N20_NAA_+1_S_ | CUGUGCUGACCAUCGACAAGGUUUUAGAGCUAGAAAUAGCAAG | 617 |
| F12E12_TtoC | UUAAAAUAAGGCUAGUCCGUUAUCAACUUGAAAAAGUGGCACC | |
| | GAGUCGGUGCAGUCCCUUUCUCGUCGAUGGUCAG | |
| N20_NAA_+1_S_ | CUGUGCUGACCAUCGACAAGGUUUUAGAGCUAGAAAUAGCAAG | 618 |
| F11E12_TtoC | UUAAAAUAAGGCUAGUCCGUUAUCAACUUGAAAAAGUGGCACC | |
| | GAGUCGGUGCAGUCCCUUUCUCGUCGAUGGUCA | |
| N20_NAA_+1_S_ | CUGUGCUGACCAUCGACAAGGUUUUAGAGCUAGAAAUAGCAAG | 619 |
| F10E12_TtoC | UUAAAAUAAGGCUAGUCCGUUAUCAACUUGAAAAAGUGGCACC | |
| | GAGUCGGUGCAGUCCCUUUCUCGUCGAUGGUC | |
| N20_NAA_+1_S_ | CUGUGCUGACCAUCGACAAGGUUUUAGAGCUAGAAAUAGCAAG | 620 |
| F9E12_TtoC | UUAAAAUAAGGCUAGUCCGUUAUCAACUUGAAAAAGUGGCACC | |
| | GAGUCGGUGCAGUCCCUUUCUCGUCGAUGGU | |
| N20_NAA_+1_S_ | CUGUGCUGACCAUCGACAAGGUUUUAGAGCUAGAAAUAGCAAG | 621 |
| F8E12_TtoC | UUAAAAUAAGGCUAGUCCGUUAUCAACUUGAAAAAGUGGCACC | |
| | GAGUCGGUGCAGUCCCUUUCUCGUCGAUGG | |
| N20_NAA_+1_S_ | CUGUGCUGACCAUCGACAAGGUUUUAGAGCUAGAAAUAGCAAG | 622 |
| F7E12_TtoC | UUAAAAUAAGGCUAGUCCGUUAUCAACUUGAAAAAGUGGCACC | |
| | GAGUCGGUGCAGUCCCUUUCUCGUCGAUG | |

¹For RNA, T is U; U are T in the sequence listing. For each entry, the spacer sequence is underlined and the extension arm is bold. The description indicates the length of the FBS (F) and ET (E) or homology region of the extension arm.

TABLE 13B
EXEMPLARY MODIFIED tagRNA SEQUENCES FOR EDITING SERPINA1

| NAME | SEQUENCE1,2 | SEQ ID NO: |
|---|---|---|
| N20_NNAGAAA_ | mA*mA*mG*rGrCrUrGrUrGrCrUrGrArCrCrArUrCrGrArGrUrUrUrUrUrG | 623 |
| +5_S_F12E8_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrUrCrGrUrCrGr | |
| | ArUrGrGrUrCrArGrC*mA*mC*mA | |
| N20_NNAGAAA_ | mA*mA*mG*rGrCrUrGrUrGrCrUrGrArCrCrArUrCrGrArGrUrUrUrUrUrG | 624 |
| +5_S_F11E8_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrUrCrGrUrCrGr | |
| | ArUrGrGrUrCrArG*mC*mA*mC | |
| N20_NNAGAAA_ | mA*mA*mG*rGrCrUrGrUrGrCrUrGrArCrCrArUrCrGrArGrUrUrUrUrUrG | 625 |
| +5_S_F10E8_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrUrCrGrUrCrGr | |
| | ArUrGrGrUrCrA*mG*mC*mA | |
| N20_NNAGAAA_ | mA*mA*mG*rGrCrUrGrUrGrCrUrGrArCrCrArUrCrGrArGrUrUrUrUrUrG | 626 |
| +5_S_F9E8_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrUrCrGrUrCrGr | |
| | ArUrGrGrUrC*mA*mG*mC | |
| N20_NNAGAAA_ | mA*mA*mG*rGrCrUrGrUrGrCrUrGrArCrCrArUrCrGrArGrUrUrUrUrUrG | 627 |
| +5_S_F8E8_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrUrCrGrUrCrGr | |
| | ArUrGrGrU*mC*mA*mG | |
| N20_NNAGAAA_ | mA*mA*mG*rGrCrUrGrUrGrCrUrGrArCrCrArUrCrGrArGrUrUrUrUrUrG | 628 |
| +5_S_F7E8_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrUrCrGrUrCrGr | |
| | ArUrGrG*mU*mC*mA | |
| N20_NNAGAAA_ | mA*mA*mG*rGrCrUrGrUrGrCrUrGrArCrCrArUrCrGrArGrUrUrUrUrUrG | 629 |
| +5_S_F12E10_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrUrUrCrUrCrGrUr | |
| | CrGrArUrGrGrUrCrArGrC*mA*mC*mA | |
| N20_NNAGAAA_ | mA*mA*mG*rGrCrUrGrUrGrCrUrGrArCrCrArUrCrGrArGrUrUrUrUrUrG | 630 |
| +5_S_F11E10_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrUrUrCrUrCrGrUr | |
| | CrGrArUrGrGrUrCrArG*mC*mA*mC | |
| N20_NNAGAAA_ | mA*mA*mG*rGrCrUrGrUrGrCrUrGrArCrCrArUrCrGrArGrUrUrUrUrUrG | 631 |
| +5_S_F10E10_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrUrUrCrUrCrGrUr | |
| | CrGrArUrGrGrUrCrA*mG*mC*mA | |
| N20_NNAGAAA_ | mA*mA*mG*rGrCrUrGrUrGrCrUrGrArCrCrArUrCrGrArGrUrUrUrUrUrG | 632 |
| +5_S_F9E10_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrUrUrCrUrCrGrUr | |
| | CrGrArUrGrGrUrC*mA*mG*mC | |
| N20_NNAGAAA_ | mA*mA*mG*rGrCrUrGrUrGrCrUrGrArCrCrArUrCrGrArGrUrUrUrUrUrG | 633 |
| +5_S_F8E10_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrUrUrCrUrCrGrUr | |
| | CrGrArUrGrGrU*mC*mA*mG | |
| N20_NNAGAAA_ | mA*mA*mG*rGrCrUrGrUrGrCrUrGrArCrCrArUrCrGrArGrUrUrUrUrUrG | 634 |
| +5_S_F7E10_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrUrUrCrUrCrGrUr | |
| | CrGrArUrGrG*mU*mC*mA | |
| N20_NNAGAAA_ | mA*mA*mG*rGrCrUrGrUrGrCrUrGrArCrCrArUrCrGrArGrUrUrUrUrUrG | 635 |
| +5_S_F12E12_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrCrCrUrUrUrCrUrCr | |
| | GrUrCrGrArUrGrGrUrCrArGrC*mA*mC*mA | |
| N20_NNAGAAA_ | mA*mA*mG*rGrCrUrGrUrGrCrUrGrArCrCrArUrCrGrArGrUrUrUrUrUrG | 636 |
| +5_S_F11R12_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrCrCrUrUrUrCrUrCr | |
| | GrUrCrGrArUrGrGrUrCrArG*mC*mA*mC | |
| N20_NNAGAAA_ | mA*mA*mG*rGrCrUrGrUrGrCrUrGrArCrCrArUrCrGrArGrUrUrUrUrUrG | 637 |
| +5_S_F10E12_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrCrCrUrUrUrCrUrCr | |
| | GrUrCrGrArUrGrGrUrCrA*mG*mC*mA | |
| N20_NNAGAAA_ | mA*mA*mG*rGrCrUrGrUrGrCrUrGrArCrCrArUrCrGrArGrUrUrUrUrUrG | 638 |
| +5_S_F9E12_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrCrCrUrUrUrCrUrCr | |
| | GrUrCrGrArUrGrGrUrC*mA*mG*mC | |
| N20_NNAGAAA_ | mA*mA*mG*rGrCrUrGrUrGrCrUrGrArCrCrArUrCrGrArGrUrUrUrUrUrG | 639 |
| +5_S_F8E12_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrCrCrUrUrUrCrUrCr | |
| | GrUrCrGrArUrGrGrU*mC*mA*mG | |
| N20_NNAGAAA_ | mA*mA*mG*rGrCrUrGrUrGrCrUrGrArCrCrArUrCrGrArGrUrUrUrUrUrG | 640 |
| +5_S_F7E12_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrCrCrUrUrUrCrUrCr | |
| | GrUrCrGrArUrGrG*mU*mC*mA | |
| N20_NNAGAAA_ | mA*mA*mG*rGrCrUrGrUrGrCrUrGrArCrCrArUrCrGrArGrUrUrUrUrUrG | 641 |
| +5_S_F12E14_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrCrCrUrUrUrCr | |
| | UrCrGrUrCrGrArUrGrGrUrCrArGrC*mA*mC*mA | |
| N20_NNAGAAA_ | mA*mA*mG*rGrCrUrGrUrGrCrUrGrArCrCrArUrCrGrArGrUrUrUrUrUrG | 642 |
| +5_S_F11R14_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrCrCrUrUrUrCr | |
| | UrCrGrUrCrGrArUrGrGrUrCrArG*mC*mA*mC | |
| N20_NNAGAAA_ | mA*mA*mG*rGrCrUrGrUrGrCrUrGrArCrCrArUrCrGrArGrUrUrUrUrUrG | 643 |
| +5_S_F10E14_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrCrCrUrUrUrCr | |
| | UrCrGrUrCrGrArUrGrGrUrCrA*mG*mC*mA | |
| N20_NNAGAAA_ | mA*mA*mG*rGrCrUrGrUrGrCrUrGrArCrCrArUrCrGrArGrUrUrUrUrUrG | 644 |
| +5_S_F9E14_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrCrCrUrUrUrCr | |
| | UrCrGrUrCrGrArUrGrGrUrC*mA*mG*mC | |
| N20_NNAGAAA_ | mA*mA*mG*rGrCrUrGrUrGrCrUrGrArCrCrArUrCrGrArGrUrUrUrUrUrG | 645 |
| +5_S_F8E14_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrCrCrUrUrUrCr | |
| | UrCrGrUrCrGrArUrGrGrU*mC*mA*mG | |
| N20_NNAGAAA_ | mA*mA*mG*rGrCrUrGrUrGrCrUrGrArCrCrArUrCrGrArGrUrUrUrUrUrG | 646 |
| +5_S_F7E14_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrUrCrCrCrUrUrUrCr | |
| | UrCrGrUrCrGrArUrGrG*mU*mC*mA | |
| N20_NNAGAAA_ | mA*mA*mG*rGrCrUrGrUrGrCrUrGrArCrCrArUrCrGrArGrUrUrUrUrUrG | 647 |
| +5_S_F12E16_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrArGrUrCrCrCrUrUr | |
| | UrCrUrCrGrUrCrGrArUrGrGrUrCrArGrC*mA*mC*mA | |
| N20_NNAGAAA_ | mA*mA*mG*rGrCrUrGrUrGrCrUrGrArCrCrArUrCrGrArGrUrUrUrUrUrG | 648 |
| +5_S_F11E16_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrArGrUrCrCrCrUrUr | |
| | UrCrUrCrGrUrCrGrArUrGrGrUrCrArG*mC*mA*mC | |
| N20_NNAGAAA_ | mA*mA*mG*rGrCrUrGrUrGrCrUrGrArCrCrArUrCrGrArGrUrUrUrUrUrG | 649 |
| +5_S_F10E16_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrArGrUrCrCrCrUrUr | |
| | UrCrUrCrGrUrCrGrArUrGrGrUrCrA*mG*mC*mA | |
| N20_NNAGAAA_ | mA*mA*mG*rGrCrUrGrUrGrCrUrGrArCrCrArUrCrGrArGrUrUrUrUrUrG | 650 |
| +5_S_F9E16_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrArGrUrCrCrCrUrUr | |
| | UrCrUrCrGrUrCrGrArUrGrGrUrC*mA*mG*mC | |
| N20_NNAGAAA_ | mA*mA*mG*rGrCrUrGrUrGrCrUrGrArCrCrArUrCrGrArGrUrUrUrUrUrG | 651 |
| +5_S_F8R16_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrArGrUrCrCrCrUrUr | |
| | UrCrUrCrGrUrCrGrArUrGrGrU*mC*mA*mG | |
| N20_NNAGAAA_ | mA*mA*mG*rGrCrUrGrUrGrCrUrGrArCrCrArUrCrGrArGrUrUrUrUrUrG | 652 |
| +5_S_F7E16_TtoC | rUrArCrUrCrUrCrArArGrArArArUrUrGrCrArGrArArGrCrUrArCrArArArG | |
| | rArUrArArGrGrCrUrUrCrArUrGrCrCrGrArArArUrCrArArCrArCrCrCrUrG | |
| | rUrCrArUrUrUrUrArUrGrGrCrArGrGrGrUrGrUrUrArGrUrCrCrCrUrUr | |
| | UrCrUrCrGrUrCrGrArUrGrG*mU*mC*mA | |
| N20_NAA_+1_S_ | mC*mU*mG*rUrGrCrUrGrArCrCrArUrCrGrArCrArArGrGrUrUrUrUrArGr | 653 |
| F12E4_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrUrCrGrUrCrGrArUrGrGrU*mC*mA*mG | |
| N20_NAA_+1_S_ | mC*mU*mG*rUrGrCrUrGrArCrCrArUrCrGrArCrArArGrGrUrUrUrUrArGr | 654 |
| F11E4_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrUrCrGrUrCrGrArUrGrG*mU*mC*mA | |
| N20_NAA_+1_S_ | mC*mU*mG*rUrGrCrUrGrArCrCrArUrCrGrArCrArArGrGrUrUrUrUrArGr | 655 |
| F10E4_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrUrCrGrUrCrGrArUrG*mG*mU*mC | |
| N20_NAA_+1_S_ | mC*mU*mG*rUrGrCrUrGrArCrCrArUrCrGrArCrArArGrGrUrUrUrUrArGr | 656 |
| F9E4_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrUrCrGrUrCrGrArU*mG*mG*mU | |
| N20_NAA_+1_S_ | mC*mU*mG*rUrGrCrUrGrArCrCrArUrCrGrArCrArArGrGrUrUrUrUrArGr | 657 |
| F8E4_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrUrCrGrUrCrGrA*mU*mG*mG | |
| N20_NAA_+1_S_ | mC*mU*mG*rUrGrCrUrGrArCrCrArUrCrGrArCrArArGrGrUrUrUrUrArGr | 658 |
| F7E4_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrUrCrGrUrCrG*mA*mU*mG | |
| N20_NAA_+1_S_ | mC*mU*mG*rUrGrCrUrGrArCrCrArUrCrGrArCrArArGrGrUrUrUrUrArGr | 659 |
| F12E6_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrUrUrCrUrCrGrUrCrGrArUrGrGrU*mC*mA | |
| | *mG | |
| N20_NAA_+1_S_ | mC*mU*mG*rUrGrCrUrGrArCrCrArUrCrGrArCrArArGrGrUrUrUrUrArGr | 660 |
| F11E6_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrUrUrCrUrCrGrUrCrGrArUrGrG*mU*mC* | |
| | mA | |
| N20_NAA_+1_S_ | mC*mU*mG*rUrGrCrUrGrArCrCrArUrCrGrArCrArArGrGrUrUrUrUrArGr | 661 |
| F10E6_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrUrUrCrUrCrGrUrCrGrArUrG*mG*mU*mC | |
| N20_NAA_+1_S_ | mC*mU*mG*rUrGrCrUrGrArCrCrArUrCrGrArCrArArGrGrUrUrUrUrArGr | 662 |
| F9E6_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrUrUrCrUrCrGrUrCrGrArU*mG*mG*mU | |
| N20_NAA_+1_S_ | mC*mU*mG*rUrGrCrUrGrArCrCrArUrCrGrArCrArArGrGrUrUrUrUrArGr | 663 |
| F8E6_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrUrUrCrUrCrGrUrCrGrA*mU*mG*mG | |
| N20_NAA_+1_S_ | mC*mU*mG*rUrGrCrUrGrArCrCrArUrCrGrArCrArArGrGrUrUrUrUrArGr | 664 |
| F7E6_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrUrUrCrUrCrGrUrCrG*mA*mU*mG | |
| N20_NAA_+1_S_ | mC*mU*mG*rUrGrCrUrGrArCrCrArUrCrGrArCrArArGrGrUrUrUrUrArGr | 665 |
| F12E8_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrCrCrUrUrUrCrUrCrGrUrCrGrArUrGrGrU*m | |
| | C*mA*mG | |
| N20_NAA_+1_S_ | mC*mU*mG*rUrGrCrUrGrArCrCrArUrCrGrArCrArArGrGrUrUrUrUrArGr | 666 |
| F11R8_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrCrCrUrUrUrCrUrCrGrUrCrGrArUrGrG*mU* | |
| | mC*mA | |
| N20_NAA_+1_S_ | mC*mU*mG*rUrGrCrUrGrArCrCrArUrCrGrArCrArArGrGrUrUrUrUrArGr | 667 |
| F10E8_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrCrCrUrUrUrCrUrCrGrUrCrGrArUrG*mG*mU | |
| | *mC | |
| N20_NAA_+1_S_ | mC*mU*mG*rUrGrCrUrGrArCrCrArUrCrGrArCrArArGrGrUrUrUrUrArGr | 668 |
| F9E8_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrCrCrUrUrUrCrUrCrGrUrCrGrArU*mG*mG* | |
| | mU | |
| N20_NAA_+1_S_ | mC*mU*mG*rUrGrCrUrGrArCrCrArUrCrGrArCrArArGrGrUrUrUrUrArGr | 669 |
| F8E8_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrCrCrUrUrUrCrUrCrGrUrCrGrA*mU*mG*mG | |
| N20_NAA_+1_S_ | mC*mU*mG*rUrGrCrUrGrArCrCrArUrCrGrArCrArArGrGrUrUrUrUrArGr | 670 |
| F7E8_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrCrCrUrUrUrCrUrCrGrUrCrG*mA*mU*mG | |
| N20_NAA_+1_S_ | mC*mU*mG*rUrGrCrUrGrArCrCrArUrCrGrArCrArArGrGrUrUrUrUrArGr | 671 |
| F12E10_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrCrCrUrUrUrCrUrCrGrUrCrGrArUrGrGr | |
| | U*mC*mA*mG | |
| N20_NAA_+1_S_ | mC*mU*mG*rUrGrCrUrGrArCrCrArUrCrGrArCrArArGrGrUrUrUrUrArGr | 672 |
| F11E10_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrCrCrUrUrUrCrUrCrGrUrCrGrArUrGrG* | |
| | mU*mC*mA | |
| N20_NAA_+1_S_ | mC*mU*mG*rUrGrCrUrGrArCrCrArUrCrGrArCrArArGrGrUrUrUrUrArGr | 673 |
| F10E10_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrCrCrUrUrUrCrUrCrGrUrCrGrArUrG*m | |
| | G*mU*mC | |
| N20_NAA_+1_S_ | mC*mU*mG*rUrGrCrUrGrArCrCrArUrCrGrArCrArArGrGrUrUrUrUrArGr | 674 |
| F9E10_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrCrCrUrUrUrCrUrCrGrUrCrGrArU*mG* | |
| | mG*mU | |
| N20_NAA_+1_S_ | mC*mU*mG*rUrGrCrUrGrArCrCrArUrCrGrArCrArArGrGrUrUrUrUrArGr | 675 |
| F8E10_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrCrCrUrUrUrCrUrCrGrUrCrGrA*mU*mG | |
| | *mG | |
| N20_NAA_+1_S_ | mC*mU*mG*rUrGrCrUrGrArCrCrArUrCrGrArCrArArGrGrUrUrUrUrArGr | 676 |
| F7E10_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrUrCrCrCrUrUrUrCrUrCrGrUrCrG*mA*mU*m | |
| | G | |
| N20_NAA_+1_S_ | mC*mU*mG*rUrGrCrUrGrArCrCrArUrCrGrArCrArArGrGrUrUrUrUrArGr | 677 |
| F12E12_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrArGrUrCrCrCrUrUrUrCrUrCrGrUrCrGrArUr | |
| | GrGrU*mC*mA*mG | |
| N20_NAA_+1_S_ | mC*mU*mG*rUrGrCrUrGrArCrCrArUrCrGrArCrArArGrGrUrUrUrUrArGr | 678 |
| F11E12_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrArGrUrCrCrCrUrUrUrCrUrCrGrUrCrGrArUr | |
| | GrG*mU*mC*mA | |
| N20_NAA_+1_S_ | mC*mU*mG*rUrGrCrUrGrArCrCrArUrCrGrArCrArArGrGrUrUrUrUrArGr | 679 |
| F10R12_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrArGrUrCrCrCrUrUrUrCrUrCrGrUrCrGrArUr | |
| | G*mG*mU*mC | |
| N20_NAA_+1_S_ | mC*mU*mG*rUrGrCrUrGrArCrCrArUrCrGrArCrArArGrGrUrUrUrUrArGr | 680 |
| F9E12_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrArGrUrCrCrCrUrUrUrCrUrCrGrUrCrGrArU* | |
| | mG*mG*mU | |
| N20_NAA_+1_S_ | mC*mU*mG*rUrGrCrUrGrArCrCrArUrCrGrArCrArArGrGrUrUrUrUrArGr | 681 |
| F8E12_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrArGrUrCrCrCrUrUrUrCrUrCrGrUrCrGrA*m | |
| | U*mG*mG | |
| N20_NAA_+1_S_ | mC*mU*mG*rUrGrCrUrGrArCrCrArUrCrGrArCrArArGrGrUrUrUrUrArGr | 682 |
| F7E12_TtoC | ArGrCrUrArGrArArArUrArGrCrArArGrUrUrArArArArUrArArGrGrCrUrA | |
| | rGrUrCrCrGrUrUrArUrCrArArCrUrUrGrArArArArArGrUrGrGrCrArCrCrG | |
| | rArGrUrCrGrGrUrGrCrArGrUrCrCrCrUrUrUrCrUrCrGrUrCrG*mA* | |
| | mU*mG | |

¹For RNA, T is U; U are T in the sequence listing. For each entry, the spacer sequence is underlined and the extension arm is bold. The description indicates the length of the FBS (F) and ET (E) or homology region of the extension arm.
²For the sequences: *, phosphorothioate modification; mN, 2′O-methyl modification; rN, unmodified ribonucleotide; i2FN, Int Fluoro modification. N = A, C, T/U, G.

TABLE 13C
EXEMPLARY SPACER AND PROTOSPACER SEQUENCES FOR EDITING
SERPINA1

| SEQ ID | | SEQ ID | |
|---|---|---|---|
| NO: | SPACER SEQUENCE¹ | NO: | PROTOSPACER SEQUENCE |
| 815 | AAGGCUGUGCUGACCAUCGA | 817 | AAGGCTGTGCTGACCATCGA |
| 816 | CUGUGCUGACCAUCGACAAG | 818 | CTGTGCTGACCATCGACAAG |
| 1273 | CAUAAGGCUGUGCUGACCAUCGA | 1270 | CATAAGGCTGTGCTGACCATCGA |
| 1274 | AUAAGGCUGUGCUGACCAUCGA | 1271 | ATAAGGCTGTGCTGACCATCGA |
| 1275 | UAAGGCUGUGCUGACCAUCGA | 1272 | TAAGGCTGTGCTGACCATCGA |

¹For RNA, U are T in the sequence listing.

TABLE 13D
EXEMPLARY FLAP BINDING SEQUENCES AND TEMPLATES FOR
EDITING SERPINA1

| | | SEQ ID | EDITING | SEQ ID |
|---|---|---|---|---|
| DESCRIPTION | FBS¹ | NO: | TEMPLATE | NO: |
| N20_NNAGAAA_+5_S_F12E8_TtoC | AUGGUCAGCACA | 825 | UCUCGUCG | |
| N20_NNAGAAA_+5_S_F11E8_TtoC | AUGGUCAGCAC | 826 | UCUCGUCG | |
| N20_NNAGAAA_+5_S_F10E8_TtoC | AUGGUCAGCA | 827 | UCUCGUCG | |
| N20_NNAGAAA_+5_S_F9E8_TtoC | AUGGUCAGC | | UCUCGUCG | |
| N20_NNAGAAA_+5_S_F8E8_TtoC | AUGGUCAG | | UCUCGUCG | |
| N20_NNAGAAA_+5_S_F7E8_TtoC | AUGGUCA | | UCUCGUCG | |
| N20_NNAGAAA_+5_S_F12E10_TtoC | AUGGUCAGCACA | 825 | UUUCUCGUCG | 819 |
| N20_NNAGAAA_+5_S_F11E10_TtoC | AUGGUCAGCAC | 826 | UUUCUCGUCG | 819 |
| N20_NNAGAAA_+5_S_F10E10_TtoC | AUGGUCAGCA | 827 | UUUCUCGUCG | 819 |
| N20_NNAGAAA_+5_S_F9E10_TtoC | AUGGUCAGC | | UUUCUCGUCG | 819 |
| N20_NNAGAAA_+5_S_F8E10_TtoC | AUGGUCAG | | UUUCUCGUCG | 819 |
| N20_NNAGAAA_+5_S_F7E10_TtoC | AUGGUCA | | UUUCUCGUCG | 819 |
| N20_NNAGAAA_+5_S_F12E12_TtoC | AUGGUCAGCACA | 825 | CCUUUCUCGUCG | 820 |
| N20_NNAGAAA_+5_S_F11E12_TtoC | AUGGUCAGCAC | 826 | CCUUUCUCGUCG | 820 |
| N20_NNAGAAA_+5_S_F10E12_TtoC | AUGGUCAGCA | 827 | CCUUUCUCGUCG | 820 |
| N20_NNAGAAA_+5_S_F9E12_TtoC | AUGGUCAGC | | CCUUUCUCGUCG | 820 |
| N20_NNAGAAA_+5_S_F8E12_TtoC | AUGGUCAG | | CCUUUCUCGUCG | 820 |
| N20_NNAGAAA_+5_S_F7E12_TtoC | AUGGUCA | | CCUUUCUCGUCG | 820 |
| N20_NNAGAAA_+5_S_F12E14_TtoC | AUGGUCAGCACA | 825 | UCCCUUUCUCGUCG | 821 |
| N20_NNAGAAA_+5_S_F11E14_TtoC | AUGGUCAGCAC | 826 | UCCCUUUCUCGUCG | 821 |
| N20_NNAGAAA_+5_S_F10E14_TtoC | AUGGUCAGCA | 827 | UCCCUUUCUCGUCG | 821 |
| N20_NNAGAAA_+5_S_F9E14_TtoC | AUGGUCAGC | | UCCCUUUCUCGUCG | 821 |
| N20_NNAGAAA_+5_S_F8E14_TtoC | AUGGUCAG | | UCCCUUUCUCGUCG | 821 |
| N20_NNAGAAA_+5_S_F7E14_TtoC | AUGGUCA | | UCCCUUUCUCGUCG | 821 |
| N20_NNAGAAA_+5_S_F12E16_TtoC | AUGGUCAGCACA | 825 | AGUCCCUUUCUCGUCG | 822 |
| N20_NNAGAAA_+5_S_F11E16_TtoC | AUGGUCAGCAC | 826 | AGUCCCUUUCUCGUCG | 822 |
| N20_NNAGAAA_+5_S_F10E16_TtoC | AUGGUCAGCA | 827 | AGUCCCUUUCUCGUCG | 822 |
| N20_NNAGAAA_+5_S_F9E16_TtoC | AUGGUCAGC | | AGUCCCUUUCUCGUCG | 822 |
| N20_NNAGAAA_+5_S_F8E16_TtoC | AUGGUCAG | | AGUCCCUUUCUCGUCG | 822 |
| N20_NNAGAAA_+5_S_F7E16_TtoC | AUGGUCA | | AGUCCCUUUCUCGUCG | 822 |
| N20_NAA_+1_S_F12E4_TtoC | GUCGAUGGUCAG | 828 | UCUC | |
| N20_NAA_+1_S_F11E4_TtoC | GUCGAUGGUCA | 829 | UCUC | |
| N20_NAA_+1_S_F10E4_TtoC | GUCGAUGGUC | 830 | UCUC | |
| N20_NAA_+1_S_F9E4_TtoC | GUCGAUGGU | | UCUC | |
| N20_NAA_+1_S_F8E4_TtoC | GUCGAUGG | | UCUC | |
| N20_NAA_+1_S_F7E4_TtoC | GUCGAUG | | UCUC | |
| N20_NAA_+1_S_F12E6_TtoC | GUCGAUGGUCAG | 828 | UUUCUC | |
| N20_NAA_+1_S_F11E6_TtoC | GUCGAUGGUCA | 829 | UUUCUC | |
| N20_NAA_+1_S_F10E6_TtoC | GUCGAUGGUC | 830 | UUUCUC | |
| N20_NAA_+1_S_F9E6_TtoC | GUCGAUGGU | | UUUCUC | |
| N20_NAA_+1_S_F8E6_TtoC | GUCGAUGG | | UUUCUC | |
| N20_NAA_+1_S_F7E6_TtoC | GUCGAUG | | UUUCUC | |
| N20_NAA_+1_S_F12E8_TtoC | GUCGAUGGUCAG | 828 | CCUUUCUC | |
| N20_NAA_+1_S_F11E8_TtoC | GUCGAUGGUCA | 829 | CCUUUCUC | |
| N20_NAA_+1_S_F10E8_TtoC | GUCGAUGGUC | 830 | CCUUUCUC | |
| N20_NAA_+1_S_F9E8_TtoC | GUCGAUGGU | | CCUUUCUC | |
| N20_NAA_+1_S_F8E8_TtoC | GUCGAUGG | | CCUUUCUC | |
| N20_NAA_+1_S_F7E8_TtoC | GUCGAUG | | CCUUUCUC | |
| N20_NAA_+1_S_F12E10_TtoC | GUCGAUGGUCAG | 828 | UCCCUUUCUC | 823 |
| N20_NAA_+1_S_F11E10_TtoC | GUCGAUGGUCA | 829 | UCCCUUUCUC | 823 |
| N20_NAA_+1_S_F10E10_TtoC | GUCGAUGGUC | 830 | UCCCUUUCUC | 823 |
| N20_NAA_+1_S_F9E10_TtoC | GUCGAUGGU | | UCCCUUUCUC | 823 |
| N20_NAA_+1_S_F8E10_TtoC | GUCGAUGG | | UCCCUUUCUC | 823 |
| N20_NAA_+1_S_F7E10_TtoC | GUCGAUG | | UCCCUUUCUC | 823 |
| N20_NAA_+1_S_F12E12_TtoC | GUCGAUGGUCAG | 828 | AGUCCCUUUCUC | 824 |
| N20_NAA_+1_S_F11E12_TtoC | GUCGAUGGUCA | 829 | AGUCCCUUUCUC | 824 |
| N20_NAA_+1_S_F10E12_TtoC | GUCGAUGGUC | 830 | AGUCCCUUUCUC | 824 |
| N20_NAA_+1_S_F9E12_TtoC | GUCGAUGGU | | AGUCCCUUUCUC | 824 |
| N20_NAA_+1_S_F8E12_TtoC | GUCGAUGG | | AGUCCCUUUCUC | 824 |
| N20_NAA_+1_S_F7E12_TtoC | GUCGAUG | | AGUCCCUUUCUC | 824 |

¹For RNA, T is U; U are T in the sequence listing. The description indicates the length of the FBS (F) and ET (E) or homology region of the extension arm.

TABLE 14A
EXEMPLARY SCAFFOLD VARIANTS

| | | | SEQ ID |
|---|---|---|---|
| NAME | DESCRIPTION | SEQUENCE1 | NO: |
| v0 | wild type | GUUUUUGUACUCUCAAGAAAUUGCAGAAGCUACAAAGAU | 683 |
| | | AAGGCUUCAUGCCGAAAUCAACACCCUGUCAUUUUAUGGC | |
| | | AGGGUGUU | |
| v1 | UA:AU to GC:CG | GUUUGCGUACUCUCAAGAAAUUGCAGAAGCUACGCAGAU | 684 |
| | | AAGGCUUCAUGCCGAAAUCAACACCCUGUCAUUUUAUGGC | |
| | | AGGGUGUU | |
| v2 | RAR trunc 1 | GUUUUUGUACUCUGAAAAGAAGCUACAAAGAUAAGGCUU | 685 |
| | | CAUGCCGAAAUCAACACCCUGUCAUUUUAUGGCAGGGUG | |
| | | UU | |
| v3 | RAR trunc2 + UUCG | GUUUUUGUACGUUCGCGCUACAAAGAUAAGGCUUCAUGC | 686 |
| | | CGAAAUCAACACCCUGUCAUUUUAUGGCAGGGUGUU | |
| v4 | UA:AU + RAR trunc1 | GUUUGCGUACUCUGAAAAGAAGCUACGCAGAUAAGGCUU | 687 |
| | | CAUGCCGAAAUCAACACCCUGUCAUUUUAUGGCAGGGUG | |
| | | UU | |
| v5 | UA:AU + RAR trunc2 | GUUUGCGUACGUUCGCGCUACGCAGAUAAGGCUUCAUGCC | 688 |
| | | GAAAUCAACACCCUGUCAUUUUAUGGCAGGGUGUU | |
| v6 | SL2_trunc1 | GUUUUUGUACUCUCAAGAAAUUGCAGAAGCUACAAAGAU | 689 |
| | | AAGGCUUCAUGCCGAAAUCAACA | |
| v7 | SL2_trunc2 | GUUUUUGUACUCUCAAGAAAUUGCAGAAGCUACAAAGAU | 690 |
| | | AAGGCUUCAUGCCGAAAUCAA | |
| v8 | SL2_trunc3 | GUUUUUGUACUCUCAAGAAAUUGCAGAAGCUACAAAGAU | 691 |
| | | AAGGCUUCAUGCCGAAAUC | |
| v9 | SL2_trunc4 | GUUUUUGUACUCUCAAGAAAUUGCAGAAGCUACAAAGAU | 692 |
| | | AAGGCUUCAUGCCGAAA | |
| v10 | SL2_trunc5 | GUUUUUGUACUCUCAAGAAAUUGCAGAAGCUACAAAGAU | 693 |
| | | AAGGCUUCAUGCCGA | |
| v11 | UA:AU to GC:CG + | GUUUGCGUACUCUCAAGAAAUUGCAGAAGCUACGCAGAU | 694 |
| | SL2_trunc1 | AAGGCUUCAUGCCGAAAUCAACA | |
| v12 | RAR trunc1 + SL2_ | GUUUUUGUACUCUGAAAAGAAGCUACAAAGAUAAGGCUU | 695 |
| | trunc1 | CAUGCCGAAAUCAACA | |
| v13 | RAR trunc2 + UUCG+ | GUUUUUGUACGUUCGCGCUACAAAGAUAAGGCUUCAUGC | 696 |
| | SL2_trunc1 | CGAAAUCAACA | |
| v14 | UA:AU+RAR trunc1 + | GUUUGCGUACUCUGAAAAGAAGCUACGCAGAUAAGGCUU | 697 |
| | SL2_trunc1 | CAUGCCGAAAUCAACA | |
| v15 | UA:AU+RAR trunc2 + | GUUUGCGUACGUUCGCGCUACGCAGAUAAGGCUUCAUGCC | 698 |
| | SL2_trunc1 | GAAAUCAACA | |
| v16 | UA:AU to GC:CG + | GUUUGCGUACUCUCAAGAAAUUGCAGAAGCUACGCAGAU | 699 |
| | SL2_trunc2 | AAGGCUUCAUGCCGAAAUCAA | |
| v17 | RAR trunc1 + | GUUUUUGUACUCUGAAAAGAAGCUACAAAGAUAAGGCUU | 700 |
| | SL2_trunc2 | CAUGCCGAAAUCAA | |
| v18 | RAR trunc2 + UUCG + | GUUUUUGUACGUUCGCGCUACAAAGAUAAGGCUUCAUGC | 701 |
| | SL2 _trunc2 | CGAAAUCAA | |
| v19 | UA:AU + RAR trunc1 + | GUUUGCGUACUCUGAAAAGAAGCUACGCAGAUAAGGCUU | 702 |
| | SL2_trunc2 | CAUGCCGAAAUCAA | |
| v20 | UA:AU+RAR trunc2+ | GUUUGCGUACGUUCGCGCUACGCAGAUAAGGCUUCAUGCC | 703 |
| | SL2_trunc2 | GAAAUCAA | |
| v21 | UA:AU to GC:CG + | GUUUGCGUACUCUCAAGAAAUUGCAGAAGCUACGCAGAU | 704 |
| | SL2_trunc3 | AAGGCUUCAUGCCGAAAUC | |
| v22 | RAR trunc1 + | GUUUUUGUACUCUGAAAAGAAGCUACAAAGAUAAGGCUU | 705 |
| | SL2_trunc3 | CAUGCCGAAAUC | |
| v23 | RAR trunc2 + UUCG + | GUUUUUGUACGUUCGCGCUACAAAGAUAAGGCUUCAUGC | 706 |
| | SL2_trunc3 | CGAAAUC | |
| v24 | UA:AU + RAR trunc1 + | GUUUGCGUACUCUGAAAAGAAGCUACGCAGAUAAGGCUU | 707 |
| | SL2_trunc3 | CAUGCCGAAAUC | |
| v25 | UA:AU + RAR trunc2 + | GUUUGCGUACGUUCGCGCUACGCAGAUAAGGCUUCAUGCC | 708 |
| | SL2_trunc3 | GAAAUC | |
| v26 | UA:AU to GC:CG + | GUUUGCGUACUCUCAAGAAAUUGCAGAAGCUACGCAGAU | 709 |
| | SL2_trunc4 | AAGGCUUCAUGCCGAAA | |
| v27 | RAR trunc 1 + | GUUUUUGUACUCUGAAAAGAAGCUACAAAGAUAAGGCUU | 710 |
| | SL2_trunc4 | CAUGCCGAAA | |
| v28 | RAR trunc2 + UUCG + | GUUUUUGUACGUUCGCGCUACAAAGAUAAGGCUUCAUGC | 711 |
| | SL2_trunc4 | CGAAA | |
| v29 | UA:AU + RAR trunc1 + | GUUUGCGUACUCUGAAAAGAAGCUACGCAGAUAAGGCUU | 712 |
| | SL2_trunc4 | CAUGCCGAAA | |
| v30 | UA:AU + RAR trunc2 + | GUUUGCGUACGUUCGCGCUACGCAGAUAAGGCUUCAUGCC | 713 |
| | SL2_trunc4 | GAAA | |
| v31 | UA:AU to GC:CG + | GUUUGCGUACUCUCAAGAAAUUGCAGAAGCUACGCAGAU | 714 |
| | SL2_trunc5 | AAGGCUUCAUGCCGA | |
| v32 | RAR trunc1 + | GUUUUUGUACUCUGAAAAGAAGCUACAAAGAUAAGGCUU | 715 |
| | SL2_trunc5 | CAUGCCGA | |
| v33 | RAR trunc2 + UUCG + | GUUUUUGUACGUUCGCGCUACAAAGAUAAGGCUUCAUGC | 716 |
| | SL2_trunc5 | CGA | |
| v34 | UA:AU + RAR trunc1 + | GUUUGCGUACUCUGAAAAGAAGCUACGCAGAUAAGGCUU | 717 |
| | SL2_trunc5 | CAUGCCGA | |
| v35 | UA:AU + RAR trunc2 + | GUUUGCGUACGUUCGCGCUACGCAGAUAAGGCUUCAUGCC | 718 |
| | SL2_trunc5 | GA | |

¹For RNA, T is U; U are T in the sequence listing.

TABLE 14B
EXEMPLARY tagRNA SCAFFOLD VARIANTS

| | | | SEQ ID |
|---|---|---|---|
| NAME | DESCRIPTION | SEQUENCE^{1,} | NO: |
| v0 | wild type | AAGGCUGUGCUGACCAUCGAGUUUUUGUACUC | 719 |
| | | UCAAGAAAUUGCAGAAGCUACAAAGAUAAGGC | |
| | | UUCAUGCCGAAAUCAACACCCUGUCAUUUUAUG | |
| | | GCAGGGUGUUUCUCGUCGAUGGUCAG | |
| v1 | UA:AU to GC:CG | AAGGCUGUGCUGACCAUCGAGUUUGCGUACUC | 720 |
| | | UCAAGAAAUUGCAGAAGCUACGCAGAUAAGGC | |
| | | UUCAUGCCGAAAUCAACACCCUGUCAUUUUAUG | |
| | | GCAGGGUGUUUCUCGUCGAUGGUCAG | |
| v2 | RAR trunc 1 | AAGGCUGUGCUGACCAUCGAGUUUUUGUACUC | 721 |
| | | UGAAAAGAAGCUACAAAGAUAAGGCUUCAUGC | |
| | | CGAAAUCAACACCCUGUCAUUUUAUGGCAGGG | |
| | | UGUUUCUCGUCGAUGGUCAG | |
| v3 | RAR trunc2 + UUCG | AAGGCUGUGCUGACCAUCGAGUUUUUGUACGU | 722 |
| | | UCGCGCUACAAAGAUAAGGCUUCAUGCCGAAA | |
| | | UCAACACCCUGUCAUUUUAUGGCAGGGUGUUU | |
| | | CUCGUCGAUGGUCAG | |
| v4 | UA:AU + RAR trunc1 | AAGGCUGUGCUGACCAUCGAGUUUGCGUACUC | 723 |
| | | UGAAAAGAAGCUACGCAGAUAAGGCUUCAUGC | |
| | | CGAAAUCAACACCCUGUCAUUUUAUGGCAGGG | |
| | | UGUUUCUCGUCGAUGGUCAG | |
| v5 | UA:AU + RAR trunc2 | AAGGCUGUGCUGACCAUCGAGUUUGCGUACGU | 724 |
| | | UCGCGCUACGCAGAUAAGGCUUCAUGCCGAAAU | |
| | | CAACACCCUGUCAUUUUAUGGCAGGGUGUUUC | |
| | | UCGUCGAUGGUCAG | |
| v6 | SL2_trunc1 | AAGGCUGUGCUGACCAUCGAGUUUUUGUACUC | 725 |
| | | UCAAGAAAUUGCAGAAGCUACAAAGAUAAGGC | |
| | | UUCAUGCCGAAAUCAACAUCUCGUCGAUGGUC | |
| | | AG | |
| v7 | SL2_trunc2 | AAGGCUGUGCUGACCAUCGAGUUUUUGUACUC | 726 |
| | | UCAAGAAAUUGCAGAAGCUACAAAGAUAAGGC | |
| | | UUCAUGCCGAAAUCAAUCUCGUCGAUGGUCAG | |
| v8 | SL2_trunc3 | AAGGCUGUGCUGACCAUCGAGUUUUUGUACUC | 727 |
| | | UCAAGAAAUUGCAGAAGCUACAAAGAUAAGGC | |
| | | UUCAUGCCGAAAUCUCUCGUCGAUGGUCAG | |
| v9 | SL2_trunc4 | AAGGCUGUGCUGACCAUCGAGUUUUUGUACUC | 728 |
| | | UCAAGAAAUUGCAGAAGCUACAAAGAUAAGGC | |
| | | UUCAUGCCGAAAUCUCGUCGAUGGUCAG | |
| v10 | SL2_trunc5 | AAGGCUGUGCUGACCAUCGAGUUUUUGUACUC | 729 |
| | | UCAAGAAAUUGCAGAAGCUACAAAGAUAAGGC | |
| | | UUCAUGCCGAUCUCGUCGAUGGUCAG | |
| v11 | UA:AU to GC:CG + | AAGGCUGUGCUGACCAUCGAGUUUGCGUACUC | 730 |
| | SL2_trunc1 | UCAAGAAAUUGCAGAAGCUACGCAGAUAAGGC | |
| | | UUCAUGCCGAAAUCAACAUCUCGUCGAUGGUC | |
| | | AG | |
| v12 | RAR trunc1 + SL2_trunc1 | AAGGCUGUGCUGACCAUCGAGUUUUUGUACUC | 731 |
| | | UGAAAAGAAGCUACAAAGAUAAGGCUUCAUGC | |
| | | CGAAAUCAACAUCUCGUCGAUGGUCAG | |
| v13 | RAR trunc2 + UUCG + | AAGGCUGUGCUGACCAUCGAGUUUUUGUACGU | 732 |
| | SL2_trunc1 | UCGCGCUACAAAGAUAAGGCUUCAUGCCGAAA | |
| | | UCAACAUCUCGUCGAUGGUCAG | |
| v14 | UA:AU + RAR trunc1 + | AAGGCUGUGCUGACCAUCGAGUUUGCGUACUC | 733 |
| | SL2_trunc1 | UGAAAAGAAGCUACGCAGAUAAGGCUUCAUGC | |
| | | CGAAAUCAACAUCUCGUCGAUGGUCAG | |
| v15 | UA:AU + RAR trunc2 + | AAGGCUGUGCUGACCAUCGAGUUUGCGUACGU | 734 |
| | SL2_trunc1 | UCGCGCUACGCAGAUAAGGCUUCAUGCCGAAAU | |
| | | CAACAUCUCGUCGAUGGUCAG | |
| v16 | UA:AU to GC:CG + | AAGGCUGUGCUGACCAUCGAGUUUGCGUACUC | 735 |
| | SL2_trunc2 | UCAAGAAAUUGCAGAAGCUACGCAGAUAAGGC | |
| | | UUCAUGCCGAAAUCAAUCUCGUCGAUGGUCAG | |
| v17 | RAR trunc1 + SL2_trunc2 | AAGGCUGUGCUGACCAUCGAGUUUUUGUACUC | 736 |
| | | UGAAAAGAAGCUACAAAGAUAAGGCUUCAUGC | |
| | | CGAAAUCAAUCUCGUCGAUGGUCAG | |
| v18 | RAR trunc2 + UUCG + | AAGGCUGUGCUGACCAUCGAGUUUUUGUACGU | 737 |
| | SL2_trunc2 | UCGCGCUACAAAGAUAAGGCUUCAUGCCGAAA | |
| | | UCAAUCUCGUCGAUGGUCAG | |
| v19 | UA:AU + RAR trunc1 + | AAGGCUGUGCUGACCAUCGAGUUUGCGUACUC | 738 |
| | SL2_trunc2 | UGAAAAGAAGCUACGCAGAUAAGGCUUCAUGC | |
| | | CGAAAUCAAUCUCGUCGAUGGUCAG | |
| v20 | UA:AU + RAR trunc2 + | AAGGCUGUGCUGACCAUCGAGUUUGCGUACGU | 739 |
| | SL2_trunc2 | UCGCGCUACGCAGAUAAGGCUUCAUGCCGAAAU | |
| | | CAAUCUCGUCGAUGGUCAG | |
| v21 | UA:AU to GC:CG + SL2_trunc3 | AAGGCUGUGCUGACCAUCGAGUUUGCGUACUC | 740 |
| | | UCAAGAAAUUGCAGAAGCUACGCAGAUAAGGC | |
| | | UUCAUGCCGAAAUCUCUCGUCGAUGGUCAG | |
| v22 | RAR trunc1 + SL2_trunc3 | AAGGCUGUGCUGACCAUCGAGUUUUUGUACUC | 741 |
| | | UGAAAAGAAGCUACAAAGAUAAGGCUUCAUGC | |
| | | CGAAAUCUCUCGUCGAUGGUCAG | |
| v23 | RAR trunc2 + UUCG + | AAGGCUGUGCUGACCAUCGAGUUUUUGUACGU | 742 |
| | SL2_trunc3 | UCGCGCUACAAAGAUAAGGCUUCAUGCCGAAA | |
| | | UCUCUCGUCGAUGGUCAG | |
| v24 | UA:AU + RAR trunc1 + | AAGGCUGUGCUGACCAUCGAGUUUGCGUACUC | 743 |
| | SL2_trunc3 | UGAAAAGAAGCUACGCAGAUAAGGCUUCAUGC | |
| | | CGAAAUCUCUCGUCGAUGGUCAG | |
| v25 | UA:AU + RAR trunc2 + | AAGGCUGUGCUGACCAUCGAGUUUGCGUACGU | 744 |
| | SL2_trunc3 | UCGCGCUACGCAGAUAAGGCUUCAUGCCGAAAU | |
| | | CUCUCGUCGAUGGUCAG | |
| v26 | UA:AU to GC:CG + SL2_trunc4 | AAGGCUGUGCUGACCAUCGAGUUUGCGUACUC | 745 |
| | | UCAAGAAAUUGCAGAAGCUACGCAGAUAAGGC | |
| | | UUCAUGCCGAAAUCUCGUCGAUGGUCAG | |
| v27 | RAR trunc1 + SL2_trunc4 | AAGGCUGUGCUGACCAUCGAGUUUUUGUACUC | 746 |
| | | UGAAAAGAAGCUACAAAGAUAAGGCUUCAUGC | |
| | | CGAAAUCUCGUCGAUGGUCAG | |
| v28 | RAR trunc2 + UUCG + | AAGGCUGUGCUGACCAUCGAGUUUUUGUACGU | 747 |
| | SL2_trunc4 | UCGCGCUACAAAGAUAAGGCUUCAUGCCGAAA | |
| | | UCUCGUCGAUGGUCAG | |
| v29 | UA:AU + RAR trunc 1 + | AAGGCUGUGCUGACCAUCGAGUUUGCGUACUC | 748 |
| | SL2_trunc4 | UGAAAAGAAGCUACGCAGAUAAGGCUUCAUGC | |
| | | CGAAAUCUCGUCGAUGGUCAG | |
| v30 | UA:AU + RAR trunc2 + | AAGGCUGUGCUGACCAUCGAGUUUGCGUACGU | 749 |
| | SL2_trunc4 | UCGCGCUACGCAGAUAAGGCUUCAUGCCGAAAU | |
| | | CUCGUCGAUGGUCAG | |
| v31 | UA:AU to GC:CG + SL2_trunc5 | AAGGCUGUGCUGACCAUCGAGUUUGCGUACUC | 750 |
| | | UCAAGAAAUUGCAGAAGCUACGCAGAUAAGGC | |
| | | UUCAUGCCGAUCUCGUCGAUGGUCAG | |
| v32 | RAR trunc1 + SL2_trunc5 | AAGGCUGUGCUGACCAUCGAGUUUUUGUACUC | 751 |
| | | UGAAAAGAAGCUACAAAGAUAAGGCUUCAUGC | |
| | | CGAUCUCGUCGAUGGUCAG | |
| v33 | RAR trunc2 + UUCG + | AAGGCUGUGCUGACCAUCGAGUUUUUGUACGU | 752 |
| | SL2_trunc5 | UCGCGCUACAAAGAUAAGGCUUCAUGCCGAUC | |
| | | UCGUCGAUGGUCAG | |
| v34 | UA:AU + RAR trunc1 + | AAGGCUGUGCUGACCAUCGAGUUUGCGUACUC | 753 |
| | SL2_trunc5 | UGAAAAGAAGCUACGCAGAUAAGGCUUCAUGC | |
| | | CGAUCUCGUCGAUGGUCAG | |
| v35 | UA:AU + RAR trunc2 + | AAGGCUGUGCUGACCAUCGAGUUUGCGUACGU | 754 |
| | SL2_trunc5 | UCGCGCUACGCAGAUAAGGCUUCAUGCCGAUCU | |
| | | CGUCGAUGGUCAG | |

¹For RNA, T is U; U are T in the sequence listing. For each entry, the spacer sequence is underlined and the extension arm is bold.

TABLE 14C
EXEMPLARY MODIFIED tagRNA SCAFFOLD VARIANTS

| | | | SEQ ID |
|---|---|---|---|
| NAME | DESCRIPTION | SEQUENCE^{1,2} | NO: |
| v0 | wild type | mA*mA*mG*rGrCrUrGrUrGrCrUrGrArCrCrArUrCrGr | 755 |
| | | ArGrUrUrUrUrUrGrUrArCrUrCrUrCrArArGrArArArUr | |
| | | UrGrCrArGrArArGrCrUrArCrArArArGrArUrArArGrGr | |
| | | CrUrUrCrArUrGrCrCrGrArArArUrCrArArCrArCrCrCrU | |
| | | rGrUrCrArUrUrUrUrArUrGrGrCrArGrGrGrUrGrUrUrU | |
| | | rCrUrCrGrUrCrGrArUrGrGrU*mC*mA*mG | |
| v1 | UA:AU to GC:CG | mA*mA*mG*rGrCrUrGrUrGrCrUrGrArCrCrArUrCrGr | 756 |
| | | ArGrUrUrUrGrCrGrUrArCrUrCrUrCrArArGrArArArUr | |
| | | UrGrCrArGrArArGrCrUrArCrGrCrArGrArUrArArGrGr | |
| | | CrUrUrCrArUrGrCrCrGrArArArUrCrArArCrArCrCrCrU | |
| | | rGrUrCrArUrUrUrUrArUrGrGrCrArGrGrGrUrGrUrUrU | |
| | | rCrUrCrGrUrCrGrArUrGrGrU*mC*mA*mG | |
| v2 | RAR trunc1 | mA*mA*mG*rGrCrUrGrUrGrCrUrGrArCrCrArUrCrGr | 757 |
| | | ArGrUrUrUrUrUrGrUrArCrUrCrUrGrArArArArGrArAr | |
| | | GrCrUrArCrArArArGrArUrArArGrGrCrUrUrCrArUrGr | |
| | | CrCrGrArArArUrCrArArCrArCrCrCrUrGrUrCrArUrUrU | |
| | | rUrArUrGrGrCrArGrGrGrUrGrUrUrUrCrUrCrGrUrCr | |
| | | GrArUrGrGrU*mC*mA*mG | |
| v3 | RAR trunc2 + UUCG | mA*mA*mG*rGrCrUrGrUrGrCrUrGrArCrCrArUrCrGr | 758 |
| | | ArGrUrUrUrUrUrGrUrArCrGrUrUrCrGrCrGrCrUrArCr | |
| | | ArArArGrArUrArArGrGrCrUrUrCrArUrGrCrCrGrArAr | |
| | | ArUrCrArArCrArCrCrCrUrGrUrCrArUrUrUrUrArUrGrG | |
| | | rCrArGrGrGrUrGrUrUrUrCrUrCrGrUrCrGrArUrGr | |
| | | GrU*mC*mA*mG | |
| v4 | UA:AU + RAR trunc1 | mA*mA*mG*rGrCrUrGrUrGrCrUrGrArCrCrArUrCrGr | 759 |
| | | ArGrUrUrUrGrCrGrUrArCrUrCrUrGrArArArArGrArAr | |
| | | GrCrUrArCrGrCrArGrArUrArArGrGrCrUrUrCrArUrGrC | |
| | | rCrGrArArArUrCrArArCrArCrCrCrUrGrUrCrArUrUrUr | |
| | | UrArUrGrGrCrArGrGrGrUrGrUrUrUrCrUrCrGrUrCr | |
| | | GrArUrGrGrU*mC*mA*mG | |
| v5 | UA:AU + RAR trunc2 | mA*mA*mG*rGrCrUrGrUrGrCrUrGrArCrCrArUrCrGr | 760 |
| | | ArGrUrUrUrGrCrGrUrArCrGrUrUrCrGrCrGrCrUrArCrG | |
| | | rCrArGrArUrArArGrGrCrUrUrCrArUrGrCrCrGrArArAr | |
| | | UrCrArArCrArCrCrCrUrGrUrCrArUrUrUrUrArUrGrGrC | |
| | | rArGrGrGrUrGrUrUrUrCrUrCrGrUrCrGrArUrGrGr | |
| | | U*mC*mA*mG | |
| v6 | SL2_trunc1 | mA*mA*mG*rGrCrUrGrUrGrCrUrGrArCrCrArUrCrGr | 761 |
| | | ArGrUrUrUrUrUrGrUrArCrUrCrUrCrArArGrArArArUr | |
| | | UrGrCrArGrArArGrCrUrArCrArArArGrArUrArArGrGr | |
| | | CrUrUrCrArUrGrCrCrGrArArArUrCrArArCrArUrCrUr | |
| | | CrGrUrCrGrArUrGrGrU*mC*mA*mG | |
| v7 | SL2_trunc2 | mA*mA*mG*rGrCrUrGrUrGrCrUrGrArCrCrArUrCrGr | 762 |
| | | ArGrUrUrUrUrUrGrUrArCrUrCrUrCrArArGrArArArUr | |
| | | UrGrCrArGrArArGrCrUrArCrArArArGrArUrArArGrGr | |
| | | CrUrUrCrArUrGrCrCrGrArArArUrCrArArUrCrUrCrG | |
| | | rUrCrGrArUrGrGrU*mC*mA*mG | |
| v8 | SL2_trunc3 | mA*mA*mG*rGrCrUrGrUrGrCrUrGrArCrCrArUrCrGr | 763 |
| | | ArGrUrUrUrUrUrGrUrArCrUrCrUrCrArArGrArArArUr | |
| | | UrGrCrArGrArArGrCrUrArCrArArArGrArUrArArGrGr | |
| | | CrUrUrCrArUrGrCrCrGrArArArUrCrUrCrUrCrGrUrC | |
| | | rGrArUrGrGrU*mC*mA*mG | |
| v9 | SL2_trunc4 | mA*mA*mG*rGrCrUrGrUrGrCrUrGrArCrCrArUrCrGr | 764 |
| | | ArGrUrUrUrUrUrGrUrArCrUrCrUrCrArArGrArArArUr | |
| | | UrGrCrArGrArArGrCrUrArCrArArArGrArUrArArGrGr | |
| | | CrUrUrCrArUrGrCrCrGrArArArUrCrUrCrGrUrCrGr | |
| | | ArUrGrGrU*mC*mA*mG | |
| v10 | SL2_trunc5 | mA*mA*mG*rGrCrUrGrUrGrCrUrGrArCrCrArUrCrGr | 765 |
| | | ArGrUrUrUrUrUrGrUrArCrUrCrUrCrArArGrArArArUr | |
| | | UrGrCrArGrArArGrCrUrArCrArArArGrArUrArArGrGr | |
| | | CrUrUrCrArUrGrCrCrGrArUrCrUrCrGrUrCrGrArUr | |
| | | GrGrU*mC*mA*mG | |
| v11 | UA:AU to GC:CG + | mA*mA*mG*rGrCrUrGrUrGrCrUrGrArCrCrArUrCrGr | 766 |
| | SL2_trunc1 | ArGrUrUrUrGrCrGrUrArCrUrCrUrCrArArGrArArArUr | |
| | | UrGrCrArGrArArGrCrUrArCrGrCrArGrArUrArArGrGr | |
| | | CrUrUrCrArUrGrCrCrGrArArArUrCrArArCrArUrCrUr | |
| | | CrGrUrCrGrArUrGrGrU*mC*mA*mG | |
| v12 | RAR trunc1 + | mA*mA*mG*rGrCrUrGrUrGrCrUrGrArCrCrArUrCrGr | 767 |
| | SL2_trunc1 | ArGrUrUrUrUrUrGrUrArCrUrCrUrGrArArArArGrArAr | |
| | | GrCrUrArCrArArArGrArUrArArGrGrCrUrUrCrArUrGr | |
| | | CrCrGrArArArUrCrArArCrArUrCrUrCrGrUrCrGrAr | |
| | | UrGrGrU*mC*mA*mG | |
| v13 | RAR trunc2 + UUCG + | mA*mA*mG*rGrCrUrGrUrGrCrUrGrArCrCrArUrCrGr | 768 |
| | SL2 trunc1 | ArGrUrUrUrUrUrGrUrArCrGrUrUrCrGrCrGrCrUrArCr | |
| | | ArArArGrArUrArArGrGrCrUrUrCrArUrGrCrCrGrArAr | |
| | | ArUrCrArArCrArUrCrUrCrGrUrCrGrArUrGrGrU* | |
| | | mC*mA*mG | |
| v14 | UA:AU + RAR trunc1 + | mA*mA*mG*rGrCrUrGrUrGrCrUrGrArCrCrArUrCrGr | 769 |
| | SL2_trunc1 | ArGrUrUrUrGrCrGrUrArCrUrCrUrGrArArArArGrArAr | |
| | | GrCrUrArCrGrCrArGrArUrArArGrGrCrUrUrCrArUrGrC | |
| | | rCrGrArArArUrCrArArCrArUrCrUrCrGrUrCrGrArU | |
| | | rGrGrU*mC*mA*mG | |
| v15 | UA:AU + RAR trunc2 + | mA*mA*mG*rGrCrUrGrUrGrCrUrGrArCrCrArUrCrGr | 770 |
| | SL2_trunc1 | ArGrUrUrUrGrCrGrUrArCrGrUrUrCrGrCrGrCrUrArCrG | |
| | | rCrArGrArUrArArGrGrCrUrUrCrArUrGrCrCrGrArArAr | |
| | | UrCrArArCrArUrCrUrCrGrUrCrGrArUrGrGrU*mC | |
| | | *mA*mG | |
| v16 | UA:AU to GC:CG + | mA*mA*mG*rGrCrUrGrUrGrCrUrGrArCrCrArUrCrGr | 771 |
| | SL2_trunc2 | ArGrUrUrUrGrCrGrUrArCrUrCrUrCrArArGrArArArUr | |
| | | UrGrCrArGrArArGrCrUrArCrGrCrArGrArUrArArGrGr | |
| | | CrUrUrCrArUrGrCrCrGrArArArUrCrArArUrCrUrCrG | |
| | | rUrCrGrArUrGrGrU*mC*mA*mG | |
| v17 | RAR trunc 1 + | mA*mA*mG*rGrCrUrGrUrGrCrUrGrArCrCrArUrCrGr | 772 |
| | SL2_trunc2 | ArGrUrUrUrUrUrGrUrArCrUrCrUrGrArArArArGrArAr | |
| | | GrCrUrArCrArArArGrArUrArArGrGrCrUrUrCrArUrGr | |
| | | CrCrGrArArArUrCrArArUrCrUrCrGrUrCrGrArUrGr | |
| | | GrU*mC*mA*mG | |
| v18 | RAR trunc2 + UUCG + | mA*mA*mG*rGrCrUrGrUrGrCrUrGrArCrCrArUrCrGr | 773 |
| | SL2_trunc2 | ArGrUrUrUrUrUrGrUrArCrGrUrUrCrGrCrGrCrUrArCr | |
| | | ArArArGrArUrArArGrGrCrUrUrCrArUrGrCrCrGrArAr | |
| | | ArUrCrArArUrCrUrCrGrUrCrGrArUrGrGrU*mC* | |
| | | mA*mG | |
| v19 | UA:AU + RAR trunc1 + | mA*mA*mG*rGrCrUrGrUrGrCrUrGrArCrCrArUrCrGr | 774 |
| | SL2_trunc2 | ArGrUrUrUrGrCrGrUrArCrUrCrUrGrArArArArGrArAr | |
| | | GrCrUrArCrGrCrArGrArUrArArGrGrCrUrUrCrArUrGrC | |
| | | rCrGrArArArUrCrArArUrCrUrCrGrUrCrGrArUrGrG | |
| | | rU*mC*mA*mG | |
| v20 | UA:AU + RAR trunc2+ | mA*mA*mG*rGrCrUrGrUrGrCrUrGrArCrCrArUrCrGr | 775 |
| | SL2_trunc2 | ArGrUrUrUrGrCrGrUrArCrGrUrUrCrGrCrGrCrUrArCrG | |
| | | rCrArGrArUrArArGrGrCrUrUrCrArUrGrCrCrGrArArAr | |
| | | UrCrArArUrCrUrCrGrUrCrGrArUrGrGrU*mC*mA | |
| | | *mG | |
| v21 | UA:AU to GC:CG + | mA*mA*mG*rGrCrUrGrUrGrCrUrGrArCrCrArUrCrGr | 776 |
| | SL2_trunc3 | ArGrUrUrUrGrCrGrUrArCrUrCrUrCrArArGrArArArUr | |
| | | UrGrCrArGrArArGrCrUrArCrGrCrArGrArUrArArGrGr | |
| | | CrUrUrCrArUrGrCrCrGrArArArUrCrUrCrUrCrGrUrC | |
| | | rGrArUrGrGrU*mC*mA*mG | |
| v22 | RAR trunc1 + | mA*mA*mG*rGrCrUrGrUrGrCrUrGrArCrCrArUrCrGr | 777 |
| | SL2_trunc3 | ArGrUrUrUrUrUrGrUrArCrUrCrUrGrArArArArGrArAr | |
| | | GrCrUrArCrArArArGrArUrArArGrGrCrUrUrCrArUrGr | |
| | | CrCrGrArArArUrCrUrCrUrCrGrUrCrGrArUrGrGrU | |
| | | *mC*mA*mG | |
| v23 | RAR trunc2 + UUCG + | mA*mA*mG*rGrCrUrGrUrGrCrUrGrArCrCrArUrCrGr | 778 |
| | SL2_trunc3 | ArGrUrUrUrUrUrGrUrArCrGrUrUrCrGrCrGrCrUrArCr | |
| | | ArArArGrArUrArArGrGrCrUrUrCrArUrGrCrCrGrArAr | |
| | | ArUrCrUrCrUrCrGrUrCrGrArUrGrGrU*mC*mA*m | |
| | | G | |
| v24 | UA:AU + RAR trunc1 + | mA*mA*mG*rGrCrUrGrUrGrCrUrGrArCrCrArUrCrGr | 779 |
| | SL2_trunc3 | ArGrUrUrUrGrCrGrUrArCrUrCrUrGrArArArArGrArAr | |
| | | GrCrUrArCrGrCrArGrArUrArArGrGrCrUrUrCrArUrGrC | |
| | | rCrGrArArArUrCrUrCrUrCrGrUrCrGrArUrGrGrU* | |
| | | mC*mA*mG | |
| v25 | UA:AU + RAR trunc2 + | mA*mA*mG*rGrCrUrGrUrGrCrUrGrArCrCrArUrCrGr | 780 |
| | SL2_trunc3 | ArGrUrUrUrGrCrGrUrArCrGrUrUrCrGrCrGrCrUrArCrG | |
| | | rCrArGrArUrArArGrGrCrUrUrCrArUrGrCrCrGrArArAr | |
| | | UrCrUrCrUrCrGrUrCrGrArUrGrGrU*mC*mA*mG | |
| v26 | UA:AU to GC:CG + | mA*mA*mG*rGrCrUrGrUrGrCrUrGrArCrCrArUrCrGr | 781 |
| | SL2_trunc4 | ArGrUrUrUrGrCrGrUrArCrUrCrUrCrArArGrArArArUr | |
| | | UrGrCrArGrArArGrCrUrArCrGrCrArGrArUrArArGrGr | |
| | | CrUrUrCrArUrGrCrCrGrArArArUrCrUrCrGrUrCrGr | |
| | | ArUrGrGrU*mC*mA*mG | |
| v27 | RAR trunc 1 + | mA*mA*mG*rGrCrUrGrUrGrCrUrGrArCrCrArUrCrGr | 782 |
| | SL2_trunc4 | ArGrUrUrUrUrUrGrUrArCrUrCrUrGrArArArArGrArAr | |
| | | GrCrUrArCrArArArGrArUrArArGrGrCrUrUrCrArUrGr | |
| | | CrCrGrArArArUrCrUrCrGrUrCrGrArUrGrGrU*mC | |
| | | *mA*mG | |
| v28 | RAR trunc2 + UUCG + | mA*mA*mG*rGrCrUrGrUrGrCrUrGrArCrCrArUrCrGr | 783 |
| | SL2_trunc4 | ArGrUrUrUrUrUrGrUrArCrGrUrUrCrGrCrGrCrUrArCr | |
| | | ArArArGrArUrArArGrGrCrUrUrCrArUrGrCrCrGrArAr | |
| | | ArUrCrUrCrGrUrCrGrArUrGrGrU*mC*mA*mG | |
| v29 | UA:AU + RAR trunc1 + | mA*mA*mG*rGrCrUrGrUrGrCrUrGrArCrCrArUrCrGr | 784 |
| | SL2_trunc4 | ArGrUrUrUrGrCrGrUrArCrUrCrUrGrArArArArGrArAr | |
| | | GrCrUrArCrGrCrArGrArUrArArGrGrCrUrUrCrArUrGrC | |
| | | rCrGrArArArUrCrUrCrGrUrCrGrArUrGrGrU*mC* | |
| | | mA*mG | |
| v30 | UA:AU + RAR trunc2 + | mA*mA*mG*rGrCrUrGrUrGrCrUrGrArCrCrArUrCrGr | 785 |
| | SL2_trunc4 | ArGrUrUrUrGrCrGrUrArCrGrUrUrCrGrCrGrCrUrArCrG | |
| | | rCrArGrArUrArArGrGrCrUrUrCrArUrGrCrCrGrArArAr | |
| | | UrCrUrCrGrUrCrGrArUrGrGrU*mC*mA*mG | |
| v31 | UA:AU to GC:CG + | mA*mA*mG*rGrCrUrGrUrGrCrUrGrArCrCrArUrCrGr | 786 |
| | SL2_trunc5 | ArGrUrUrUrGrCrGrUrArCrUrCrUrCrArArGrArArArUr | |
| | | UrGrCrArGrArArGrCrUrArCrGrCrArGrArUrArArGrGr | |
| | | CrUrUrCrArUrGrCrCrGrArUrCrUrCrGrUrCrGrArUr | |
| | | GrGrU*mC*mA*mG | |
| v32 | RAR trunc1 + | mA*mA*mG*rGrCrUrGrUrGrCrUrGrArCrCrArUrCrGr | 787 |
| | SL2_trunc5 | ArGrUrUrUrUrUrGrUrArCrUrCrUrGrArArArArGrArAr | |
| | | GrCrUrArCrArArArGrArUrArArGrGrCrUrUrCrArUrGr | |
| | | CrCrGrArUrCrUrCrGrUrCrGrArUrGrGrU*mC*mA | |
| | | *mG | |
| v33 | RAR trunc2 + UUCG + | mA*mA*mG*rGrCrUrGrUrGrCrUrGrArCrCrArUrCrGr | 788 |
| | SL2_trunc5 | ArGrUrUrUrUrUrGrUrArCrGrUrUrCrGrCrGrCrUrArCr | |
| | | ArArArGrArUrArArGrGrCrUrUrCrArUrGrCrCrGrArUr | |
| | | CrUrCrGrUrCrGrArUrGrGrU*mC*mA*mG | |
| v34 | UA:AU + RAR trunc1 + | mA*mA*mG*rGrCrUrGrUrGrCrUrGrArCrCrArUrCrGr | 789 |
| | SL2_trunc5 | ArGrUrUrUrGrCrGrUrArCrUrCrUrGrArArArArGrArAr | |
| | | GrCrUrArCrGrCrArGrArUrArArGrGrCrUrUrCrArUrGrC | |
| | | rCrGrArUrCrUrCrGrUrCrGrArUrGrGrU*mC*mA* | |
| | | mG | |
| v35 | UA:AU + RAR trunc2 + | mA*mA*mG*rGrCrUrGrUrGrCrUrGrArCrCrArUrCrGr | 790 |
| | SL2 trunc5 | ArGrUrUrUrGrCrGrUrArCrGrUrUrCrGrCrGrCrUrArCrG | |
| | | rCrArGrArUrArArGrGrCrUrUrCrArUrGrCrCrGrArUrCr | |
| | | UrCrGrUrCrGrArUrGrGrU*mC*mA*mG | |

¹For RNA, T is U; U are T in the sequence listing. For each entry, the spacer sequence is underlined and the extension arm is bold. 2 For the sequences: *, phosphorothioate modification; mN, 2′O-methyl modification; rN, unmodified ribonucleotide; i2FN, Int Fluoro modification. N = A, C, T/U, G.

In some embodiments, the gRNAs or tagRNAs described herein can be produced by in vitro transcription (IVT), synthetic and/or chemical synthesis methods, or a combination thereof. One or more of enzymatic IVT, solid-phase, liquid-phase, combined synthetic methods, small region synthesis, and ligation methods can be utilized. In some embodiments, the gRNAs or tagRNAs are made using IVT enzymatic synthesis methods. Methods of making polynucleotides by IVT are known in the art and are described in WO2013/151666. Polynucleotides constructs and vectors can be used to in vitro transcribe a gRNA or tagRNA described herein.

In some embodiments, a nucleic acid encoding a RT editor is administered to the subject. In some embodiments, the nucleic acid can be generated by an in vitro transcription reaction. In some embodiments, generating in vitro transcribed RNA comprises incubating a linear DNA template with an RNA polymerase and a nucleotide mixture under conditions to allow (run-off) RNA in vitro transcription. The nucleotide mixture can be part of an in vitro transcription mix (IVT-mix). In some embodiments, the RNA polymerase is a T7 RNA polymerase.

The nucleotide mixture used in RNA in vitro transcription can additionally contain modified nucleotides as defined below. In some embodiments, the nucleotide mixture (e.g., the fraction of each nucleotide in the mixture) used for RNA in vitro transcription reactions can be optimized for the given RNA sequence (optimized NTP mix). Such methods are described, for example in WO2015/188933. RNA obtained by a process using an optimized NTP mix is, in some embodiments, characterized by reduced immune stimulatory properties.

In various embodiments the nucleotide mixture in an in vitro transcription reaction comprises a cap analog. Accordingly, in some embodiments the cap analog is a cap0, cap1, cap2, a modified cap0 or a modified cap1 analog, or a cap1 analog as described below.

The term “cap analog” or “5′-cap structure” as used herein can refer to the 5′ structure of the RNA, particularly a guanine nucleotide, positioned at the 5′-end of an RNA, e.g., an mRNA. In some embodiments, the 5′-cap structure is connected via a 5′-5′-triphosphate linkage to the RNA. In some embodiments, a “5′-cap structure” or a “cap analogue” is not considered to be a “modified nucleotide” or “chemically modified nucleotides”. 5′-cap structures which may be suitable include cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modARCA (e.g., phosphothioate modARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure can be formed in chemical RNA synthesis, using capping enzymes, or in RNA in vitro transcription (co-transcriptional capping) using cap analogs. The term “cap analog” as used herein can refer to a non-polymerizable di-nucleotide or tri-nucleotide that has cap functionality in that it facilitates translation or localization, and/or prevents degradation of the RNA when incorporated at the 5′-end of the RNA. Non-polymerizable means that the cap analogue will be incorporated only at the 5′-terminus because it does not have a 5′ triphosphate and therefore cannot be extended in the 3′-direction by a template-dependent polymerase, (e.g., a DNA-dependent RNA polymerase). Examples of cap analogues include m7GpppG, m7GpppA, m7GpppC; unmethylated cap analogues (e.g., GpppG); dimethylated cap analogue (e.g., m2,7GpppG), trimethylated cap analogue (e.g. m2,2,7GpppG), dimethylated symmetrical cap analogues (e.g. m7Gpppm7G), or anti reverse cap analogues (e.g., ARCA; m7,2′OmeGpppG, m7,2′dGpppG, m7,3′OmeGpppG, m7,3′dGpppG and their tetraphosphate derivatives). Further cap analogues have been described previously, e.g., WO2008/016473, WO2008/157688, WO2009/149253, WO2011/015347, and WO2013/059475. Further suitable cap analogues in that context are described in, e.g., WO2017/066793, WO2017/066781, WO2017/066791, WO2017/066789, WO2017/053297, WO2017/066782, WO2018/075827 and WO2017/066797 wherein the disclosures relating to cap analogues are incorporated herewith by reference.

In some embodiments, a cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. For example, any cap analog derivable from the structure disclosed in claim 1-5 of WO2017/053297 may be suitably used to co-transcriptionally generate a cap1 structure. In some embodiments, any cap analog derivable from the structure described in WO2018/075827 can be suitably used to co-transcriptionally generate a cap1 structure. In some embodiments, the cap1 analog is a cap1 trinucleotide cap analog. In some embodiments, the cap1 structure of the in vitro transcribed RNA is formed using co-transcriptional capping using tri-nucleotide cap analog m7G(5′)ppp(5′)(2′OMeA)pG or m7G(5′)ppp(5′)(2′OMeG)pG. In some embodiments, the cap1 analog is m7G(5′)ppp(5′)(2′OMeA)pG.

In some embodiments, the RNA (e.g., mRNA) comprises a 5′-cap structure, e.g., a cap1 structure. In some embodiments, the 5′ cap structure can improve stability and/or expression of the mRNA. A cap1 structure comprising mRNA (produced by, e.g., in vitro transcription) has several advantageous features including an increased translation efficiency and a reduced stimulation of the innate immune system. In some embodiments, the in vitro transcribed RNA comprises at least one coding sequence encoding at least one peptide or protein. In some embodiments, the protein is an RNA-guided endonuclease. In some embodiments, the RNA-guided endonuclease is Cas9 or a derivative thereof.

In some embodiments, the mRNA can comprise at least one chemically modified nucleoside and/or nucleotide. In some embodiments, the chemically modified nucleoside and/or nucleotide is selected from pseudouridine, N1-methylpseudouridine, and 5-methoxyuridine. In some embodiments, the chemically modified nucleoside is N1-methylpseudouridine (e.g., 1-methylpseudouridine). In some embodiments, at least about 80% or more (e.g., about 85%, 90%, 91%, 92%, 93%, 94%, 95%, 96%, 97%, 98%, 99%) of uridines in the mRNA are modified or replaced with N1-methylpseudouridine. In some embodiments, 100% of the uridines (e.g., uracils) in the mRNA are modified or replaced with N1-methylpseudouridine. In some embodiments, the disclosure provides an mRNA comprising a nucleotide sequence that is at least 85% or more (e.g., 86%, 87%, 88%, 89%, 90%, 91%, 92%, 93%, 94%, 95%, 96%, 97%, 98%, 99%, 100%) identical to the nucleotide sequence of SEQ ID NO: 13, wherein 100% of the uridines or uracils of the mRNA are modified or replaced with N1-methylpseudouridine. In some embodiments, two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800 or more) of the uridine or uracil residues are N1-methylpseudouridine.

In some embodiments, the disclosure provides an mRNA comprising a nucleotide sequence that is 100% identical to the nucleotide sequence of SEQ ID NO: 57 or 58, wherein 100% of the uridines (e.g., uracils) of the mRNA are modified or replaced with N1-methylpseudouridine. Optimized mRNAs encoding, for example Cas9, are also described in US20210355463A1, which is hereby incorporated by reference in its entirety.

### Compositions and Therapeutic Applications

Provided herein also includes compositions. A composition can include one or more gRNA(s) (e.g., tagRNA and egRNA), and a RT editor protein described herein. A composition can include one or more gRNA(s) (e.g., tagRNA and egRNA), and a nucleic acid encoding a RT editor protein described herein. The compositions can include one or more of the RT editing complexes of the disclosure. Also provided herein are ribonucleoprotein (RNP) complexes comprising any of the RT editing complexes disclosed herein, or a component thereof. In some embodiments, the RNP comprises, e.g., a RT editor (e.g., a polypeptide or a polynucleotide encoding a RT editor) and a tagRNA. In some embodiments, the RNP comprises, e.g., a RT editor (e.g., a polypeptide or a polynucleotide encoding a RT editor) and an egRNA. In some embodiments, the RNP comprises, e.g., a RT editor (e.g., a polypeptide or a polynucleotide encoding a RT editor) and both a tagRNA and an egRNA.

Disclosed herein include lipid nanoparticles (LNPs). In some embodiments, the LNP comprises any of the RT editing systems disclosed herein, or a component thereof. The LNP can comprise the tagRNA and the nucleic acid encoding the RT editor. The nucleic acid encoding the RT editor can be mRNA. The LNP can comprise the egRNA. The lipid nanoparticle can comprise one or more neutral lipids, charged lipids, ionizable lipids, steroids, and polymers conjugated lipids. The lipid nanoparticle can comprise cholesterol, a polyethylene glycol (PEG) lipid, or both.

In some embodiments, the composition can comprise one or more gRNA(s) (e.g., tagRNA and/or egRNA) each comprising a spacer complementary to a genomic sequence within or near (for example, within any of 1, 2, 3, 4, 5, 6, 7, 8, 9, 10, 15, 20, 25, 30, 35, 40, 45, 50, 55, 60, 65, 70, 75, 80, 85, 90, 95, 100 or more bases from) any exon of the ATPB7 gene. In some embodiments, the gRNA targets a sequence within exon 14 of the ATPB7 gene. The gRNA can comprise a spacer sequence complementary or identical to a target sequence within exon 14 of the ATPB7 gene. In some embodiments, a gRNA comprises a spacer sequence of any one of SEQ ID NOs: 50-52 and 133-136 or a variant thereof have one, two or three mismatches relative to any one of SEQ ID NOs: 50-52 and 133-136. In some embodiments, the gRNA comprises a spacer comprising or consisting of the sequence of SEQ ID NOs: 50-52 and 133-136.

In some embodiments, the RT editor comprises a DNA-binding domain of a Cas12a, Cas12b, Cas12c, Cas12d, Cas12e, Cas14a, Cas14b, Cas14c, Cas14d, Cas14e, Cas14f, Cas14g, Cas14h, Cas14u, Casϕendonuclease, Ssch1Cas9, Sro1Cas9, Sha4Cas9, SsuCas9, iSpyMacCas9, Ssi5Cas9, Ssi8Cas9, Ssci4Cas9, Shy1Cas9, Sag3Cas9, Slutr1Cas9, Ssch3Cas9, SpRYCas9, SpRYcCas9, Sma2Cas9, SsaCas9, EvoCjCas9, or iSpyMac or a functional derivative thereof. In some embodiments, the DNA endonuclease domain is Cas9. In some embodiments, the Cas9 is from Streptococcus pyogenes (SPYCas9). In some embodiments, the Cas9 is from Staphylococcus lugdunensis (SluCas9). In some embodiments, a DNA sequence that is transcribed to the nucleic acid encoding the RT editor is codon optimized. In some embodiments, the nucleic acid encoding the RT editor (e.g., an mRNA) comprises a 5′ CAP structure and 3′ polyA tail. In some embodiments, the nucleic acid encoding the DNA endonuclease is linked to the gRNA via a covalent bond.

In some embodiments, each of the components of a RT editing system can be separately formulated into lipid nanoparticles, or are all co-formulated into one lipid nanoparticle.

In some embodiments, RT editor mRNA can be formulated in a lipid nanoparticle, while gRNAs (e.g., tagRNA and/or egRNA) can be delivered in an AAV vector.

Options are available to deliver the RT editor as a DNA plasmid, as mRNA or as a protein. A guide RNA (e.g., tagRNA and/or egRNA) can be expressed from the same DNA, or can also be delivered as an RNA. The RNA can be chemically modified to alter or improve its half-life, or decrease the likelihood or degree of immune response. The endonuclease protein can be complexed with the gRNA prior to delivery. In some embodiments, the plasmid comprises or consists of the sequence of SEQ ID NO: 56 or a sequence having at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to the sequence of SEQ ID NO: 56.

Disclosed herein include isolated cells. In some embodiments, the isolated cell comprises any of the tagRNAs, the RT editing systems, the RT editing complexes, the RNPs, the LNPs, or the vectors provided herein. The cell can be a mammalian cell. In some embodiments, the cell can be a human cell. The cell can be a primary cell. The cell can be a hepatocyte. In some embodiments, the cell has or is suspected of having a mutation that can be corrected via gene editing. In some embodiments, the gene editing system is an RT editing system. The cell can be from a subject having Wilson's disease. The cell can be from a subject having A1ATD disease or disorder. The cell can be from a subject having phenylketonuria or hyperphenylalaninemia. The subject can be a human.

Disclosed herein include compositions. In some embodiments, the composition is a pharmaceutical composition. In some embodiments, the pharmaceutical composition comprises: any of the tagRNAs, the RT editing systems, the RT editing complexes, the RNPs, the LNPs, the polynucleotides, the vectors, or the cells disclosed herein; and (ii) a pharmaceutically acceptable carrier.

A composition described above can further have one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a composition can also include one or more components that can be used to facilitate or enhance the on-target binding or the cleavage of DNA by the endonuclease, or improve the specificity of targeting.

One or more components of a composition can be formulated with pharmaceutically acceptable excipients such as carriers, solvents, stabilizers, adjuvants, diluents, etc., depending upon the particular mode of administration and dosage form. In some embodiments, guide RNA compositions are generally formulated to achieve a physiologically compatible pH, and range from a pH of about 3 to a pH of about 11, about pH 3 to about pH 7, depending on the formulation and route of administration. In some embodiments, the pH is adjusted to a range from about pH 5 to about pH 8.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition will depend on the nature of the disorder or condition, and can be determined by standard clinical techniques.

The terms “stable” or “stability” as used herein can refer to the ability of the compounds herein described (e.g., the RT editor or a nucleic acid encoding the RT editor and/or gRNA (e.g., tagRNA and/or egRNA)) to maintain therapeutic efficacy (e.g., all or the majority of its intended biological activity and/or physiochemical integrity) over extended periods of time. The stability of one or more of the compounds described herein (e.g., the RT editor or a nucleic acid encoding the RT editor and/or gRNA (e.g., tagRNA and/or egRNA)) can be 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, or more than 3 years. The temperature of storage can vary. For example, the storage temperature can be, can be about, can be at least, or can be at least about −80° C., −65° C., −20° C., 5° C., or a number or range between any two of these values. In some embodiments, the storage temperature is less than or equal to −65° C.

In some embodiments, the compounds herein described (e.g., the RT editor or a nucleic acid encoding the RT editor and/or gRNA (e.g., tagRNA and/or egRNA)) of a composition can be delivered via transfection such as calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. In some embodiments, the composition is introduced to the cells via lipid-mediated transfection using a lipid nanoparticle.

Disclosed herein include methods for editing an target gene. In some embodiments, the method comprises contacting the target gene with (i) any of the tagRNAs of the disclosure and a RT editor comprising a DNA binding domain, a DNA endonuclease domain and a DNA polymerase domain or (ii) any of the RT editing systems disclosed herein. In some embodiments, the tagRNA directs the RT editor to incorporate the intended nucleotide edit in the target gene, thereby editing the target gene.

Disclosed herein include methods for editing a target gene. In some embodiments, the method comprises contacting the target gene with any of the RT editing complexes disclosed herein, wherein the tagRNA directs the RT editor to incorporate the intended nucleotide edit in the ATP7B gene, thereby editing the target gene.

In some embodiments, the RT editor synthesizes a single stranded DNA encoded by the editing template, wherein the single stranded DNA replaces the editing target sequence and results in incorporation of the intended nucleotide edit into a region corresponding to the editing target in the target gene. The target gene can be in a cell. The cell can be a mammalian cell. The cell can be a human cell. The cell can be a primary cell. The cell can be a hepatocyte. The cell can be in a subject. The subject can be a human. In some embodiments, the cell has or is suspected of having a mutation that can be corrected via gene editing. In some embodiments, the gene editing system is an RT editing system. The cell can be from a subject having a disease or disorder. The cell can be from a subject having Wilson's disease. The cell can be from a subject having A1ATD disease or disorder. The cell can be from a subject having phenylketonuria or hyperphenylalaninemia. The method can comprise administering the cell to the subject after incorporation of the intended nucleotide edit. Also provided herein are cells generated by any of the methods disclosed herein. Disclosed herein include populations of cells generated by any of the methods of the disclosure.

Disclosed herein include methods for treating a disease or disorder in a subject in need thereof via gene editing. In some embodiments, the gene editing system is an RT editing system. Disclosed herein include methods for treating Wilson's disease in a subject in need thereof. Disclosed herein include methods for treating A1ATD disease in a subject in need thereof. Disclosed herein include methods for treating phenylketonuria or hyperphenylalaninemia in a subject in need thereof. In some embodiments, the method comprises administering to the subject (i) any of the tagRNAs of the disclosure and a RT editor comprising a DNA binding domain, a DNA endonuclease domain and a DNA polymerase domain or (ii) any of the RT editing systems disclosed herein. In some embodiments, the tagRNA directs the RT editor to incorporate the intended nucleotide edit in the mutated gene in the subject, thereby treating the disease or disorder in the subject. In some embodiments, the tagRNA directs the RT editor to incorporate the intended nucleotide edit in the ATP7B gene in the subject, thereby treating Wilson's disease in the subject. In some embodiments, the tagRNA directs the RT editor to incorporate the intended nucleotide edit in the SERPINA1 gene in the subject, thereby treating A1ATD disease or disorder in the subject. In some embodiments, the tagRNA directs the RT editor to incorporate the intended nucleotide edit in the PAH gene in the subject, thereby treating phenylketonuria or hyperphenylalaninemia in the subject.

Disclosed herein include methods for treating a disease in a subject in need thereof. In some embodiments, the method comprises administering to the subject any of the RT editing complexes, any of the RNPs, any of the LNPs, or any of the pharmaceutical compositions disclosed herein. In some embodiments, the tagRNA directs the RT editor to incorporate the intended nucleotide edit in the target gene in the subject, thereby treating the disease or disorder in the subject. The subject can be a human.

In some embodiments, editing efficiency of the RT editing compositions and methods described herein can be measured by calculating the percentage of edited target genes in a population of cells introduced with the RT editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., ATP7B gene, PAH gene, or SERPINA1 gene within the genome of a cell) to a RT editing composition (e.g., LNP). In some embodiments, the population of cells introduced with the RT editing composition is ex vivo. In some embodiments, the population of cells introduced with the RT editing composition is in vitro. In some embodiments, the population of cells introduced with the RT editing composition is in vivo. In some embodiments, the RT editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control.

In some embodiments, the RT editing compositions provided herein are capable of incorporated one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., ATP7B gene, PAH gene, or SERPINA1 gene within the genome of a cell) to a RT editing composition.

In some embodiments, the RT editing methods disclosed hereto have an editing efficiency of at least about 95% and an indel frequency of less than 1% to a target cell, e.g., a human primary cell or hepatocyte, to some embodiments, the RT editing methods disclosed hereto have an editing efficiency of at least about 95% and an indel frequency of less than 0.5% to a target cell, e.g., a human primary cell or hepatocyte, to some embodiments, the RT editing methods disclosed hereto have an editing efficiency of at least about 95% and an indel frequency of less than 0.1% to a target cell, e.g., a human primary cell or hepatocyte, to some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., ATP7B gene, PAH gene, or SERPINA1 gene within the genome of a cell) to a RT editing composition, to some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a ATP7B gene, PAH gene, or SERPINA1 gene within the genome of a cell) to a RT editing composition.

In some embodiments, the target tissue for the compositions and methods described herein is liver tissue. In some embodiments, the target cells for the compositions and methods described herein is hepatocyte.

In some embodiments, the pharmaceutical composition thereof can be administered by aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, and/or intradermal injection, or any combination thereof. The administration can be local or systemic. The systemic administration includes enteral and parenteral administration. In some embodiments, more than one administration can be employed to achieve the desired level of gene expression over a period of various intervals, e.g., daily, weekly, monthly, or yearly.

The pharmaceutical composition thereof can be administered to a subject in need thereof at a pharmaceutically effective amount. The term “pharmaceutically effective amount” as used herein means that the amount of the pharmaceutical composition that will elicit a desired therapeutic effect and/or biological or medical responses of a tissue, system, animal or human. The administration can result in a desired correction in target gene such restoration of wild-type activity of the protein.

In certain embodiments, disease models for screening of the RT editing system and guide RNAs are used to demonstrate efficacy and tolerability. In some embodiments, efficacy and tolerability are demonstrated in vitro. In some embodiments, efficacy and tolerability are demonstrated in vivo. In some embodiments, the in vitro system is a cell line. In certain embodiments, the cell line is a human hepatocyte cell line, for instance, Huh7 cell line wherein the specific disease mutation has been incorporated. In certain embodiments, the in vitro cell line are primary hepatocytes, for instance primary murine hepatocytes and primary human hepatocytes. In certain embodiments, the in vivo system are humanized mouse and rat models, wherein the specific disease mutation has been incorporated. Such disease models are well established in literature wherein the data may be extrapolated to efficacy and tolerability in humans.

### EXAMPLES

Some aspects of the embodiments discussed above are disclosed in further detail in the following examples, which are not in any way intended to limit the scope of the present disclosure.

### Example 1

### Methods and Compositions for Gene Editing

Provided in this Example are methods and compositions related to the RT editing systems of the disclosure. Also provided are methods and compositions of editing of ATP7B for treating Wilson's disease. Disclosed herein are methods of correction of the disease mutation using Reverse Transcriptase (RT) editing. Also provided in this Example are studies of systems for gRNA-based targeting and gRNA-based introduction of nucleotide edit(s) of interest, such as the impact of including, excluding, and/or modifying components of such systems, including, for example, the gRNA segment that binds a nicked target site, the gRNA segment that is reverse transcribed, the subsequent gRNA segment having target homology, and a second gRNA that nicks an opposite strand as said gRNA. The components of an RT editing system include: mRNA encoding the editor (e.g., nicking Cas9 fused to reverse transcriptase); long guide RNA encoding the edit (e.g., tagRNA in RT editing); and an optional second guide RNA (e.g., egRNA) to increase editing efficiency. In some embodiments, the components can be formulated in LNP for therapeutic delivery.

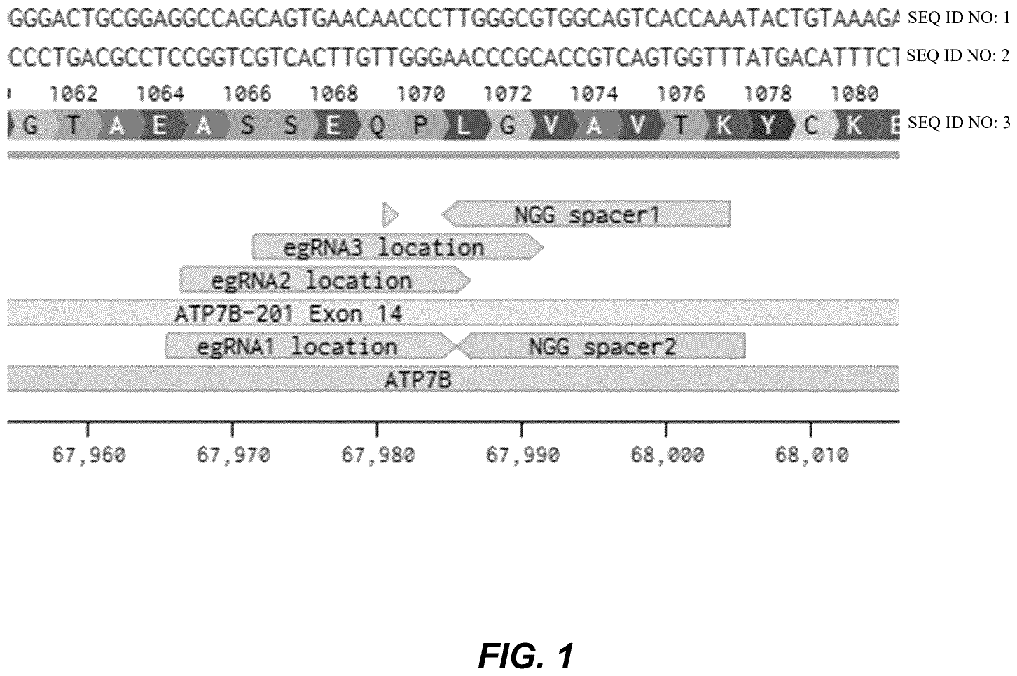

Cell lines (HEK293T and Huh7 cell lines) were engineered to contain the disease mutation, then used as models for mutation correction experiments. NGG spacer1 (SEQ ID NO: 50) and NGG spacer2 (SEQ ID NO: 133) are options for the long gRNA (FIG. 1). As shown in FIG. 1, egRNA1 (SEQ ID NO: 51), egRNA2 (SEQ ID NO: 52), and egRNA3 (SEQ ID NO: 134) are options for the second gRNA (e.g., egRNA).

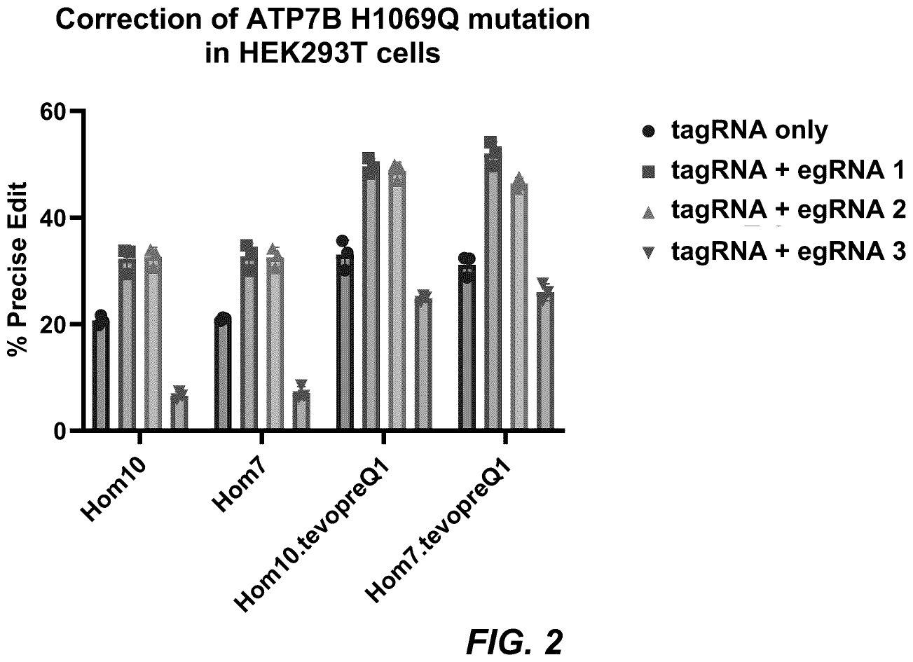

FIG. 2 displays data of editing efficiency in HEK293T cells. In this experiment, the editing system components were delivered by plasmid transfection, and the flap binding sequence (FB sequence; FBS) was 13 nucleotides in length. Experiments using only the long gRNA (e.g., tagRNA) or with a second, egRNA were performed.

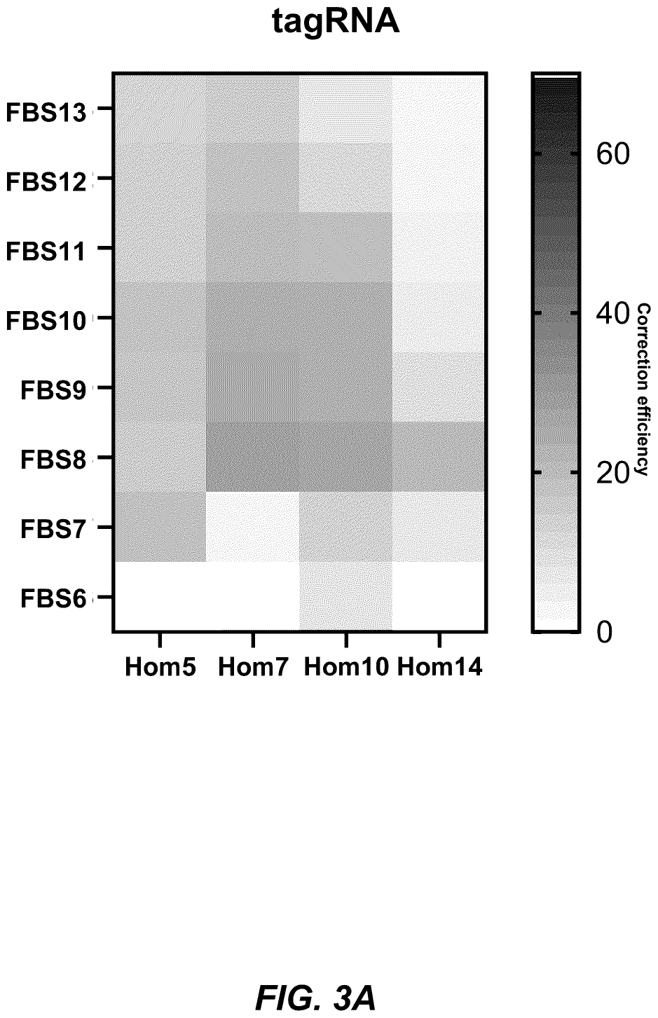

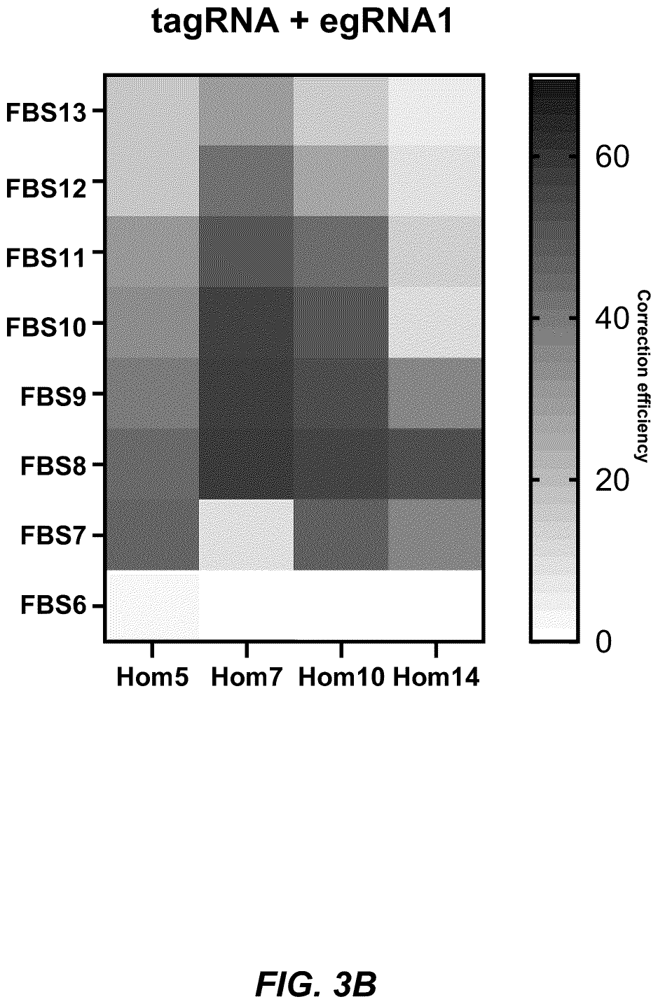

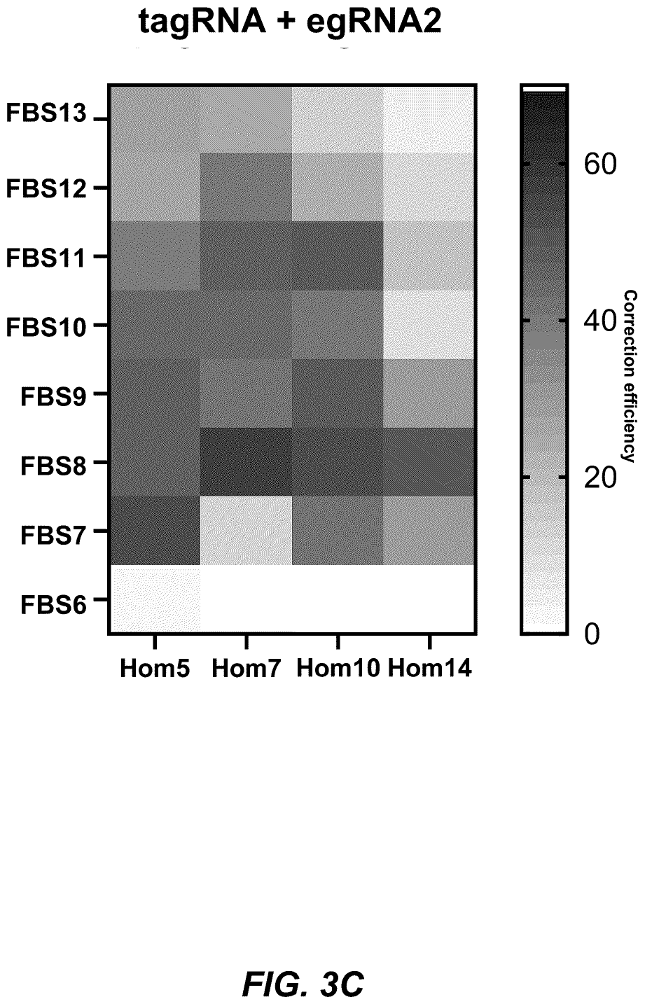

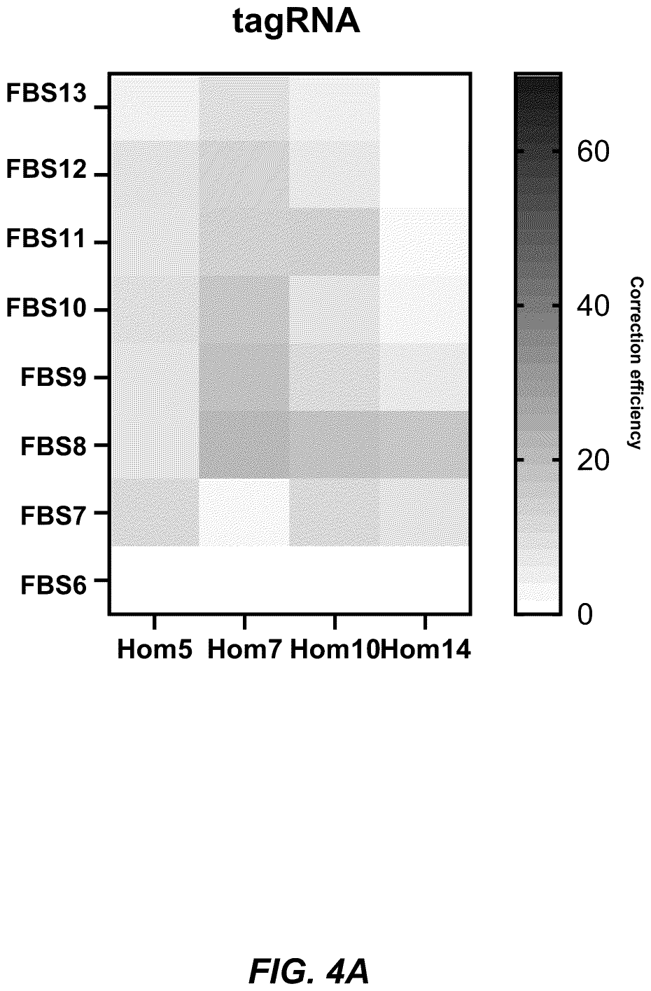

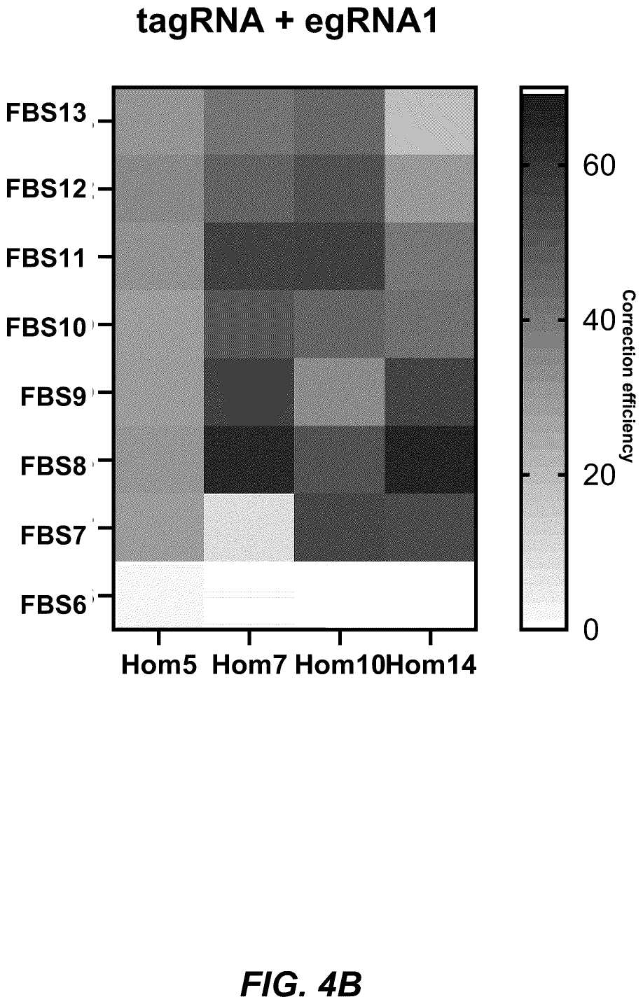

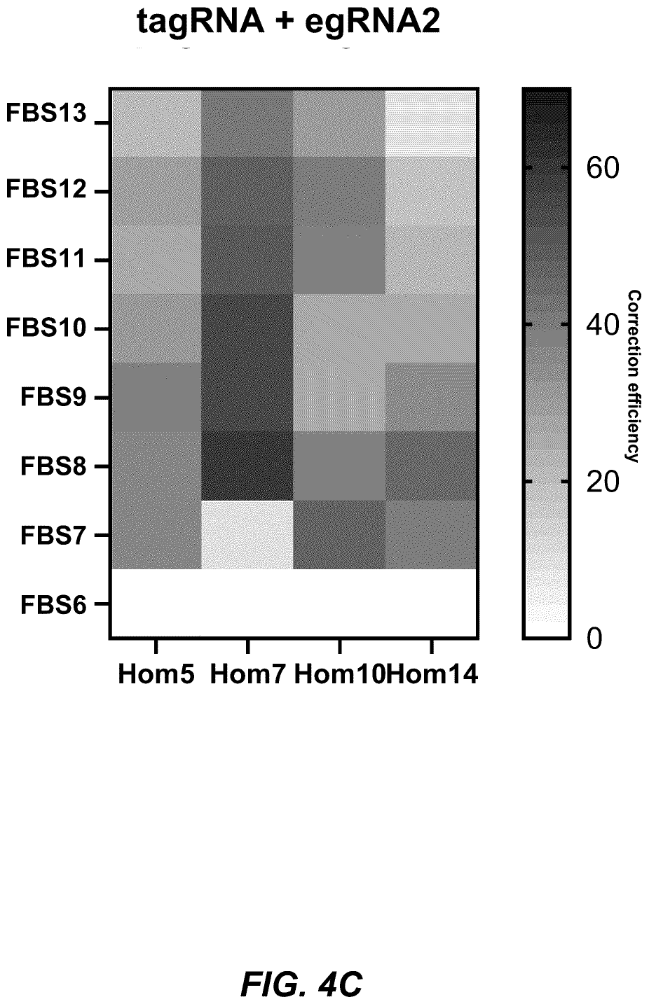

Shown in FIG. 3A-FIG. 4C are data from experiments using mRNA (encoding the RT editor) and synthetic gRNAs. For NGG2 tagRNAs, maximum 15% correction with tagRNA+egRNA system was observed. With regards to surrogate mutations, the following was determined: correction is +7 T>G; surrogate in WT genome was +7 G>A (maximum 5% editing); and surrogate in H1069Q genome was +7 T>A (maximum 6.4% editing). In some embodiments, typically, systems employing only the tagRNA were better than using both a tagRNA and egRNA. In some embodiments, model systems are employed that have a wildtype human genome, and there is no disease mutation. In some such embodiments, a user cannot make the correction mutation, but a user can make a different nearby mutation instead (termed a surrogate mutation). In some embodiments a similar strategy/surrogate mutation (correction) can be used to evaluate toxicity in NEPs.

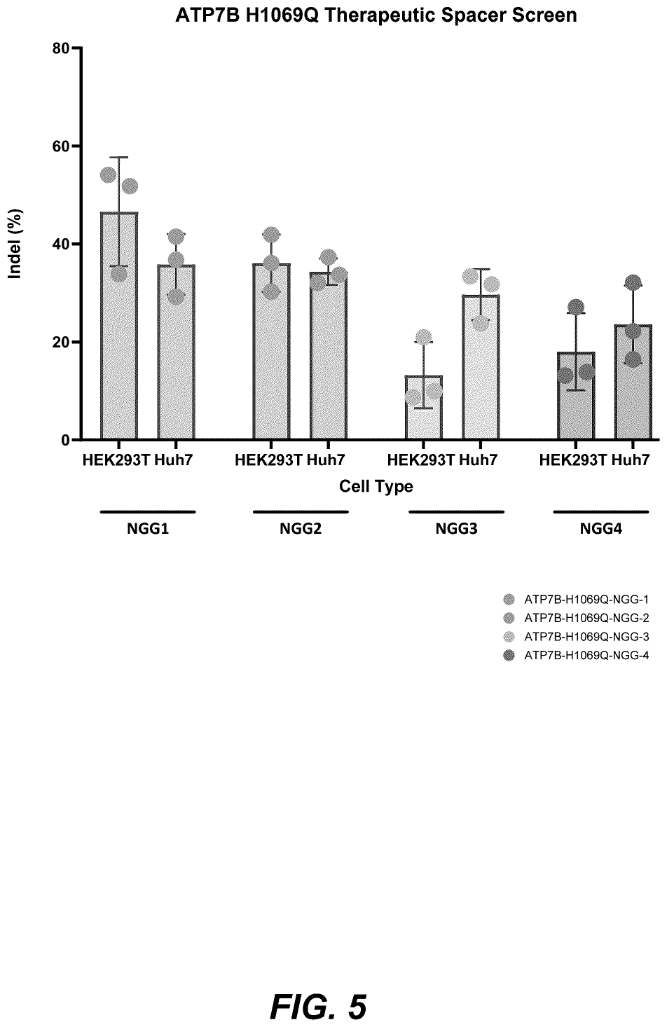

FIG. 5 displays results from a screen of spacer sequences for editing ATP7B3 gene performed in BTEK293T and Huh7 cells. Cas9 mRNA plus synthetic gRNA were provided in these experiments.

Table 15 (Huh7 cells) and Table 16 (HEK293T cells) below display editing efficiencies for the RT editing gRNAs of the disclosure (e.g., tagRNA alone or with a second, egRNA). The editing efficiency shown in the tables are the average of 3 independent experiments.

TABLE 15
EDITING EFFICIENCY IN Huh7 CELLS

| tagRNA | SEQ | egRNA | SEQ | Editing |
|---|---|---|---|---|
| Name | ID NO: | Name | ID NO: | (%) |
| BK g050 | 99 | N/A | N/A | 1.8 |
| BK g051 | 100 | N/A | N/A | 2.5 |
| BK g052 | 101 | N/A | N/A | 3.2 |
| BK g053 | 102 | N/A | N/A | 4.4 |
| BK g054 | 103 | N/A | N/A | 8.2 |
| BK g055 | 104 | N/A | N/A | 16.7 |
| BK g056 | 105 | N/A | N/A | 8.8 |
| BK g057 | 106 | N/A | N/A | 0.1 |
| BK g058 | 107 | N/A | N/A | 5.7 |
| BK g059 | 108 | N/A | N/A | 8.0 |
| BK g060 | 109 | N/A | N/A | 13.3 |
| BK g061 | 110 | N/A | N/A | 9.7 |
| BK g062 | 111 | N/A | N/A | 12.0 |
| BK g063 | 112 | N/A | N/A | 19.3 |
| BK g064 | 113 | N/A | N/A | 13.0 |
| BK g065 | 114 | N/A | N/A | 0.2 |
| BK g066 | 115 | N/A | N/A | 11.0 |
| BK g067 | 116 | N/A | N/A | 14.5 |
| BK g068 | 117 | N/A | N/A | 15.2 |
| BK g069 | 118 | N/A | N/A | 18.3 |
| BK g070 | 119 | N/A | N/A | 20.2 |
| BK g071 | 120 | N/A | N/A | 22.6 |
| BK g072 | 121 | N/A | N/A | 3.9 |
| BK g073 | 122 | N/A | N/A | 0.4 |
| BK g074 | 123 | N/A | N/A | 6.2 |
| BK g075 | 124 | N/A | N/A | 10.0 |
| BK g076 | 125 | N/A | N/A | 9.6 |
| BK g077 | 126 | N/A | N/A | 11.6 |
| BK g078 | 127 | N/A | N/A | 9.3 |
| BK g079 | 128 | N/A | N/A | 10.7 |
| BK g080 | 129 | N/A | N/A | 12.3 |
| BK g081 | 130 | N/A | N/A | 0.5 |
| BK g050 | 99 | BK g040 | 131 | 24.4 |
| BK g051 | 100 | BK g040 | 131 | 28.3 |
| BK g052 | 101 | BK g040 | 131 | 35.6 |
| BK g053 | 102 | BK g040 | 131 | 39.3 |
| BK g054 | 103 | BK g040 | 131 | 51.0 |
| BK g055 | 104 | BK g040 | 131 | 55.7 |
| BK g056 | 105 | BK g040 | 131 | 50.1 |
| BK g057 | 106 | BK g040 | 131 | 1.2 |
| BK g058 | 107 | BK g040 | 131 | 39.8 |
| BK g059 | 108 | BK g040 | 131 | 46.4 |
| BK g060 | 109 | BK g040 | 131 | 50.8 |
| BK g061 | 110 | BK g040 | 131 | 41.6 |
| BK g062 | 111 | BK g040 | 131 | 34.5 |
| BK g063 | 112 | BK g040 | 131 | 52.5 |
| BK g064 | 113 | BK g040 | 131 | 55.9 |
| BK g065 | 114 | BK g040 | 131 | 1.7 |
| BK g066 | 115 | BK g040 | 131 | 41.8 |
| BK g067 | 116 | BK g040 | 131 | 52.2 |
| BK g068 | 117 | BK g040 | 131 | 55.8 |
| BK g069 | 118 | BK g040 | 131 | 57.0 |
| BK g070 | 119 | BK g040 | 131 | 61.0 |
| BK g071 | 120 | BK g040 | 131 | 67.9 |
| BK g072 | 121 | BK g040 | 131 | 12.5 |
| BK g073 | 122 | BK g040 | 131 | 3.0 |
| BK g074 | 123 | BK g040 | 131 | 32.8 |
| BK g075 | 124 | BK g040 | 131 | 38.6 |
| BK g076 | 125 | BK g040 | 131 | 37.7 |
| BK g077 | 126 | BK g040 | 131 | 36.6 |
| BK g078 | 127 | BK g040 | 131 | 36.9 |
| BK g079 | 128 | BK g040 | 131 | 39.8 |
| BK g080 | 129 | BK g040 | 131 | 38.6 |
| BK g081 | 130 | BK g040 | 131 | 4.2 |
| BK g050 | 99 | BK g041 | 132 | 11.1 |
| BK g051 | 100 | BK g041 | 132 | 19.3 |
| BK g052 | 101 | BK g041 | 132 | 23.6 |
| BK g053 | 102 | BK g041 | 132 | 28.7 |
| BK g054 | 103 | BK g041 | 132 | 34.2 |
| BK g055 | 104 | BK g041 | 132 | 43.2 |
| BK g056 | 105 | BK g041 | 132 | 39.6 |
| BK g057 | 106 | BK g041 | 132 | 0.9 |
| BK g058 | 107 | BK g041 | 132 | 31.3 |
| BK g059 | 108 | BK g041 | 132 | 39.9 |
| BK g060 | 109 | BK g041 | 132 | 39.5 |
| BK g061 | 110 | BK g041 | 132 | 28.3 |
| BK g062 | 111 | BK g041 | 132 | 27.4 |
| BK g063 | 112 | BK g041 | 132 | 40.6 |
| BK g064 | 113 | BK g041 | 132 | 48.1 |
| BK g065 | 114 | BK g041 | 132 | 1.4 |
| BK g066 | 115 | BK g041 | 132 | 41.2 |
| BK g067 | 116 | BK g041 | 132 | 47.0 |
| BK g068 | 117 | BK g041 | 132 | 51.0 |
| BK g069 | 118 | BK g041 | 132 | 56.3 |
| BK g070 | 119 | BK g041 | 132 | 56.6 |
| BK g071 | 120 | BK g041 | 132 | 60.9 |
| BK g072 | 121 | BK g041 | 132 | 11.1 |
| BK g073 | 122 | BK g041 | 132 | 1.9 |
| BK g074 | 123 | BK g041 | 132 | 27.6 |
| BK g075 | 124 | BK g041 | 132 | 34.2 |
| BK g076 | 125 | BK g041 | 132 | 27.2 |
| BK g077 | 126 | BK g041 | 132 | 31.8 |
| BK g078 | 127 | BK g041 | 132 | 38.4 |
| BK g079 | 128 | BK g041 | 132 | 34.4 |
| BK g080 | 129 | BK g041 | 132 | 38.8 |
| BK g081 | 130 | BK g041 | 132 | 3.0 |

TABLE 16
EDITING EFFICIENCY IN HEK293T

| tagRNA | SEQ | egRNA | SEQ | Editing |
|---|---|---|---|---|
| Name | ID NO: | Name | ID NO: | (%) |
| BK g050 | 99 | N/A | N/A | 4.3 |
| BK g051 | 100 | N/A | N/A | 4.5 |
| BK g052 | 101 | N/A | N/A | 6.0 |
| BK g053 | 102 | N/A | N/A | 6.9 |
| BK g054 | 103 | N/A | N/A | 11.3 |
| BK g055 | 104 | N/A | N/A | 22.0 |
| BK g056 | 105 | N/A | N/A | 8.6 |
| BK g057 | 106 | N/A | N/A | 0.2 |
| BK g058 | 107 | N/A | N/A | 8.8 |
| BK g059 | 108 | N/A | N/A | 12.8 |
| BK g060 | 109 | N/A | N/A | 20.8 |
| BK g061 | 110 | N/A | N/A | 25.4 |
| BK g062 | 111 | N/A | N/A | 25.3 |
| BK g063 | 112 | N/A | N/A | 28.3 |
| BK g064 | 113 | N/A | N/A | 15.3 |
| BK g065 | 114 | N/A | N/A | 9.2 |
| BK g066 | 115 | N/A | N/A | 16.5 |
| BK g067 | 116 | N/A | N/A | 20.0 |
| BK g068 | 117 | N/A | N/A | 21.4 |
| BK g069 | 118 | N/A | N/A | 25.0 |
| BK g070 | 119 | N/A | N/A | 26.4 |
| BK g071 | 120 | N/A | N/A | 29.8 |
| BK g072 | 121 | N/A | N/A | 4.4 |
| BK g073 | 122 | N/A | N/A | 0.585 |
| BK g074 | 123 | N/A | N/A | 14.3 |
| BK g075 | 124 | N/A | N/A | 15.5 |
| BK g076 | 125 | N/A | N/A | 15.6 |
| BK g077 | 126 | N/A | N/A | 19.6 |
| BK g078 | 127 | N/A | N/A | 18.7 |
| BK g079 | 128 | N/A | N/A | 16.7 |
| BK g080 | 129 | N/A | N/A | 20.3 |
| BK g081 | 130 | N/A | N/A | 1.2 |
| BK g050 | 99 | BK g040 | 131 | 7.6 |
| BK g051 | 100 | BK g040 | 131 | 10.5 |
| BK g052 | 101 | BK g040 | 131 | 16.1 |
| BK g053 | 102 | BK g040 | 131 | 11.9 |
| BK g054 | 103 | BK g040 | 131 | 37.5 |
| BK g055 | 104 | BK g040 | 131 | 53.9 |
| BK g056 | 105 | BK g040 | 131 | 37.9 |
| BK g057 | 106 | BK g040 | 131 | 0.6 |
| BK g058 | 107 | BK g040 | 131 | 16.8 |
| BK g059 | 108 | BK g040 | 131 | 28.3 |
| BK g060 | 109 | BK g040 | 131 | 45.3 |
| BK g061 | 110 | BK g040 | 131 | 51.4 |
| BK g062 | 111 | BK g040 | 131 | 54.4 |
| BK g063 | 112 | BK g040 | 131 | 56.9 |
| BK g064 | 113 | BK g040 | 131 | 48.9 |
| BK g065 | 114 | BK g040 | 131 | 1.1 |
| BK g066 | 115 | BK g040 | 131 | 31.1 |
| BK g067 | 116 | BK g040 | 131 | 44.2 |
| BK g068 | 117 | BK g040 | 131 | 51.6 |
| BK g069 | 118 | BK g040 | 131 | 58.1 |
| BK g070 | 119 | BK g040 | 131 | 58.9 |
| BK g071 | 120 | BK g040 | 131 | 59.6 |
| BK g072 | 121 | BK g040 | 131 | 11.1 |
| BK g073 | 122 | BK g040 | 131 | 2.1 |
| BK g074 | 123 | BK g040 | 131 | 18.2 |
| BK g075 | 124 | BK g040 | 131 | 18.2 |
| BK g076 | 125 | BK g040 | 131 | 32.5 |
| BK g077 | 126 | BK g040 | 131 | 35.5 |
| BK g078 | 127 | BK g040 | 131 | 41.0 |
| BK g079 | 128 | BK g040 | 131 | 46.1 |
| BK g080 | 129 | BK g040 | 131 | 47.7 |
| BK g081 | 130 | BK g040 | 131 | 4.2 |
| BK g050 | 99 | BK g041 | 132 | 6.4 |
| BK g051 | 100 | BK g041 | 132 | 13.3 |
| BK g052 | 101 | BK g041 | 132 | 19.5 |
| BK g053 | 102 | BK g041 | 132 | 11.2 |
| BK g054 | 103 | BK g041 | 132 | 31.0 |
| BK g055 | 104 | BK g041 | 132 | 51.2 |
| BK g056 | 105 | BK g041 | 132 | 31.1 |
| BK g057 | 106 | BK g041 | 132 | 0.6 |
| BK g058 | 107 | BK g041 | 132 | 15.7 |
| BK g059 | 108 | BK g041 | 132 | 25.7 |
| BK g060 | 109 | BK g041 | 132 | 51.0 |
| BK g061 | 110 | BK g041 | 132 | 41.9 |
| BK g062 | 111 | BK g041 | 132 | 50.6 |
| BK g063 | 112 | BK g041 | 132 | 54.6 |
| BK g064 | 113 | BK g041 | 132 | 43.4 |
| BK g065 | 114 | BK g041 | 132 | 1.2 |
| BK g066 | 115 | BK g041 | 132 | 27.4 |
| BK g067 | 116 | BK g041 | 132 | 41.5 |
| BK g068 | 117 | BK g041 | 132 | 48.8 |
| BK g069 | 118 | BK g041 | 132 | 46.1 |
| BK g070 | 119 | BK g041 | 132 | 42.8 |
| BK g071 | 120 | BK g041 | 132 | 58.7 |
| BK g072 | 121 | BK g041 | 132 | 14.4 |
| BK g073 | 122 | BK g041 | 132 | 2.5 |
| BK g074 | 123 | BK g041 | 132 | 29.1 |
| BK g075 | 124 | BK g041 | 132 | 28.3 |
| BK g076 | 125 | BK g041 | 132 | 40.6 |
| BK g077 | 126 | BK g041 | 132 | 46.6 |
| BK g078 | 127 | BK g041 | 132 | 49.4 |
| BK g079 | 128 | BK g041 | 132 | 49.3 |
| BK g080 | 129 | BK g041 | 132 | 55.0 |
| BK g081 | 130 | BK g041 | 132 | 4.3 |

### Editing in HEK293T Disease Cell Line Using Transfection of Plasmid DNA

HEK293T cell lines were engineered to contain the R408W disease mutation in the PAH gene, then used as models for mutation correction experiments. Furthermore, primary human hepatocytes were also used as model cells to install a surrogate mutation.

20,000 HEK293T cells carrying the R408W mutation were transfected using MessengerMax with an mRNA encoding RT Editor, a chemically synthesized tagRNA, and in some embodiments a chemically synthesized second guide RNA (egRNA). Specifically, 125 ng of mRNA, 1 pmol tagRNA and 0.3 pmol of egRNA (where applicable) were diluted to 10 μl and mixed with 30 μl Opti-MEM containing 0.5 μl MessengerMax. The lipoplexes were mixed with the cells in suspension and plated into 96-well plates. After transfection, cells were grown at 37° C., 500 CO2 in DMEM media supplemented with 10% serum for 3 days prior to cell lysis and genomic DNA extraction with QuickExtract solution. Editing of the PAH target locus was assessed using amplicon sequencing using primers flanking the PAH gene.

Table 17 shows the editing efficiency (average of 2 independent experiments and indel percentages.

TABLE 17
EDITING AND INDEL EFFICIENCY IN HEK293T CELLS

| | SEQ | Editing | indel |
|---|---|---|---|
| tagRNA1 Name | ID NO: | (%) | % |
| hPAH_NGG1_F15E6_TtoC | 409 | 0.7 | 0.3 |
| hPAH_NGG1_F14E6_TtoC | 410 | 1.6 | 0.2 |
| hPAH_NGG1_F13E6_TtoC | 411 | 2.7 | 0.3 |
| hPAH_NGG1_F12E6_TtoC | 412 | 3.9 | 0.3 |
| hPAH_NGG1_F11E6_TtoC | 413 | 4.3 | 0.3 |
| hPAH_NGG1_F10E6_TtoC | 414 | 2.1 | 0.3 |
| hPAH_NGG1_F9E6_TtoC | 415 | 2.0 | 0.3 |
| hPAH_NGG1_F8E6_TtoC | 416 | 0.3 | 0.3 |
| hPAH_NGG1_F7E6_TtoC | 417 | 1.4 | 0.2 |
| hPAH_NGG1_F15E8_TtoC | 418 | 0.1 | 0.3 |
| hPAH_NGG1_F14E8_TtoC | 419 | 0.2 | 0.2 |
| hPAH_NGG1_F13E8_TtoC | 420 | 0.1 | 0.3 |
| hPAH_NGG1_F12E8_TtoC | 421 | 0.4 | 0.3 |
| hPAH_NGG1_F11E8_TtoC | 422 | 0.6 | 0.2 |
| hPAH_NGG1_F10E8_TtoC | 423 | 0.7 | 0.2 |
| hPAH_NGG1_F9E8_TtoC | 424 | 0.6 | 0.3 |
| hPAH_NGG1_F8E8_TtoC | 425 | 0.8 | 0.4 |
| hPAH_NGG1_F7E8_TtoC | 426 | 1.1 | 0.3 |
| hPAH_NGG1_F15E10_TtoC | 427 | 1.0 | 0.2 |
| hPAH_NGG1_F14E10_TtoC | 428 | 2.0 | 0.2 |
| hPAH_NGG1_F13E10_TtoC | 429 | 2.5 | 0.3 |
| hPAH_NGG1_F12E10_TtoC | 430 | 3.9 | 0.4 |
| hPAH_NGG1_F11E10_TtoC | 431 | 4.8 | 0.3 |
| hPAH_NGG1_F10E10_TtoC | 432 | 5.6 | 0.4 |
| hPAH_NGG1_F9E10_TtoC | 433 | 3.8 | 0.4 |
| hPAH_NGG1_F8E10_TtoC | 434 | 3.8 | 0.4 |
| hPAH_NGG1_F7E10_TtoC | 435 | 4.9 | 0.4 |
| hPAH_NGG1_F14E12_TtoC | 436 | 1.6 | 0.3 |
| hPAH_NGG1_F13E12_TtoC | 437 | 2.3 | 0.3 |
| hPAH_NGG1_F12E12_TtoC | 438 | 3.6 | 0.4 |
| hPAH_NGG1_F11E12_TtoC | 439 | 4.2 | 0.3 |
| hPAH_NGG1_F10E12_TtoC | 440 | 5.7 | 0.3 |
| hPAH_NGG1_F9E12_TtoC | 441 | 4.8 | 0.4 |
| hPAH_NGG1_F8E12_TtoC | 442 | 4.2 | 0.4 |
| hPAH_NGG1_F7E12_TtoC | 443 | 4.3 | 0.4 |
| hPAH_NGG1_F15E14_TtoC | 444 | 0.04 | 0.3 |
| hPAH_NGG1_F14E14_TtoC | 445 | 0.1 | 0.3 |
| hPAH_NGG1_F13E14_TtoC | 446 | 0.1 | 0.2 |
| hPAH_NGG1_F12E14_TtoC | 447 | 0.2 | 0.3 |
| hPAH_NGG1_F11E14_TtoC | 448 | 0.2 | 0.2 |
| hPAH_NGG1_F10E14_TtoC | 449 | 0.6 | 0.3 |
| hPAH_NGG1_F9E14_TtoC | 450 | 0.8 | 0.4 |
| hPAH_NGG1_F8E14_TtoC | 451 | 1.1 | 0.3 |
| hPAH_NGG1_F7E14_TtoC | 452 | 1.2 | 0.3 |
| hPAH_NGG2_F15E7_AtoG | 453 | 0.4 | 0.2 |
| hPAH_NGG2_F14E7_AtoG | 454 | 0.6 | 0.4 |
| hPAH_NGG2_F13E7_AtoG | 455 | 0.6 | 0.3 |
| hPAH_NGG2_F12E7_AtoG | 456 | 0.8 | 0.4 |
| hPAH_NGG2_F11E7_AtoG | 457 | 0.9 | 0.5 |
| hPAH_NGG2_F10E7_AtoG | 458 | 0.7 | 0.5 |
| hPAH_NGG2_F9E7_AtoG | 459 | 0.6 | 0.6 |
| hPAH_NGG2_F8E7_AtoG | 460 | 0.2 | 0.4 |
| hPAH_NGG2_F7E7_AtoG | 461 | 0.1 | 0.4 |
| hPAH_NGG2_F15E9_AtoG | 462 | 1.4 | 0.3 |
| hPAH_NGG2_F14E9_AtoG | 463 | 2.1 | 0.4 |
| hPAH_NGG2_F13E9_AtoG | 464 | 3.0 | 0.3 |
| hPAH_NGG2_F12E9_AtoG | 465 | 3.9 | 0.5 |
| hPAH_NGG2_F11E9_AtoG | 466 | 4.4 | 0.5 |
| hPAH_NGG2_F10E9_AtoG | 467 | 2.5 | 0.4 |
| hPAH_NGG2_F9E9_AtoG | 468 | 1.9 | 0.5 |
| hPAH_NGG2_F8E9_AtoG | 469 | 0.6 | 0.4 |
| hPAH_NGG2_F7E9_AtoG | 470 | 0.1 | 0.4 |
| hPAH_NGG2_F15E11_AtoG | 471 | 1.1 | 0.3 |
| hPAH_NGG2_F14E11_AtoG | 472 | 1.6 | 0.4 |
| hPAH_NGG2_F13E11_AtoG | 473 | 3.2 | 0.5 |
| hPAH_NGG2_F12E11_AtoG | 474 | 3.6 | 0.5 |
| hPAH_NGG2_F11E11_AtoG | 475 | 4.5 | 0.7 |
| hPAH_NGG2_F10E11_AtoG | 476 | 2.4 | 0.4 |
| hPAH_NGG2_F9E11_AtoG | 477 | 2.2 | 0.6 |
| hPAH_NGG2_F8E11_AtoG | 478 | 0.4 | 0.4 |
| hPAH_NGG2_F7E11_AtoG | 479 | 0.1 | 0.3 |
| hPAH_NGG2_F15E13_AtoG | 480 | 0.1 | 0.3 |
| hPAH_NGG2_F14E13_AtoG | 481 | 0.4 | 0.3 |
| hPAH_NGG2_F13E13_AtoG | 482 | 1.0 | 0.4 |
| hPAH_NGG2_F12E13_AtoG | 483 | 1.2 | 0.4 |
| hPAH_NGG2_F11E13_AtoG | 484 | 1.6 | 0.6 |
| hPAH_NGG2_F10E13_AtoG | 485 | 2.2 | 0.4 |
| hPAH_NGG2_F9E13_AtoG | 486 | 1.7 | 0.5 |
| hPAH_NGG2_F8E13_AtoG | 487 | 1.2 | 0.4 |
| hPAH_NGG2_F7E13_AtoG | 488 | 0.2 | 0.3 |
| hPAH_NGG2_F15E15_AtoG | 489 | 0.1 | 0.3 |
| hPAH_NGG2_F14E15_AtoG | 490 | 0.01 | 0.3 |
| hPAH_NGG2_F13E15_AtoG | 491 | 0.02 | 0.3 |
| hPAH_NGG2_F12E15_AtoG | 492 | 0.1 | 0.4 |
| hPAH_NGG2_F11E15_AtoG | 493 | 0.03 | 0.5 |
| hPAH_NGG2_F10E15_AtoG | 494 | 0.9 | 0.5 |
| hPAH_NGG2_F9E15_AtoG | 495 | 1.1 | 0.4 |
| hPAH_NGG2_F8E15_AtoG | 496 | 0.1 | 0.3 |
| hPAH_NGG2_F7E15_AtoG | 497 | 0.1 | 0.5 |

In primary human hepatocytes, 50,000 cells were transfected with 250 ng of mRNA, 50 ng of tagRNA and 15 ng of egRNA formulated in 10 μL of Opti-MEM and 0.75 uL of Messenger Max. The transfection mixes and cells were added to 96-well plates and incubated at 37° C., 500 CO2 for 72 hirs prior to genomic DNA extraction via QuickExtract. Editing efficacy was measured using amplicon sequencing using primers flanking the PAH gene.

Table 17 shows the editing efficacies achieved with NGG1 and NGG2 tagRNAs. Overall, both NGG1 and NGG2 tagRNA compositions successfully corrected the R408W mutation in HTEK293T cells with minimal indels. Certain compositions performed better than others.

Table 18B shows the editing efficacies achieved with various egRNAs in primary human hepatocytes (tagRNA sequences are shown in Table 18A). Some egRNAs compositions increase editing efficiency relative to others. The editing efficiencies and indel percentages shown in the table are the average of 2 independent experiments.

TABLE 18A
SURROGATE tagRNA

| NAME | SEQUENCE^{1,2} | SEQ ID NO: |
|---|---|---|
| hPAH_NGG1 | mU*mA*mG*rCrGrArArCrUrGrArGrArArGrGrGrCrCrGrGrUrUrUrUrAr | 791 |
| | GrAmGmCmUmAmGmAmAmAmUmAmGmCrArArGrUrUrArArArArUr | |
| | ArArGrGrCrUrArGrUrCrCrGrUrUrArUrCmAmAmCmUmUmGmAmAmA | |
| | mAmAmGmUmGmGmCmAmCmCmGmAmGmUmCmGmGmUmGmCrA | |
| | rCrArArUrArCrCrCrCrGrGrCrCrCrUrUrCrU*mC*mA*mG | |
| hPAH_NGG2 | mA*mC*mU*rUrUrGrCrUrGrCrCrArCrArArUrArCrCrUrGrUrUrUrUrArG | 792 |
| | rAmGmCmUmAmGmAmAmAmUmAmGmCrArArGrUrUrArArArArUrAr | |
| | ArGrGrCrUrArGrUrCrCrGrUrUrArUrCmAmAmCmUmUmGmAmAmAm | |
| | AmAmGmUmGmGmCmAmCmCmGmAmGmUmCmGmGmUmGmCrGr | |
| | ArGrArArGrGrGrGrCrGrArGrGrUrArUrUrGrUrG*mG*mC*mA | |

¹For RNA, T is U; U are T in the sequence listing. For each entry, the spacer sequence is underlined and the extension arm is bold.
²For the sequences: *, phosphorothioate modification; mN, 2′O-methyl modification; rN, unmodified ribonucleotide; i2FN, Int Fluoro modification. N = A, C, T/U, G.

TABLE 18B
EDITING AND INDEL EFFICIENCY IN
PRIMARY HUMAN HEPATOCYTE CELLS

| | SEQ | | SEQ | Editing | Indels |
|---|---|---|---|---|---|
| tagRNA Name | ID NO: | egRNA Name | ID NO: | (%) | (%) |
| hPAH_NGG1 | 791 | hPAH_egRNA_118038 | 527 | 6 | 1.2 |
| hPAH_NGG1 | 791 | hPAH_egRNA_118030 | 528 | 11 | 0.7 |
| hPAH_NGG1 | 791 | hPAH_egRNA_117991 | 529 | 13 | 0.3 |
| hPAH_NGG1 | 791 | hPAH_egRNA_117964 | 530 | 11 | 0.6 |
| hPAH_NGG1 | 791 | hPAH_egRNA_117958 | 531 | 13 | 1.8 |
| hPAH_NGG1 | 791 | hPAH_egRNA_117951 | 532 | 11 | 0.5 |
| hPAH_NGG1 | 791 | hPAH_egRNA_117891 | 533 | 15 | 2.2 |
| hPAH_NGG1 | 791 | hPAH_egRNA_117884 | 534 | 13 | 0.5 |
| hPAH_NGG1 | 791 | hPAH_egRNA_117878 | 535 | 20 | 1.8 |
| hPAH_NGG1 | 791 | hPAH_egRNA_117856 | 536 | 12 | 1.3 |
| hPAH_NGG2 | 792 | hPAH_egRNA_117802 | 537 | 12 | 0.5 |
| hPAH_NGG2 | 792 | hPAH_egRNA_117927 | 538 | 16 | 0.4 |
| hPAH_NGG2 | 792 | hPAH_egRNA_118009 | 539 | 13 | 0.6 |
| hPAH_NGG2 | 792 | hPAH_egRNA_117809 | 540 | 11 | 0.7 |
| hPAH_NGG2 | 792 | hPAH_egRNA_117928 | 541 | 15 | 0.6 |
| hPAH_NGG2 | 792 | hPAH_egRNA_118032 | 542 | 9 | 0.7 |
| hPAH_NGG2 | 792 | hPAH_egRNA_117829 | 543 | 20 | 0.8 |
| hPAH_NGG2 | 792 | hPAH_egRNA_117947 | 544 | 9 | 0.7 |
| hPAH_NGG2 | 792 | hPAH_egRNA_118042 | 545 | 11 | 0.5 |
| hPAH_NGG2 | 792 | hPAH_egRNA_117853 | 546 | 19 | 1.1 |
| hPAH_NGG2 | 792 | hPAH_egRNA_117948 | 547 | 18 | 1.1 |
| hPAH_NGG2 | 792 | hPAH_egRNA_117860 | 548 | 22 | 1.2 |
| hPAH_NGG2 | 792 | hPAH_egRNA_117955 | 549 | 22 | 0.8 |
| hPAH_NGG2 | 792 | hPAH_egRNA_117870 | 550 | 16 | 1.8 |
| hPAH_NGG2 | 792 | hPAH_egRNA_117956 | 551 | 23 | 1.0 |
| hPAH_NGG2 | 792 | hPAH_egRNA_117871 | 552 | 14 | 2.7 |
| hPAH_NGG2 | 792 | hPAH_egRNA_117982 | 553 | 14 | 0.5 |
| hPAH_NGG2 | 792 | hPAH_egRNA_117883 | 554 | 20 | 1.2 |
| hPAH_NGG2 | 792 | hPAH_egRNA_117983 | 555 | 7 | 0.3 |

### Example 2

### tagRNAs with Chemical Modifications

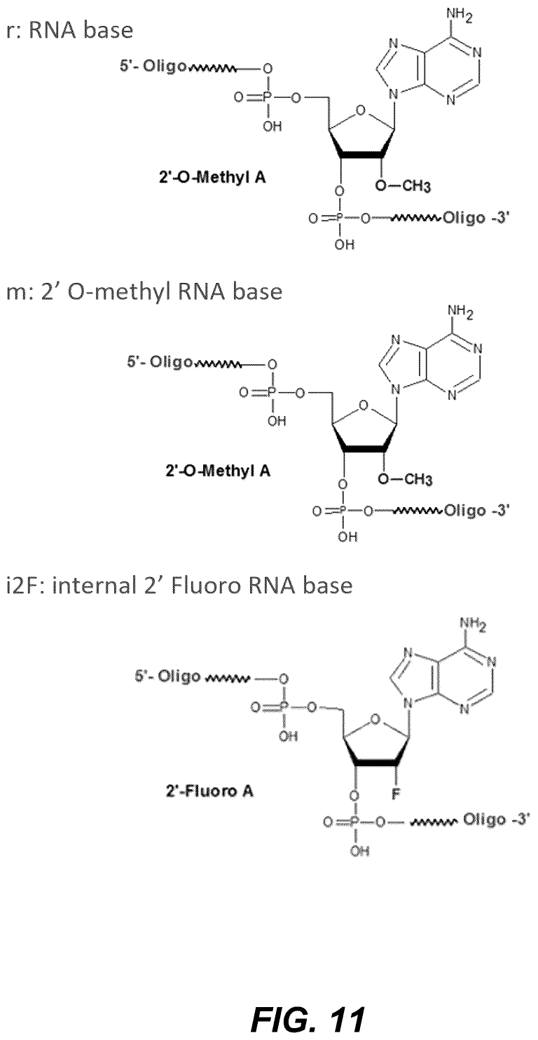

tagRNAs targeting an ATP71B gene with chemical modifications at the Editing Template (“ET”) region and/or FBS region were contemplated, as shown in FIG. 11. Chemically modified tagRNAs generated are presented in the Table 19 below. ‘in’ indicates 2′ O-methyl RNA base; ‘r’ indicates RNA base; ‘i2F’ indicates internal 2′ Fluoro RNA base. The tagRNAs were tested in two hepatic cell lines, HTEK293T and Huh7 and the data is presented below in Table 20. Certain chemical modifications increased editing efficiency of the tagRNAs.

TABLE 19
CHEMICALLY MODIFIED tagRNAs

| | | SEQ | |
|---|---|---|---|
| tagRNA | ET | ID NO: | FBS |
| PG g060 | mGmUmGmAmAmCmAmCmCmCmCmUmUmG | 556 | rGrGrCrGrUrGrGrC |
| PG g061 | rGrUrGrArArCrArCrCrCrCrUrUrG | 557 | mGmGmCmGmUmGmGmC |
| PG g062 | mGmUmGmAmAmCmAmCmCmCmCmUmUmG | 556 | mGmGmCmGmUmGmGmC |
| PG g063 | rGmUrGmArAmCrAmCrCmCrCmUrUmG | 558 | rGrGrCrGrUrGrGrC |
| PG g064 | rGrUrGrArArCrArCrCrCrCrUrUrG | 557 | rGmGrCmGrUmGrGmC |
| PG g065 | rGmUrGmArAmCrAmCrCmCrCmUrUmG | 558 | rGmGrCmGrUmGrGmC |
| PG g066 | rGrUmGrArAmCrArCmCrCrCmUrUrG | 559 | rGrGrCrGrUrGrGrC |
| PG g067 | rGrUrGrArArCrArCrCrCrCrUrUrG | 557 | mGrGrCmGrUrGmGrC |
| PG g068 | rGrUmGrArAmCrArCmCrCrCmUrUrG | 559 | mGrGrCmGrUrGmGrC |
| PG g069 | /i2FG//i2FU//i2FG//i2FA//i2FA//i2FC// | 560 | rGrGrCrGrUrGrGrC |
| | i2FA//i2FC//i2FC//i2FC//i2FC//i2FU// | | |
| | i2FU//i2FG/ | | |
| PG g070 | rGrUrGrArArCrArCrCrCrCrUrUrG | 557 | /i2FG//i2FG//i2FC//i2FG//i2FU// |
| | | | i2FG//i2FG//i2FC/ |
| PG g071 | /i2FG//i2FU//i2FG//i2FA//i2FA//i2FC// | 560 | /i2FG//i2FG//i2FC//i2FG//i2FU// |
| | i2FA//i2FC//i2FC//i2FC//i2FC//i2FU// | | i2FG//i2FG//i2FC/ |
| | i2FU//i2FG/ | | |
| PG g072 | rG/i2FU/rG/i2FA/rA/i2FC/rA/i2FC/rC/i2FC/ | 561 | rGrGrCrGrUrGrGrC |
| | rC/i2FU/rU/i2FG | | |
| PG g073 | rGrUrGrArArCrArCrCrCrCrUrUrG | 557 | rG/i2FG/rC/i2FG/rU/i2FG/rG/i2FC |
| PG g074 | rG/i2FU/rG/i2FA/rA/i2FC/rA/i2FC/rC/i2FC/ | 561 | rG/i2FG/rC/i2FG/rU/i2FG/rG/i2FC |
| | rC/i2FU/rU/i2FG | | |
| PG g075 | rGrU/i2FG/rArA/i2FC/rArC/i2FC/rCrC/i2FU/ | 562 | rGrGrCrGrUrGrGrC |
| | rUrG | | |
| PG g076 | rGrUrGrArArCrArCrCrCrCrUrUrG | 557 | /i2FG/rGrC/i2FG/rUrG/i2FG/rC |
| PG g077 | rGrU/i2FG/rArA/i2FC/rArC/i2FC/rCrC/i2FU/ | 562 | /i2FG/rGrC/i2FG/rUrG/i2FG/rC |
| | rUrG | | |
| BK g071 | GUGAACACCCCUUG | 97 | GGCGUGGC |

TABLE 20
EDITING EFFICIENCY WITH CHEMICALLY
MODIFIED tagRNAs

| tagRNA | Modification Type | HEK293T | Huh7 |
|---|---|---|---|
| PG g060 | ET14OMe | 0.1% | 0.2% |
| PG g061 | FBS8OMe | 0.1% | 0.3% |
| PG g062 | ET14OMe•FBS8OMe | 0.2% | 0.2% |
| PG g063 | ET7OMe | 0.2% | 0.1% |
| PG g064 | FBS4OMe | 20.9% | 5.8% |
| PG g065 | ET7OMe•FBS4OMe | 0.1% | 0.2% |
| PG g066 | ET4OMe | 0.1% | 0.2% |
| PG g067 | FBS3OMe | 14.4% | 5.3% |
| PG g068 | ET4OMe•FBS3OMe | 0.1% | 0.2% |
| PG g069 | ET14F | 0.1% | 0.1% |
| PG g070 | FBS8F | 22.0% | 7.9% |
| PG g071 | ET14F•FBS8F | 0.1% | 3.1% |
| PG g072 | ET7F | 0.5% | 0.1% |
| PG g073 | FBS4F | 33.8% | 11.4% |
| PG g074 | ET7F•FBS4F | 0.1% | 0.2% |
| PG g075 | ET4F | 25.4% | 7.8% |
| PG g076 | FBS3F | 30.6% | 7.9% |
| PG g077 | ET4F•FBS3F | 42.4% | 10.6% |
| BK g071 | — | 32.8% | 9.9% |

### Example 3

### Methods and Compositions for Editing the A1ATD Gene

The lack of NGG PAMs around the mutation site of the SERPINA1 (also referred to herein as A1ATD) gene necessitated a search for alternative Cas9 proteins as a component for the RT editing system.

A comprehensive screening was done of the various alternative Cas9s that are capable of the E342K correction via RT editing. The results are shown in Table 21 and are the average indel % of each Cas9 with a tool gRNA was assessed from three independent experiments with replicates. Cas9 proteins that were considered for further evaluation are Slutr1Cas9, Ssch1Cas9, Ssch3Cas9, Ssi5Cas9, SpRYCas9, SpRYcCas9, and SSACas9.

TABLE 21
EFFICIENCIES OF CAS PROTEINS

| | | SEQ ID | Indel |
|---|---|---|---|
| Cas9 name | PAM | NO: | (%) |
| iSpyMac | NAA | 147 | 2.3 |
| Sag3Cas9 | NNaAAa | 152 | 2.2 |
| Sha4Cas9 | NNGRC | 144 | 43.3 |
| Shy1Cas9 | NNaAAa | 151 | 2.6 |
| Slutr1Cas9 | NNGAa | 153 | 87.9 |
| Sma2Cas9 | NHGYNAN(A > B) | 157 | 2.1 |
| SpRYCas9 | NNN | 155 | 74.3 |
| SpRYcCas9 | NNN | 156 | 74.3 |
| Sro1Cas9 | NNGA | 143 | 2.1 |
| SSACas9 | NNAGAAA | 158 | 88.6 |
| Ssch1Cas9 | NNGA | 142 | 45.6 |
| Ssch3Cas9 | NNGA | 154 | 42.3 |
| Ssci4Cas9 | NNGA | 150 | 2.0 |
| Ssi5Cas9 | NNGA | 148 | 46.2 |
| Ssi8Cas9 | NNGA | 149 | 3.7 |
| SsuCas9 | NNGAAAa | 145 | 1.6 |

Huh7 cell line was engineered to contain the E342K disease mutation in the SERPINA1 gene, then used as model for mutation correction experiments. Twenty spacers close to the mutation site were designed with two scaffold variants. One sgRNA can be tested with multiple of the Cas9 proteins shown above. A total of 57 combinations with 3 replicates were tested.

20,000 HuH7 cells carrying the E342K mutation were transfected using MessengerMax with an mRNA encoding the RT Editor, and a chemically synthesized tagRNA. Specifically, 125 ng of mRNA, and 1 pmol tagRNA were diluted to 10 μl and mixed with 30 μl Opti-MEM containing 0.5 μl MessengerMax. The lipoplexes were mixed with the cells in suspension and plated into 96-well plates. After transfection, cells were grown at 37° C., 5% CO2 in DMEM media supplemented with 10% serum for 3 days prior to cell lysis and genomic DNA extraction with QuickExtract solution. Editing of the SERPINA target locus was assessed using amplicon sequencing using primers flanking the gene.

Table 22A and Table 22B show information related to the editing efficacies (average of 3 independent experiments) achieved with SpRYcCas9-based RT Editor (pAM033; mRNA sequence SEQ TD NO: 1330, protein sequence SEQ ID NO: 1329) and SsaCas9-based RT Editor (pAM035; mRNA sequence SEQ TD NO: 1037, protein sequence SEQ ID NO: 1036).

TABLE 22A
EDITING EFFICIENCIES

| | | SEQ | Edit | Indel |
|---|---|---|---|---|
| RT Editor | tagRNA Name | ID NO: | (%) | (%) |
| SsaCas9 RT Editor | N20_NNAGAAA_+5_S_F12E8_TtoC | 623 | 8 | 0.3 |
| SsaCas9 RT Editor | N20_NNAGAAA_+5_S_F11E8_TtoC | 624 | 13 | 0.5 |
| SsaCas9 RT Editor | N20_NNAGAAA_+5_S_F10E8_TtoC | 625 | 11 | 0.4 |
| SsaCas9 RT Editor | N20_NNAGAAA_+5_S_F9E8_TtoC | 626 | 15 | 0.7 |
| SsaCas9 RT Editor | N20_NNAGAAA_+5_S_F8E8_TtoC | 627 | 19 | 1.0 |
| SsaCas9 RT Editor | N20_NNAGAAA_+5_S_F7E8_TtoC | 628 | 14 | 0.7 |
| SsaCas9 RT Editor | N20_NNAGAAA_+5_S_F12E10_TtoC | 629 | 10 | 0.3 |
| SsaCas9 RT Editor | N20_NNAGAAA_+5_S_F11E10_TtoC | 630 | 16 | 0.5 |
| SsaCas9 RT Editor | N20_NNAGAAA_+5_S_F10E10_TtoC | 631 | 10 | 0.4 |
| SsaCas9 RT Editor | N20_NNAGAAA_+5_S_F9E10_TtoC | 632 | 15 | 0.4 |
| SsaCas9 RT Editor | N20_NNAGAAA_+5_S_F8E10_TtoC | 633 | 15 | 0.6 |
| SsaCas9 RT Editor | N20_NNAGAAA_+5_S_F7E10_TtoC | 634 | 12 | 0.6 |
| SsaCas9 RT Editor | N20_NNAGAAA_+5_S_F12E12_TtoC | 635 | 9 | 2.0 |
| SsaCas9 RT Editor | N20_NNAGAAA_+5_S_F11E12_TtoC | 636 | 10 | 3.0 |
| SsaCas9 RT Editor | N20_NNAGAAA_+5_S_F10E12_TtoC | 637 | 9 | 1.8 |
| SsaCas9 RT Editor | N20_NNAGAAA_+5_S_F9E12_TtoC | 638 | 12 | 2.9 |
| SsaCas9 RT Editor | N20_NNAGAAA_+5_S_F8E12_TtoC | 639 | 11 | 2.7 |
| SsaCas9 RT Editor | N20_NNAGAAA_+5_S_F7E12_TtoC | 640 | 9 | 1.8 |
| SsaCas9 RT Editor | N20_NNAGAAA_+5_S_F12E14_TtoC | 641 | 8 | 2.0 |
| SsaCas9 RT Editor | N20_NNAGAAA_+5_S_F11E14_TtoC | 642 | 8 | 2.4 |
| SsaCas9 RT Editor | N20_NNAGAAA_+5_S_F10E14_TtoC | 643 | 7 | 1.6 |
| SsaCas9 RT Editor | N20_NNAGAAA_+5_S_F9E14_TtoC | 644 | 8 | 1.8 |
| SsaCas9 RT Editor | N20_NNAGAAA_+5_S_F8E14_TtoC | 645 | 10 | 1.5 |
| SsaCas9 RT Editor | N20_NNAGAAA_+5_S_F7E14_TtoC | 646 | 8 | 1.1 |
| SsaCas9 RT Editor | N20_NNAGAAA_+5_S_F12E16_TtoC | 647 | 11 | 2.3 |
| SsaCas9 RT Editor | N20_NNAGAAA_+5_S_F11E16_TtoC | 648 | 10 | 2.5 |
| SsaCas9 RT Editor | N20_NNAGAAA_+5_S_F10E16_TtoC | 649 | 8 | 2.2 |
| SsaCas9 RT Editor | N20_NNAGAAA_+5_S_F9E16_TtoC | 650 | 10 | 3.8 |
| SsaCas9 RT Editor | N20_NNAGAAA_+5_S_F8E16_TtoC | 651 | 11 | 3.6 |
| SsaCas9 RT Editor | N20_NNAGAAA_+5_S_F7E16_TtoC | 652 | 10 | 2.4 |
| SpRYcCas9 RT Editor | N20_NAA_+1_S_F12E4_TtoC | 653 | 31 | 0.2 |
| SpRYcCas9 RT Editor | N20_NAA_+1_S_F11E4_TtoC | 654 | 31 | 0.2 |
| SpRYcCas9 RT Editor | N20_NAA_+1_S_F10E4_TtoC | 655 | 47 | 0.4 |
| SpRYcCas9 RT Editor | N20_NAA_+1_S_F9E4_TtoC | 656 | 39 | 0.2 |
| SpRYcCas9 RT Editor | N20_NAA_+1_S_F8E4_TtoC | 657 | 49 | 0.2 |
| SpRYcCas9 RT Editor | N20_NAA_+1_S_F7E4_TtoC | 658 | 36 | 0.2 |
| SpRYcCas9 RT Editor | N20_NAA_+1_S_F12E6_TtoC | 659 | 57 | 0.6 |
| SpRYcCas9 RT Editor | N20_NAA_+1_S_F11E6_TtoC | 660 | 63 | 0.5 |
| SpRYcCas9 RT Editor | N20_NAA_+1_S_F10E6_TtoC | 661 | 68 | 0.5 |
| SpRYcCas9 RT Editor | N20_NAA_+1_S_F9E6_TtoC | 662 | 51 | 0.6 |
| SpRYcCas9 RT Editor | N20_NAA_+1_S_F8E6_TtoC | 663 | 68 | 0.6 |
| SpRYcCas9 RT Editor | N20_NAA_+1_S_F7E6_TtoC | 664 | 49 | 0.3 |
| SpRYcCas9 RT Editor | N20_NAA_+1_S_F12E8_TtoC | 665 | 21 | 0.8 |
| SpRYcCas9 RT Editor | N20_NAA_+1_S_F11E8_TtoC | 666 | 33 | 1.5 |
| SpRYcCas9 RT Editor | N20_NAA_+1_S_F10E8_TtoC | 667 | 48 | 2.2 |
| SpRYcCas9 RT Editor | N20_NAA_+1_S_F9E8_TtoC | 668 | 44 | 1.5 |
| SpRYcCas9 RT Editor | N20_NAA_+1_S_F8E8_TtoC | 669 | 65 | 2.7 |
| SpRYcCas9 RT Editor | N20_NAA_+1_S_F7E8_TtoC | 670 | 47 | 1.7 |
| SpRYcCas9 RT Editor | N20_NAA_+1_S_F12E10_TtoC | 671 | 47 | 2.3 |
| SpRYcCas9 RT Editor | N20_NAA_+1_S_F11E10_TtoC | 672 | 45 | 1.8 |
| SpRYcCas9 RT Editor | N20_NAA_+1_S_F10E10_TtoC | 673 | 52 | 1.6 |
| SpRYcCas9 RT Editor | N20_NAA_+1_S_F9E10_TtoC | 674 | 44 | 1.5 |
| SpRYcCas9 RT Editor | N20_NAA_+1_S_F8E10_TtoC | 675 | 63 | 2.2 |
| SpRYcCas9 RT Editor | N20_NAA_+1_S_F7E10_TtoC | 676 | 31 | 0.9 |
| SpRYcCas9 RT Editor | N20_NAA_+1_S_F12E12_TtoC | 677 | 61 | 2.3 |
| SpRYcCas9 RT Editor | N20_NAA_+1_S_F11E12_TtoC | 678 | 46 | 2.7 |
| SpRYcCas9 RT Editor | N20_NAA_+1_S_F10E12_TtoC | 679 | 57 | 1.7 |
| SpRYcCas9 RT Editor | N20_NAA_+1_S_F9E12_TtoC | 680 | 25 | 1.0 |
| SpRYcCas9 RT Editor | N20_NAA_+1_S_F8E12_TtoC | 681 | 52 | 1.5 |
| SpRYcCas9 RT Editor | N20_NAA_+1_S_F7E12_TtoC | 682 | 20 | 0.5 |

TABLE 22B
RT EDITOR SEQUENCES

| | Protein Sequence | |
|---|---|---|
| Name | (SEQ ID NO:) | mRNA Sequence (SEQ ID NO:) |
| pAM035 | 1036 | 1037 |
| pAM033 | MKRTADGSEFESPKKKRKVE | AGGAAATAAGAGAGAAAAGAAGAGTAAGAAGAAATAT |
| | KKYSIGLDIGTNSVGWAVITD | AAGAGCCACCATGAAACGGACAGCCGACGGAAGCGAG |
| | DYKVPSKKFKVLGNTNRKSI | TTCGAGTCACCAAAGAAGAAGCGGAAAGTCgaaaaaaagtaca |
| | KKNLMGALLFDSGETAEATR | gcatcggcctagacatcggaaccaacagcgtgggctgggccgtgatcacggacgactaca |
| | LKRTARRRYTRRKNRIRYLQE | aggtgcccagcaagaagttcaaagtgctgggcaacacaaacagaaagagcatcaaaaaga |
| | IFANEMAKLDDSFFQRLEESF | acctgatgggcgccctgctgttcgacagcggcgagacagccgaggccaccagactgaagc |
| | LVEEDKKNERHPIFGNLADEV | ggaccgccaggagacggtacacccgccggaagaacagaataagatacctgcaggagattt |
| | AYHRNYPTIYHLRKKLADSPE | tcgccaacgaaatggccaagctggacgacagcttctttcagaggctggaagagagcttcctg |
| | KADLRLIYLALAHIIKFRGHFL | gtggaggaggataagaagaacgaaagacaccccatattcggcaacctggccgacgaggtg |
| | IEGKLNAENSDVAKLFYQLIQ | gcgtaccacagaaactaccctaccatctaccacctgcgaaagaagctggcggacagcccc |
| | TYNQLFEESPLDEIEVDAKGIL | gagaaggccgacctgagactgatctacctggccctagcccacatcatcaagtttcgcggaca |
| | SARLSKSKRLEKLIAVFPNEK | tttcctgatcgagggcaaactgaacgccgagaacagcgacgtggccaaactgttctaccagc |
| | KNGLFGNIIALALGLTPNFKS | tgattcagacctacaaccagctgttcgaggagagccctctggacgaaatcgaagtggacgc |
| | NFDLTEDAKLQLSKDTYDDD | caaaggcatcctcagcgcacggctgagcaagagcaagcggctggaaaagctgatcgctgt |
| | LDELLGQIGDQYADLFSAAK | gttccctaacgagaagaaaaacggcctgttcggcaacataatcgccctggccctgggcctga |
| | NLSDAILLSDILRSNSEVTKAP | caccaaacttcaagagcaacttcgacctgacagaggatgccaagctgcaactgtccaaggat |
| | LSASMVKRYDEHHQDLALLK | acatacgacgacgatctggacgaactgctgggccagatcggcgaccagtacgccgatctgt |
| | TLVRQQFPEKYAEIFKDDTKN | ttagcgccgccaagaacctgagcgacgccatcctgctgagcgacattctgcgaagcaattcc |
| | GYAGYVGADKKLRKRSGKL | gaggtcaccaaggcccccctaagcgccagcatggtgaagagatacgacgagcaccacca |
| | ATEEEFYKFIKPILEKMDGAE | ggacctggcactgctcaagaccctggtcagacagcaattccctgagaaatacgctgaaatct |
| | ELLAKLNRDDLLRKQRTFDN | tcaaggacgacaccaagaacggatacgcaggctacgtcggagccgacaagaagctgagaa |
| | GSIPHQIHLKELHAILRRQEEF | agagatcaggaaagctggccaccgaggaggagttctacaagttcatcaaacccatcctgga |
| | YPFLKENREKIEKILTFRIPYY | gaaaatggacggcgccgaggagctgctggccaagctgaaccgggacgacctgctgcgga |
| | VGPLARGNSRFAWLTRKSEE | aacagagaaccttcgacaatggcagcatcccccaccagatccacctgaaggagctccacgc |
| | AITPWNFEEVVDKGASAQSFI | catcctgagaagacaggaggagttctacccattcctgaaggagaatagagaaaaaatcgag |
| | ERMTNFDEQLPNKKVLPKHS | aagatcctcaccttcagaatcccatactacgtgggacccctggccagaggcaacagccggtt |
| | LLYEYFTVYNELTKVKYVTE | tgcatggctgacaagaaagagcgaggaagcgatcaccccgtggaacttcgaggaggtggt |
| | RMRKPEFLSGEQKKAIVDLLF | ggacaagggggccagcgcccagagcttcatcgagcggatgaccaacttcgacgagcagct |
| | KTNRKVTVKQLKEDYFKKIE | gcctaacaagaaagtgctacctaagcacagcctactgtacgagtactttacagtgtacaacg |
| | CFDSVEIIGVEDRFNASLGTY | agctgacaaaggtcaagtacgtgaccgaaagaatgagaaagcctgaattcctgagcggcga |
| | HDLLKIIKDKDFLDNEENEDIL | gcagaagaaagccatcgtggacctcctgttcaagaccaacagaaaagtgacagtaaagcag |
| | EDIVLTLTLFEDREMIEERLKT | ctgaaggaggactacttcaagaagatcgaatgcttcgacagcgtggaaatcatcggcgtaga |
| | YAHLFDDKVMKQLKRRHYT | agaccgattcaacgcaagcctgggcacctaccacgacctgctgaaaataatcaaggataag |
| | GWGRLSRKMINGIRDKQSGK | gatttcctggataacgaggaaaacgaagacattctggaagacatagtgctgacactgactct |
| | TILDFLKSDGFSNRNFMQLIH | gttcgaagacagagagatgatcgaggaacggctgaagacctatgcccacctgtttgacgata |
| | DDSLTFKEEIEKAQVSGQGDS | aagtgatgaagcaactcaagaggcggcactacaccggctggggcagactgagcagaaaaat |
| | LHEQIADLAGSPAIKKGILQT | gatcaacggaatcagagacaagcagagcggcaagacgatcctggattttctaaaatccgac |
| | VKIVDELVKVMGHKPENIVIE | ggcttcagcaacaggaacttcatgcaactaatccacgacgattccctgacattcaaggaaga |
| | MARENQTTTKGLQQSRERKK | gatcgaaaaggcccaggtaagcggccagggcgacagcctccacgaacagatcgcggacc |
| | RIEEGIKELESQILKENPVENT | tggccggcagcccagccatcaaaaaaggcatcctgcagaccgtcaagatcgtggacgagc |
| | QLQNEKLYLYYLQNGRDMY | tggtcaaagtaatgggccacaagccagagaatatagtgatagagatggcaagagaaaacca |
| | VDQELDINRLSDYDVDAIVPQ | gaccacaacaaaaggcctgcaacagtccagagaacgaaagaagagaatcgaagagggca |
| | SFIKDDSIDNKVLTRSVENRG | tcaaagagctggaaagccagatactgaaggagaaccccgtggagaacacccagctgcaga |
| | KSDNVPSEEVVKKMKNYWR | acgagaagctgtacctgtactacctacagaacggacgggacatgtacgtggaccaggagct |
| | QLLNAKLITQRKFDNLTKAER | ggacattaaccggctaagcgactacgacgtggacgccatcgtgcctcagagctttatcaagg |
| | GGLSEADKAGFIKRQLVETRQ | atgactcgatcgacaacaaggtgctcaccagatcggtggagaacaggggcaagagcgaca |
| | ITKHVARILDSRMNTKRDKN | acgtgccttccgaggaggtggtgaagaagatgaagaactattggagacagctgctgaacgc |
| | DKPIREVKVITLKSKLVSDFR | caaactcatcacccagagaaagttcgacaacctgacaaaggccgagagaggcggcctgag |
| | KDFQLYKVRDINNYHHAHDA | cgaagccgacaaggccggcttcataaaaagacaactggtggagacaagacagatcacaaa |
| | YLNAVVGTALIKKYPKLESEF | gcacgtggccagaatcctagactcccggatgaacaccaagagagacaagaacgacaagc |
| | VYGDYKVYDVRKMIAKSEQE | ctatacgggaggtgaaggtgatcacactgaaaagcaagctcgtgagcgatttccggaagga |
| | IGKATAKRFFYSNIMNFFKTE | cttccagctgtacaaagtgcgggacatcaacaactaccaccacgcccacgacgcttacctga |
| | VKLANGEIRKRPLIETNGETG | atgctgtggtgggaaccgcactcatcaaaaagtacccaaagctggaatccgaattcgtgtac |
| | EVVWNKEKDFATVRKVLAM | ggcgactacaaggtgtacgacgtgcgcaagatgatcgccaagagcgaacaggaaatcggc |
| | PQVNIVKKTEVQTGGFSKESI | aaagccacagccaagagattcttctacagcaacatcatgaactttttcaagacagaagtgaa |
| | RPKRNSDKLIARKKDWDPKK | gctggccaacggcgagatcaggaagaggcccctcattgaaaccaacggggaaaccggcga |
| | YGGFLWPTVAYSVLVVAKVE | ggtggtgtggaacaaagagaaggacttcgccacagtgagaaaggtcctggccatgcccca |
| | KGKSKKLKSVKELLGITIMER | ggtgaacatcgtcaaaaagaccgaagtgcagaccgggggattcagcaaagagtccattaga |
| | SSFEKNPIDFLEAKGYKEVKK | cctaagagaaacagcgacaaactgatcgcacggaagaaggactgggatcctaaaaaatac |
| | DLIIKLPKYSLFELENGRKRM | ggaggattcctgtggccgaccgtggcctacagcgtgctggtggtagccaaggtggaaaag |
| | LASAKQLQKGNELALPSKYV | ggcaaaagcaagaagctgaagtccgtgaaggaactgctgggcatcacaatcatggaacgg |
| | NFLYLASHYEKLKGSPEDNE | agcagctttgaaaagaacccaatcgacttcctggaagccaagggctacaaggaggtgaaaa |
| | QKQLFVEQHKHYLDEIIEQISE | aggacttaatcatcaagctgcctaagtacagcctgttcgagctggagaacggccggaagag |
| | FSKRVILADANLDKVLSAYN | aatgctggccagtgccaagcagctgcaaaagggcaatgagctggccctgcccagcaagtat |
| | KHRDKPIREQAENIIHLFTLTR | gttaacttcctgtacctggccagccactacgagaaactcaaaggcagccctgaagacaacga |
| | LGAPRAFKYFDTTIDPKQYRS | acagaagcaactcttcgtggagcaacacaaacactacctggatgagataatcgagcaaatct |
| | TKEVLDATLIHQSITGLYETRI | ccgagttcagcaagcgggtcatcctagcggacgccaacctggacaaggtgctgagcgcct |
| | DLSQLGGDSGGSSGGSKRTA | acaacaagcaccgggacaaacctatccgcgagcaggcagagaacatcatccacctgttcac |
| | DGSEFESPKKKRKVSGGSSGG | cctgacccggctgggcgcacctcgggccttcaaatacttcgacaccacaatcgaccctaagc |
| | STLNIEDEYRLHETSKEPDVSL | agtacagaagcacaaaggaagtgctggacgccaccctgatccaccagagcattacgggac |
| | GSTWLSDFPQAWAETGGMG | tatacgaaacacgcatcgatctgtcccagctgggaggcgacTCCGGCGGAAGCT |
| | LAVRQAPLIIPLKATSTPVSIK | CTGGTGGCAGCAAGCGGACCGCCGACGGCTCTGAATTC |
| | QYPMSQEARLGIKPHIQRLLD | GAGAGCCCTAAGAAGAAAAGAAAGGTGAGCGGAGGCT |
| | QGILVPCQSPWNTPLLPVKKP | CTAGCGGCGGAAGCACCCTGAACATTGAAGACGAGTAT |
| | GTNDYRPVQDLREVNKRVED | AGACTGCATGAAACAAGCAAGGAACCCGACGTGTCCCT |
| | IHPTVPNPYNLLSGLPPSHQW | GGGCTCCACCTGGCTGTCCGACTTTCCCCAGGCCTGGGC |
| | YTVLDLKDAFFCLRLHPTSQP | CGAGACAGGAGGAATGGGCCTGGCCGTGCGGCAGGCAC |
| | LFAFEWRDPEMGISGQLTWT | CCCTGATCATCCCTCTGAAGGCCACCTCTACACCCGTGA |
| | RLPQGFKNSPTLFNEALHRDL | GCATCAAGCAGTACCCTATGTCTCAGGAGGCCAGACTG |
| | ADFRIQHPDLILLQYVDDLLL | GGCATCAAGCCTCACATCCAGAGGCTGCTGGACCAGGG |
| | AATSELDCQQGTRALLQTLG | CATCCTGGTGCCATGCCAGAGCCCCTGGAACACACCACT |
| | NLGYRASAKKAQICQKQVKY | GCTGCCCGTGAAGAAGCCAGGCACCAATGACTATAGAC |
| | LGYLLKEGQRWLTEARKETV | CCGTGCAGGATCTGAGAGAGGTGAACAAGAGGGTGGAG |
| | MGQPTPKTPRQLREFLGKAG | GATATCCACCCCACCGTGCCCAACCCTTACAATCTGCTG |
| | FCRLFIPGFAEMAAPLYPLTK | TCCGGCCTGCCCCCTTCTCACCAGTGGTATACAGTGCTG |
| | PGTLFNWGPDQQKAYQEIKQ | GACCTGAAGGATGCCTTCTTTTGTCTGAGACTGCACCCT |
| | ALLTAPALGLPDLTKPFELFV | ACCAGCCAGCCACTGTTCGCCTTTGAGTGGAGGGACCCT |
| | DEKQGYAKGVLTQKLGPWR | GAGATGGGCATCTCTGGCCAGCTGACCTGGACACGCCT |
| | RPVAYLSKKLDPVAAGWPPC | GCCTCAGGGCTTCAAGAATAGCCCAACACTGTTTAACG |
| | LRMVAAIAVLTKDAGKLTMG | AGGCCCTGCACCGCGACCTGGCAGATTTCCGGATCCAG |
| | QPLVILAPHAVEALVKQPPDR | CACCCAGATCTGATCCTGCTGCAGTACGTGGACGATCTG |
| | WLSNARMTHYQALLLDTDR | CTGCTGGCCGCCACCAGCGAGCTGGATTGCCAGCAGGG |
| | VQFGPVVALNPATLLPLPEEG | AACACGCGCCCTGCTGCAGACCCTGGGAAACCTGGGAT |
| | LQHNCLDILAEAHGTRPDLTD | ATAGGGCATCCGCCAAGAAGGCCCAGATCTGTCAGAAG |
| | QPLPDADHTWYTDGSSLLQE | CAGGTGAAGTACCTGGGCTATCTGCTGAAGGAGGGCCA |
| | GQRKAGAAVTTETEVIWAKA | GAGATGGCTGACAGAGGCCAGGAAGGAGACAGTGATG |
| | LPAGTSAQRAELIALTQALKM | GGCCAGCCAACACCCAAGACCCCAAGACAGCTGAGGGA |
| | AEGKKLNVYTDSRYAFATAH | GTTCCTGGGCAAAGCAGGATTTTGCAGGCTGTTCATCCC |
| | IHGEIYRRRGWLTSEGKEIKN | AGGATTCGCAGAGATGGCAGCACCTCTGTACCCACTGA |
| | KDEILALLKALFLPKRLSIIHC | CCAAGCCGGGCACCCTGTTTAATTGGGGCCCTGACCAGC |
| | PGHQKGHSAEARGNRMADQ | AGAAGGCCTATCAGGAGATCAAGCAGGCCCTGCTGACA |
| | AARKAAITETPDTSTLLIENSS | GCACCAGCCCTGGGCCTGCCAGACCTGACCAAGCCTTTC |
| | PSGGSKRTADGSEFESPKKKR | GAGCTGTTTGTGGATGAGAAGCAGGGCTACGCCAAGGG |
| | KVGSGPAAKRVKLD (SEQ ID | CGTGCTGACCCAGAAGCTGGGACCATGGAGACGGCCCG |
| | NO: 1329) | TGGCCTATCTGTCCAAGAAGCTGGACCCAGTGGCAGCA |
| | | GGATGGCCACCATGCCTGAGGATGGTGGCAGCAATCGC |
| | | CGTGCTGACAAAGGATGCCGGCAAGCTGACCATGGGAC |
| | | AGCCACTGGTCATCCTGGCACCACACGCAGTGGAGGCC |
| | | CTGGTGAAGCAGCCTCCAGATCGCTGGCTGTCTAACGCC |
| | | CGGATGACACACTACCAGGCCCTGCTGCTGGACACCGA |
| | | TCGCGTGCAGTTTGGCCCTGTGGTGGCCCTGAATCCAGC |
| | | CACCCTGCTGCCTCTGCCAGAGGAGGGCCTGCAGCACA |
| | | ACTGTCTGGACATCCTGGCAGAGGCACACGGAACAAGG |
| | | CCAGACCTGACCGATCAGCCCCTGCCTGACGCCGATCAC |
| | | ACATGGTATACCGATGGAAGCTCCCTGCTGCAGGAGGG |
| | | CCAGAGGAAGGCAGGAGCAGCAGTGACCACAGAGACA |
| | | GAAGTGATCTGGGCCAAGGCCCTGCCAGCAGGCACATC |
| | | CGCCCAGCGGGCCGAGCTGATCGCCCTGACCCAGGCCC |
| | | TGAAGATGGCCGAGGGCAAGAAGCTGAACGTGTACACA |
| | | GACTCCAGATATGCCTTCGCCACCGCACACATCCACGGA |
| | | GAGATCTACAGGCGCCGGGGCTGGCTGACCTCTGAGGG |
| | | CAAGGAGATCAAGAACAAGGATGAGATCCTGGCCCTGC |
| | | TGAAGGCCCTGTTTCTGCCCAAGCGGCTGAGCATCATCC |
| | | ACTGTCCTGGACACCAGAAGGGACACTCCGCCGAGGCA |
| | | AGGGGCAATCGGATGGCCGACCAGGCCGCCAGAAAGGC |
| | | TGCTATTACTGAAACTCCCGACACTTCCACTCTGCTGAT |
| | | TGAAAACTCCTCCCCTTCTGGCGGCTCAAAAAGAACCGC |
| | | CGACGGCAGCGAATTCGAGTCTCCCAAGAAGAAGAGGA |
| | | AAGTCGGCTCTGGCCCTGCCGCTAAGAGAGTGAAGCTG |
| | | GACtaatagTGAGCTGGAGCCTCGGTGGCCATGCTTCTTGC |
| | | CCCTTGGGCCTCCCCCCAGCCCCTCCTCCCCTTCCTGCA |
| | | CCCGTACCCCCGTGGTCTTTGAATAAAGTCTGAGTGGGC |
| | | GGCAATCAACCTCTGGATTACAAAATTTGTGAAAGATTG |
| | | ACTGATATTCTTAACTATGTTGCTCCTTTTACGCTGTGTG |
| | | GATATGCTGCTTTAATGCCTCTGTATCATGCTATTGCTTC |
| | | CCGTACGGCTTTCGTTTTCTCCTCCTTGTATAAATCCTGG |
| | | TTGCTGTCTCTTTATGAGGAGTTGTGGCCCGTTGTCCGTC |
| | | AACGTGGCGTGGTGTGCTCTGTGTTTGCTGACGCAACCC |
| | | CCACTGGCTGGGGCATTGCCACCACCTGTCAACTCCTTT |
| | | CTGGGACTTTCGCTTTCCCCCTCCCGATCGCCACGGCAG |
| | | AACTCATCGCCGCCTGCCTTGCCCGCTGCTGGACAGGGG |
| | | CTAGGTTGCTGGGCACTGATAATTCCGTGGTGTTGTCGG |
| | | GGAAGCTGACGTCCTTTCCAgGGCTGCTCGCCTGTGTTG |
| | | CCAACTGGATCCTGCGCGGGACGTCCTTCTGCTACGTCC |
| | | CTTCGGCTCTCAATCCAGCGGACCTCCCTTCCCGAGGCC |
| | | TTCTGCCGGTTCTGCGGCCTCTCCCGCGTCTTCGCTTTCG |
| | | GCCTCCGACGAGTCGGATCTCCCTTTGGGCCGCCTCCCC |
| | | GCCTGAAAAAAAAAAAAAAACAAAAAAAAAAAAAAAA |
| | | AAAAAAAAAAACCAAAAAAAAAAAAAAAAAAAAAAAA |
| | | AAAAAAAAAAACCCCAAAAAAAAAAAAAAAAAAAAAA |
| | | AAAAAAAAAAAAAAAAAAAAAAAA (SEQ ID NO: 1330) |

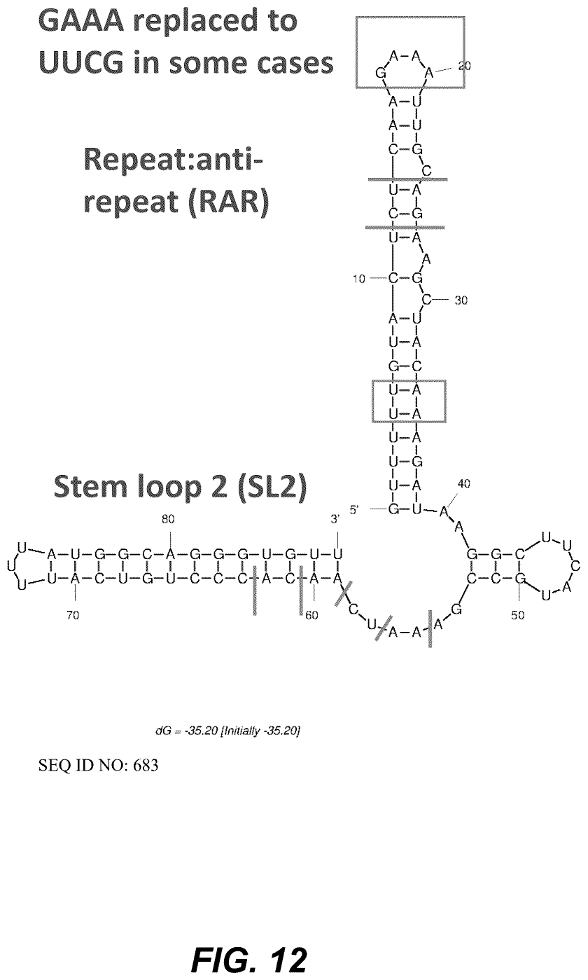

Next, the tagRNA scaffold (for binding of certain Cas9 proteins) was engineered to optimize the molecule for additional efficiency in editing. A representative scaffold structure is shown in FIG. 12, wherein certain portions of the scaffold are edited and/or replaced. The truncations tested are presented in the Table below. In this instance, SSACas9 was used. The truncated versions were tested in vitro and the data, as the average of triplicate runs, is presented below. It was observed that truncations of the scaffold increase precise editing of the RT editing system. Similar optimizations for other Cas9s, for instance, SpRYcCas9, are contemplated.

TABLE 23
SCAFFOLD VARIANTS

| | | | | % Precise |
|---|---|---|---|---|
| | SEQ | | | editing |
| Version | ID NO: | Description | Length | (T > C) |
| v0 | 755 | wild type | 123 | 0.27 |
| v1 | 756 | UA:AU to GC:CG | 123 | 23.02 |
| v2 | 757 | RAR trunc1 | 116 | 20.47 |
| v3 | 758 | RAR trunc2 + UUCG | 111 | 5.71 |
| v4 | 759 | UA:AU + RAR trunc1 | 116 | 0.05 |
| v5 | 760 | UA:AU + RAR trunc2 | 111 | 9.79 |
| v6 | 761 | SL2_trunc1 | 98 | 0.06 |
| v7 | 762 | SL2_trunc2 | 96 | 54.69 |
| v8 | 763 | SL2_trunc3 | 94 | 0.09 |
| v9 | 764 | SL2_trunc4 | 92 | 0.06 |
| v10 | 765 | SL2_trunc5 | 90 | 0.11 |
| v11 | 766 | UA:AU to GC:CG + SL2_trunc1 | 98 | 38.69 |
| v12 | 767 | RAR trunc1 + SL2_trunc1 | 91 | 0.19 |
| v13 | 768 | RAR trunc2 + UUCG + SL2_trunc1 | 86 | 12.80 |
| v14 | 769 | UA:AU + RAR trunc1 + SL2_trunc1 | 91 | 38.80 |
| v15 | 770 | UA:AU + RAR trunc2 + SL2_trunc1 | 86 | 3.46 |
| v16 | 771 | UA:AU to GC:CG + SL2_trunc2 | 96 | 69.12 |
| v17 | 772 | RAR trunc1 + SL2_trunc2 | 89 | 71.13 |
| v18 | 773 | RAR trunc2 + UUCG + SL2_trunc2 | 84 | 29.48 |
| v19 | 774 | UA:AU + RAR trunc1 + SL2_trunc2 | 89 | 60.29 |
| v20 | 775 | UA:AU + RAR trunc2 + SL2_trunc2 | 84 | 10.93 |
| v21 | 776 | UA:AU to GC:CG + SL2_trunc3 | 94 | 0.08 |
| v22 | 777 | RAR trunc1 + SL2_trunc3 | 87 | 0.06 |
| v23 | 778 | RAR trunc2 + UUCG + SL2_trunc3 | 82 | 4.02 |
| v24 | 779 | UA:AU + RAR trunc1 + SL2_trunc3 | 87 | 19.13 |
| v25 | 780 | UA:AU + RAR trunc2 + SL2_trunc3 | 82 | 1.88 |
| v26 | 781 | UA:AU to GC:CG + SL2_trunc4 | 92 | 1.00 |
| v27 | 782 | RAR trunc1 + SL2_trunc4 | 85 | 2.68 |
| v28 | 783 | RAR trunc2 + UUCG + SL2_trunc4 | 80 | 0.05 |
| v29 | 784 | UA:AU + RAR trunc1 + SL2_trunc4 | 85 | 0.09 |
| v30 | 785 | UA:AU + RAR trunc2 + SL2_trunc4 | 80 | 0.06 |
| v31 | 786 | UA:AU to GC:CG + SL2_trunc5 | 90 | 0.04 |
| v32 | 787 | RAR trunc1 + SL2_trunc5 | 83 | 0.11 |
| v33 | 788 | RAR trunc2 + UUCG + SL2_trunc5 | 78 | 0.03 |
| v34 | 789 | UA:AU + RAR trunc1 + SL2_trunc5 | 83 | 0.05 |
| v35 | 790 | UA:AU + RAR trunc2 + SL2_trunc5 | 78 | 0.04 |

### Example 4

### tagRNA Optimization

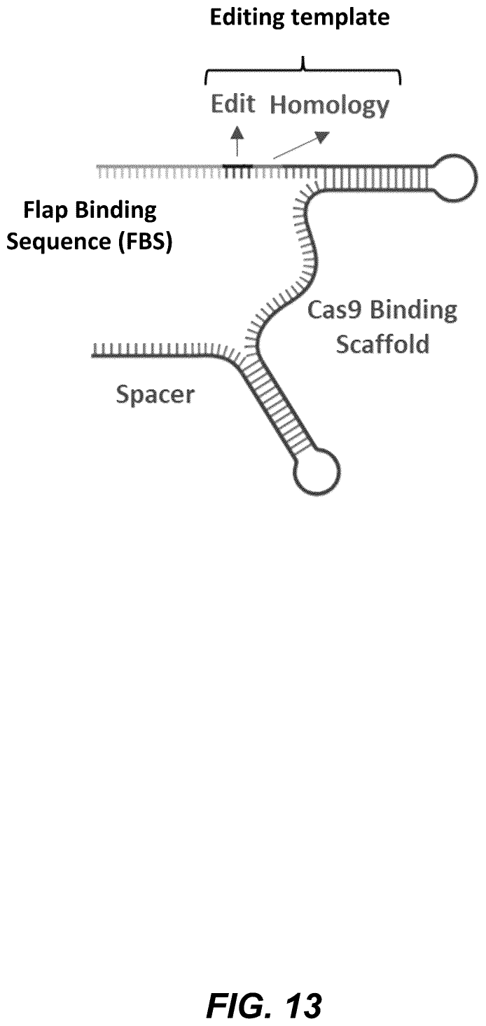

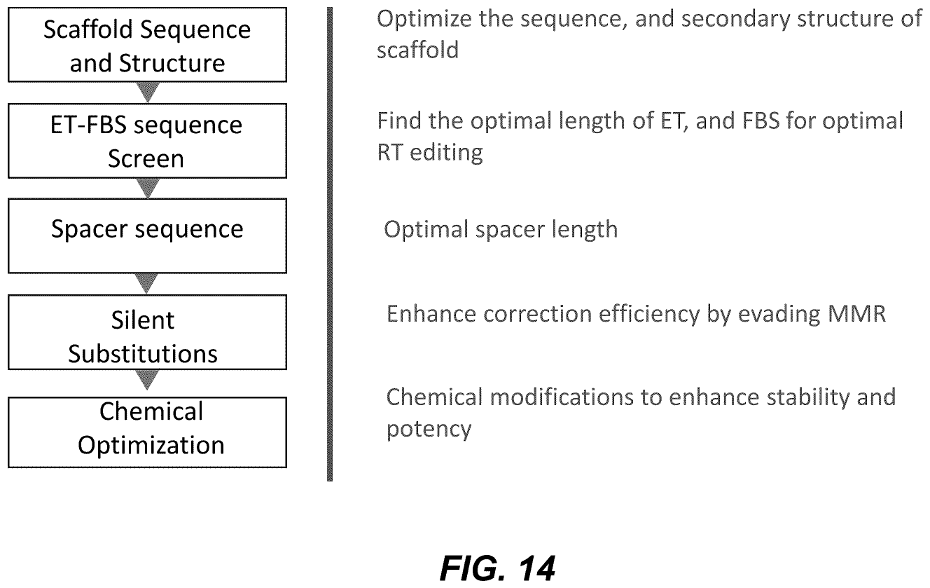

Provided in this example are optimizations of the tagRNA. Parameters for optimization included sequence (nucleotide) optimization (e.g., spacer length, FBS composition, homology length, and edits (silent mutations); (2) structure optimization (e.g., scaffold structure); and (3) chemical optimization (e.g., backbone chemical modifications to improve stability and/or potency). See FIGS. 13-15. Editing experiments were performed using methods as described above in Example 3 (except as otherwise noted) in Huh7 cells carrying the E342K mutation.

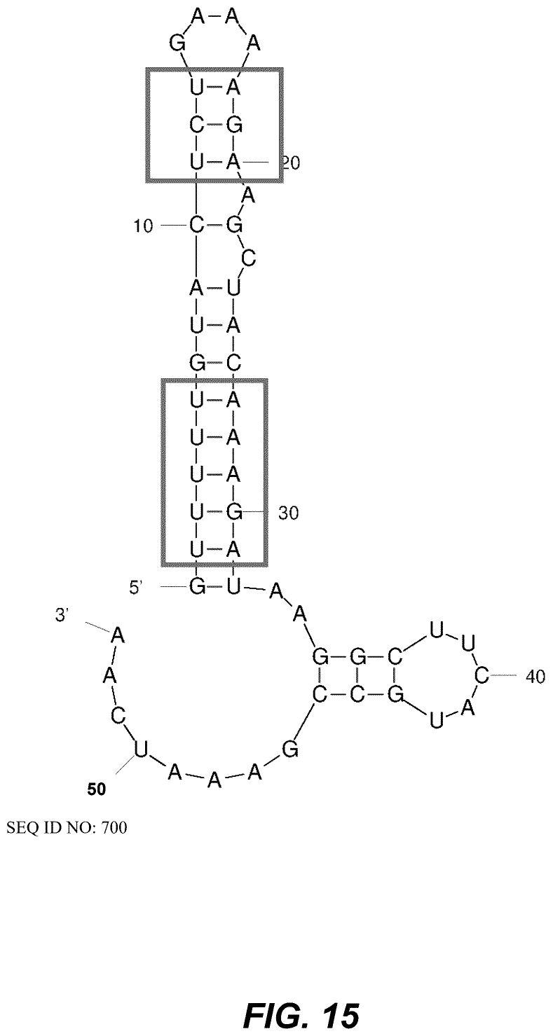

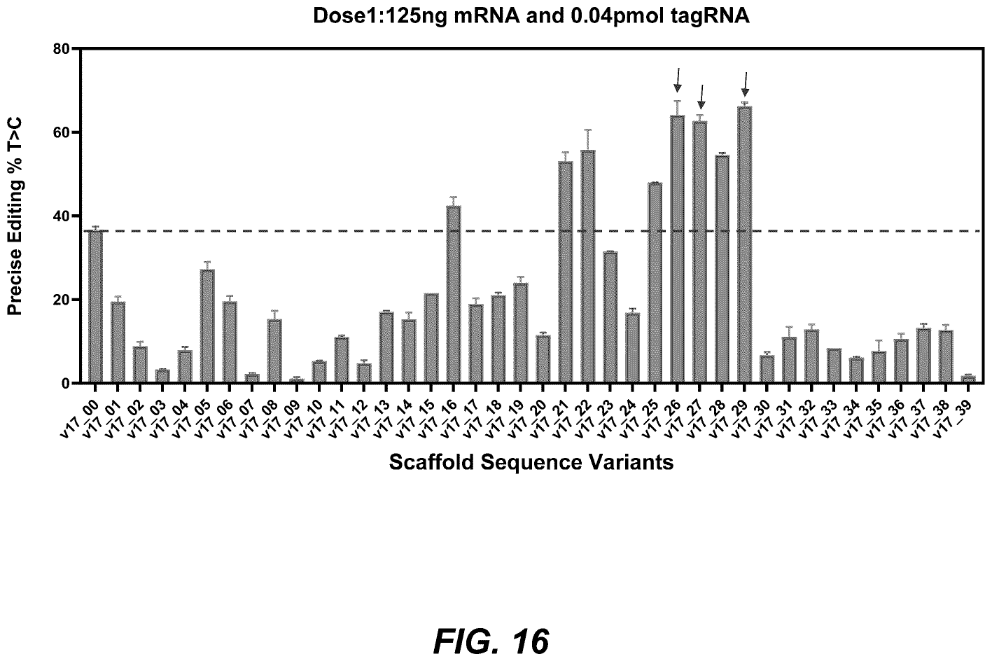

UA pairs in the tagRNA scaffold (See FIG. 15) were replaced with GC pairs within the hairpin structure to strengthen the repeat anti-repeat region (RAR). These tagRNA variants can, in some embodiments, result in increased precise editing of target sequence (e.g., E342K disease mutation in the SERPINA1) as shown in FIG. 16. In these experiments, 125 ng of mRNA encoding the RT editor and 0.04 pmol tagRNA was used. Among the positions tested included positions 13, 18, 21, 40, 50, and 52-53 relative to v17 tagRNA scaffold (SEQ ID NO: 700). In some cases, the U at position 13 is changed to a C, the A at position 18 is changed to a G, the A at position 21 is changed to a C or a U, the C at position 40 is changed to a U or an A or a G, the U at position 50 is changed to a C or an A or a G, the A at position 52 is changed to a C or a U or a G, the A at position 53 is changed to a C or a U or a G, and any combination of these changes. Table 24 provides exemplary sequences of tagRNA scaffold variants according to the present disclosure.

TABLE 24
SCAFFOLD VARIANTS

| | | SEQ ID |
|---|---|---|
| NAME | SEQUENCE¹ | NO: |
| v17_00 | GUUUUUGUACUCUGAAAAGAAGCUACAAAGAUAAGGCUUCAUGCCGAAAUCAA | 700 |
| v17_01 | GUUUUUGUACUCCGAAAGGAAGCUACAAAGAUAAGGCUUAAUGCCGAAAACAA | 964 |
| v17_02 | GUUUUUGUACUCCGAAAGGACGCUACAAAGAUAAGGCUUAAUGCCGAAAACAA | 965 |
| v17_03 | GUUUUUGUACUCCGAAAGGAUGCUACAAAGAUAAGGCUUAAUGCCGAAAACAA | 966 |
| v17_04 | GUUUUUGUACUCCGAAAGGAAGCUACAAAGAUAAGGCUUGAUGCCGAAAACAA | 967 |
| v17_05 | GUUUUUGUACUCCGAAAGGAAGCUACAAAGAUAAGGCUUUAUGCCGAAAACAA | 968 |
| v17_06 | GUUUUUGUACUCCGAAAGGAAGCUACAAAGAUAAGGCUUCAUGCCGAAAACAA | 969 |
| v17_07 | GUUUUUGUACUCCGAAAGGACGCUACAAAGAUAAGGCUUGAUGCCGAAAACAA | 970 |
| v17_08 | GUUUUUGUACUCCGAAAGGACGCUACAAAGAUAAGGCUUUAUGCCGAAAACAA | 971 |
| v17_09 | GUUUUUGUACUCCGAAAGGAUGCUACAAAGAUAAGGCUUGAUGCCGAAAACAA | 972 |
| v17_10 | GUUUUUGUACUCCGAAAGGAUGCUACAAAGAUAAGGCUUUAUGCCGAAAACAA | 973 |
| v17_11 | GUUUUUGUACUCCGAAAGGACGCUACAAAGAUAAGGCUUCAUGCCGAAAACAA | 974 |
| v17_12 | GUUUUUGUACUCCGAAAGGAUGCUACAAAGAUAAGGCUUCAUGCCGAAAACAA | 975 |
| v17_13 | GUUUUUGUACUCCGAAAGGAAGCUACAAAGAUAAGGCUUAAUGCCGAAAACAC | 976 |
| v17_14 | GUUUUUGUACUCCGAAAGGAAGCUACAAAGAUAAGGCUUAAUGCCGAAAACAG | 977 |
| v17_15 | GUUUUUGUACUCCGAAAGGAAGCUACAAAGAUAAGGCUUAAUGCCGAAAACAU | 978 |
| v17_16 | GUUUUUGUACUCCGAAAGGAAGCUACAAAGAUAAGGCUUAAUGCCGAAAACCA | 979 |
| v17_17 | GUUUUUGUACUCCGAAAGGAAGCUACAAAGAUAAGGCUUAAUGCCGAAAACGA | 980 |
| v17_18 | GUUUUUGUACUCCGAAAGGAAGCUACAAAGAUAAGGCUUAAUGCCGAAAACUA | 981 |
| v17_19 | GUUUUUGUACUCCGAAAGGAAGCUACAAAGAUAAGGCUUAAUGCCGAAACCAA | 982 |
| v17_20 | GUUUUUGUACUCCGAAAGGAAGCUACAAAGAUAAGGCUUAAUGCCGAAAGCAA | 983 |
| v17_21 | GUUUUUGUACUCCGAAAGGAAGCUACAAAGAUAAGGCUUAAUGCCGAAAUCAA | 984 |
| v17_22 | GUUUUUGUACUCCGAAAGGAAGCUACAAAGAUAAGGCUUCAUGCCGAAAUCAA | 985 |
| v17_23 | GUUUUUGUACUCCGAAAGGAAGCUACAAAGAUAAGGCUUCAUGCCGAAACCAA | 986 |
| v17_24 | GUUUUUGUACUCCGAAAGGAAGCUACAAAGAUAAGGCUUCAUGCCGAAAGCAA | 987 |
| v17_25 | GUUUUUGUACUCCGAAAGGAAGCUACAAAGAUAAGGCUUGAUGCCGAAAUCAA | 988 |
| V17_26 | GUUUUUGUACUCCGAAAGGAAGCUACAAAGAUAAGGCUUUAUGCCGAAAUCAA | 894 |
| V17_27 | GUUUUUGUACUCCGAAAGGAAGCUACAAAGAUAAGGCUUCAUGCCGAAAUCAC | 895 |
| v17_28 | GUUUUUGUACUCCGAAAGGAAGCUACAAAGAUAAGGCUUCAUGCCGAAAUCAG | 989 |
| V17_29 | GUUUUUGUACUCCGAAAGGAAGCUACAAAGAUAAGGCUUCAUGCCGAAAUCAU | 896 |
| v17_30 | GUUUUUGUACUCCGAAAGGACGCUACAAAGAUAAGGCUUAAUGCCGAAAACCC | 990 |
| v17_31 | GUUUUUGUACUCCGAAAGGACGCUACAAAGAUAAGGCUUAAUGCCGAAAACCG | 991 |
| v17_32 | GUUUUUGUACUCCGAAAGGACGCUACAAAGAUAAGGCUUAAUGCCGAAAACCU | 992 |
| v17_33 | GUUUUUGUACUCCGAAAGGACGCUACAAAGAUAAGGCUUAAUGCCGAAAACGC | 993 |
| v17_34 | GUUUUUGUACUCCGAAAGGACGCUACAAAGAUAAGGCUUAAUGCCGAAAACGG | 994 |
| v17_35 | GUUUUUGUACUCCGAAAGGACGCUACAAAGAUAAGGCUUAAUGCCGAAAACGU | 995 |
| v17_36 | GUUUUUGUACUCCGAAAGGACGCUACAAAGAUAAGGCUUAAUGCCGAAAACUC | 996 |
| v17_37 | GUUUUUGUACUCCGAAAGGACGCUACAAAGAUAAGGCUUAAUGCCGAAAACUG | 997 |
| v17_38 | GUUUUUGUACUCCGAAAGGACGCUACAAAGAUAAGGCUUAAUGCCGAAAACUU | 998 |
| v17_39 | GUUUUUGUACUCCGAAAGGAUGCUACAAAGAUAAGGCUUAAUGCCGAAAACCC | 999 |

¹For RNA, T is U; U are T in the sequence listing

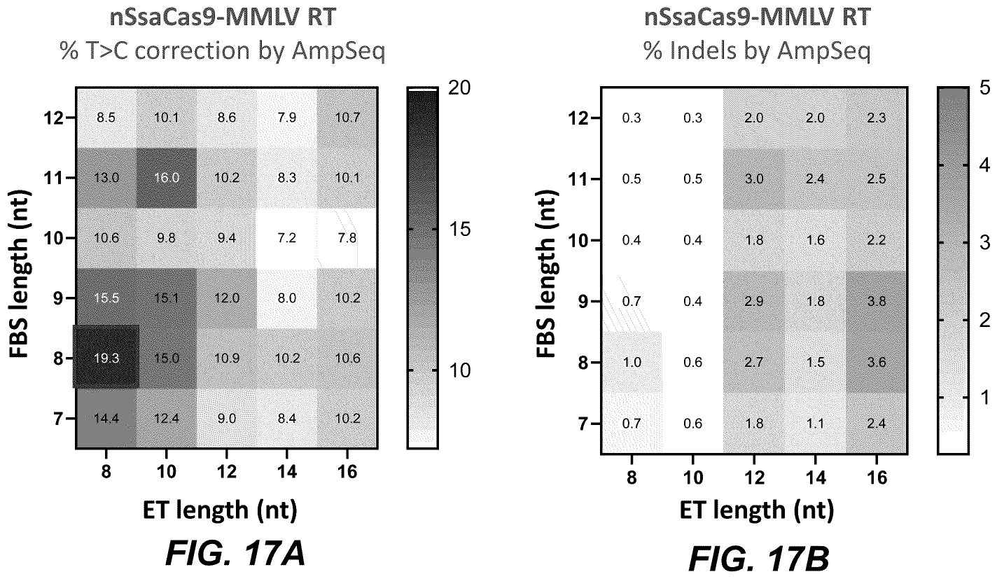

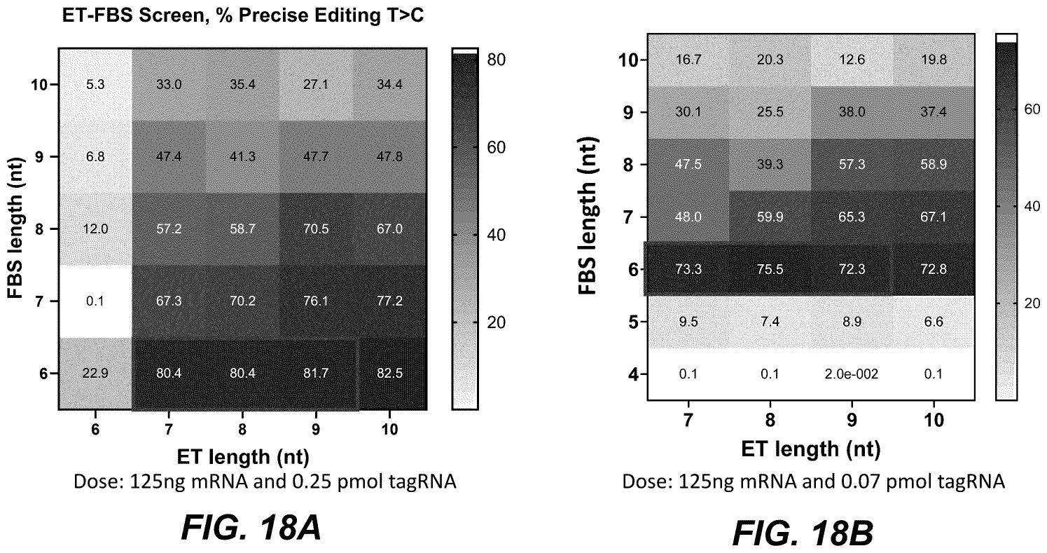

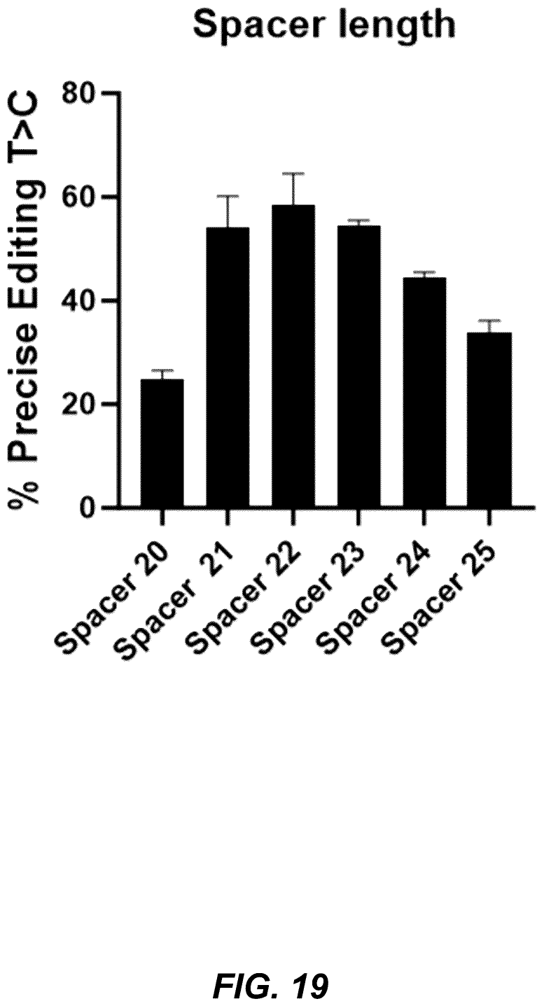

The lengths of the editing template, FBS, and spacer were also optimized. As shown in FIGS. 17-18, the editing template and FBS lengths were optimized via screening. It was found that an optimal FBS length is 6 nucleotides, while an optimal editing template length is 6-9 nucleotides. In the experiments shown in FIGS. 17-18, 0.07 or 0.25 pmol (for FIGS. 18A-18B, respectively) or 1 pmol (for FIGS. 17A-17B) of tagRNA and 125 ng mRNA encoding an RT editor (e.g., SsaCas9) were introduced into target cells. As shown in FIG. 19, the length of the spacer was also optimized via screening. Spacer lengths of 18 to 25 nucleotides were tested. It was determined that a spacer length of 21-23 nucleotides is optimal in some embodiments. In these experiments, 0.07 pmol of tagRNA and 125 ng of mRNA encoding an RT editor was used.

In some embodiments, and without being bound by any particular theory, adding silent substitutions at or near the target site can also improve editing. RT editing can be highly dependent on mismatch repair (MMR), and strategies to evade/redirect MMR can include introducing a second nick (egRNA) and/or silent mutation(s). In some embodiments, silent mutations exploit the various biases of the MMR machinery (See Chen et al. Cell, 2021), and some mutations can be preferred over others. For example, bigger bulges (e.g., multiple mutations in a row) can perform better. Accordingly, in some embodiments, the editing template comprises one or more silent nucleotide edits (not altering the amino acid sequence of the protein encoded by the double stranded target DNA) compared to the region corresponding to the editing target in the double stranded target DNA. The one or more silent nucleotide edits can comprise a substitution of 2 to 5 contiguous nucleotides.

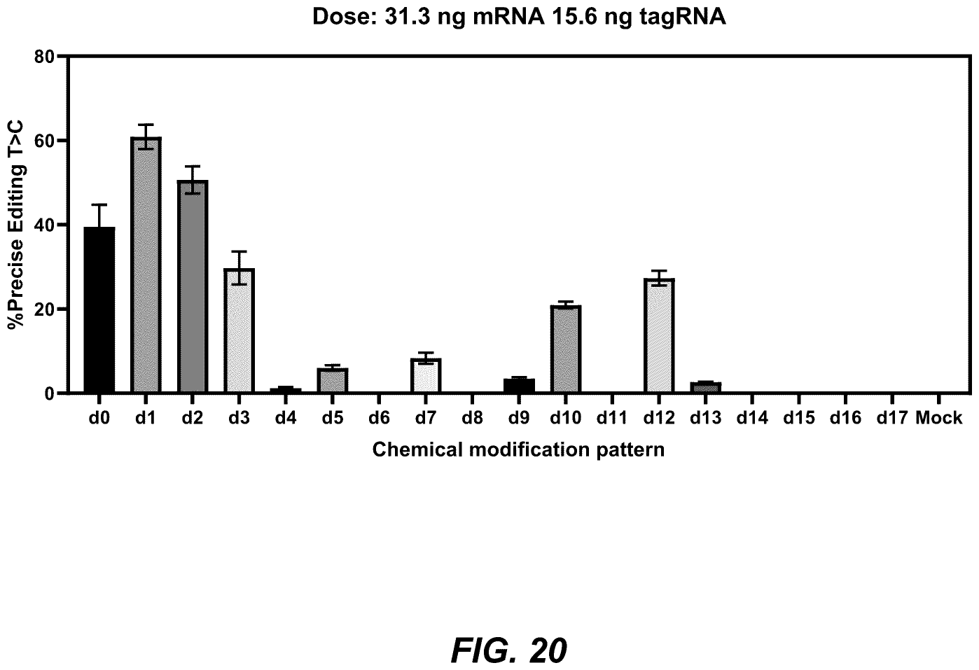

As provided in this example and described herein, chemical modifications to the tagRNA scaffold can, in some embodiments, enhance in vivo stability of the guide to prevent degradation before editing is able to occur. Chemical modifications adding nuclease stability without compromising activity were explored. Different scaffold modification patterns were tested and the results are shown in FIG. 20. In these experiments, 31.3 ng of mRNA encoding an RT editor (e.g., SsaCas9) and 15.6 ng tagRNA was used. The flap binding sequence (FBS) was 8 nt in length and the editing template was 8 nt in length. As shown in FIG. 20, the D1 scaffold modification pattern resulted in significant improvement in editing. The chemical modification can include 2′-O-methyl modification. The chemical modification pattern can comprise modifications at positions 5-9, 12-21, 29, 37-38, 40, 50, 52-53 of the tagRNA scaffold (e.g., relative to v17), and any combination of these modifications. Exemplary chemical modifications of the tagRNA scaffold are shown in Table 25 below (based on V17 variant scaffold described above).

TABLE 25
SCAFFOLD CHEMICAL MODIFICATIONS

| NAME | SEQUENCE¹ | SEQ ID NO: |
|---|---|---|
| D0 | GUUUUUGUACUCUGAAAAGAAGCUACAAAGAUAAGGCUUCAUGCCGAAA | 700 |
| (v17) | UCAA | |
| D1² | rGrUrUrUmUmUmGmUmArCrUmCmUmGmAmAmAmAmGmAmArGrCrUrArCrAr | 897 |
| | AmArGrArUrArArGrGmCmUrUmCrArUrGrCrCrGrArArAmU | |
| | rCmAmA | |
| D2² | rGrUrUrUmUmUmGmUmArCrUmCmUmGmAmAmAmAmGmAmArGrCrUrArCrAr | 898 |
| | ArArGrArUrArArGrGrCrUrUrCrArUrGrCrCrGrArArArUrCmAmA | |
| D3² | rGrUrUrUrUrUrGrUrArCrUmCmUmGmAmAmAmAmGrArArGrCrUrArCrArArArGr | 1000 |
| | ArUrArArGrGrCrUrUrCrArUrGrCrCrGrArArArUrCmAmA | |
| D4² | mGrUmUmUmUmUmGmUmArCrUmCmUmGmAmAmAmAmGmAmAmGmCmUm | 1001 |
| | AmCmArAmAmGmAmUrArArGmGmCmUrUmCrArUrGmCmCrGrArAmAmUrCm | |
| | AmA | |
| D5² | mGrUmUrUmUmUmGmUmArCrUmCmUmGmAmAmAmAmGmAmArGmCrUmAr | 1002 |
| | CmArAmArGmArUrArArGrGmCmUrUmCrArUrGrCrCrGrArArAmUrCmAmA | |
| D6² | mGrUmUrUmUmUmGmUmArCrUmCmUmGmAmAmAmAmGmAmAmGrCmUrA | 1003 |
| | mCrArArAmGrAmUrArArGrGmCmUrUmCrArUrGrCrCrGrArArAmUrCmAmA | |
| D7² | mGrUmUrUmUrUmGrUmArCrUmCmUmGmAmAmAmAmGmAmArGmCrUmArC | 1004 |
| | mArAmArGmArUrArArGrGmCmUrUmCrArUrGrCrCrGrArArAmUrCmAmA | |
| D8² | mGrUmUrUmUrUmGrUmArCrUmCmUmGmAmAmAmAmGmAmAmGrCmUrAmC | 1005 |
| | rArArAmGrAmUrArArGrGmCmUrUmCrArUrGrCrCrGrArArAmUrCmAmA | |
| D9² | rGrUrUrUmUrUmGrUmArCrUmCmUmGmAmAmAmAmGmAmArGmCrUmArCm | 1006 |
| | ArAmArGmArUrArArGrGmCmUrUmCrArUrGrCrCrGrArArAmUrCmAmA | |
| D10² | rGrUrUrUmUrUmGrUmArCrUmCmUmGmAmAmAmAmGmAmAmGrCmUrAmCr | 1007 |
| | ArArAmGrAmUrArArGrGmCmUrUmCrArUrGrCrCrGrArArAmUrCmAmA | |
| D11² | rGrUrUrUmUrUmGrUmArCrUmCmUmGmAmAmAmAmGrArArGrCrUrArCrArArA | 1008 |
| | rGrArUrArArGrGrCrUrUrCrArUrGrCrCrGrArArArUrCmAmA | |
| D12² | fGrUfUfUmUmUmGmUmArCrUmCmUmGmAmAmAmAmGmAmAfGfCrUfAfCfAr | 1009 |
| | AmAfGfAfUrArArGfGmCmUrUmCrAfUrGfCfCrGrArAfAmUrCmAmA | |
| D13² | fGrUfUfUmUmUmGmUmArCrUmCmUmGmAmAmAmAmGmAmAfGfCrUfAfCfAr | 1010 |
| | AfAfGfAfUrArArGfGfCfUrUfCrAfUrGfCfCrGrArAfAfUrCmAmA | |
| D14² | fGrUfUfUmUfUmGfUmArCrUmCmUmGmAmAmAmAmGfAmAfGmCrUfAmCfAr | 1011 |
| | AmAfGmAfUrArArGfGmCfUrUmCrAfUrGmCfCrGrArAfAmUrCmAmA | |
| D15² | rGfUrUrUmUmUmGmUmAfCfUmCmUmGmAmAmAmAmGmAmArGrCfUrArCrAf | 1012 |
| | AmArGrArUrArAfGrGmCmUrUmCfArUfGrCrCfGrArArAmUfCmAmA | |
| D16² | rGfUrUrUmUmUmGmUmAfCfUmCmUmGmAmAmAmAmGmAmArGrCfUrArCrAf | 1013 |
| | ArArGrArUrArAfGrGrCrUrUrCfArUfGrCrCfGrArArArUfCmAmA | |
| D17² | rGfUrUrUrUrUrGrUrAfCfUmCmUmGmAmAmAmAmGrArArGrCfUrArCrAfArArGr | 1014 |
| | ArUrArAfGrGrCrUrUrCfArUfGrCrCfGrArArArUfCmAmA | |

¹For RNA, T is U; U are T in the sequence listing
²For the sequences: *, phosphorothioate modification; mN, 2′O-methyl modification; rN, unmodified ribonucleotide; fN indicates internal (2′) Fluoro modification. N = A, C, T/U,G.

### Example 5

### Reverse Transcriptase Identification and Screening

Described in this Example is disclosure related to identification and optimization of improved reverse transcriptases derived from, e.g., birds and bats.

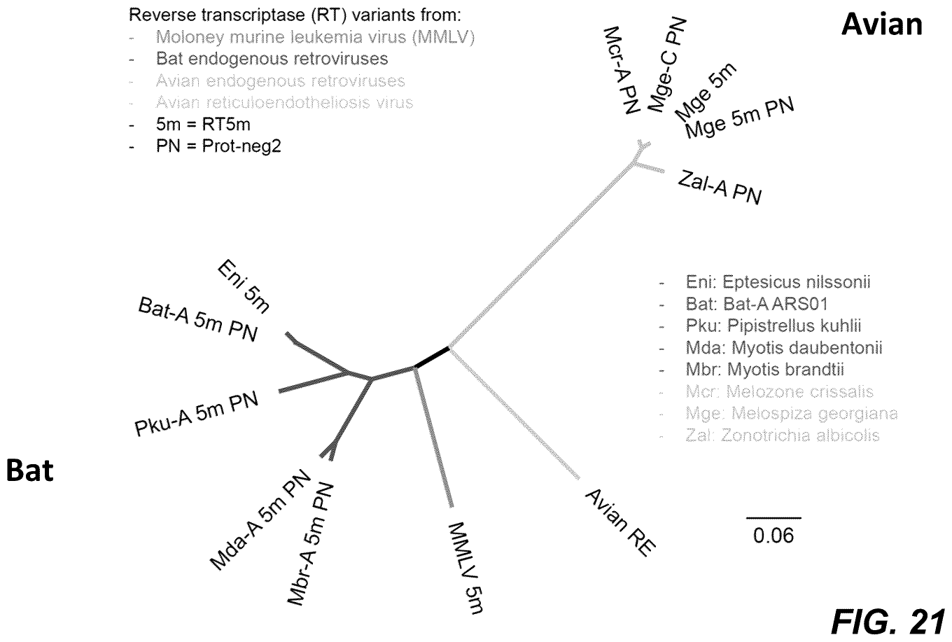

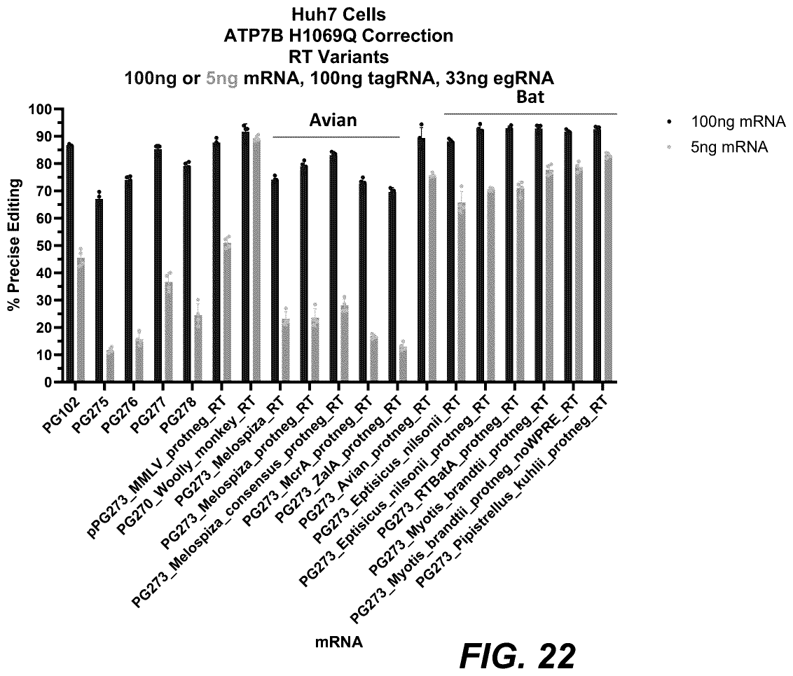

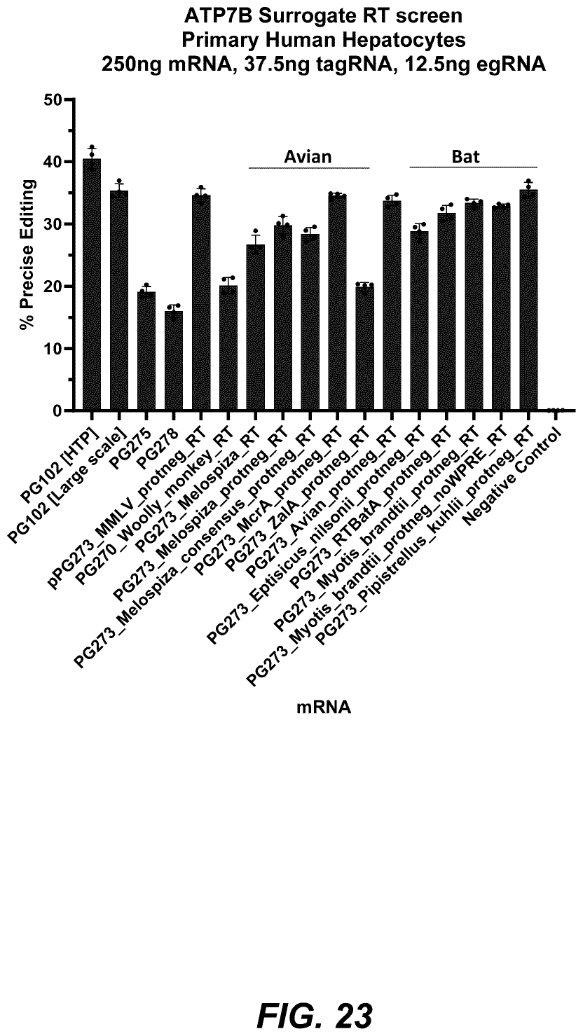

Shown in FIG. 21 is an exemplary phylogenetic tree of various RTs of the disclosure. FIG. 22-FIG. 23 display editing efficiencies in Huh7 cells or primary human hepatocytes for the indicated RT. For experiments shown in FIG. 22, the RT editor was provided at an amount of 100 or 5 ng mRNA as indicated. The tagRNA was provided in an amount of 100 ng, and the egRNA in an amount of 33 ng. Huh7 cells were transfected with Lipofectomine Messenger Max reagent and editing was assessed after 72 hrs. For experiments shown in FIG. 23, the RT editor was provided at an amount of 250 ng mRNA as indicated. The tagRNA was provided in an amount of 37.5 ng, and the egRNA in an amount of 12.5 ng. Primary human hepatocyte cells were transfected with Lipofectomine Messenger Max reagent and editing was assessed after 72 hrs. The SEQ ID NOs for reverse transcriptases that were tested are shown in Table 26 below. Sequences of the SEQ ID NOs referenced below can be found in Table 11 above. PG102, PG275, PG276, PG277, and PG278 in FIG. 21 are mRNAs encoding MMLV RT as a control.

TABLE 26
EXEMPLARY REVERSE TRANSCRIPTASES

| | | *SEQ |
|---|---|---|
| **Name | Origin | ID NO: |
| pPG273_MMLV_protneg_RT | | 1015 |
| PG270_Woolly_monkey_RT | | 1016 |
| PG273_Melospiza_RT | Bird | 141 |
| PG273_Melospiza_consensus_protneg_RT | Bird | 1017 |
| PG273_Melospiza_protneg_RT | Bird | 260 |
| PG273_McrA_protneg_RT | Bird | 257 |
| PG273_ZalA_protneg_RT | Bird | 319 |
| PG273_Avian_protneg_RT | Bird | 1018 |
| PG273_Eptisicus_nilsonii_RT | Bat | 252 |
| PG273_RTBatA_protneg_RT | Bat | 251 |
| PG273_Myotis_brandtii_protneg_RT | Bat | 255 |
| PG273_Myotis_brandtii_protneg_noWPRE_RT | Bat | 255 |
| PG273_Pipistrellus_kuhlii_protneg_RT | Bat | 267 |
| PG273_Eptisicus_nilsonii_protneg_RT | Bat | 315 |

*SEQ ID NO refers to the amino acid sequence of the RT
**PolyA nomenclature: pPG270/275, SegPolyA_1; pPG271/276, SegPolyA_2; pPG272/277, SegPoly A_3; pPG273/278, SegPolyA_4

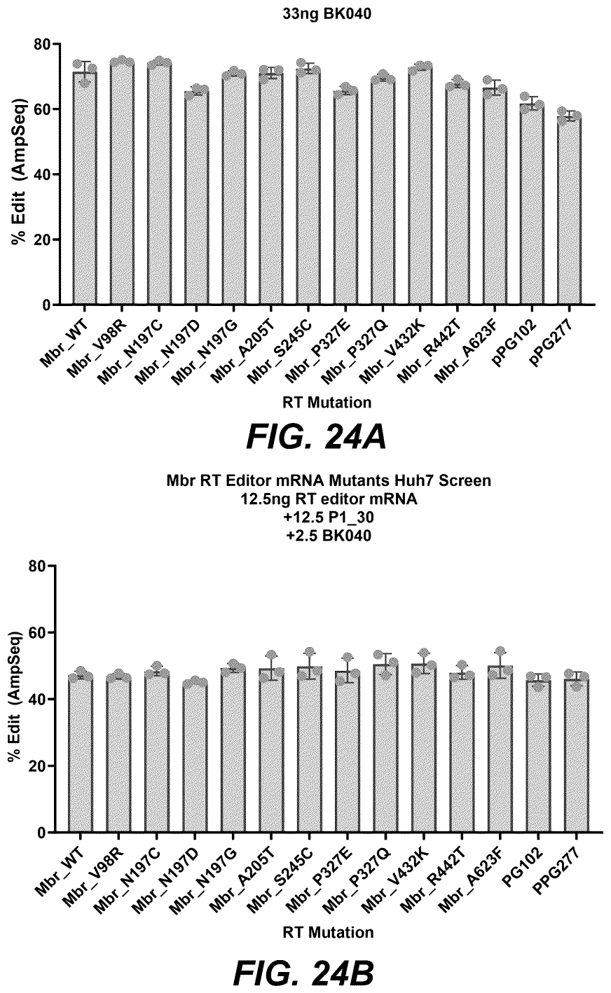

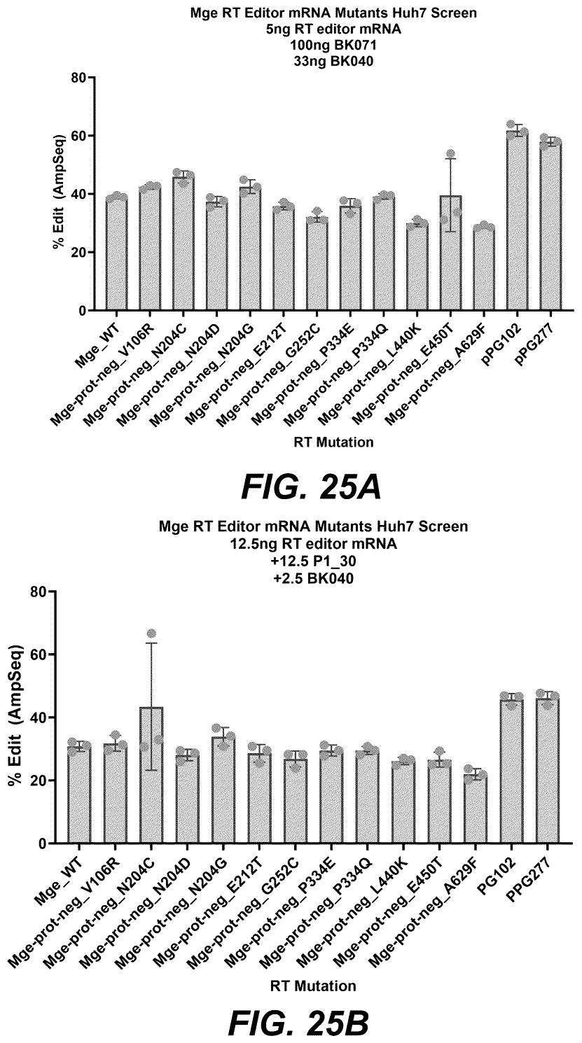

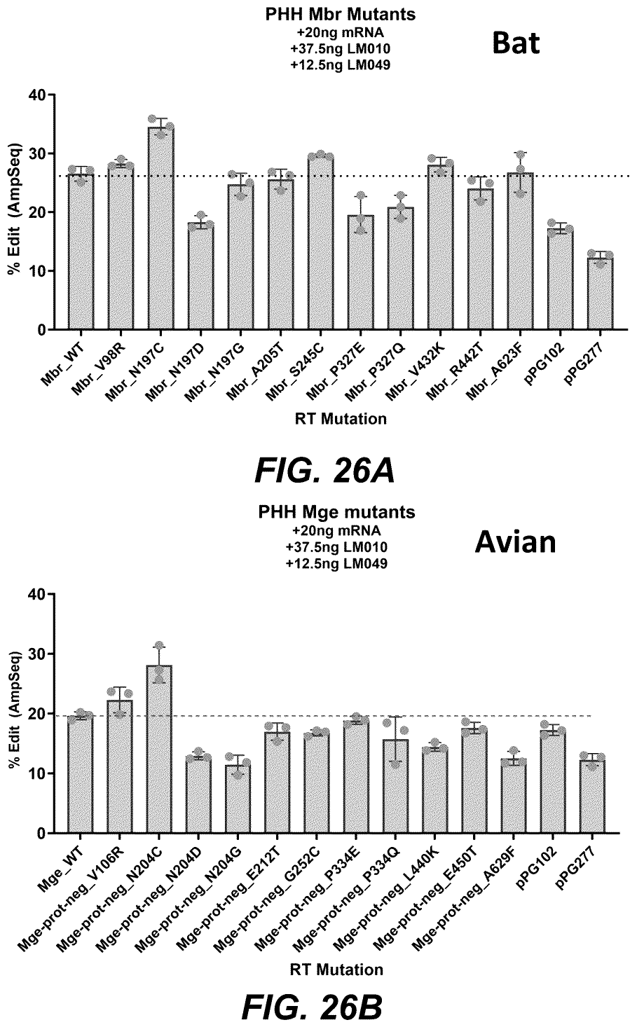

A mutant screen was performed to optimize the reverse transcriptases. Twelve previously described MMLV-RT mutations were introduced in Melospiza and Myotis Brandtii RTs. Each RT was aligned to MMLV-RT and the analogous mutation was made. Sequences were cloned into IVT backbone (pPG277 segmented PolyA 3). Nucleic acids were midiprepped and mRNA was made from the midiprepped plasmids using IVT. Variants were tested in PHH cells (reduced mRNA dose, FIG. 26A-FIG. 26B) and Huh7 cells (reduced mRNA dose and reduced all RNA dose, FIG. 24A-FIG. 25B). In FIG. 24A, low mRNA dose, full dose tagRNA and egRNA, and a standard tagRNA was used. In FIG. 24B3, lower dose of all RNA components and tagRNA with improved chemical modifications was used. In FIG. 25A, low mRNA dose, full dose tagRNA and egRNA, and a standard tagRNA was used. In FIG. 25B3, lower dose of all RNA components and tagRNA with improved chemical modifications was used. PG102 and PG277 of FIG. 24A-FIG. 26B are mRNAs encoding MMLV RT as a control.

Tables 27A and 27B provide a full list of the mutants designed. For Table 27A, the M. brandtii (M. br.) sequence is SEQ TD NO: 255. For Table 27B3, the Melospiza sequence (Mge-prot-neg) is SEQ ID NO: 260.

TABLE 27A
MUTANT LIST (M. BRANDTII)

| | | | Current | | | | |
|---|---|---|---|---|---|---|---|
| Mut. in | Mut. in | AA in | AA in | Position in | Position in | | |
| MMLV | Species | MML VRT | Species | MML VRT | Species | New AA | Species |
| NA | NA | NA | NA | NA | NA | NA | Mbr WT |
| V101R | V98R | V | V | 101 | 98 | R | Mbr |
| N200C | N197C | N | N | 200 | 197 | C | Mbr |
| N200D | N197D | N | N | 200 | 197 | D | Mbr |
| N200G | N197G | N | N | 200 | 197 | G | Mbr |
| A208T | A205T | A | A | 208 | 205 | T | Mbr |
| G248C | S245C | G | S | 248 | 245 | C | Mbr |
| P330E | P327E | T | P | 330 | 327 | E | Mbr |
| P330Q | P327Q | T | P | 330 | 327 | Q | Mbr |
| L435K | V432K | L | V | 435 | 432 | K | Mbr |
| K445T | R442T | K | R | 445 | 442 | T | Mbr |
| A623F | A623F | A | A | 623 | 623 | F | Mbr |

TABLE 27B
MUTANT LIST (M. GEORGIANA)

| | | | Current | | | | |
|---|---|---|---|---|---|---|---|
| Mut. in | Mut. in | AA in | AA in | Position in | Position in | New | |
| MMLV | Species | MML VRT | Species | MML VRT | Species | AA | Species |
| NA | NA | NA | NA | NA | NA | NA | Mge WT |
| V101R | V106R | V | V | 101 | 106 | R | Mge-prot-neg |
| N200C | N204C | N | N | 200 | 204 | C | Mge-prot-neg |
| N200D | N204D | N | N | 200 | 204 | D | Mge-prot-neg |
| N200G | N204G | N | N | 200 | 204 | G | Mge-prot-neg |
| A208T | E212T | A | E | 208 | 212 | T | Mge-prot-neg |
| G248C | G252C | G | G | 248 | 252 | C | Mge-prot-neg |
| P330E | P334E | T | P | 330 | 334 | E | Mge-prot-neg |
| P330Q | P334Q | T | P | 330 | 334 | Q | Mge-prot-neg |
| L435K | L440K | L | L | 435 | 440 | K | Mge-prot-neg |
| K445T | E450T | K | E | 445 | 450 | T | Mge-prot-neg |
| A623F | A629F | A | A | 623 | 629 | F | Mge-prot-neg |

TABLE 27C
Exemplary gRNAs

| NAME | SEQUENCE^{1,2} | SEQ ID NO: |
|---|---|---|
| LM10 | mU*mU*mG*rGrUrGrArCrUrGrCrCrArCrGrCrCrCrArArGrUrUrUrUrArGrAmGmC | 1267 |
| | mUmAmGmAmAmAmUmAmGmCrArArGrUrUrArArArArUrArArGrGrCrUrArGrUr | |
| | CrCrGrUrUrArUrCmAmAmCmUmUmGmAmAmAmAmAmGmUmGmGmCmAmC | |
| | mCmGmAmGmUmCmGmGmUmGmCrArGrUrGrArArCrcrCrCrCrCrUrUrGrGrGrCr | |
| | GrUrGrGrCrArGmU*mU*mU*mU | |
| LM49 | mG*mG*mC*rCrArGrCrArGrUrGrArArCrCrCrCrCrCrUrGrUrUrUrUrArGrAmGmC | 1268 |
| | mUmAmGmAmAmAmUmAmGmCrArArGrUrUrArArArArUrArArGrGrCrUrArGrUr | |
| | CrCrGrUrUrArUrCmAmAmCmUmUmGmAmAmAmAmAmGmUmGmGmCmAmC | |
| | mCmGmAmGmUmCmGmGmUmGmCmU*mU*mU*mU | |

¹For RNA, T is U; U are T in the sequence listing
²For the sequences: *, phosphorothioate modification; mN, 2′O-methyl modification; rN, unmodified ribonucleotide. N = A, C, T/U, G.

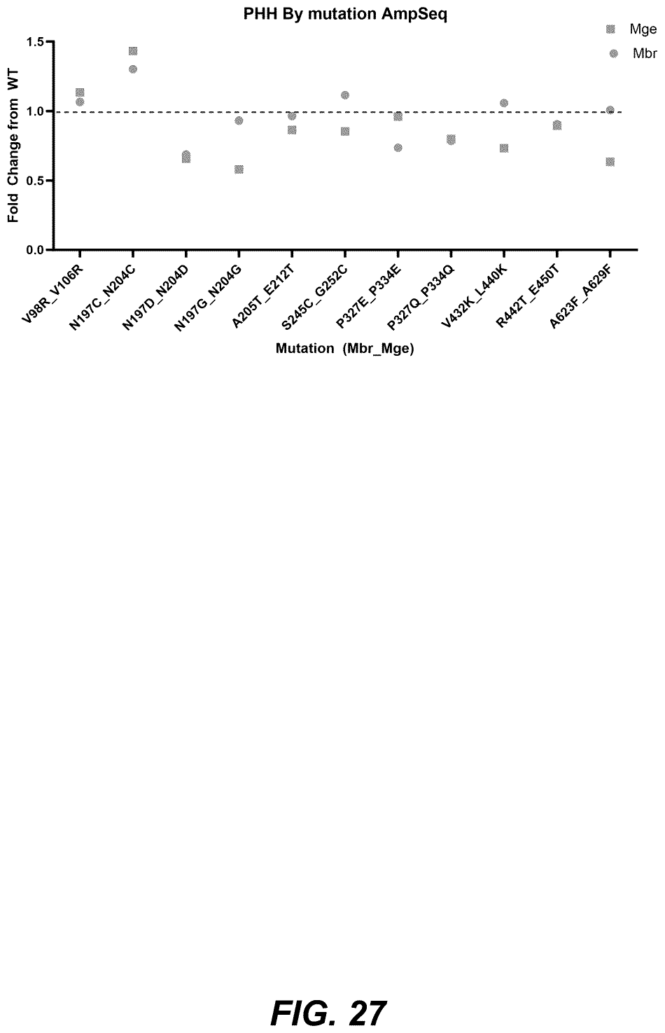

FIG. 27 displays mutant fold change from wild-type RT in primary human hepatocytes. N197C (Mbr)/N204C (Mge) was the strongest mutant in both species (N200C in MMLV RT). V98R (Mbr)/V16R (Mge) was second best (V101R in MMVLV RT).

### Example 6

### UTR mRNA Variant Screens

Described in this example are screens of 5′UTR sequences in RT mRNAs to enhance RT editing. Shown in Table 28 below are the components of the vectors used for testing 5′ UTRs.

TABLE 28
UTR VECTOR COMPONENTS

| | pPG102 | pPG278 | Myotis_RT_ctrl | UTR variants |
|---|---|---|---|---|
| 5′UTR | Control (SEQ ID | Control (SEQ ID | Control (SEQ ID | Variable (See, |
| | NO: 193) | NO: 193) | NO: 193) | Table 29) |
| RT | MMLV | MMLV | Myotis | Myotis |
| WPRE | No | Yes (SEQ ID NO: | No | No |
| | | 189) | | |
| PolyA | 120A (SEQ ID | SegpolyA_4 (SEQ | Segpoly A_4 (SEQ | Segpoly A_4 (SEQ |
| | NO: 186) | ID NO: 185) | ID NO: 185) | ID NO: 185) |

Provided in Table 29 are the sequences of the 5′UTR variants tested.

TABLE 29
EXEMPLARY 5′ UTR VARIANTS

| | | | SEQ ID |
|---|---|---|---|
| NAME | SEQUENCE¹ | Length | NO: |
| Control UTR | AGGAAAUAAGAGAGAAAAGAAGAGUAAGAAGAAAUAUA | 47 | 193 |
| | AGAGCCACC | | |
| NCA7d | AGCAAAAAUCAAAAUCAAUCAUCAUCACAACAUCAACA | 70 | 910 |
| | AUCAAUCAUCAACACAUCAUCAAGACACCACC | | |
| NCA7d-50nt | AGCAUCACAACAUCAACAAUCAAUCAUCAACACAUCAUC | 50 | 911 |
| | AAGACACCACC | | |
| HBB | AGAUUUGCUUCUGACACAACUGUGUUCACUAGCAACCU | 50 | 912 |
| | CAAACAGACACC | | |
| RV-UML-m002 | AGGUCCGUUAUAUUAUUUAUCUUGCAGAUCAAACUUCA | 50 | 913 |
| | GAGAGGAGGGCC | | |
| RV-UML-m314 | AGGGAACAGUGGAGCACAAUAGUUGUCACAAACGCGUG | 50 | 914 |
| | ACCUGUACCUUA | | |
| RV-UML-m017 | AGAGAAAGAGUUUCGAGAAAGUUGCGAUACACACCGAC | 50 | 915 |
| | CUAAUCCGUGUC | | |
| RV-UML-m349 | AGACUGAGUAGCGUUGUCGAUUGCUGUUUUCAUAACCG | 50 | 916 |
| | UACCGAGCCUGC | | |
| RV-UML-m286 | AGUGGAGAGCGCAAUCCAGUUUCCUUAGCAAAGCAUAU | 50 | 917 |
| | CACCCUUCGGCU | | |
| RV-UML-m302 | AGUUAUUUGCGAUAAACAGUAGGGAGACAAUUGACCUG | 50 | 918 |
| | CACAGCGUGGCC | | |
| RV-UML-m318 | AGCCCUUACACGAAUUCUCGAAAAUCGUGCUUCAAGAA | 50 | 919 |
| | ACGUACAACGGC | | |
| RV-UML-m348 | AGCCUUCGCACAAUACUCGCGCGUCCUAAGCAAAAUCGG | 50 | 920 |
| | AACAAGUACUG | | |
| RV-UML-m025 | AGGCGAAGUCUUCGACUACCACUACCCUCGCCGUCGCGG | 50 | 921 |
| | UCUUUGCUGUC | | |
| RV-UML-m006 | AGGCCGAGUCAUUAUCAUACUUGGUUCAAUCUAUUGAA | 50 | 922 |
| | CGAAGUAUUGCU | | |
| RV-UML-m004 | AGGGACCAAGAGUUCGAUACCUCAUCGAACUGCGAGUC | 50 | 923 |
| | AUAAGCAGGGCC | | |
| RV-UML-m007 | AGGGAUUUACCGAGAAUCUAGUUACAGAACAACCUCUU | 50 | 924 |
| | CCUAAAAGGGCC | | |
| RV-UML-m290 | AGGUUGCACUUUACCCUCCCUUCCCGUGGGAACGCAUUG | 50 | 925 |
| | CGAAAAUCGCA | | |
| RV-UML-m038 | AGCCGGAACACGGUAUCUGUGCGUUCGUAAUCAGUACU | 50 | 926 |
| | AACUCUAUCCAG | | |
| RV-UML-m291 | AGUGGAGAUUCGGGAUCUGCAAAUCACCGAUAAAACAU | 50 | 927 |
| | CCCUUCGGGAGG | | |
| RV-UML-m091 | AGAACGCGAGUUCCUUCUUCACUGGUACCUCACACCCAA | 50 | 928 |
| | UAGCAAUUCCA | | |
| RV-UML-m280 | AGACGGUAGACAAGGAAUCGAAAGACUGCUCCUCACUG | 50 | 929 |
| | CUUACGUUUACC | | |
| RV-UML-m078 | AGGUGGUCUUCUUGUUGCUACAGGAACCCACGGACUUA | 50 | 930 |
| | UUCGAUAGCGAC | | |
| RV-UML-m354 | AGUGAGUCCCCGUCAACCGUAUCGGGAGGGCGAUCGAC | 50 | 931 |
| | AAUUCUCUACCA | | |
| RV-UML-m076 | AGUGCCGCAUUGUCUUCUUGUGUUCGCUUGCCGCCGACG | 50 | 932 |
| | AACAACUGGAA | | |
| RV-UML-m358 | AGAAGGUUCCGCGACGCUCCCGCCUUCUAUCCCCUUCUU | 50 | 933 |
| | UAAAGUCGCUG | | |

¹For RNA, T is U; U are T in the sequence listing

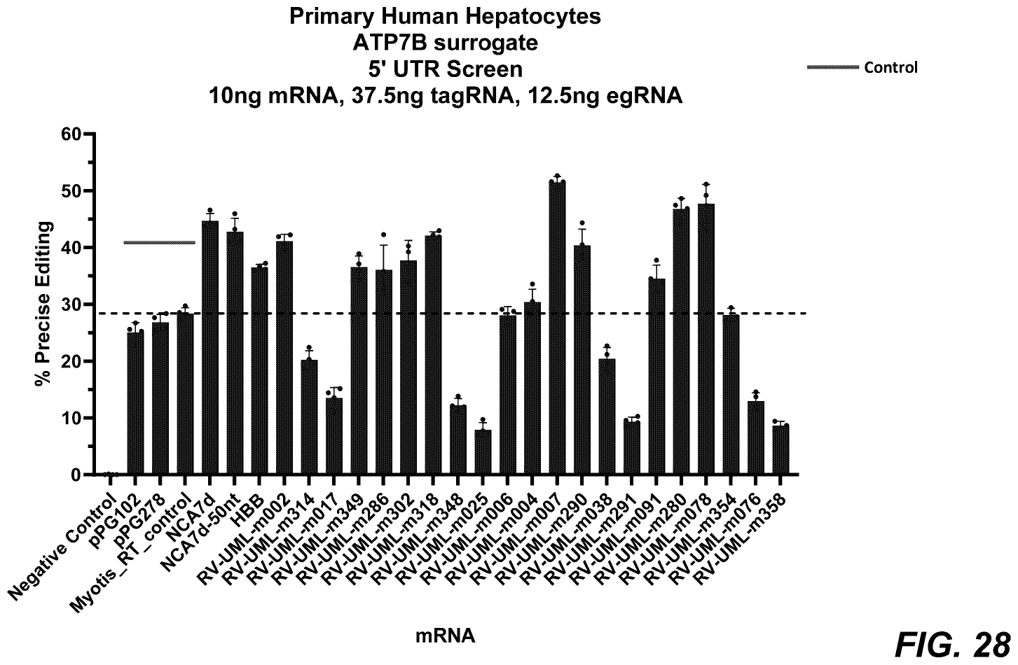

FIG. 28 displays results of the 5′UTR screen. RV-UML-m007 displayed the greatest improvement in editing efficiency. Table 30 below shows the components and full-length sequence of the RV-UML-m007 mRNA.

TABLE 30
RV-UML-M007 mRNA SEQUENCES

| | | ¹SEQ |
|---|---|---|
| | Length | ID NO: |
| 5′UTR | 50 | 924 |
| Start codon | 3 | |
| NLS | 54 | 934 |
| nCas9 | 4101 | 935 |
| Linker | 102 | 936 |
| RT | 2007 | 937 |
| NLS | 102 | 938 |
| Stop codon | 3 | |
| 3′UTR | 110 | 188 |
| PolyA | 130 | 185 |
| Full length | 6662 | 939 |

¹For RNA, T is U; U are T in the sequence listing

### Example 7

### In Vivo Studies

### Two In Vivo AA TD Studies

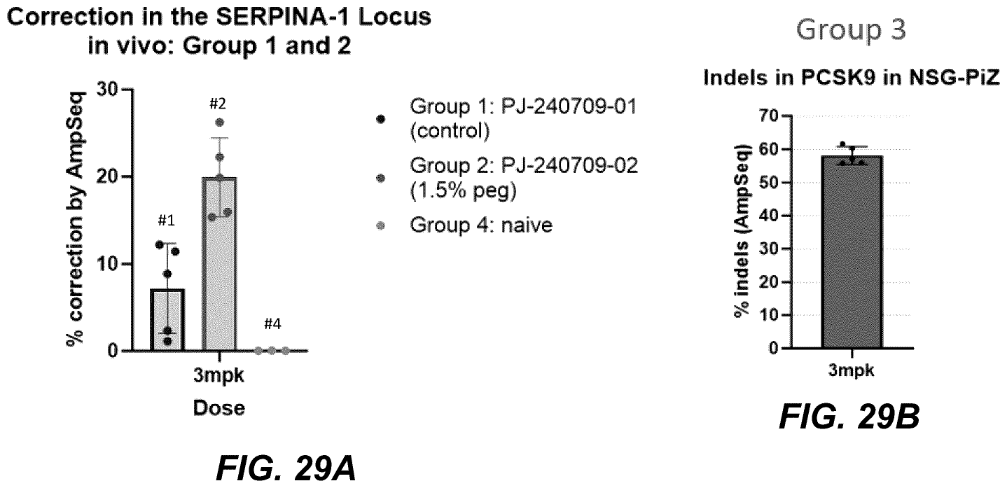

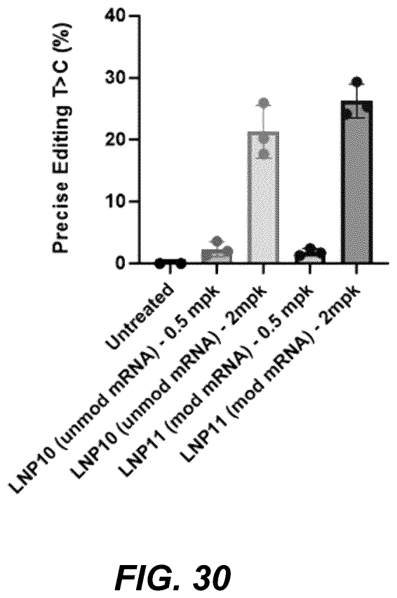

An AATD mouse model (Jackson Laboratory, 028842) was employed in two in vivo studies. FIGS. 29A-29B depicts non-limiting exemplary data related to editing results in Groups 1, 2, and 4 (FIG. 29A) and Group 3 (FIG. 29B) of a first in vivo proof of concept study performed in a humanized AATD mouse model. FIG. 30 depicts non-limiting exemplary data related to editing results in a second in vivo proof of concept study performed in a humanized AATD mouse model.

The payload of Groups 1 and 2 was the same for both (mRNA:pAM035, and guide: Ssa_s20_v17d1_F6E7_TtoC—sequences are in Tables 34-35 below).

mRNAs encoding ssaCas9 RT editor (pAM035) and tagRNA was mixed at a 1:1 w/w ratio in an aqueous buffer. The lipid mix composed of a cationic lipid, a helper lipid, a PEG-lipid and a cholesterol at optimized ratio of 47.5:10:40:2.5 (PJ-240709-01) and of 47.5:10:41:1.5 (PJ-240709-02) were mixed in ethanol (for Study 1). (For study 2 formulation was only 47.5:10:40:2.5). The total RNA in aqueous phase and the lipid in ethanol phase was mixed using a Precision Ignite system with 3:1 aqueous: ethanol ratio at 12 ml/min speed. The formulated LNP was diluted with PBS and dialyzed against PBS overnight.

A total RNA dose of 3 mg/kg (Study 1) or 0.5 mg/kg or 2 mg/kg (for Study 2), formulated of the LNPs was delivered intravenously to NSG-PiZ (Jackson Laboratory, 028842) mice carrying the E342K (PiZ) mutation. Seven days post-administration, the mice were sacrificed, and liver samples were collected for analysis. Genomic DNA was then extracted from the liver, and primers targeting the E342K locus were used to amplify this region. The resulting amplicons were analyzed via short-read sequencing using an Illumina MiSeq. Successful editing was indicated by the conversion of a T/A nucleotide to a C/G nucleotide.

TABLE 31
FIRST AATD STUDY

| | | | | IV | # of NSG- | |
|---|---|---|---|---|---|---|
| Group | LNP (ALC307) | Payload | Identifier | Dose | PiZZ | Readouts |
| 1 | PJ-240709-01 | SsaCas9-RT | 2.5% peg | 3 mpk | 5 | D7: liver |
| | (2.5% peg) | mRNA (pAM35) | (cntrl) | | | gDNA |
| 2 | PJ-240709-02 | Ssa_s20_v17d1 | 1.5% peg | 3 mpk | 5 | D7: liver |
| | (1.5% peg) | F6E7_TtoC | | | | gDNA |
| | | 1:1 ratio | | | | |
| 3 | PJ-240715-01 | SpyCas9-RT | mPCSK9 | 3 mpk | 5 | D7: liver |
| | (mPCSK9-2) | mRNA, mPCSK9-2 | | | | gDNA |
| | | sgRNA 1:1 ratio | | | | |
| 4 | — | | PBS | | 3 | D7: liver |
| | | | | | | gDNA |
| | | | | | 18 total mice | |

In the second in vivo study (RT editing in NSG-PiZ (SERPINA1-E342K) homozygous mice), modified mRNAs were employed. End-protected mRNA (InvdT) were tested. Unmodified mRNA vs. InvdT mRNA, pAM088 (no WPRE, MMLV-RT); control group (PBS); n=3 per cohort. tagRNA F6E7, in-house_s23, v17-d1_29, 1×fluoro FBS_1 (tagRNA showed ˜95% purity by 90 min analytical method). The study employed a dose of 0.5 mpk, 2 mpk; 2.5% PEG-lipid. Injection on Day 1 was followed by takedown on Day 8 (7 days post-injection), and sequence analysis was subsequently performed.

TABLE 32
SECOND AATD STUDY

| | | | | | | Concen- | Encapsu- |
|---|---|---|---|---|---|---|---|
| | | | | | Ratio | tration | lation |
| P | Size | PDI | mRNA | tagRNA | (m:m) | (ug/mL) | Efficiency |
| NVD 10 | 79.72 | 0.07917 | SsaCas9- | Ssa_s23_v17d1_29_1F1_OMe3_F6E7 | 1:1 | 483 | 96% |
| | | | RT with | | | | |
| | | | AG cap | | | | |
| NVD 11 | 78.54 | 0.01989 | SsaCas9- | Ssa_s23_v17d1_29_1F1_OMe3_F6E7 | 1:1 | 384 | 95% |
| | | | RT with | | | | |
| | | | AG cap, | | | | |
| | | | mod 1 | | | | |

### In Vivo PKU Study

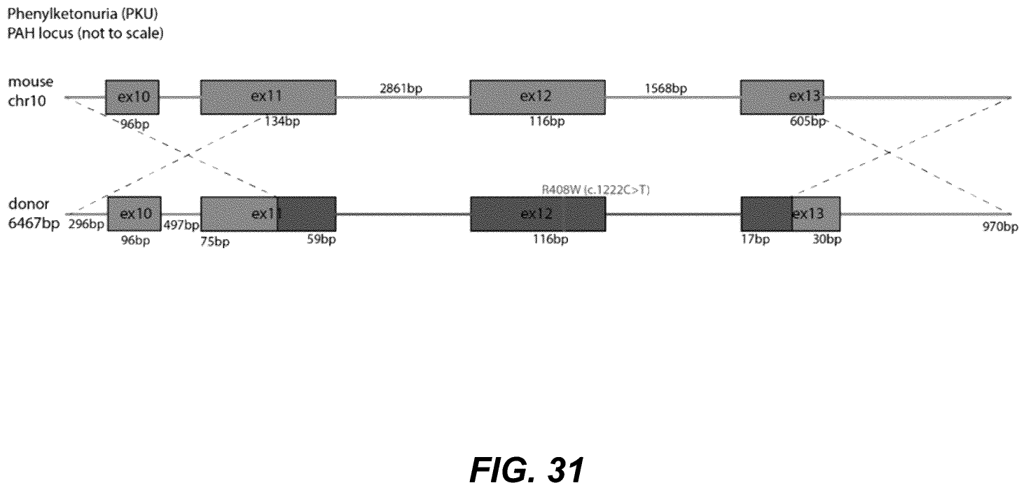

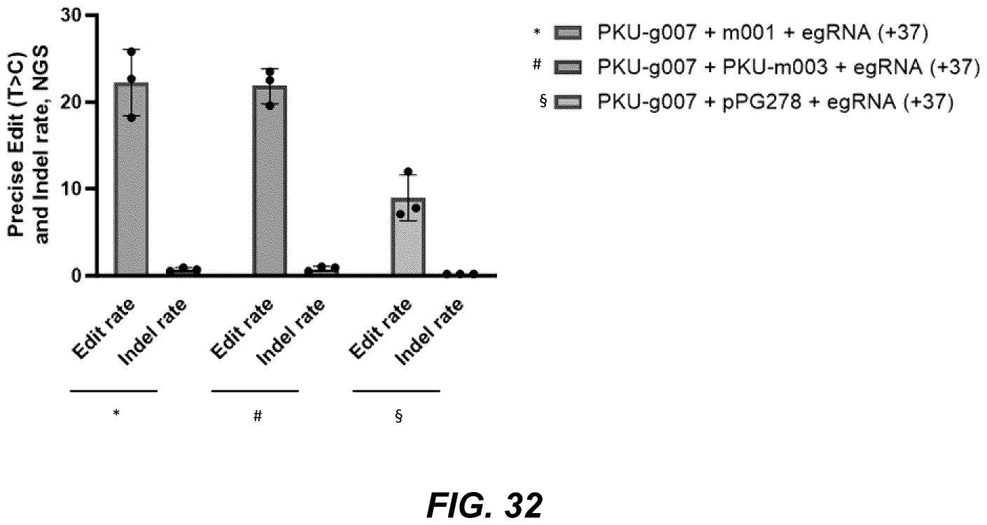

An in vivo proof of concept study was performed in humanized PKU mice. A mouse model was generated wherein the mouse exon 12 was replaced with the human exon 12 as shown in FIG. 31. FIG. 32 depicts non-limiting exemplary data related to editing results in an in vivo proof of concept study performed in humanized PKU mice.

RT Editor mRNAs (pTPRT298, pTPRT493, and pPG278), tagRNA and an egRNA were mixed at a 1:1:0.3 w/w ratio in an aqueous buffer. The lipid mix composed of a cationic lipid, a helper lipid, a PEG-lipid and a cholesterol at optimized ratio of 47.5:10:41:1.5 PJ-240709-02 were mixed in ethanol. The total RNA in aqueous phase and the lipid in ethanol phase was mixed using a Precision Ignite system with 3:1 aqueous: ethanol ratio at 12 ml/min speed. The formulated LNP was diluted with PBS and dialyzed against PBS overnight.

A total RNA dose of 2 mg/kg, formulated of the above LNPs was delivered intravenously to humanized heterozygous PKU mice where the mouse Pah exon 12 was replaced with human PAH exon 12 carrying the R408W mutation. Seven days post-administration, the mice were sacrificed, and liver samples were collected for analysis. Genomic DNA was then extracted from the liver, and primers targeting the R408W locus were used to amplify this region.

The resulting amplicons were analyzed via short-read sequencing using an Illumina MiSeq. Successful editing was indicated by the conversion of a T nucleotide to a C nucleotide.

TABLE 33
PKU STUDY

| Reagent Name | Description |
|---|---|
| PKU-g007 tagRNA | NGG1 spacer with 2′-F and 2′-oMe |
| | mods in FBS + D34 scaffold mod |
| PKU-m001 mRNA | pTPRT-U-298: custom 5′UTR with STH |
| | and segmented poly A 4 |
| PKU-m003 mRNA | pTPRT-U-493: PAPAP-8 linker, WPRE with |
| | segmented poly A 4 |
| pPG278 mRNA | pPG102 with WPRE and segmented poly A 4 |

### Materials

TABLE 34A
mRNA AND PROTEIN SEQUENCES OF CONSTRUCTS

| | Protein Sequence | mRNA Sequence |
|---|---|---|
| Name | (SEQ ID NO:) | (SEQ ID NO:) |
| pAM035 | 1036 | 1037 |
| pAM088 | 1038 | 1039 |
| pTPRT-U-298 | 1040 | 1041 |
| pTPRT-U-493 (contains a | 1042 | 1043 |
| different linker) | | |
| pPG278 | 1044 | 1045 |

TABLE 34B
GUIDES

| Name | Sequence | Sequence with chemical modifications |
|---|---|---|
| PKU-g007 | UAGCGAACUGAGAAGGGCCAGU | mU*mA*mG*rCrGrArArCrUrGrArGrArArGrGrGrCrCrAr |
| (Fp15_D34_ | UUUAGAGCUAGAAAUAGCAAGU | GrUrUrUrUrArGrAmGmCmUmAmGmAmAmAmUmAm |
| NGG1_F10E12) | UAAAAUAAGGCUAGUCCGUUAU | GrCrArArGrUrUrArArArArUrArArGrGrCrUrArGrUrCrCr |
| | CAACUUGAAAAAGUGGCACCGA | GrUrUrArUrCrArAmCmUmUmGmAmArArAmArGrUrGr |
| | GUCGGUGCACAAUACCUCGGCCC | GrCrArCmCrGrArGrUrCrGrGrUrGmCrArCrArArUrArCr |
| | UUCUCAG (SEQ ID NO: 1046) | CrUrCrGrG/i2FC/rC/i2FC/rUmUmCmU*mC*mA*mG |
| | | (SEQ ID NO: 1047) |
| PKU | UUCACUCAAGCCUGUGGUUUGU | mU*mU*mC*rArCrUrCrArArGrCrCrUrGrUrGrGrUrUrUr |
| egRNA | UUUAGAGCUAGAAAUAGCAAGU | GrUrUrUrUrArGrAmGmCmUmAmGmAmAmAmUmAm |
| +37 | UAAAAUAAGGCUAGUCCGUUAU | GmCrArArGrUrUrArArArArUrArArGrGrCrUrArGrUrCrC |
| (PKU_NGG1_ | CAACUUGAAAAAGUGGCACCGA | rGrUrUrArUrCmAmAmCmUmUmGmAmAmAmAmAm |
| plus37_ | GUCGGUGCUUUU (SEQ ID NO: | GmUmGmGmCmAmCmCmGmAmGmUmCmGmGmUm |
| D00) | 1048) | GmCmU*mU*mU*mU (SEQ ID NO: 1049) |
| Ssa_s20_ | AAGGCUGUGCUGACCAUCGAGU | mA*mA*mG*rGrCrUrGrUrGrCrUrGrArCrCrArUrCrGrAr |
| v17d1_F6E7_ | UUUUGUACUCUGAAAAGAAGCU | GrUrUrUmUmUmGmUmArCrUmCmUmGmAmAmAmA |
| TtoC | ACAAAGAUAAGGCUUCAUGCCG | mGmAmArGrCrUrArCrArAmArGrArUrArArGrGmCmUr |
| | AAAUCAACUCGUCGAUGGUC | UmCrArUrGrCrCrGrArArAmUrCmAmArCrUrCrGrUrCr |
| | (SEQ ID NO: 1050) | GrArUrG*mG*mU*mC (SEQ ID NO: 1051) |
| Ssa_s23_ | CAUAAGGCUGUGCUGACCAUCG | mC*mA*mU*rArArGrGrCrUrGrUrGrCrUrGrArCrCrArUr |
| v17d1_29_ | AGUUUUUGUACUCCGAAAGGAA | CrGrArGrUrUrUmUmUmGmUmArCrUmCmCmGmAmA |
| 1F1_OMe3_ | GCUACAAAGAUAAGGCUUCAUG | mAmGmGmAmArGrCrUrArCrArAmArGrArUrArArGrG |
| F6E7 | CCGAAAUCAUCUCGUCGAUGGU | mCmUrUmCrArUrGrCrCrGrArArAmUrCmAmUrCrUrCr |
| | C (SEQ ID NO: 1052) | GrUrCrG/i2FA/rUmG*mG*mU*mC (SEQ ID NO: 1053) |

M = 2′OMe, r = ribose, /i2F/ = 2′fluoro

### Example 8

### Codon Optimization

RT editor in pAM053 was used to generate three new codon optimizations (pAM197, 198, 199, Table 35 below). These four sequences were made into mRNA and tested in primary human hepatocytes using the ATP7B surrogate mutation and the PAH surrogate mutation.

TABLE 35
RT EDITOR CODON OPTIMIZATION

| | SEQ |
|---|---|
| Name/Description | ID NO: |
| pAM053 | 1085 |
| pAM197 | 1086 |
| pAM198 | 1087 |
| pAM199 | 1088 |

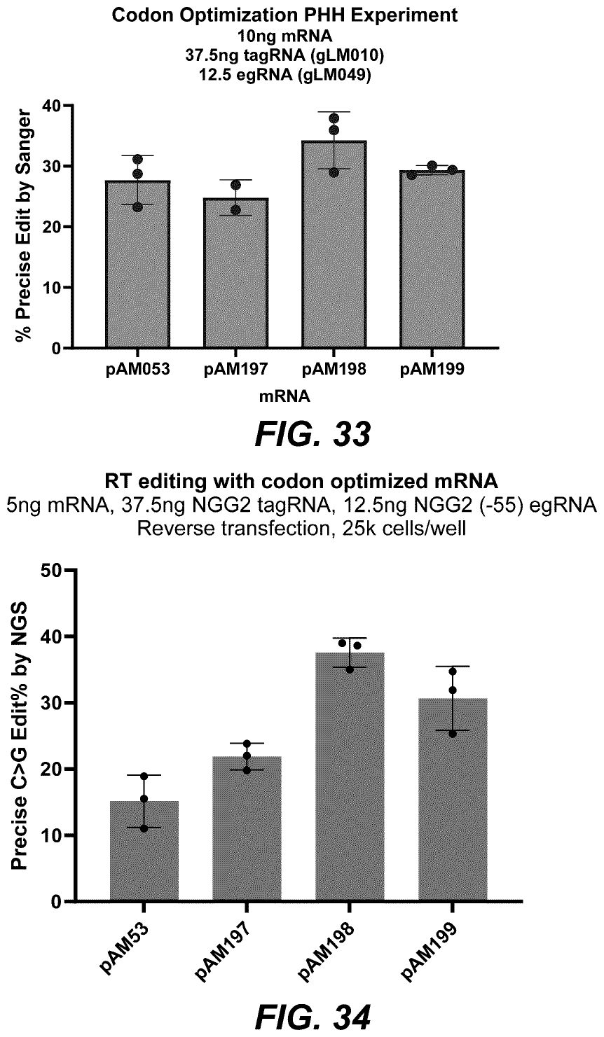

The codon optimized sequence pAM198 outperformed the other three codon optimizations at both genomic targets (FIG. 33-FIG. 34).

### Example 9

### Accessory Domains

Provided in this Example are RT editors comprising accessory domains. Accessory domains can include stabilizing domains (e.g., stabilons) and/or single strand binding (SSB) proteins to enhance the potency and/or processivity of RT editing.

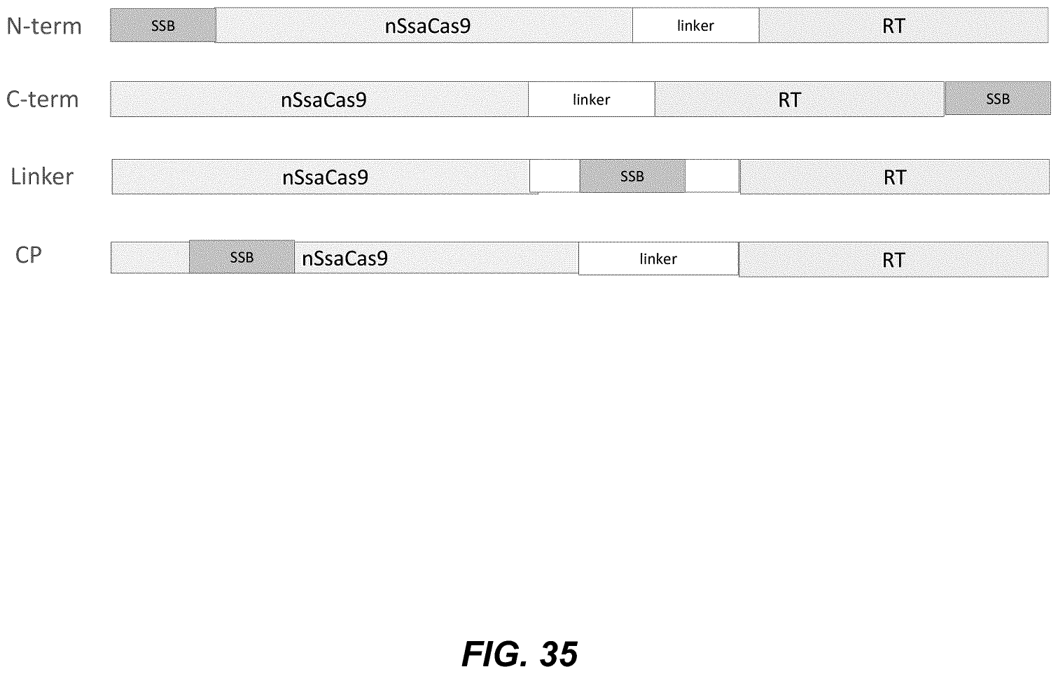

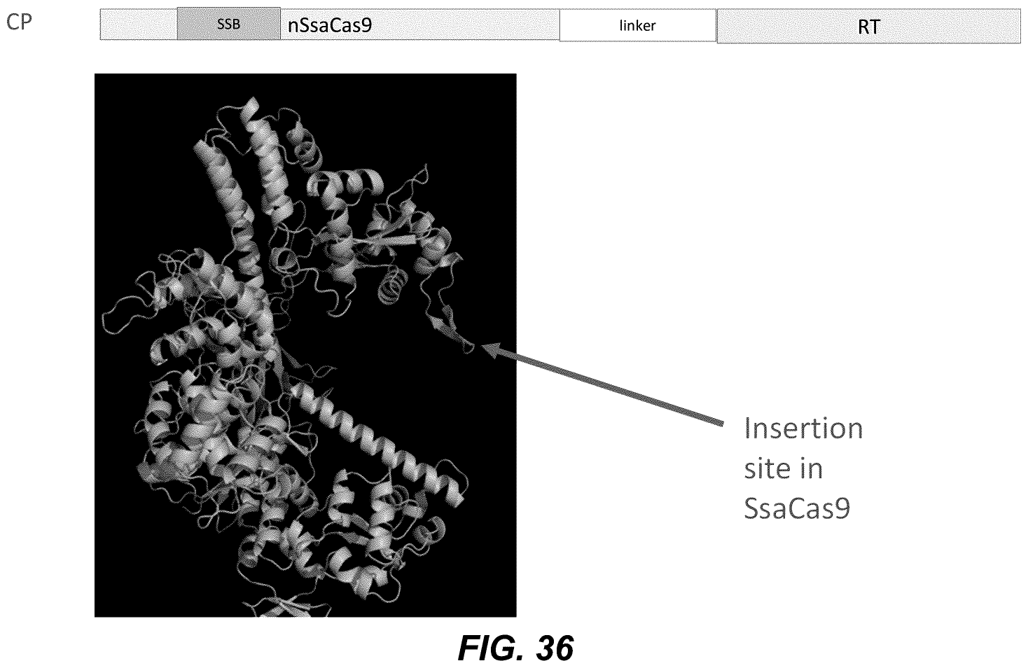

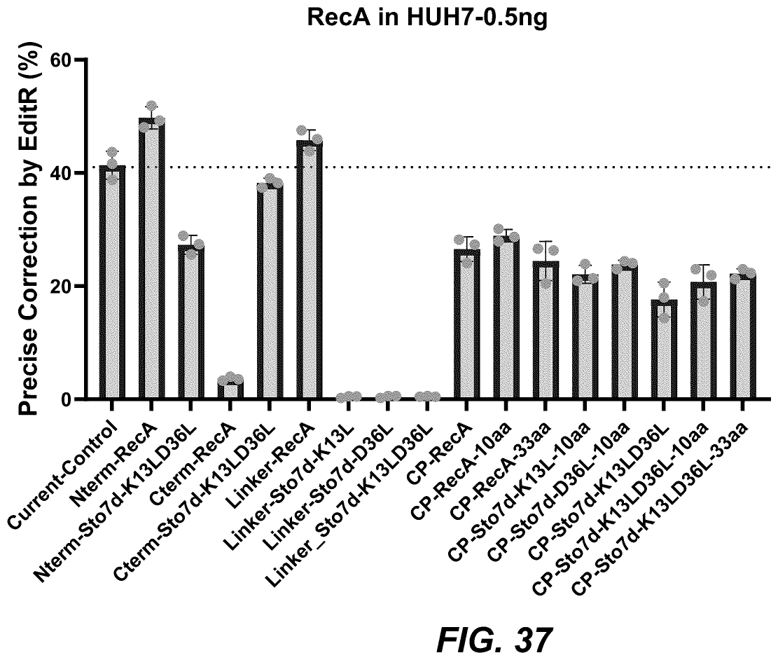

Accessory domains can enhance the potency and processivity of RT editing. RecA peptides have previously been shown to improve Cas9-based HDR. RecA peptides are known to promote homologous DNA pairing. Sto7d has been used to increase the processivity of various polymerases. It has been shown that Stabilon sequences can enhance the activity of BxB1 by increasing protein and mRNA stability. Peptide insertion strategies are shown in FIG. 35-FIG. 36. RecA and Sto7d binding protein insertions were tested and results are shown in FIG. 37. Nterm-RecA and Linker-RecA shows improvement in editing. Sto7d does not show improvement.

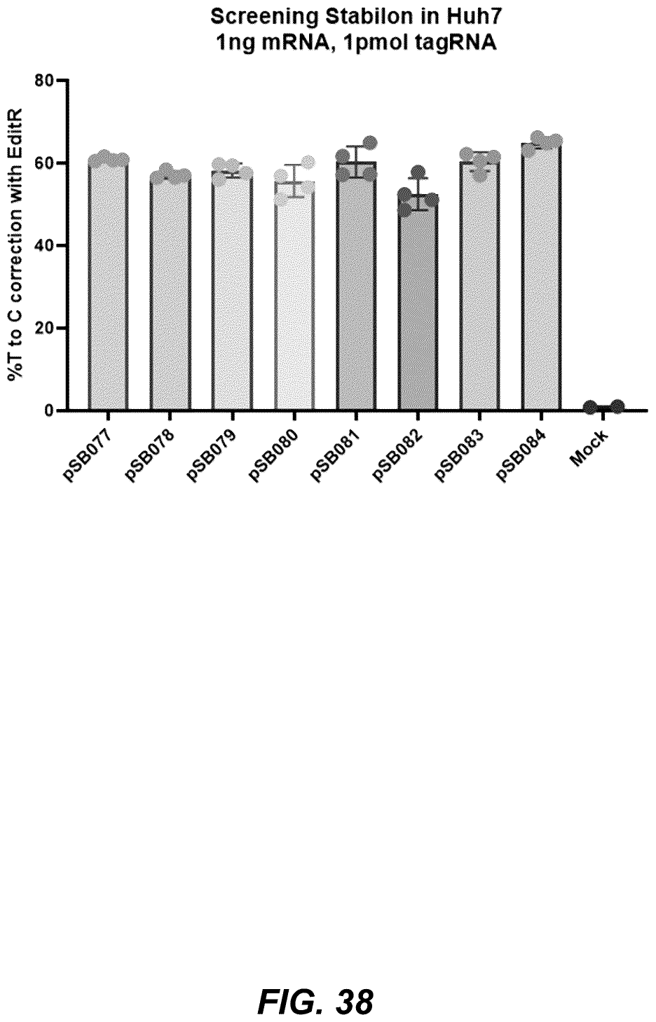

A Stabilion screen was performed in Huh7 cells, the results of which are shown in FIG. 38. No improvement was found compared to the control (FIG. 38). SEQ ID NOs of the various RT-editors comprising accessory domains are provided in Table 36.

TABLE 36A
EXEMPLARY STABILON SEQUENCES

| | Protein | mRNA |
|---|---|---|
| | Sequence | Sequence |
| Name | (SEQ ID NO) | (SEQ ID NO) |
| Control (pAM088) | 1038 | 1039 |
| Nterm-RecACP-Sto7d-K13L-10aa | 1276 | 1301 |
| NtermCP-Sto7d-K13LD36LD36L-10aa | 1277 | 1302 |
| Cterm-RecACP-Sto7d-K13LD36L | 1278 | 1303 |
| CtermCP-Sto7d-K13LD36L-10aa | 1279 | 1304 |
| Linker-RecACP-Sto7d-K13LD36L-33aa | 1280 | 1305 |
| Linker-Sto7d-K13LCP-RecA | 1281 | 1306 |
| CP-RecA-10aa | 1282 | 1307 |
| Linker_Sto7d-K13LD36LCP-RecA-33aa | 1283 | 1308 |
| Nterm-Sto7d-K13LD36L | 1284 | 1309 |
| CPNterm-RecA-10aa | 1285 | 1310 |
| CP-RecA-33aaCterm-Sto7d-K13LD36L | 1286 | 1311 |
| CP-Sto7d-K13L-10aaCterm-RecA | 1287 | 1312 |
| CPLinker-Sto7d-D36L-10aaK13LD36L | 1288 | 1313 |
| CP-Sto7d-K13LD36LLinker-RecA | 1289 | 1314 |
| CPLinker-Sto7d-K13LD36L- 10aaK13L | 1290 | 1315 |
| CPLinker-Sto7d-K13LD36L- 33aaD36L | 1291 | 1316 |
| NLS-STABILION1-SsaCas9-w/ trunc M-MLV (pSB077) | 1292 | 1317 |
| NLS-STABILION2-SsaCas9-w/ trunc M-MLV (pSB078) | 1293 | 1318 |
| NLS-STABILION3-SsaCas9-w/ trunc M-MLV (pSB079) | 1294 | 1319 |
| NLS-STABILION4-SsaCas9-w/ trunc M-MLV (pSB080) | 1295 | 1320 |
| NLS-STABILION5-SsaCas9-w/ trunc M-MLV (pSB081) | 1296 | 1321 |
| NLS-STABILION6-SsaCas9-w/ trunc M-MLV (pSB082) | 1297 | 1322 |
| NLS-STABILION7-SsaCas9-w/ trunc M-MLV (pSB083) | 1298 | 1323 |
| Control, pAM088-w/ Trunc M-MLV (pSB084) | 1299 | 1324 |
| pSB046 | 1300 | 1325 |

TABLE 36B
EXEMPLARY gRNA SEQUENCES

| NAME | SEQUENCE^{1,2} | SEQ ID NO: |
|---|---|---|
| Ssa_s22_29d1_ | rGrArUrArArGrGmCmUrUmCrArUrGrCrCrGrArArAmUrCmAmUrCrUrCr | 1269 |
| F6E7_1F_TtoC | mA*mU*mA*rArGrGrCrUrGrUrGrCrUrGrArCrCrArUrCrGrArGrUrUrUmU | |
| | mUmGmUmArCrUmCmCmGmAmAmAmGmGmAmArGrCrUrArCrArAmA | |
| | GrUrCrG/i2FA/rUrG*mG*mU*mC | |

¹For RNA, T is U; U are T in the sequence listing. For each entry, the spacer sequence is underlined and the extension arm is bold.
²For the sequences: * , phosphorothioate modification; mN, 2′O-methyl modification; rN, unmodified ribonucleotide; i2FN, Int Fluoro modification. N = A, C, T/U, G.

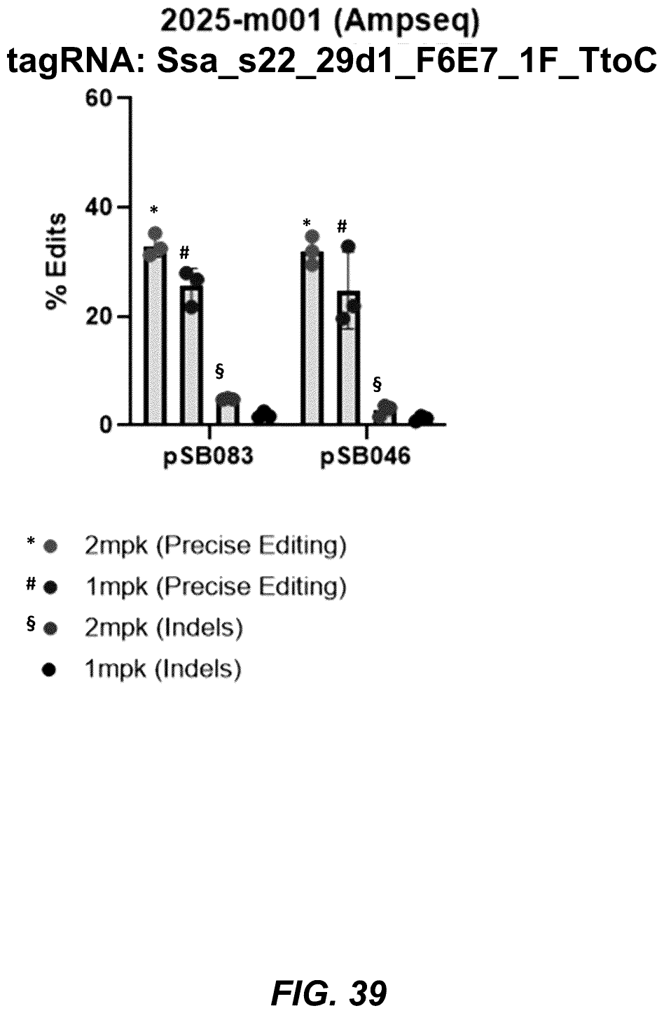

An in vivo study was performed. pSB083 is an N-term Stabilion fusion. pSB046 contains lead UTRs (5′CES1var4 and 3′ extended HBB UTRs). Data from in vivo screening of mRNAs is shown in FIG. 39. Very low Indels were observed at 1mpk dose.

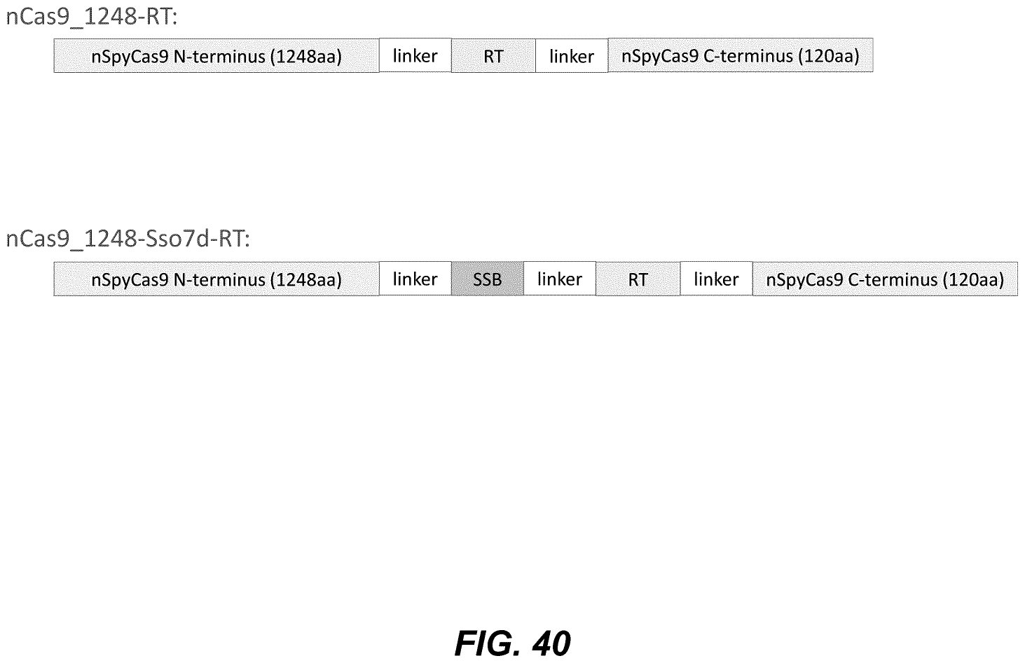

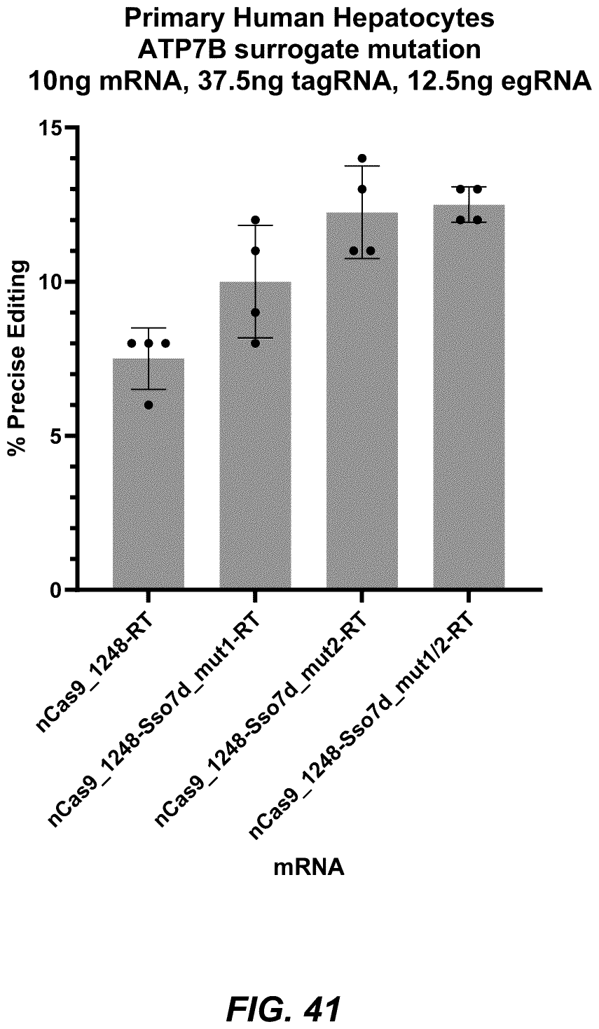

The employment of constructs comprising Sso7d was tested in the context of, e.g., Wilson's disease. Sso7d is a 64aa DNA-binding protein from Saccharolobus solfataricus. Two mutations are thought to ablate the RNase activity of Sso7d: K12L (also referred to herein as mut1) and E35L (also referred to herein as mut2). “mut1/2” means both mutations were incorporated. Sto7d from Sulfolobus tokodaii is an orthologue of Sso7d. Sto7d was tested for AATD and Sso7d was tested for WD. In this experiment, nSpyCas9 was split into two parts between amino acids 1248 and 1249. The RT and the Sso7d were inserted between the two portions of Cas9 (FIG. 40). As shown in FIG. 41, fusion of Sso7d to RT editor can increase editing efficiency. Table 37 below displays SEQ ID NOs of exemplary Sso7d related sequences of the disclosure.

TABLE 37
EXEMPLARY SSO7D SEQUENCES

| | Protein |
|---|---|
| Name | Sequence |
| Sso7d_mut1/2 | 1205 |
| NLS-nCas9_1248-RT-NLS | 1206 |
| NLS-nCas9_1248-Sso7d_mut1-RT-NLS | 1207 |
| NLS-nCas9_1248-Sso7d_mut2-RT-NLS | 1208 |
| NLS-nCas9_1248-Sso7d_mut1/2-RT-NLS | 1209 |

### Example 10

### Wilson's Disease mRNA Screen

In this Example, mRNAs were screened for comparison of lead constructs containing different linkers, NLSs, UTRs, RTs, stabilizing elements, and/or polyA. Table 38A-Table 38B provides the sequences and design of mRNAs tested. Sequences of components listed below can be found, for example, in Table 1, Table 2A-Table 2C, Table 9A-Table 9C, Table 11, and Table 29.

TABLE 38A
RT EDITORS (PROTEIN SEQUENCES)

| Name | Nterm NLS | Cas9 | Linker | RT | Cterm NLS |
|---|---|---|---|---|---|
| pLM147 | SEQ ID NO: 5 | SpCas9 R221K | GS-NLS-GS_4-gs | Mbr_WT | SEQ ID NO: 11 + SEQ |
| | | N394K H840A | | | ID NO: 12 |
| pLM149 | SEQ ID NO: 5 | SpCas9 R221K | GS-NLS-GS_4-gs | Mbr_N197C* | SEQ ID NO: 11 + SEQ |
| | | N394K H840A | | | ID NO: 12 |
| pMR031 | SEQ ID NO: 5 | SpCas9 R221K | PAPAP-4-Ala | MMLV_truncated | SEQ ID NO: 11 + SEQ |
| | | N394K H840A | | | ID NO: 12 |
| pMR032 | SEQ ID NO: 5 | SpCas9 R221K | PAPAP-8-Ala | MMLV_truncated | SEQ ID NO: 11 + SEQ |
| | | N394K H840A | | | ID NO: 12 |
| pPG102 | SEQ ID NO: 5 | SpCas9 R221K | GS-NLS-GS_4-gs | MMLV (SEQ ID NO: | SEQ ID NO: 11 + SEQ |
| | | N394K H840A | | 8) | ID NO: 12 |
| pPG307 | SEQ ID NO: 5 | SpCas9 R221K | PAPAP-8-Ala | Mbr_WT | SEQ ID NO: 11 + SEQ |
| | | N394K H840A | | | ID NO: 12 |
| pTPRT- | SEQ ID NO: 5 | SpCas9 R221K | GS-NLS-GS_4-gs | MMLV (SEQ ID NO: | SEQ ID NO: 11 + SEQ |
| U-294 | | N394K H840A | | 8) | ID NO: 12 |
| PTPRT- | SEQ ID NO: 5 | SpCas9 R221K | GS-NLS-GS_4-gs | MMLV (SEQ ID NO: | SEQ ID NO: 11 + SEQ |
| U-298 | | N394K H840A | | 8) | ID NO: 12 |
| pTPRT- | SEQ ID NO: 5 | SpCas9 R221K | GS-NLS-GS_4-gs | MMLV (SEQ ID NO: | SEQ ID NO: 11 + SEQ |
| U-323 | | N394K H840A | | 8) | ID NO: 12 |
| PTPRT- | SEQ ID NO: 5 | SpCas9 R221K | GS-NLS-GS_4-gs | MMLV (SEQ ID NO: | SEQ ID NO: 11 + SEQ |
| U-331 | | N394K H840A | | 8) | ID NO: 12 |
| pTPRT- | SEQ ID NO: 5 | SpCas9 R221K | GS-NLS-GS_4-gs | MMLV (SEQ ID NO: | SEQ ID NO: 11 + SEQ |
| U-334 | | N394K H840A | | 8) | ID NO: 12 |
| pTPRT- | SEQ ID NO: 5 | SpCas9 R221K | GS-NLS-GS_4-gs | MMLV (SEQ ID NO: | SEQ ID NO: 11 + SEQ |
| U-375 | | N394K H840A | | 8) | ID NO: 12 |
| pTPRT- | SEQ ID NO: 5 | SpCas9 R221K | PAPAP-8-Ala | MMLV (SEQ ID NO: | SEQ ID NO: 11 + SEQ |
| U-493 | | N394K H840A | | (8) | ID NO: 12 |
| pTPRT- | SEQ ID NO: 5 | SpCas9 R221K | GS-NLS-GS 4-gs | MMLV (SEQ ID NO: | SEQ ID NO: 5 |
| U-504 | | N394K H840A | | 8) | |
| PTPRT- | SEQ ID NO: 5 | SpCas9 R221K | PAPAP-8-Ala | MMLV (SEQ ID NO: | SEQ ID NO: 5 |
| U-505 | | N394K H840A | | 8) | |
| pTPRT- | NLS of | SpCas9 R221K | GS-NLS-GS_4-gs | MMLV (SEQ ID NO: | NLS of SV40 + NLS of |
| U-516 | Nucleoplasmin | N394K H840A | | 8) | C-Myc |
| | + Hybrid NLS | | | | |
| PTPRT- | NLS of | SpCas9 R221K | PAPAP-8-Ala | MMLV (SEQ ID NO: | NLS of SV40 + NLS of |
| U-517 | Nucleoplasmin | N394K H840A | | 8) | C-Myc |
| | + Hybrid NLS | | | | |
| PTPRT- | Hybrid NLS + | SpCas9 R221K | GS-NLS-GS_4-gs | MMLV (SEQ ID NO: | NLS of SV40 + NLS of |
| U-520 | NLS of SV40 | N394K H840A | | 8) | C-Myc |
| pTPRT- | NLS of | SpCas9 R221K | PAPAP-8-Ala | MMLV (SEQ ID NO: | SEQ ID NO: 5 |
| U-525 | Nucleoplasmin | N394K H840A | | 8) | |
| | + Hybrid NLS | | | | |
| pTPRT- | VirD NLS | SpCas9 R221K | PAPAP-8-Ala | MMLV (SEQ ID NO: | SEQ ID NO: 11 + SEQ |
| U-529 | | N394K H840A | | 8) | ID NO: 12 |
| pTPRT- | SEQ ID NO: 5 | SpCas9 R221K | PAPAP-8-Ala | MMLV (SEQ ID NO: | SEQ ID NO: 11 + SEQ |
| U-540 | | N394K H840A | | 8) | ID NO: 12 |
| PTPRT- | SEQ ID NO: 5 | SpCas9 R221K | PAPAP-8-Ala | MMLV (SEQ ID NO: | SEQ ID NO: 11 + SEQ |
| U-543 | | N394K H840A | | 8) | ID NO: 12 |
| PVC254 | SEQ ID NO: 5 | SpCas9 R221K | PAPAP-8-Ala | Mbr_truncated | SEQ ID NO: 11 + SEQ |
| | | N394K H840A | | | ID NO: 12 |
| PVC267 | SEQ ID NO: 5 | SpCas9 R221K | PAPAP-4-Ala | Pipistrellus_ | SEQ ID NO: 11 + SEQ |
| | | N394K H840A | | truncated | ID NO: 12 |
| PVC268 | SEQ ID NO: 5 | SpCas9 R221K | PAPAP-8-Ala | Pipistrellus_ | SEQ ID NO: 11 + SEQ |
| | | N394K H840A | | truncated | ID NO: 12 |
| PVC271 | SEQ ID NO: 5 | SpCas9 R221K | GGS-AEAK- | Pipistrellus_ | SEQ ID NO: 11 + SEQ |
| | | N394K H840A | ALEA_mod1-gs | truncated | ID NO: 12 |
| PVC299 | SEQ ID NO: 5 | SpCas9 R221K | GS-NLS-GS_4-gs | Mbr_WT | SEQ ID NO: 11 + SEQ |
| | | N394K H840A | | | ID NO: 12 |
| PVC302 | SEQ ID NO: 5 | SpCas9 R221K | GS-NLS-GS_4-gs | Mbr_WT | SEQ ID NO: 11 + SEQ |
| | | N394K H840A | | | ID NO: 12 |
| PVC313 | SEQ ID NO: 5 | SpCas9 R221K | GS-NLS-GS 4-gs | Mbr_WT | SEQ ID NO: 11 + SEQ |
| | | N394K H840A | | | ID NO: 12 |
| PVC318 | SEQ ID NO: 5 | SpCas9 R221K | GS-NLS-GS_4-gs | Mbr_WT | SEQ ID NO: 11 + SEQ |
| | | N394K H840A | | | ID NO: 12 |
| PVC319 | SEQ ID NO: 5 | SpCas9 R221K | GS-NLS-GS_4-gs | Mbr_WT | SEQ ID NO: 11 + SEQ |
| | | N394K H840A | | | ID NO: 12 |

*Mbr_N197C is SEQ ID NO: 255 comprising an N197C substitution (See, also, Table 27A above)

TABLE 38B
EXEMPLARY MRNAS ENCODING RT EDITORS (NON-CODING)

| | | | Stabilizing | |
|---|---|---|---|---|
| Name | 5′ UTR | 3′UTR | Element | Poly A |
| pLM147 | Syn_5′UTR | hHBA | WPRE | Seg_PolyA_3 |
| pLM149 | Syn_5′UTR | hHBA | WPRE | Seg_PolyA_3 |
| pMR031 | Syn_5′UTR | hHBA | WPRE | Seg_PolyA_3 |
| pMR032 | Syn_5′UTR | hHBA | WPRE | Seg_PolyA_3 |
| pPG102 | Syn_5′UTR | mHBA | None | 120_PolyA |
| pPG307 | Syn_5′UTR | hHBA | WPRE | Seg_PolyA_3 |
| pTPRT-U-294 | Apt17_custom 5′UTR | hHBA | STH | Seg_PolyA_4 |
| pTPRT-U-298 | custom 5′UTR | hHBA | STH | Seg_PolyA_4 |
| pTPRT-U-323 | SAA1 5′UTR | hHBA | WPRE | Seg_PolyA_4 |
| pTPRT-U-331 | SERPINA1 5′UTR | hHBA | WPRE | Seg_PolyA_4 |
| pTPRT-U-334 | SERPINA1 5′UTR | hHBA | STH | Seg_PolyA_4 |
| pTPRT-U-375 | CES1 var2 | hHBA | WPRE | Seg_PolyA_4 |
| pTPRT-U-493 | Syn_5′UTR | hHBA | WPRE | Seg_PolyA_4 |
| pTPRT-U-504 | Syn_5′UTR | hHBA | WPRE | Seg_PolyA_4 |
| pTPRT-U-505 | Syn_5′UTR | hHBA | WPRE | Seg_PolyA_4 |
| pTPRT-U-516 | Syn_5′UTR | hHBA | WPRE | Seg_PolyA_4 |
| pTPRT-U-517 | Syn_5′UTR | hHBA | WPRE | Seg_PolyA_4 |
| pTPRT-U-520 | Syn_5′UTR | hHBA | WPRE | Seg_PolyA_4 |
| pTPRT-U-525 | Syn_5′UTR | hHBA | WPRE | Seg_PolyA_4 |
| pTPRT-U-529 | Syn_5′UTR | hHBA | WPRE | Seg_PolyA_4 |
| pTPRT-U-540 | custom 5′UTR | hHBA | STH | Seg_PolyA_4 |
| pTPRT-U-543 | ALB 5′UTR | hHBA | None | Seg_PolyA_4 |
| pVC254 | Syn_5′UTR | hHBA | None | Seg_PolyA_4 |
| pVC267 | Syn_5′UTR | hHBA | None | Seg_PolyA_4 |
| pVC268 | Syn_5′UTR | hHBA | None | Seg_PolyA_4 |
| pVC271 | Syn_5′UTR | hHBA | None | Seg_PolyA_4 |
| pVC299 | NCA7d | hHBA | None | Seg_PolyA_4 |
| pVC302 | m002 | hHBA | None | Seg_PolyA_4 |
| pVC313 | m007 | hHBA | None | Seg_PolyA_4 |
| pVC318 | m280 | hHBA | None | Seg_PolyA_4 |
| pVC319 | m078 | hHBA | None | Seg_PolyA_4 |

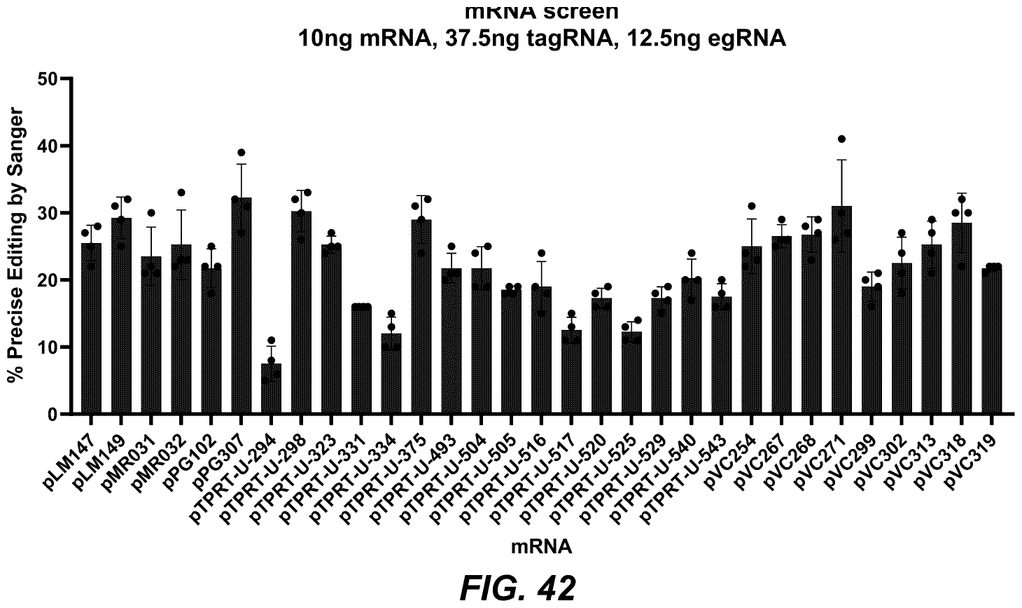

Editing data in primary human hepatocytes are shown for correction of ATP71B surrogate mutation in FIG. 42.

### Example 11

### Chemical Modifications

Described in this Example are additional chemical modifications of tagRNAs disclosed herein. The tagRNA modifications were tested for correction of ATP71B H1069Q mutation in Huh7 cells. Table 39A-Table 39C provide the chemically modified tagRNAs. In some of the RNA sequences listed below, a position may be modified as a deoxyribonucleotide. Such position are not preceded by an ‘r’ and, when appropriate, are annotated as “T”.

TABLE 39A
EXEMPLARY MODIFIED tagRNAS (ATP7B)

| NAME | SEQUENCE^{1,2} | SEQ ID NO: |
|---|---|---|
| ChemMod_Set2_1 | mU*mU*mG*rGrUrGrArCrUrGrCrCrArCrGrCrCrCrArArGrUrUrUrUrArGr | 1089 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrG/i2FU/ | |
| | rGrA/i2FA/rCrA/i2FC/rCrC/i2FC/rUrU/i2FG/rGrG/i2FC/rGrU/i2FG/ | |
| | rGrCmU*mU*mU*mU | |
| ChemMod_Set2_2 | mU*mU*mG*rGrUrGrArCrUrGrCrCrArCrGrCrCrCrArArGrUrUrUrUrArGr | 1090 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrG/i2FC/rGrU/i2FG/rGrCmU*mU*mU*m | |
| | U | |
| ChemMod_Set2_3 | mU*mU*mG*rGrUrGrArCrUrGrCrCrArCrGrCrCrCrArArGrUrUrUrUrArGr | 1091 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrG/i2FU/ | |
| | rGrA/i2FA/rCrA/i2FC/rCrC/i2FC/rUrU/i2FG/rGrGrCrGrUrGrGrCmU* | |
| | mU*mU*mU | |
| ChemMod_Set2_4 | mU*mU*mG*rGrUrGrArCrUrGrCrCrArCrGrCrCrCrArArGrUrUrUrUrArGr | 1092 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmC/i2FG/ | |
| | rUrG/i2FA/rArC/i2FA/rCrC/i2FC/rCrU/i2FU/rGrG/i2FG/rCrG/i2FU/ | |
| | rGrG/i2FC/mU*mU*mU*mU | |
| ChemMod_Set2_5 | mU*mU*mG*rGrUrGrArCrUrGrCrCrArCrGrCrCrCrArArGrUrUrUrUrArGr | 1093 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrG/i2FG/rCrG/i2FU/rGrG/i2FC/mU*mU*mU | |
| | *mU | |
| ChemMod_Set2_6 | mU*mU*mG*rGrUrGrArCrUrGrCrCrArCrGrCrCrCrArArGrUrUrUrUrArGr | 1094 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmC/i2FG/rUrG/ | |
| | i2FA/rArC/i2FA/rCrC/i2FC/rCrU/i2FU/rGrGrGrCrGrUrGrGrCmU* | |
| | mU*mU*mU | |
| ChemMod_Set2_7 | mU*mU*mG*rGrUrGrArCrUrGrCrCrArCrGrCrCrCrArArGrUrUrUrUrArGr | 1095 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrGrGrG/i2FC/rGrU/i2FG/rGrCmU | |
| | *mU*mU*mU | |
| ChemMod_Set2_8 | mU*mU*mG*rGrUrGrArCrUrGrCrCrArCrGrCrCrCrArArGrUrUrUrUrArGr | 1096 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrGrG/i2FG/rCrG/i2FU/rGrG/i2FC/ | |
| | mU*mU*mU*mU | |
| ChemMod_Set2_9 | mU*mU*mG*rGrUrGrArCrUrGrCrCrArCrGrCrCrCrArArGrUrUrUrUrArGr | 1097 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrG/i2FG/mGrC/i2FG/mUrG/i2FG/ | |
| | mCmU*mU*mU*mU | |
| ChemMod_Set2_10 | mU*mU*mG*rGrUrGrArCrUrGrCrCrArCrGrCrCrCrArArGrUrUrUrUrArGr | 1098 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrG/i2FG/rGmC/i2FG/rUmG/i2FG/ | |
| | rCmU*mU*mU*mU | |
| ChemMod_Set2_11 | mU*mU*mG*rGrUrGrArCrUrGrCrCrArCrGrCrCrCrArArGrUrUrUrUrArGr | 1099 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrG/i2FG/mGmC/i2FG/mUmG/ | |
| | i2FG/mCmU*mU*mU*mU | |
| ChemMod_Set2_12 | mU*mU*mG*rGrUrGrArCrUrGrCrCrArCrGrCrCrCrArArGrUrUrUrUrArGr | 1100 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrGrG/i2FG/rC/i2FG/rU/i2FG/rG/ | |
| | i2FC/mU*mU*mU*mU | |
| ChemMod_Set2_13 | mU*mU*mG*rGrUrGrArCrUrGrCrCrArCrGrCrCrCrArArGrUrUrUrUrArGr | 1101 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrG/i2FU/ | |
| | rGrA/i2FA/rCrA/i2FC/rCrC/i2FC/rUrU/i2FG/rG/i2FG/rC/i2FG/rU/ | |
| | i2FG/rG/i2FC/mU*mU*mU*mU | |
| ChemMod_Set2_14 | mU*mU*mG*rGrUrGrArCrUrGrCrCrArCrGrCrCrCrArArGrUrUrUrUrArGr | 1102 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmC/i2FG/ | |
| | rUrG/i2FA/rArC/i2FA/rCrC/i2FC/rCrU/i2FU/rGrG/i2FG/rC/i2FG/rU/ | |
| | i2FG/rG/i2FC/mU*mU*mU*mU | |
| ChemMod_Set2_15 | mU*mU*mG*rGrUrGrArCrUrGrCrCrArCrGrCrCrCrArArGrUrUrUrUrArGr | 1103 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rG/i2FC/rG/i2FU/rG/i2FG/rCmU*mU* | |
| | mU*mU | |
| ChemMod_Set2_16 | mU*mU*mG*rGrUrGrArCrUrGrCrCrArCrGrCrCrCrArArGrUrUrUrUrArGr | \|1104 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrG/i2FG/rG/i2FC/rG/i2FU/rG/ | |
| | i2FG/rCmU*mU*mU*mU | |
| ChemMod_Set2_17 | mU*mU*mG*rGrUrGrArCrUrGrCrCrArCrGrCrCrCrArArGrUrUrUrUrArGr | 1105 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrG/i2FU/ | |
| | rGrA/i2FA/rCrA/i2FC/rCrC/i2FC/rUrU/i2FG//i2FG/rG/i2FC/rG/i2FU/ | |
| | rG/i2FG/rCmU*mU*mU*mU | |
| ChemMod_Set2_18 | mU*mU*mG*rGrUrGrArCrUrGrCrCrArCrGrCrCrCrArArGrUrUrUrUrArGr | 1106 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmC/i2FG/rUrG/ | |
| | i2FA/rArC/i2FA/rCrC/i2FC/rCrU/i2FU/rG/i2FG/rG/i2FC/rG/i2FU/rG/ | |
| | i2FG/rCmU*mU*mU*mU | |
| ChemMod_Set2_19 | mU*mU*mG*rGrUrGrArCrUrGrCrCrArCrGrCrCrCrArArGrUrUrUrUrArGr | \|1107 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrGrGmGrCmGrUmGrGmCmU*m | |
| | U*mU*mU | |
| ChemMod_Set2_20 | mU*mU*mG*rGrUrGrArCrUrGrCrCrArCrGrCrCrCrArArGrUrUrUrUrArGr | 1108 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrG/i2FU/ | |
| | rGrA/i2FA/rCrA/i2FC/rCrC/i2FC/rUrU/i2FG/rGmGrCmGrUmGrGmCm | |
| | U*mU*mU*mU | |
| ChemMod_Set2_21 | mU*mU*mG*rGrUrGrArCrUrGrCrCrArCrGrCrCrCrArArGrUrUrUrUrArGr | 1109 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmC/i2FG/rUrG/ | |
| | i2FA/rArC/i2FA/rCrC/i2FC/rCrU/i2FU/rGrGmGrCmGrUmGrGmCm | |
| | U*mU*mU*mU | |
| ChemMod_Set2_22 | mU*mU*mG*rGrUrGrArCrUrGrCrCrArCrGrCrCrCrArArGrUrUrUrUrArGr | 1110 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGmGrGmCrGmUrGmGrCmU*mU*mU*mU | |
| ChemMod_Set2_23 | mU*mU*mG*rGrUrGrArCrUrGrCrCrArCrGrCrCrCrArArGrUrUrUrUrArGr | 1111 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG | |
| | /rArA/i2FC/rArC/i2FC/rCrC/i2FU/rUrGmGrGmCrGmUrGmGrCmU*m | |
| | U*mU*mU | |
| ChemMod_Set2_24 | mU*mU*mG*rGrUrGrArCrUrGrCrCrArCrGrCrCrCrArArGrUrUrUrUrArGr | 1112 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrG/i2FU/ | |
| | rGrA/i2FA/rCrA/i2FC/rCrC/i2FC/rUrU/i2FG/mGrGmCrGmUrGmGrCm | |
| | U*mU*mU*mU | |
| ChemMod_Set2_25 | mU*mU*mG*rGrUrGrArCrUrGrCrCrArCrGrCrCrCrArArGrUrUrUrUrArGr | 1113 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmC/i2FG/rUrG/ | |
| | i2FA/rArC/i2FA/rCrC/i2FC/rCrU/i2FU/rGmGrGmCrGmUrGmGrCm | |
| | U*mU*mU*mU | |
| ChemMod_Set2_26 | mU*mU*mG*rGrUrGrArCrUrGrCrCrArCrGrCrCrCrArArGrUrUrUrUrArGr | 1114 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrG/i2 | |
| | FA/rArCrA/i2FC/rCrCrC/i2FU/rUrGrG/i2FG/rCrGrU/i2FG/rGrCmU*m | |
| | U*mU*mU | |
| ChemMod_Set2_27 | mU*mU*mG*rGrUrGrArCrUrGrCrCrArCrGrCrCrCrArArGrUrUrUrUrArGr | 1115 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrG/i2FG/rCrGrU/i2FG/rGrCmU*mU*mU*m | |
| | U | |
| ChemMod_Set2_28 | mU*mU*mG*rGrUrGrArCrUrGrCrCrArCrGrCrCrCrArArGrUrUrUrUrArGr | 1116 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrG/i2 | |
| | FA/rArCrA/i2FC/rCrCrC/i2FU/rUrGrGrGrCrGrUrGrGrCmU*mU*mU | |
| | *mU | |
| ChemMod_Set2_29 | mU*mU*mG*rGrUrGrArCrUrGrCrCrArCrGrCrCrCrArArGrUrUrUrUrArGr | 1117 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA/ | |
| | i2FA/rCrArCrC/i2FC/rCrUrUrG/i2FG/rGrCrGrU/i2FG/rGrCmU*mU* | |
| | mU*mU | |
| ChemMod_Set2_30 | mU*mU*mG*rGrUrGrArCrUrGrCrCrArCrGrCrCrCrArArGrUrUrUrUrArGr | 1118 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/rGrCmU*mU*mU*m | |
| | U | |
| ChemMod_Set2_31 | mU*mU*mG*rGrUrGrArCrUrGrCrCrArCrGrCrCrCrArArGrUrUrUrUrArGr | 1119 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA/ | |
| | i2FA/rCrArCrC/i2FC/rCrUrUrGrGrGrCrGrUrGrGrCmU*mU*mU*mU | |
| ChemMod_Set2_32 | rUrUrGrGrUrGrArCrUrGrCrCrArCrGrCrCrCrArArGrUrUrUrUrArGrArGrCr | 1120 |
| | UrArGrArArArUrArGrCrArArGrUrUrArArArArUrArArGrGrCrUrArGrUrCr | |
| | CrGrUrUrArUrCrArArCrUrUrGrArArArArArGrUrGrGrCrArCrCrGrArGrUr | |
| | CrGrGrUrGrCrGrUrGrArArCrArCrCrCrCrUrUrGrGrGrCrGrUrGrGr | |
| | CrUrUrUrU | |
| ChemMod_Set2_33 | mU*mU*mG*rGrUrGrArCrUrGrCrCrArCrGrCrCrCrArArGrUrUrUrUrArGr | 1121 |
| | ArGrCrUrArGrArArArUrArGrCrArArGrUrUrArArArArUrArArGrGrCrUrAr | |
| | GrUrCrCrGrUrUrArUrCrArArCrUrUrGrArArArArArGrUrGrGrCrArCrCrGr | |
| | ArGrUrCrGrGrUrGrCrGrUrGrArArCrArCrCrCrCrUrUrGrGrGrCrGrU | |
| | rGrGrCmU*mU*mU*mU | |
| ChemMod_Set2_34 | mU*mU*mG*rGrUrGrArCrUrGrCrCrArCrGrCrCrCrArArGrUrUrUrUrArGr | 1122 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrG/i2FG/rGrC/i2FG/rUrG/i2FG/ | |
| | rCmU*rU*rU*rU | |
| ChemMod_Set2_35 | mU*mU*mG*rGrUrGrArCrUrGrCrCrArCrGrCrCrCrArArGrUrUrUrUrArGr | 1123 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrG/i2FG/rGrC/i2FG/rUrG/i2FG/rC | |
| | mU*mU*rU*rU | |
| ChemMod_Set2_36 | mU*mU*mG*rGrUrGrArCrUrGrCrCrArCrGrCrCrCrArArGrUrUrUrUrArGr | 1124 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG | |
| | /rArA/i2FC/rArC/i2FC/rCrC/i2FU/rUrG/i2FG/rGrC/i2FG/rUrG/i2FG/rC | |
| | mU*mU*mU*rU | |
| ChemMod_Set2_37 | mU*mU*mG*rGrUrGrArCrUrGrCrCrArCrGrCrCrCrArArGrUrUrUrUrArGr | 1125 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG | |
| | /rArA/i2FC/rArC/i2FC/rCrC/i2FU/rUrG/i2FG/rGrC/i2FG/rUrG/i2FG/rC | |
| | mU*mU*mU | |
| ChemMod_Set2_38 | mU*mU*mG*rGrUrGrArCrUrGrCrCrArCrGrCrCrCrArArGrUrUrUrUrArGr | 1126 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG | |
| | /rArA/i2FC/rArC/i2FC/rCrC/i2FU/rUrG/i2FG/rGrC/i2FG/rUrG/i2FG/rC | |
| | rUrU*mU*mU*mUrU | |
| ChemMod_Set2_39 | mU*mU*mG*rGrUrGrArCrUrGrCrCrArCrGrCrCrCrArArGrUrUrUrUrArGr | 1127 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG | |
| | /rArA/i2FC/rArC/i2FC/rCrC/i2FU/rUrG/i2FG/rGrC/i2FG/rUrG/i2FG/rC | |
| | rUrUmU*mU*mU*rU | |
| ChemMod_Set2_40 | mU*mU*mG*rGrUrGrArCrUrGrCrCrArCrGrCrCrCrArArGrUrUrUrUrArGr | 1128 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG | |
| | /rArA/i2FC/rArC/i2FC/rCrC/i2FU/rUrG/i2FG/rGrC/i2FG/rUrG/i2FG/rC | |
| | rUmU*mU*mU*rUrU | |
| ChemMod_Set2_41 | mU*mU*mG*rGrUrGrArCrUrGrCrCrArCrGrCrCrCrArArGrUrUrUrUrArGr | 1129 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG/ | |
| | rArA/i2FC/rArC/i2FC/rCrC/i2FU/rUrG/i2FG/rGrC/i2FG/rUrG/i2FG/rC | |
| | mU*mU*mU*rUrUrU | |
| ChemMod_Set2_42 | mU*mU*mG*rGrUrGrArCrUrGrCrCrArCrGrCrCrCrArArGrUrUrUrUrArGr | 1130 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrU/i2FG | |
| | /rArA/i2FC/rArC/i2FC/rCrC/i2FU/rUrG/i2FG/rGrC/i2FG/rUrG/i2FG/rC | |
| | rU*rU*U*mU | |

¹For RNA, U are T in the sequence listing. For each entry, the spacer sequence is underlined and the extension arm is bold.
²For the sequences: *, phosphorothioate modification; mN, 2′O-methyl modification; rN, unmodified ribonucleotide; i2FN, Int Fluoro modification. A position lacking, e.g., r or m (or positions noted as T) are DNA positions within the RNA. N = A, C, T/U, G.

TABLE 39B
EXEMPLARY MODIFIED tagRNAS (ATP7B)

| NAME | SEQUENCE^{1,2} | SEQ ID NO: |
|---|---|---|
| ChemMod_Set3_1 | mU*mU*mG*rGrUrGrArCrUrGrCrCrArCrGrCrCrCrArArGrUrUrUrUrArGr | 1131 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGrUrGrG/i2FC/mU*mU*mU*mU | |
| ChemMod_Set3_2 | mU*mU*mG*rGrUrGrArCrUrGrCrCrArCrGrCrCrCrArArGrUrUrUrUrArGr | 1132 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGrUrG/i2FG/rCmU*mU*mU*mU | |
| ChemMod_Set3_3 | mU*mU*mG*rGrUrGrArCrUrGrCrCrArCrGrCrCrCrArArGrUrUrUrUrArGr | 1133 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGrU/i2FG/rGrCmU*mU*mU*mU | |
| ChemMod_Set3_4 | mU*mU*mG*rGrUrGrArCrUrGrCrCrArCrGrCrCrCrArArGrUrUrUrUrArGr | 1134 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrG/i2FU/rGrGrCmU*mU*mU*mU | |
| ChemMod_Set3_5 | mU*mU*mG*rGrUrGrArCrUrGrCrCrArCrGrCrCrCrArArGrUrUrUrUrArGr | 1135 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrC/i2FG/rUrGrGrCmU*mU*mU*mU | |
| ChemMod_Set3_6 | mU*mU*mG*rGrUrGrArCrUrGrCrCrArCrGrCrCrCrArArGrUrUrUrUrArGr | 1136 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrG/i2FC/rGrUrGrGrCmU*mU*mU*mU | |
| ChemMod_Set3_7 | mU*mU*mG*rGrUrGrArCrUrGrCrCrArCrGrCrCrCrArArGrUrUrUrUrArGr | 1137 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrG/i2FG/rCrGrUrGrGrCmU*mU*mU*mU | |
| ChemMod_Set3_8 | mU*mU*mG*rGrUrGrArCrUrGrCrCrArCrGrCrCrCrArArGrUrUrUrUrArGr | 1138 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrUrGrGrCmU*mU*mU*mU | |
| ChemMod_Set3_9 | mU*mU*mG*rGrUrGrArCrUrGrCrCrArCrGrCrCrCrArArGrUrUrUrUrArGr | 1139 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrU/i2FG/rGrGrCrGrUrGrGrCmU*mU*mU*mU | |
| ChemMod_Set3_10 | mU*mU*mG*rGrUrGrArCrUrGrCrCrArCrGrCrCrCrArArGrUrUrUrUrArGr | 1140 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrU/i2FG//i2FG//i2FG/rCrGrUrGrGrCmU*mU*mU | |
| | *mU | |
| ChemMod_Set3_11 | mU*mU*mG*rGrUrGrArCrUrGrCrCrArCrGrCrCrCrArArGrUrUrUrUrArGr | 1141 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrU/i2FG//i2FG/rGrCrGrUrGrGrCmU*mU*mU*m | |
| | U | |
| ChemMod_Set3_12 | mU*mU*mG*rGrUrGrArCrUrGrCrCrArCrGrCrCrCrArArGrUrUrUrUrArGr | 1142 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrU/i2FG/rG/i2FG/rCrGrUrGrGrCmU*mU*mU*m | |
| | U | |
| ChemMod_Set3_13 | mU*mU*mG*rGrUrGrArCrUrGrCrCrArCrGrCrCrCrArArGrUrUrUrUrArGr | 1143 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG//i2FG/rCrGrUrGrGrCmU*mU*mU*m | |
| | U | |
| ChemMod_Set3_14 | mU*mU*mG*rGrUrGrArCrUrGrCrCrArCrGrCrCrCrArArGrUrUrUrUrArGr | 1144 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrU/i2FG//i2FG//i2FG/rC/i2FG/rU/i2FG//i2FG/rCm | |
| | U*mU*mU*mU | |
| ChemMod_Set3_15 | mU*mU*mG*rGrUrGrArCrUrGrCrCrArCrGrCrCrCrArArGrUrUrUrUrArGr | 1145 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG//i2FG/rC/i2FG/rU/i2FG//i2FG/rCmU*m | |
| | U*mU*mU | |
| ChemMod_Set3_16 | mU*mU*mG*rGrUrGrArCrUrGrCrCrArCrGrCrCrCrArArGrUrUrUrUrArGr | 1146 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/rGmCmU*mU*mU*m | |
| | U | |
| ChemMod_Set3_17 | mU*mU*mG*rGrUrGrArCrUrGrCrCrArCrGrCrCrCrArArGrUrUrUrUrArGr | 1147 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/mGmCmU*mU*mU* | |
| | mU | |
| ChemMod_Set3_18 | mU*mU*mG*rGrUrGrArCrUrGrCrCrArCrGrCrCrCrArArGrUrUrUrUrArGr | 1148 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGmU/i2FG/mGmCmU*mU*mU* | |
| | mU | |
| ChemMod_Set3_19 | mU*mU*mG*rGrUrGrArCrUrGrCrCrArCrGrCrCrCrArArGrUrUrUrUrArGr | 1149 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCmGmU/i2FG/mGmCmU*mU*mU | |
| | *mU | |
| ChemMod_Set3_20 | mU*mU*mG*rGrUrGrArCrUrGrCrCrArCrGrCrCrCrArArGrUrUrUrUrArGr | 1150 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGmCmGmU/i2FG/mGmCmU*mU*mU | |
| | *mU | |
| ChemMod_Set3_21 | mU*mU*mG*rGrUrGrArCrUrGrCrCrArCrGrCrCrCrArArGrUrUrUrUrArGr | 1151 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/mGmCmGmU/i2FG/mGmCmU*mU*m | |
| | U*mU | |
| ChemMod_Set3_22 | mU*mU*mG*rGrUrGrArCrUrGrCrCrArCrGrCrCrCrArArGrUrUrUrUrArGr | 1152 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/mGrCmU*mU*mU*m | |
| | U | |
| ChemMod_Set3_23 | mU*mU*mG*rGrUrGrArCrUrGrCrCrArCrGrCrCrCrArArGrUrUrUrUrArGr | 1153 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGmU/i2FG/rGrCmU*mU*mU*m | |
| | U | |
| ChemMod_Set3_24 | mU*mU*mG*rGrUrGrArCrUrGrCrCrArCrGrCrCrCrArArGrUrUrUrUrArGr | 1154 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCmGrU/i2FG/rGrCmU*mU*mU*m | |
| | U | |
| ChemMod_Set3_25 | mU*mU*mG*rGrUrGrArCrUrGrCrCrArCrGrCrCrCrArArGrUrUrUrUrArGr | 1155 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGmCrGrU/i2FG/rGrCmU*mU*mU*m | |
| | U | |
| ChemMod_Set3_26 | mU*mU*mG*rGrUrGrArCrUrGrCrCrArCrGrCrCrCrArArGrUrUrUrUrArGr | 1156 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/mGrCrGrU/i2FG/rGrCmU*mU*mU*m | |
| | U | |
| ChemMod_Set3_27 | mU*mU*mG*rGrUrGrArCrUrGrCrCrArCrGrCrCrCrArArGrUrUrUrUrArGr | 1157 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUGrAr | |
| | ACrArCCrCrCTrUrG/i2FG/rGrCrGrU/i2FG/rGrCmU*mU*mU*mU | |
| ChemMod_Set3_28 | mU*mU*mG*rGrUrGrArCrUrGrCrCrArCrGrCrCrCrArArGrUrUrUrUrArGr | 1158 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/rGrCmU*mU*mU*m | |
| | U*mU | |
| ChemMod_Set3_29 | mU*mU*mG*rGrUrGrArCrUrGrCrCrArCrGrCrCrCrArArGrUrUrUrUrArGr | 1159 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/rGrCmU*mU*mU*m | |
| | U*mU*mU | |
| ChemMod_Set3_30 | mU*mU*mG*rGrUrGrArCrUrGrCrCrArCrGrCrCrCrArArGrUrUrUrUrArGr | 1160 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/rGrCmU*mU*mU*m | |
| | U*mU*mU*mU | |

¹For RNA, U are T in the sequence listing. For each entry, the spacer sequence is underlined and the extension arm is bold.
²For the sequences: *, phosphorothioate modification; mN, 2′O-methyl modification; rN, unmodified ribonucleotide; i2FN, Int Fluoro modification. A position lacking, e.g., r or m (or positions noted as T) are DNA positions within the RNA. N = A ,C, T/U, G.

TABLE 39C
EXEMPLARY MODIFIED tagRNAS (ATP7B)

| NAME | SEQUENCE^{1,2} | SEQ ID NO: |
|---|---|---|
| Set4_v1 | mU*mU*mG*rGrUrGrArCrUrGrCrCrArCrGrCrCrCrArArGrUrUrUrUrArGr | 1161 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGrUrGrG/i2MOErC/mU*mU*mU* | |
| | mU | |
| Set4_v2 | mU*mU*mG*rGrUrGrArCrUrGrCrCrArCrGrCrCrCrArArGrUrUrUrUrArGr | 1162 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGrUrG/i2MOErG/rCmU*mU*mU* | |
| | mU | |
| Set4_v3 | mU*mU*mG*rGrUrGrArCrUrGrCrCrArCrGrCrCrCrArArGrUrUrUrUrArGr | 1163 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGrU/i2MOErG/rGrCmU*mU*mU* | |
| | mU | |
| Set4_v4 | mU*mU*mG*rGrUrGrArCrUrGrCrCrArCrGrCrCrCrArArGrUrUrUrUrArGr | 1164 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrG/i2MOErT/rGrGrCmU*mU*mU*m | |
| | U | |
| Set4_v5 | mU*mU*mG*rGrUrGrArCrUrGrCrCrArCrGrCrCrCrArArGrUrUrUrUrArGr | 1165 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrC/i2MOErG/rUrGrGrCmU*mU*mU* | |
| | mU | |
| Set4_v6 | mU*mU*mG*rGrUrGrArCrUrGrCrCrArCrGrCrCrCrArArGrUrUrUrUrArGr | 1166 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrG/i2MOErC/rGrUrGrGrCmU*mU*mU* | |
| | mU | |
| Set4_v7 | mU*mU*mG*rGrUrGrArCrUrGrCrCrArCrGrCrCrCrArArGrUrUrUrUrArGr | 1167 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrG/i2MOErG/rCrGrUrGrGrCmU*mU*mU* | |
| | mU | |
| Set4_v8 | mU*mU*mG*rGrUrGrArCrUrGrCrCrArCrGrCrCrCrArArGrUrUrUrUrArGr | 1168 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2MOErG/rGrCrGrUrGrGrCmU*mU*mU* | |
| | mU | |
| Set4_v9 | mU*mU*mG*rGrUrGrArCrUrGrCrCrArCrGrCrCrCrArArGrUrUrUrUrArGr | 1169 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2MOErG/rGrCrGrU/i2MOErG/rGrCmU*m | |
| | U*mU*mU | |
| Set4_v10 | mU*mU*mG*rGrUrGrArCrUrGrCrCrArCrGrCrCrCrArArGrUrUrUrUrArGr | 1170 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGrUrGrGCmU*mU*mU*mU | |
| Set4_v11 | mU*mU*mG*rGrUrGrArCrUrGrCrCrArCrGrCrCrCrArArGrUrUrUrUrArGr | 1171 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGrUrGGrCmU*mU*mU*mU | |
| Set4_v12 | mU*mU*mG*rGrUrGrArCrUrGrCrCrArCrGrCrCrCrArArGrUrUrUrUrArGr | 1172 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGrUGrGrCmU*mU*mU*mU | |
| Set4_v13 | mU*mU*mG*rGrUrGrArCrUrGrCrCrArCrGrCrCrCrArArGrUrUrUrUrArGr | 1173 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrGTrGrGrCmU*mU*mU*mU | |
| Set4_v14 | mU*mU*mG*rGrUrGrArCrUrGrCrCrArCrGrCrCrCrArArGrUrUrUrUrArGr | 1174 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCGrUrGrGrCmU*mU*mU*mU | |
| Set4_v15 | mU*mU*mG*rGrUrGrArCrUrGrCrCrArCrGrCrCrCrArArGrUrUrUrUrArGr | 1175 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGCrGrUrGrGrCmU*mU*mU*mU | |
| Set4_v16 | mU*mU*mG*rGrUrGrArCrUrGrCrCrArCrGrCrCrCrArArGrUrUrUrUrArGr | 1176 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGGrCrGrUrGrGrCmU*mU*mU*mU | |
| Set4_v17 | mU*mU*mG*rGrUrGrArCrUrGrCrCrArCrGrCrCrCrArArGrUrUrUrUrArGr | 1177 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGGrGrCrGrUrGrGrCmU*mU*mU*mU | |
| Set4_v18 | mU*mU*mG*rGrUrGrArCrUrGrCrCrArCrGrCrCrCrArArGrUrUrUrUrArGr | 1178 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGGrGrCrGrUGrGrCmU*mU*mU*mU | |
| Set4_v19 | mU*mU*mG*rGrUrGrArCrUrGrCrCrArCrGrCrCrCrArArGrUrUrUrUrArGr | 1179 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrG/i2FU//i2FG/rGrCmU*mU*mU | |
| | *mU | |
| Set4_v20 | mU*mU*mG*rGrUrGrArCrUrGrCrCrArCrGrCrCrCrArArGrUrUrUrUrArGr | 1180 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCmG/i2FU//i2FG/mGmCmU*mU*m | |
| | U*mU | |
| Set4_v21 | mU*mU*mG*rGrUrGrArCrUrGrCrCrArCrGrCrCrCrArArGrUrUrUrUrArGr | 1181 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrG/i2FU/rGrGrCmU*mU*mU*/3Invd | |
| | T/ | |
| Set4_v22 | mU*mU*mG*rGrUrGrArCrUrGrCrCrArCrGrCrCrCrArArGrUrUrUrUrArGr | 1182 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrG/i2FU/mG*mG*mC*/3InvdT/ | |
| Set4_v23 | mU*mU*mG*rGrUrGrArCrUrGrCrCrArCrGrCrCrCrArArGrUrUrUrUrArGr | 1183 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/rGrCmU*mU*mU*/ | |
| | 3InvdT/ | |
| Set4_v24 | mU*mU*mG*rGrUrGrArCrUrGrCrCrArCrGrCrCrCrArArGrUrUrUrUrArGr | 1184 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/mGmC*mU*mU*/ | |
| | 3InvdT/ | |
| Set4_v25 | mU*mU*mG*rGrUrGrArCrUrGrCrCrArCrGrCrCrCrArArGrUrUrUrUrArGr | 1185 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/mG*mC*mU*/3InvdT/ | |
| Set4_v26 | mU*mU*mG*rGrUrGrArCrUrGrCrCrArCrGrCrCrCrArArGrUrUrUrUrArGr | 1186 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrG/i2FU//i2FG/*mG*mC*/3InvdT/ | |
| Set4_v27 | mU*mU*mG*rGrUrGrArCrUrGrCrCrArCrGrCrCrCrArArGrUrUrUrUrArGr | 1187 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/*mG*mC*/3InvdT/ | |
| Set4_v28 | mU*mU*mG*rGrUrGrArCrUrGrCrCrArCrGrCrCrCrArArGrUrUrUrUrArGr | 1188 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/mG*mC*/3InvdT/ | |
| Set4_v29 | mU*mU*mG*rGrUrGrArCrUrGrCrCrArCrGrCrCrCrArArGrUrUrUrUrArGr | 1189 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/rGmC*mU*mU*mU | |
| Set4_v30 | mU*mU*mG*rGrUrGrArCrUrGrCrCrArCrGrCrCrCrArArGrUrUrUrUrArGr | 1190 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/mG*mC*mU*mU | |
| Set4_v31 | mU*mU*mG*rGrUrGrArCrUrGrCrCrArCrGrCrCrCrArArGrUrUrUrUrArGr | 1191 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGrU/i2FG/*mG*mC*mU | |
| Set4_v32 | mU*mU*mG*rGrUrGrArCrUrGrCrCrArCrGrCrCrCrArArGrUrUrUrUrArGr | 1192 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/rGrCrGmU*/i2FG/*mG*mC | |
| Set4_v33 | mU*mU*mG*rGrUrGrArCrUrGrCrCrArCrGrCrCrCrArArGrUrUrUrUrArGr | 1193 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCrG/i2FU/mGmGmCmU*mU*mU*mU | |
| Set4_v34 | mU*mU*mG*rGrUrGrArCrUrGrCrCrArCrGrCrCrCrArArGrUrUrUrUrArGr | 1194 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGrCmG/i2FU/mGmGmCmU*mU*mU*m | |
| | U | |
| Set4_v35 | mU*mU*mG*rGrUrGrArCrUrGrCrCrArCrGrCrCrCrArArGrUrUrUrUrArGr | 1195 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGrGmCrG/i2FU/rGrGrCmU*mU*mU*mU | |
| Set4_v36 | mU*mU*mG*rGrUrGrArCrUrGrCrCrArCrGrCrCrCrArArGrUrUrUrUrArGr | 1196 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGrGmGrCrG/i2FU/rGrGrCmU*mU*mU*mU | |
| Set4_v37 | mU*mU*mG*rGrUrGrArCrUrGrCrCrArCrGrCrCrCrArArGrUrUrUrUrArGr | 1197 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrGmGrGrCrG/i2FU/rGrGrCmU*mU*mU*mU | |
| Set4_v38 | mU*mU*mG*rGrUrGrArCrUrGrCrCrArCrGrCrCrCrArArGrUrUrUrUrArGr | 1198 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG//i2FG/rCrGmUmGmGmCmU*mU*mU | |
| | *mU | |
| Set4_v39 | mU*mU*mG*rGrUrGrArCrUrGrCrCrArCrGrCrCrCrArArGrUrUrUrUrArGr | 1199 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG//i2FG/rCrGmUrGmGmCmU*mU*mU* | |
| | mU | |
| Set4_v40 | mU*mU*mG*rGrUrGrArCrUrGrCrCrArCrGrCrCrCrArArGrUrUrUrUrArGr | 1200 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG//i2FG/rCmGmUmGmGmCmU*mU*mU | |
| | *mU | |
| Set4_v41 | mU*mU*mG*rGrUrGrArCrUrGrCrCrArCrGrCrCrCrArArGrUrUrUrUrArGr | 1201 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG//i2FG/rCmGmUrGmGmCmU*mU*mU | |
| | *mU | |
| Set4_v42 | mU*mU*mG*rGrUrGrArCrUrGrCrCrArCrGrCrCrCrArArGrUrUrUrUrArGr | 1202 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG//i2FG/mCrGrUrGrGrCmU*mU*mU*m | |
| | U | |
| Set4_v43 | mU*mU*mG*rGrUrGrArCrUrGrCrCrArCrGrCrCrCrArArGrUrUrUrUrArGr | 1203 |
| | AmGmCmUmAmGmAmAmAmUmAmGmCrArArGrUrUrArArArArUrArAr | |
| | GrGrCrUrArGrUrCrCrGrUrUrArUrCmAmAmCmUmUmGmAmAmAmAmA | |
| | mGmUmGmGmCmAmCmCmGmAmGmUmCmGmGmUmGmCrGrUrGrA | |
| | rArCrArCrCrCrCrUrUrG/i2FG/mGmCrGrU/i2FG/rGrCmU*mU*mU* | |
| | mU | |

¹For RNA, U are T in the sequence listing. For each entry, the spacer sequence is underlined and the extension arm is bold.
²For the sequences: *, phosphorothioate modification; mN, 2′O-methyl modification; rN, unmodified ribonucleotide; i2FN, Int Fluoro modification; i2MOErN, internal 2-O-Methyoxyethyl modification. A position lacking, e.g., r or m (or positions noted as T) are DNA positions within the RNA. N = A, C, T/U, G.

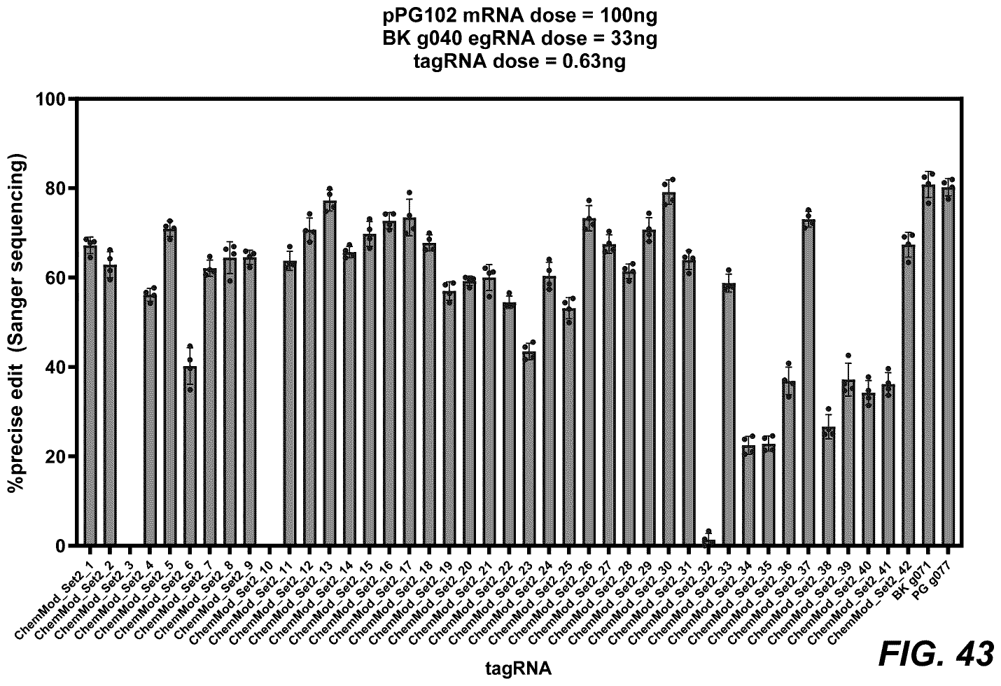

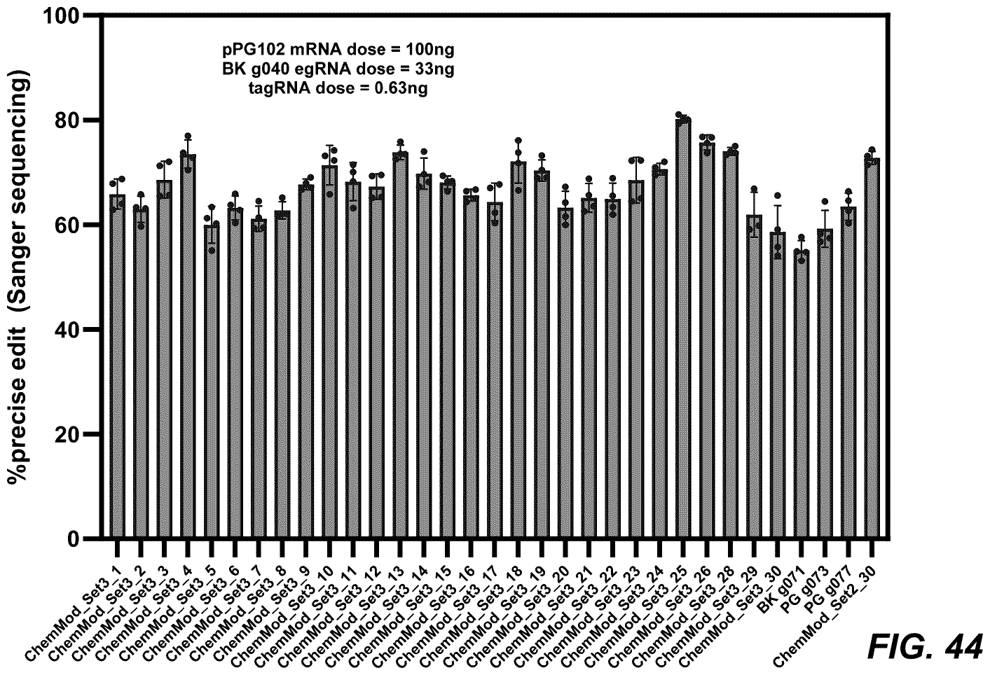

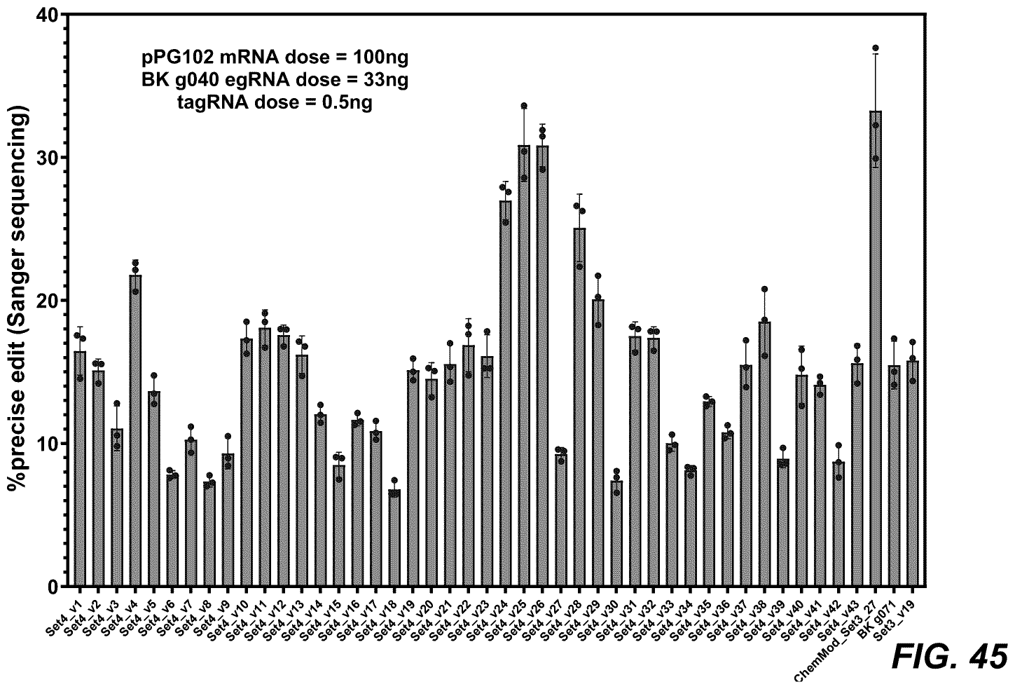

Editing efficiencies using the modified tagRNAs are shown in FIG. 43-FIG. 45.

### Example 12

### In Vivo Data

This Example provides exemplary in vivo data derived from studies of the disclosed compositions and methods.

### Wilson's Disease Mouse Model Study

LNP delivery of RT editing components was performed in mouse engineered with humanized ATP7B3 gene with the H1069Q mutation. Table 40 below displays components of the mouse studies.

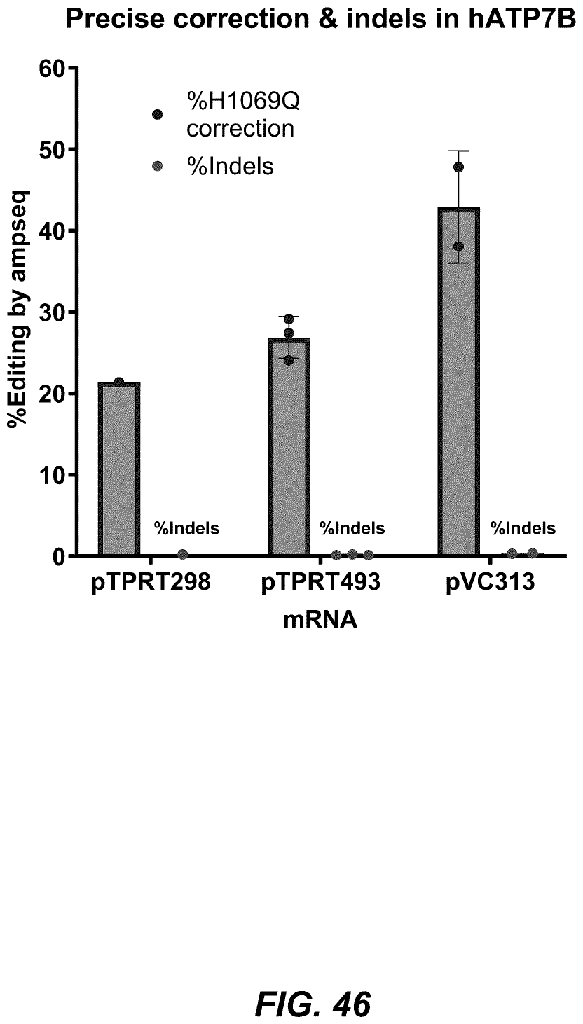

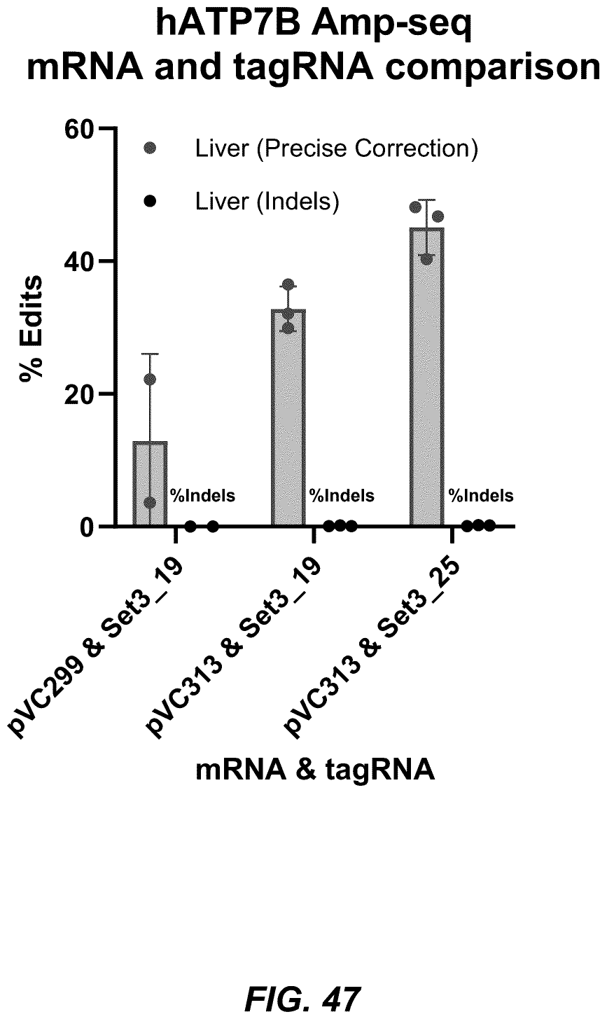

TABLE 40
MOUSE STUDY

| | |
|---|---|
| mRNA | |
| (name in FIG. 46-FIG. 47) | mRNA alias |

Study 1^{a}

| | |
|---|---|
| pTPRT298 | pTPRT-U-298; |
| (SEQ ID NO: 1041) | PKU-m001 mRNA |
| pTPRT493 | pTPRT-U-493; |
| (SEQ ID NO: 1043) | PKU-m003 mRNA |
| pVC313 | mRNA containing RV-UML-m007 |
| (SEQ ID NO: 939) | 5′UTR (see Table 30) |

Study 2^{b}

| | |
|---|---|
| pVC299 | Contains NCA7d 5′UTR, |
| (SEQ ID NO: 1204) | Seq ID NO: 910 in Table 29 |
| pVC313 | mRNA containing RV-UML-m007 |
| (SEQ ID NO: 939) | 5′UTR (see Table 30) |

^{a}tagRNA, ChemMod_Set3_19; egRNA, BK g040
^{b}tagRNA, ChemMod_Set3_19 or ChemMod_Set3_25; egRNA, BK g040

Table 41 below provides additional information regarding the mRNAs used in the mouse study shown in FIG. 46. In FIG. 46, mice were dosed at 2 mpk.

TABLE 41
mRNAs of MOUSE STUDY

| | pTPRT298* | pTPRT493* | PVC313 |
|---|---|---|---|
| 5′UTR | Custom 5′UTR | Syn_5′UTR | m007 |
| Cas9 | SpCas9 R221K | SpCas9 R221K | SpCas9 R221K |
| | N394K H840A | N394K H840A | N394K H840A |
| Linker | GS-bpNLS-GS | PAPAP-8 | GS-bpNLS-GS |
| RT | MMLV | MMLV | Myotis brandtii |
| 3′UTR | HBA1 | HBA1 | HBA1 |
| Stabilizing element | STH | WPRE | None |
| Poly A | Seg. PolyA4 | Seg. PolyA4 | Seg. PolyA4 |

*See PKU study above in Example 7

pVC313 mRNA and S3_19 tagRNA were used above, at 2 mpk (FIG. 46). As shown in FIG. 47, two mRNAs were compared. pVC299 has NCA7d 5′UTR and pVC313 has m007 5′UTR. Two tagRNAs were compared. S3_19 has four extra 2′OMe in the FBS, while S3_25 has one extra 2′OMe in the FBS (different position from S3_19 2′OMe positions). All 3 LNPs were dosed at 1 mpk. The results indicate that pVC313 was the most effective (˜56%) out of the three mRNAs in precise correction of the H1069Q mutation.

### PKU Disease Mouse Model Study

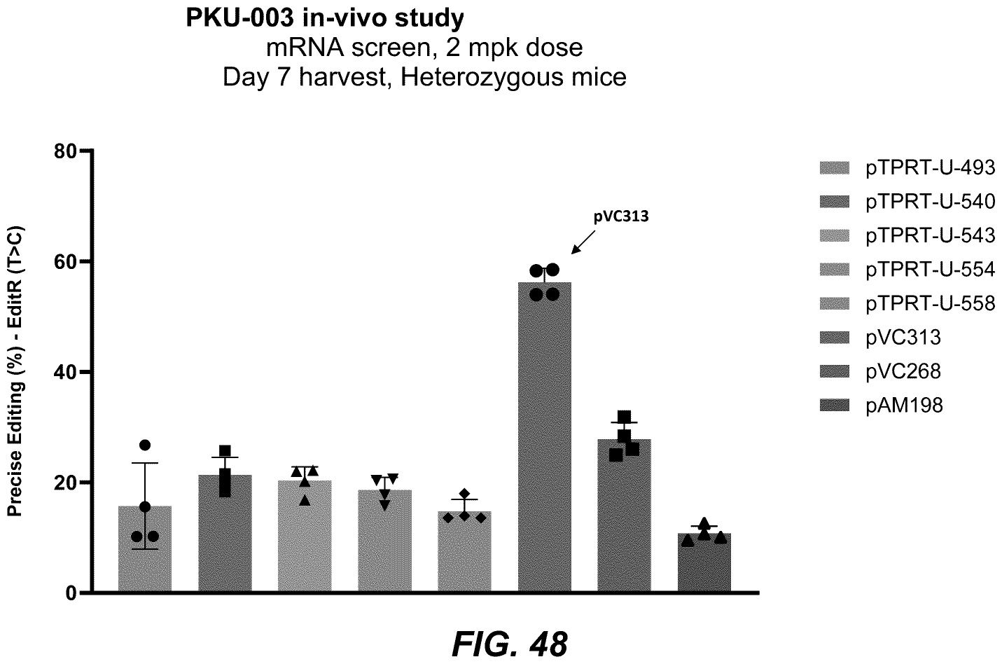

Next, corrections of the R408W mutation in the PAH gene (PAH-R408W) was tested in vivo. Table 42A shows some of the sequences used in this study. Precise correction of PAH-R408W in liver of humanized mice (het) at 2 mpk was assessed. In these experiments, the indicated mRNA with PKU-g007 NGG1 tagRNA and NGG1 (+37) egRNA was used. Delivery was performed using a lipid nanoparticle formulation (1.5% PEG, 1-step, acetate pH 4). The results indicate that pVC313, which also worked best for Wilson's Disease (see above), showed the best precise editing efficiency of the PAH-R408W mutation, demonstrating consistency across at least two indications (loci, spacers). Shown in Table 42B below and FIG. 48 are the results of the study.

TABLE 42A
mRNAs RELATED TO PKU MOUSE STUDY

| | Protein | mRNA |
|---|---|---|
| | Sequence | Sequence |
| Name | (SEQ ID NO:) | (SEQ ID NO:) |
| pTPRT-U-554 | 1326 | 1327 |
| pTPRT-U-558 | 1326 | 1328 |

TABLE 42B
PRECISE EDITING

| mRNA | % Integrity |
|---|---|
| pTPRT-U-493 | 73.1 |
| pTPRT-U-540 | 78.2 |
| pTPRT-U-543 | 75 |
| pTPRT-U-554 | 72.5 |
| pTPRT-U-558 | 78.6 |
| pVC313 | 84.1 |
| pVC268 | 80.8 |
| pAM198 | 85.6 |

### Example 13

### SpyCas9 gRNA Scaffolds

Described in this Example are SpyCas9 scaffold variants designed with different sequences and different chemical modification patterns. Below in Table 43 are the designed scaffolds.

TABLE 43
EXEMPLARY MODIFIED tagRNAS (ATP7B)

| NAME | SEQUENCE^{1,2} | SEQ ID NO: |
|---|---|---|
| D00 | rGrUrUrUrUrArGrAmGmCmUmAmGmAmAmAmUmAmGmCrArArGrUrUrArArAr | 1210 |
| | ArUrArArGrGrCrUrArGrUrCrCrGrUrUrArUrCmAmAmCmUmUmGmAmAmAmAm | |
| | AmGmUmGmGmCmAmCmCmGmAmGmUmCmGmGmUmGmC | |
| D01 | rGrUrUrUrUrArGrArGrCrUrArGrArArArUrArGrCrArArGrUrUrArArArArUrArArGr | 1211 |
| | GrCrUrArGrUrCrCrGrUrUrArUrCrArArCrUrUrGrArArArArArGrUrGrGrCrArCrCrG | |
| | rArGrUrCrGrGrUrGrC | |
| D02 | rGrUrUrUrUrArGrAmGmCmUmAmGmAmAmAmUmAmGmCrArArGrUrUrArArAr | 1212 |
| | ArUrArArGrGrCrUrArGrUrCrCrGrUrUrArUrCrAmAmCmUmUmGmAmAmAmAm | |
| | AmGmUmGmGmCmAmCmCmGmAmGmUmCmGmGmUmGmC | |
| D03 | rGrUrUrUrUrAmGmAmGmCmUmAmGmAmAmAmUmAmGrCrArArGrUrUmArAr | 1213 |
| | ArArUrArArGmGrCrUmArGrUrCmCrGrUrUrArUrCrAmAmCmUmUmGmAmAmA | |
| | mAmAmGmUrGrGrCrAmCmCmGrArGrUrCrGmGmUmGmC | |
| D04 | rGrUrUrUrUrArGrAmGmCmUmAmGmAmAmAmUmAmGmCrArArGrUrUmArArA | 1214 |
| | rArUrArArGmGrCrUmArGrUrCmCrGrUrUrArUrCrAmAmCmUmUmGmAmAmAm | |
| | AmAmGmUrGrGrCrAmCmCmGrArGrUrCrGmGmUmGmC | |
| D05 | mGrUrUrUrUrAmGmAmGmCmUmAmGmAmAmAmUmAmGrCrArArGrUrUmArAr | 1215 |
| | ArArUrArArGmGrCrUmArGrUrCmCrGrUrUrArUrCrAmAmCmUmUmGmAmAmA | |
| | mAmAmGmUrGrGrCrAmCmCmGrArGrUrCrGmGmUmGmC | |
| D06 | mGrUrUrUrUrArGrAmGmCmUmAmGmAmAmAmUmAmGmCrArArGrUrUmArAr | 1216 |
| | ArArUrArArGmGrCrUmArGrUrCmCrGrUrUrArUrCrAmAmCmUmUmGmAmAmA | |
| | mAmAmGmUrGrGrCrAmCmCmGrArGrUrCrGmGmUmGmC | |
| D07 | rGrUrUrUrUrArGrAmGmCmUmAmGmAmAmAmUmAmGrCrArArGrUrUrArArArA | 1217 |
| | rUrArArGrGrCrUrArGrUrCrCrGrUrUrArUrCrArAmCmUmUmGmAmAmAmAmAm | |
| | GmUrGrGmCmAmCmCmGmAmGmUrCmGmGmUmGmC | |
| D08 | rGrUrUrUrUrArGrAmGmCmUmAmGmAmAmAmUmAmGrCrArArGrUrUrArArArA | 1218 |
| | rUrArArGrGrCrUrArGrUrCrCrGrUrUrArUrCrArAmCmUmUmGmAmAmAmAmAm | |
| | GmUmGrGmCmAmCmCmGmAmGmUmCmGmGmUmGmC | |
| D09 | mGrUrUrUrUrArGmAmGmCmUmAmGmAmAmAmUmAmGmCmAmArGrUrUmAr | 1219 |
| | AmArArUrAmAmGmGmCmUmArGrUmCmCrGrUrUrAmUmCrArAmCmUmUmGm | |
| | AmAmAmAmAmGmUmGrGmCmAmCmCmGmAmGmUmCmGmGmUmGmC | |
| D10 | rGrUrUrUrUrArGrAmGmCmUmAmGmAmAmAmUmAmGmCrArArGrUrUmArArA | 1220 |
| | rArUrArArGmGrCrUmArGrUrCmCrGrUrUrArUrCrArAmCmUmUmGmAmAmAmA | |
| | mAmGmUmGrGmCmAmCmCmGmAmGmUmCmGmGmUmGmC | |
| D11 | rGrUrUrUrUrArGrAmGmCmUmAmGmAmAmAmUmAmGrCrArArGrUrUmArArAr | 1221 |
| | ArUrArArGmGrCrUmArGrUrCmCrGrUrUrArUrCrArAmCmUmUmGmAmAmAmA | |
| | mAmGmUmGrGmCmAmCmCmGmAmGmUmCmGmGmUmGmC | |
| D12 | mGrUrUrUrUrArGrAmGmCmUmAmGmAmAmAmUmAmGmCrArArGrUrUmArAr | 1222 |
| | ArArUrArArGmGrCrUmArGrUrCmCrGrUrUrArUrCrArAmCmUmUmGmAmAmAm | |
| | AmAmGmUmGrGmCmAmCmCmGmAmGmUmCmGmGmUmGmC | |
| D13 | mGrUrUrUrUrArGrAmGmCmUmAmGmAmAmAmUmAmGrCrArArGrUrUmArArA | 1223 |
| | rArUrArArGmGrCrUmArGrUrCmCrGrUrUrArUrCrArAmCmUmUmGmAmAmAmA | |
| | mAmGmUmGrGmCmAmCmCmGmAmGmUmCmGmGmUmGmC | |
| D14 | rGrUrUrUrUrArGrAmGmCmUmAmGmAmAmAmUmAmGmCrArArGrUrUrArArAr | 1224 |
| | ArUrArArGrGrCrUrArGrUrCrCrGrUrUrArUrCrArAmCmUmUmGmAmAmAmAmA | |
| | mGrUrGmGmCmAmCmCmGmAmGmUmCmGmGmUmGmC | |
| D15 | rGrUrUrUrUrArGrArGmCmUmAmGmAmAmAmUmAmGrCrArArGrUrUrArArArAr | 1225 |
| | UrArArGrGrCrUrArGrUrCrCrGrUrUrArUrCmAmAmCmUmUmGmAmAmAmAmA | |
| | mGmUmGmGmCmAmCmCmGmAmGmUmCmGmGmUmGmC | |
| D16 | rGrUrUrUrUrArGrAmGmCmUmAmGmAmAmAmUmAmGmCrArArGrUrUrArArAr | 1226 |
| | ArUrArArGrGrCrUrArGrUrCrCrGrUrUrArUrCmAmAmCmUmUmGmAmAmAmAm | |
| | AmGmUmGmGmCmAmCmCmGrArGrUrCrGmGmUmGmC | |
| D17 | rGrUrUrUrUrArGrArGmCmUmAmGmAmAmAmUmAmGrCrArArGrUrUrArArArAr | 1227 |
| | UrArArGrGrCrUrArGrUrCrCrGrUrUrArUrCrArAmCmUmUmGmAmAmAmAmAm | |
| | GrUrGmGmCmAmCmCmGmAmGmUmCmGmGmUmGmC | |
| D18 | rGrUrUrUrUrArGrAmGmCmUmAmGmAmAmAmUmAmGmCrArArGrUrUrArArAr | 1228 |
| | ArUrArArGrGrCrUrArGrUrCrCrGrUrUrArUrCrArAmCmUmUmGmAmAmAmAmA | |
| | mGrUrGmGmCmAmCmCmGrArGrUrCrGmGmUmGmC | |
| D19 | rGrUrUrUrUrArGrArGmCmUmAmGmAmAmAmUmAmGrCrArArGrUrUrArArArAr | 1229 |
| | UrArArGrGrCrUrArGrUrCrCrGrUrUrArUrCrArAmCmUmUmGmAmAmAmAmAm | |
| | GrUrGmGmCmAmCmCmGrArGrUrCrGmGmUmGmC | |
| D20 | rGrUrUrUrUrArGrA/i2FG/mC/i2FU/mAmGmAmAmAmU/i2FA/mG/i2FC/rArArGrUr | 1230 |
| | UrArArArArUrArArGrGrCrUrArGrUrCrCrGrUrUrArUrC/i2FA/mA/i2FC/mU/i2FU/ | |
| | mGmAmAmA/i2FA/mA/i2FG/mU/i2FG/mG/i2FC/mA/i2FC/mC/i2FG/mAmGmU/i2FC/ | |
| | mG/i2FG/mU/i2FG/mC | |
| D21 | rGrUrUrUrUrArGrA/i2FG/mC/i2FU/mAmGmAmAmAmU/i2FA/mG/i2FC/rArArGrUr | 1231 |
| | UrArArArArUrArArGrGrCrUrArGrUrCrCrGrUrUrArUrC/i2FA/mA/i2FC/mU/i2FU/ | |
| | mGmAmAmA/i2FA/mA/i2FG/mU/i2FG/mG/i2FC/mA/i2FC/mC/i2FG//i2FA//12FG// | |
| | i2FU//i2FC/mG/i2FG/mU/i2FG/mC | |
| D26 | rGrUrUrUrUrArGrAmGmCmUmAmGmAmAmAmUmAmGrCrArArGrUrUrArArArA | 1232 |
| | rUrArArGrGrCrUrArGrUrCrCrGrUrUrArUrCrArAmCmUmUmGmAmAmAmAmAm | |
| | GrUrGmGrCrAmCmCmGrArGrUrCrGrGmUmGmC | |
| D27 | rGrUrUrUrUrArGrAmGmCmUmAmGmAmAmAmUmAmGrCrArArGrUrUrArArArA | 1233 |
| | rUrArArGmGmCrUrArGrUrCrCrGrUrUrArUrCrArAmCmUmUmGmAmAmAmAmA | |
| | mGrUrGmGrCrAmCmCmGrArGrUrCrGrGmUmGmC | |
| D28 | rGrUrUrUrUrArGrAmGmCmUmAmGmAmAmAmUmAmGrCrArArGrUrUrArArArA | 1234 |
| | rUrArArGrGrCrUrArGrUrCrCrGrUrUrArUrCrArAmCmUmUmGmAmAmArAmAmG | |
| | rUrGrGrCrArCmCrGrArGrUrCrGrGmUmGmC | |
| D29 | rGrUrUrUrUrArGrAmGmCmUmAmGmAmAmAmUmAmGrCrArArGrUrUrArArArA | 1235 |
| | rUrArArGmGmCrUrArGrUrCrCrGrUrUrArUrCrArAmCmUmUmGmAmAmArAmA | |
| | mGrUrGrGrCrArCmCrGrArGrUrCrGrGmUmGmC | |
| D30 | rGrUrUrUrUrArGrArGmCmUmAmGmAmAmAmUmAmGrCrArArGrUrUrArArArAr | 1236 |
| | UrArArGrGrCrUrArGrUrCrCrGrUrUrArUrCrArAmCmUmUmGmAmAmAmAmAm | |
| | GrUrGmGrCrAmCmCmGrArGrUrCrGrGmUmGmC | |
| D31 | rGrUrUrUrUrArGrArGmCmUmAmGmAmAmAmUmAmGrCrArArGrUrUrArArArAr | 1237 |
| | UrArArGmGmCrUrArGrUrCrCrGrUrUrArUrCrArAmCmUmUmGmAmAmAmAmA | |
| | mGrUrGmGrCrAmCmCmGrArGrUrCrGrGmUmGmC | |
| D32 | rGrUrUrUrUrArGrArGmCmUmAmGmAmAmAmUmAmGrCrArArGrUrUrArArArAr | 1238 |
| | UrArArGrGrCrUrArGrUrCrCrGrUrUrArUrCrArAmCmUmUmGmAmAmArAmAmGr | |
| | UrGrGrCrArCmCrGrArGrUrCrGrGmUmGmC | |
| D33 | rGrUrUrUrUrArGrArGmCmUmAmGmAmAmAmUmAmGrCrArArGrUrUrArArArAr | 1239 |
| | UrArArGmGmCrUrArGrUrCrCrGrUrUrArUrCrArAmCmUmUmGmAmAmArAmAm | |
| | GrUrGrGrCrArCmCrGrArGrUrCrGrGmUmGmC | |
| D34 | rGrUrUrUrUrArGrAmGmCmUmAmGmAmAmAmUmAmGrCrArArGrUrUrArArArA | 1240 |
| | rUrArArGrGrCrUrArGrUrCrCrGrUrUrArUrCrArAmCmUmUmGmAmArArAmArGr | |
| | UrGrGrCrArCmCrGrArGrUrCrGrGrUrGmC | |
| D35 | rGrUrUrUrUrArGrAmGmCmUmAmGmAmAmAmUmAmGrCrArArGrUrUrArArArA | 1241 |
| | rUrArArGmGmCrUrArGrUrCrCrGrUrUrArUrCrArAmCmUmUmGmAmArArAmArG | |
| | rUrGrGrCrArCmCrGrArGrUrCrGrGrUrGmC | |
| d00_seq1 | rGrUrUrUrUrArGrAmGmCmUmAmGmAmAmAmUmAmGmCrArArGrUrUrArArAr | 1242 |
| | ArUrArArGrGrCrUrArGrUrCrCrGrUrUrCrUrCmAmAmCmGmGmAmAmAmCmCm | |
| | GmUmGmGmCmAmCmCmGmAmGmUmCmGmGmUmGmC | |
| d00_seq2 | rGrUrUrUrUrGrGrAmCmCmUmAmGmAmAmAmUmAmGmGrArCrGrUrCrArArAr | 1243 |
| | ArUrArArGrCrCrUrArGrUrGrCrGrUrGrCrUrCmAmAmCmCmUmGmAmAmAmUm | |
| | GmGmUmGmGmCmAmCmCmGmAmGmUmCmGmGmUmGmC | |
| d00_seq3 | rGrUrUrUrUrArGrCmGmCmCmAmAmAmAmAmAmGmGmCrArArGrUrUrArArAr | 1244 |
| | ArUrArArGrGrCrGrArGrUrCrCrGrCrCrArCrCmAmAmCmCmUmGmAmAmAmCm | |
| | GmGmUmGmGmCmAmCmCmGmAmGmUmCmGmGmUmGmC | |
| d00_seq4 | rGrUrUrUrUrArGrCmGmCmCmAmAmAmAmAmAmAmGmCrArArGrUrUrArArAr | 1245 |
| | ArUrArArGrGrCrGrArGrUrCrCrGrCrUrArUrCmAmAmCmCmUmGmAmAmAmCm | |
| | GmGmUmGmGmCmAmCmCmGmAmGmUmCmGmGmUmGmC | |
| d00_seq5 | rGrUrUrUrUrArUrAmGmCmUmAmGmCmAmAmUmAmGmCrArArGrUrUrArArAr | 1246 |
| | ArUrArArGrGrCrUrArGrUrCrCrGrUrUrArUrGmAmAmCmGmUmGmAmAmAmCm | |
| | CmGmUmGmGmCmAmCmCmGmAmGmUmCmGmGmUmGmC | |
| d00_seq6 | rGrUrUrUrUrArCrAmGmCmUmAmGmAmGmAmUmAmGmCrArArGrUrUrArArAr | 1247 |
| | ArUrArArGrGrCrUrArGrUrUrCrGrUrUrArCrCmAmAmCmGmAmGmAmAmCmAm | |
| | CmGmUmGmGmCmAmCmCmGmAmGmUmCmGmGmUmGmC | |
| d00_seq7 | rGrUrArUrUrCrGrAmGmUmCmAmGmAmAmAmUmGmGmCrArCrGrUrGrArArUr | 1248 |
| | ArUrArArGrArCrUrArGrUrUrCrGrUrArCrUrCmAmAmCmUmGmGmCmAmAmGm | |
| | CmGmUmGmGmCmAmCmCmGmAmGmUmCmGmGmUmGmC | |
| N1_D00 | rGrUrUrUrUrArGrAmGmCmUmGmGmAmAmAmCmAmGmCrArArGrUrUrArArAr | 1249 |
| | ArUrArArGrGrCrUrArGrUrCrCrGrUrUrArUrCmAmAmCmUmUmGmAmAmAmAm | |
| | AmGmUmGmGmCmAmCmCmGmAmGmUmCmGmGmUmGmC | |
| N2_D00 | rGrUrUrUrUrArGrAmGmCmUmGmAmGmAmAmAmUmCmArGrCrArArGrUrUrAr | 1250 |
| | ArArArUrArArGrGrCrUrArGrUrCrCrGrUrUrAmUmCmAmAmCmUmUmGmAmAm | |
| | AmAmAmGmUmGmGmCmAmCmCmGmAmGmUmCmGmGmUrGrC | |
| N3_D00 | rGrUrUrUrUrArGrAmGmCmUmAmGmAmAmAmUmAmGmCrArArGrUrUrArArAr | 1251 |
| | ArUrArArGrGrCrUrArGrUrCrCrGrUrUrArUrCmAmAmCmUmGmUmGmAmAmAm | |
| | AmCmAmGmUmGmGmCmAmCmCmGmAmGmUmCmGmGmUrGrC | |
| N4_D00 | rGrUrUrUrUrArGrAmGmCmUmAmGmAmAmAmUmAmGmCrArArGrUrUrArArAr | 1252 |
| | ArUrArArGrGrCrUrArGrUrCrCrGrUrUrArUrCmAmAmCmUmGmGmUmGmAmAm | |
| | AmAmCmCmAmGmUmGmGmCmAmCmCmGmAmGmUmCmGrGrUrGrC | |
| N5_D00 | rGrUrUrUrUrArGrAmGmCmUmGmAmGmAmAmAmUmCmArGrCrArArGrUrUrAr | 1253 |
| | ArArArUrArArGrGrCrUrArGrUrCrCrGrUrUrAmUmCmAmAmCmUmGmUmGmAm | |
| | AmAmAmCmAmGmUmGmGmCmAmCmCmGmAmGmUmCmGrGrUrGrC | |
| N6_D00 | rGrUrUrUrUrArGrAmGmCmUmGmGmAmAmAmCmAmGmCrArArGrUrUrArArAr | 1254 |
| | ArUrArArGrGrCrUrArGrUrCrCrGrUrUrArUrCmAmAmCmUmGmUmGmAmAmAm | |
| | AmCmAmGmUmGmGmCmAmCmCmGmAmGmUmCmGmGmUrGrC | |
| PG1 | rGrUrUrUrUrArGrAmGmCmUmAmGmAmAmAmUmAmGmCrArArGrUrUrArArAr | 1255 |
| | ArUrArArGrGrCrUrArGrUrCrCrGrUrUrArUrCmAmAmCmUmGmUmGmAmAmAm | |
| | AmCmAmGmUmGmGmCmAmCmCmGmAmGmUmCmGmGmUmGmC | |
| PG2 | rGrUrUrUrUrArGrAmGmCmUmAmGmAmAmAmUmAmGrCrArArGrUrUrArArArA | 1256 |
| | rUrArArGrGrCrUrArGrUrCrCrGrUrUrArUrCrArAmCmUmGmUmGmAmAmAmAm | |
| | CmAmGmUrGrGmCmAmCmCmGmAmGmUrCmGmGmUmGmC | |
| PG3 | rGrUrUrUrUrArGrAmGmCmUmAmGmAmAmAmUmAmGrCrArArGrUrUmArArAr | 1257 |
| | ArUrArArGmGrCrUmArGrUrCmCrGrUrUrArUrCrArAmCmUmGmUmGmAmAmA | |
| | mAmCmAmGmUmGrGmCmAmCmCmGmAmGmUmCmGmGmUmGmC | |
| PG4 | rGrUrUrUrUrArGrAmGmCmUmAmGmAmAmAmUmAmGmCrArArGrUrUrArArAr | 1258 |
| | ArUrArArGrGrCrUrArGrUrCrCrGrUrUrArUrCrArAmCmUmGmUmGmAmAmAmA | |
| | mCmAmGrUrGmGmCmAmCmCmGrArGrUrCrGmGmUmGmC | |
| PG5 | rGrUrUrUrUrArGrArGmCmUmAmGmAmAmAmUmAmGrCrArArGrUrUrArArArAr | 1259 |
| | UrArArGrGrCrUrArGrUrCrCrGrUrUrArUrCrArAmCmUmGmUmGmAmAmAmAm | |
| | CmAmGrUrGmGmCmAmCmCmGrArGrUrCrGmGmUmGmC | |
| PG6 | rGrUrUrUrUrArGrAmGmCmUmAmGmAmAmAmUmAmGrCrArArGrUrUrArArArA | 1260 |
| | rUrArArGrGrCrUrArGrUrCrCrGrUrUrArUrCrArAmCmUmGmUmGmAmAmAmAm | |
| | CmAmGrUrGmGrCrAmCmCmGrArGrUrCrGrGmUmGmC | |
| PG7 | rGrUrUrUrUrArGrAmGmCmUmAmGmAmAmAmUmAmGrCrArArGrUrUrArArArA | 1261 |
| | rUrArArGmGmCrUrArGrUrCrCrGrUrUrArUrCrArAmCmUmGmUmGmAmAmAmA | |
| | mCmAmGrUrGmGrCrAmCmCmGrArGrUrCrGrGmUmGmC | |
| PG8 | rGrUrUrUrUrArGrAmGmCmUmAmGmAmAmAmUmAmGrCrArArGrUrUrArArArA | 1262 |
| | rUrArArGrGrCrUrArGrUrCrCrGrUrUrArUrCrArAmCmUmGmUmGmAmAmArAmC | |
| | mAmGrUrGrGrCrArCmCrGrArGrUrCrGrGmUmGmC | |
| PG9 | rGrUrUrUrUrArGrArGmCmUmAmGmAmAmAmUmAmGrCrArArGrUrUrArArArAr | 1263 |
| | UrArArGmGmCrUrArGrUrCrCrGrUrUrArUrCrArAmCmUmGmUmGmAmAmAmA | |
| | mCmAmGrUrGmGrCrAmCmCmGrArGrUrCrGrGmUmGmC | |
| PG10 | rGrUrUrUrUrArGrArGmCmUmAmGmAmAmAmUmAmGrCrArArGrUrUrArArArAr | 1264 |
| | UrArArGmGmCrUrArGrUrCrCrGrUrUrArUrCrArAmCmUmGmUmGmAmAmArAm | |
| | CmAmGrUrGrGrCrArCmCrGrArGrUrCrGrGmUmGmC | |
| PG11 | rGrUrUrUrUrArGrAmGmCmUmAmGmAmAmAmUmAmGrCrArArGrUrUrArArArA | 1265 |
| | rUrArArGrGrCrUrArGrUrCrCrGrUrUrArUrCrArAmCmUmGmUmGmAmArArAmC | |
| | mArGrUrGrGrCrArCmCrGrArGrUrCrGrGrUrGmC | |
| PG12 | rGrUrUrUrUrArGrAmGmCmUmAmGmAmAmAmUmAmGrCrArArGrUrUrArArArA | 1266 |
| | rUrArArGmGmCrUrArGrUrCrCrGrUrUrArUrCrArAmCmUmGmUmGmAmArArAm | |
| | CmArGrUrGrGrCrArCmCrGrArGrUrCrGrGrUrGmC | |

¹For RNA, T is U; U are T in the sequence listing.
²For the sequences: *, phosphorothioate modification; mN, 2′O-methyl modification; rN, unmodified ribonucleotide; i2FN, Int Fluoro modification. N = A, C, T/U, G.

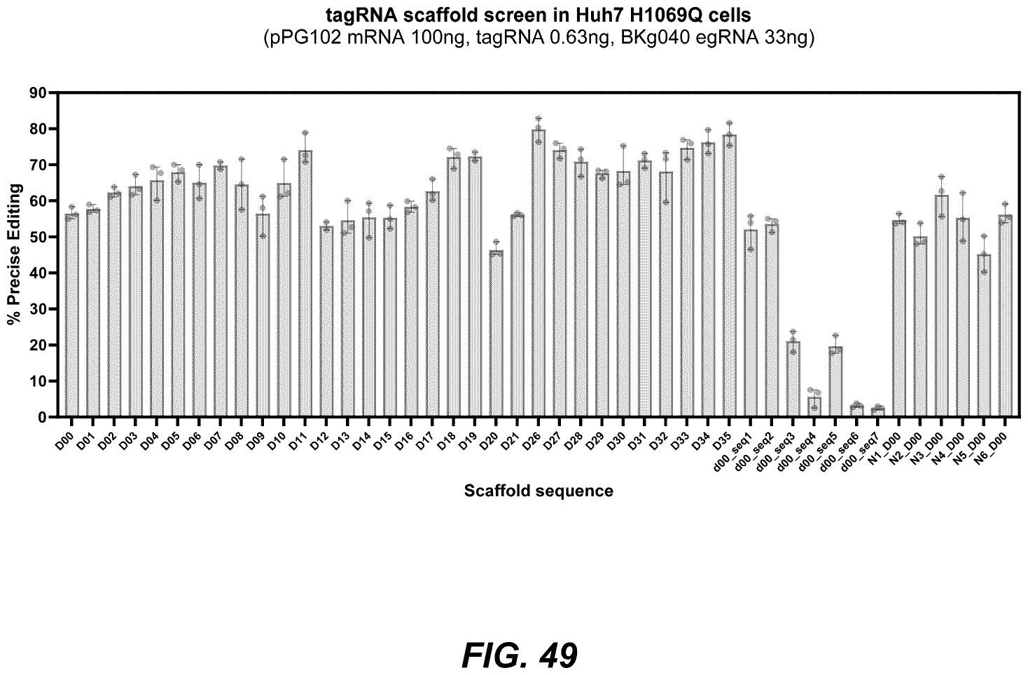

The benchmark scaffold was design DOO. In the experiment of FIG. 49, the scaffold variants were tested with a Wilson's disease tagRNA in Huh7 H1069Q cells. The tagRNA was transfected at a limiting dose (0.63 ng) to identify scaffold sequences with higher potency. Many scaffold variants outperformed the benchmark DOO (FIG. 49).

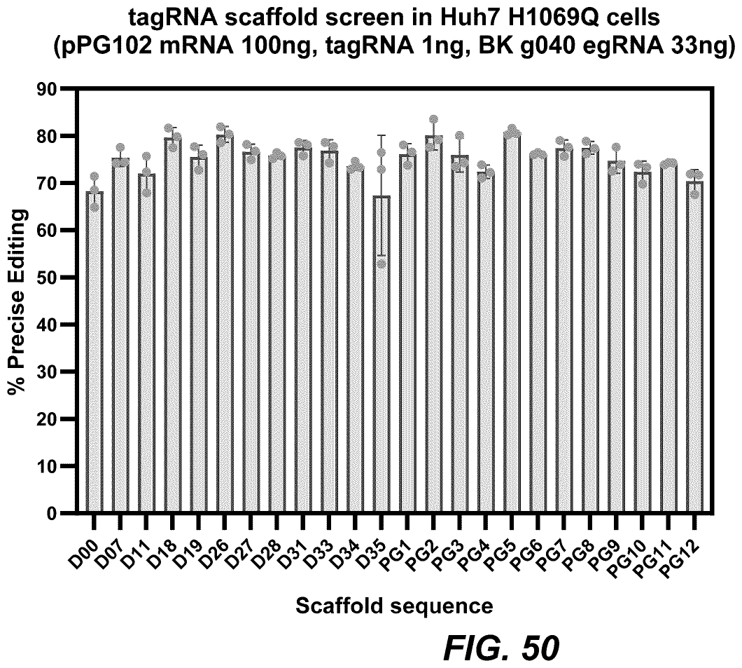

Next, twelve more SpyCas9 scaffold variants were designed and compared to the top sequences from the first set. The benchmark scaffold was design DOO. In this experiment (FIG. 50), the scaffold variants were tested in a Wilson's disease tagRNA in Huh7 H1069Q cells. Many scaffold variants outperformed the benchmark DOO.

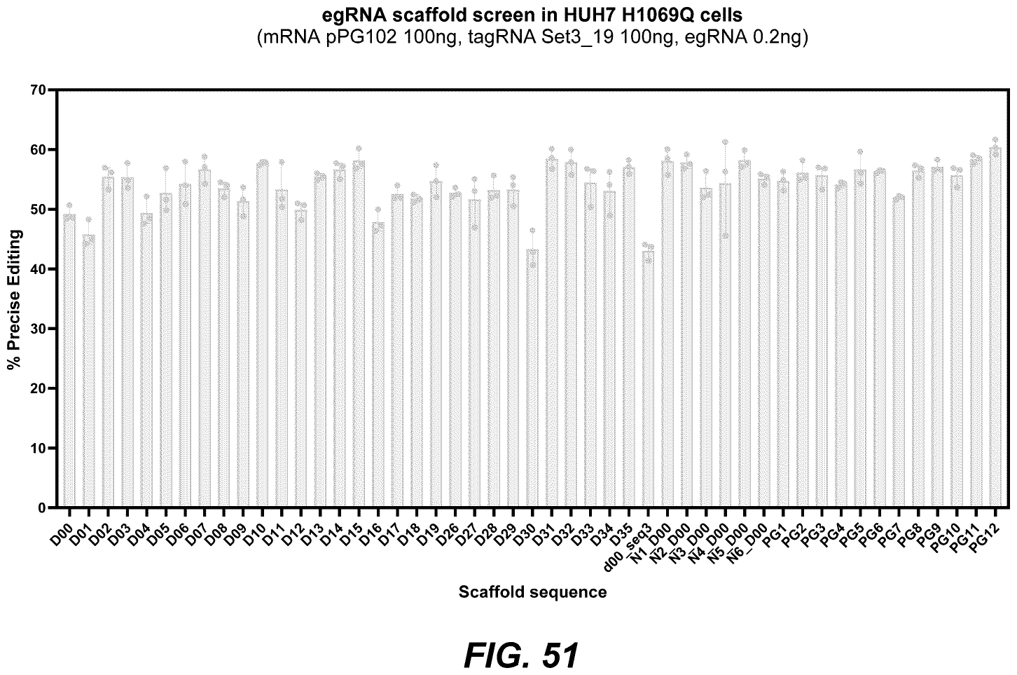

Finally, modifications to the egRNA scaffold were tested. SpyCas9 scaffold variants were designed with different sequences and different chemical modification patterns. The benchmark scaffold was design DOO. In this experiment, the scaffold variants were tested in a Wilson's disease egRNA in Huh7 H1069Q cells. The egRNA was transfected at a limiting dose (0.2 ng) to identify scaffold sequences with higher potency. As shown in FIG. 51, many scaffold variants outperformed the benchmark DOO.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A reverse transcriptase (RT) editing system comprising:
a fusion protein comprising a Cas9 nickase and a reverse transcriptase, and
a template armed guide RNA (tagRNA) comprising from 5′ to 3′ a spacer sequence, a scaffold sequence, an editing template and a flap binding sequence.

2. The RT editing system of claim 1, wherein the RT editing system further comprises an enhancer guide RNA (egRNA).

3. A reverse transcriptase (RT) editor comprising:
a DNA binding domain, a DNA endonuclease domain and a DNA polymerase domain,
wherein the DNA polymerase domain, the DNA binding domain, and the DNA endonuclease domain are fused or linked to form a fusion protein,
wherein the DNA polymerase domain comprises a reverse transcriptase, optionally a reverse transcriptase comprising an amino acid sequence that is at least 80% identical to any one of SEQ ID NOs: 141, 250-269, 315-319, 899-909, and 1015-1018, optionally an amino acid sequence having one, two, three, four, or five mismatches relative to the sequence of any one of SEQ ID NOs: 141, 250-269, 315-319, 899-909, and 1015-1018, and
wherein the DNA endonuclease domain comprises a Cas9 nickase, optionally a Cas9 nickase comprising an amino acid sequence that is at least 80% identical to any one of SEQ ID NOs: 142-163 and 270-314, optionally an amino acid sequence having one, two, three, four, or five mismatches relative to the sequence of any one of SEQ ID NOs: 142-163 and 270-314.

4. A template armed guide RNA (tagRNA) comprising:
a spacer that is complementary to a search target sequence on a first strand of a double stranded target DNA;
an editing template that comprises a region of complementarity to an editing target sequence on a second strand of the double stranded target DNA; and
a scaffold sequence that associates with a reverse transcriptase (RT) editor comprising a DNA binding domain, a DNA endonuclease domain and a DNA polymerase domain, and
wherein the first strand and the second strand are complementary to each other.

5. The tagRNA of claim 4, wherein the tagRNA comprises a flap binding sequence at least partially complementary to the spacer.

6. The tagRNA of any one of claims 4-5, wherein the scaffold sequence is between the spacer and the editing template.

7. The tagRNA of any one of claims 5-6, comprising from 5′ to 3′: the spacer, the scaffold sequence, the editing template, and the flap binding sequence.

8. The tagRNA of any one of claims 5-7, wherein the spacer, the scaffold sequence, the editing template, and the flap binding sequence form a contiguous sequence in a single molecule.

9. The tagRNA of any one of claims 4-8, wherein the editing template comprises an intended nucleotide edit compared to the double stranded target DNA.

10. The tagRNA of claim 9, wherein the tagRNA guides the RT editor to incorporate the intended nucleotide edit into the double stranded target DNA when the tagRNA is contacted with the double stranded target DNA.

11. The tagRNA of any one of claims 9-10, wherein the RT editor synthesizes a single stranded DNA encoded by the editing template, wherein the single stranded DNA replaces the editing target sequence and results in incorporation of the intended nucleotide edit into a region corresponding to the editing target in the double stranded target DNA.

12. The tagRNA of any one of claims 4-11, wherein the search target sequence is complementary to a protospacer sequence in the double stranded target DNA, and wherein the protospacer sequence is adjacent to a protospacer adjacent motif (PAM) in the double stranded target DNA.

13. The tagRNA of claim 12, wherein the tagRNA results in incorporation of a nucleotide edit in the PAM when contacted with the double stranded target DNA.

14. The tagRNA of any one of claims 4-13, wherein the spacer of the tagRNA is from 16 to 25 nucleotides in length, optionally 20 nucleotides in length or 21-23 nucleotides in length.

15. The tagRNA of any one of claims 5-14, wherein the flap binding sequence is about 2 to 20 nucleotides in length, optionally about 8 to 16 nucleotides in length or 6 nucleotides in length.

16. The tagRNA of any one of claims 4-15, wherein the editing template is about 4 to 30 nucleotides in length, optionally about 10 to 30 nucleotides in length, further optionally 6 to 9 nucleotides in length.

17. The tagRNA of any one of claims 9-16, wherein the tagRNA results in incorporation of the intended nucleotide edit about 0 to 30 base pairs downstream of the nickase cleavage site.

18. The tagRNA of any one of claims 9-17, wherein the intended nucleotide edit comprises a single nucleotide substitution compared to the region corresponding to the editing target in the double stranded target DNA, optionally, the single nucleotide substitution is a T>G or T>C substitution.

19. The tagRNA of any one of claims 9-18, wherein the intended nucleotide edit comprises an insertion compared to the region corresponding to the editing target in the double stranded target DNA, optionally an insertion of a nucleotide sequence at least 50, at least 45, at least 40, at least 35, at least 30, at least 25, at least 20, at least 15, at least 10, or at least 5, nucleotides in length.

20. The tagRNA of any one of claims 9-19, wherein the intended nucleotide edit comprises a deletion compared to the region corresponding to the editing target in the double stranded target DNA.

21. The tagRNA of any one of claims 4-20, wherein the editing template comprises one or more silent nucleotide edits compared to the region corresponding to the editing target in the double stranded target DNA, optionally said silent nucleotide edits do not alter the amino acid sequence of the protein encoded by the double stranded target DNA, further optionally said one or more silent nucleotide edits comprise a substitution of 2 to 5 contiguous nucleotides.

22. The tagRNA of any one of claims 4-21, wherein the editing template comprises a wild type double stranded target DNA sequence.

23. The tagRNA of claim 22, wherein the tagRNA results in correction of a mutation when contacted with the double stranded target DNA.

24. The tagRNA of any one of claims 4-23, wherein the tagRNA comprises any one of the sequences of SEQ ID NOs: 16-47, 99-130, 164-181, 320-497, 563-682, 791-792, 1046-1053, and 1089-1203 or a sequence that exhibits at least about 85% identity to any one of the sequences of SEQ ID NOs: 16-47, 99-130, 164-181, 320-497, 563-682, 791-792, 1046-1053, and 1089-1203.

25. The tagRNA of any one of claims 4-24, further comprising:
3′ mN*mN*mN*N and 5′ mN*mN*mN* modifications, where m indicates that the nucleotide contains a 2′-O-Me modification and a * indicates the presence of a phosphorothioate bond; and/or
a structural motif at the 3′ terminus selected from the group consisting of: an inverted-dT, a prequeosine1-1 riboswitch aptamer (evopreQ1) and variants thereof, a frameshifting pseudoknot from Moloney murine leukemia virus (MMLV) (mpknot), G-quadruplexes, hairpin structures, xrRNA, and a P4-P6 domain of the group I intron; optionally, the structural motif is evopreQ1 or a variant thereof comprising a nucleotide sequence selected from SEQ ID NOs: 84-90.

26. A reverse transcriptase (RT) editing system comprising:
a template armed guide RNA (tagRNA), or a nucleic acid encoding the tagRNA, wherein the tagRNA comprises:
a spacer that is complementary to a search target sequence on a first strand of a nucleic acid molecule;
an editing template that comprises a region of complementarity to an editing target sequence on a second strand of the nucleic acid molecule; and
a scaffold sequence that associates with a reverse transcriptase (RT) editor comprising a DNA binding domain, a DNA endonuclease domain and a DNA polymerase domain; and
a reverse transcriptase (RT) editor comprising a DNA binding domain, a DNA endonuclease domain and a DNA polymerase domain, or a nucleic acid encoding the RT editor,
wherein the DNA polymerase domain comprises a reverse transcriptase, optionally a reverse transcriptase comprising an amino acid sequence that is at least 80% identical to any one of SEQ ID NOs: 141, 250-269, 315-319, 899-909, and 1015-1018, optionally an amino acid sequence having one, two, three, four, or five mismatches relative to the sequence of any one of SEQ ID NOs: 141, 250-269, 315-319, 899-909, and 1015-1018, and
wherein the DNA endonuclease domain comprises a Cas9 nickase, optionally a Cas9 nickase comprising an amino acid sequence that is at least 80% identical to any one of SEQ ID NOs: 142-163 and 270-314, optionally an amino acid sequence having one, two, three, four, or five mismatches relative to the sequence of any one of SEQ ID NOs: 142-163 and 270-314.

27. A reverse transcriptase (RT) editing system comprising:
the tagRNA according to any one of claims 4-25, or a nucleic acid encoding the tagRNA; and
a reverse transcriptase (RT) editor comprising a DNA binding domain, a DNA endonuclease domain and a DNA polymerase domain, or a nucleic acid encoding the RT editor.

28. The RT editing system of any one of claims 26-27, further comprising: an enhancer guide RNA (egRNA), or a nucleic acid encoding the egRNA, wherein the egRNA comprises a egRNA spacer that is complementary to a second search target sequence in the double stranded target DNA, optionally the egRNA comprises a scaffold sequence.

29. The RT editing system of claim 28, wherein the second search target sequence is on the second strand of the double stranded target DNA.

30. The RT editing system of any one of claims 28-29, wherein the egRNA spacer is from 16 to 22 nucleotides in length, optionally 20 nucleotides in length.

31. The RT editing system of any one of claims 26-30, wherein the intended nucleotide edit incorporation rate of the RT editing system is greater than at least about 30%, about 40%, about 50%, about 60%, about 70%, or about 80%.

32. A reverse transcriptase (RT) editing complex comprising:
template armed guide RNA (tagRNA) comprising:
a spacer that is complementary to a search target sequence on a first strand of a nucleic acid molecule;
an editing template that comprises a region of complementarity to an editing target sequence on a second strand of the nucleic acid molecule; and
a scaffold sequence that associates with a reverse transcriptase (RT) editor comprising a DNA binding domain, a DNA endonuclease domain and a DNA polymerase domain; and
a reverse transcriptase (RT) editor comprising a DNA binding domain, a DNA endonuclease domain and a DNA polymerase domain, or a nucleic acid encoding the RT editor,
wherein the DNA polymerase domain comprises a reverse transcriptase, optionally a reverse transcriptase comprising an amino acid sequence that is at least 80% identical to any one of SEQ ID NOs: 141, 250-269, 315-319, 899-909, and 1015-1018, optionally an amino acid sequence having one, two, three, four, or five mismatches relative to the sequence of any one of SEQ ID NOs: 141, 250-269, 315-319, 899-909, and 1015-1018, and
wherein the DNA endonuclease domain comprises a Cas9 nickase, optionally a Cas9 nickase comprising an amino acid sequence that is at least 80% identical to any one of SEQ ID NOs: 142-163 and 270-314, optionally an amino acid sequence having one, two, three, four, or five mismatches relative to the sequence of any one of SEQ ID NOs: 142-163 and 270-314.

33. A reverse transcriptase (RT) editing complex comprising:
(i) the tagRNA of any one of claims 4-25 and a reverse transcriptase (RT) editor comprising a DNA binding domain, a DNA endonuclease domain and a DNA polymerase domain; or
(ii) the RT editing system of any one of claims 26-31.

34. The RT editing complex of any one of claims 32-33, wherein the intended nucleotide edit incorporation rate of the RT editing complex is greater than at least about 30%, about 40%, about 50%, about 60%, about 70%, or about 80%.

35. The RT editor, the tagRNA, the RT editing system, or the RT editing complex of any one of claims 1-34, wherein the DNA endonuclease domain is a CRISPR associated (Cas) protein domain.

36. The RT editor, the tagRNA, the RT editing system, or the RT editing complex of claim 35, wherein the Cas protein domain has nickase activity.

37. The RT editor, the tagRNA, the RT editing system, or the RT editing complex of any one of claims 35-36, wherein the Cas protein domain is a Cas9.

38. The RT editor, the tagRNA, the RT editing system, or the RT editing complex of claim 37, wherein the Cas9 comprises a mutation in an HNH domain.

39. The RT editor, the tagRNA, the RT editing system, or the RT editing complex of any one of claims 37-38, wherein the Cas9 comprises an H840A mutation in the HNH domain.

40. The RT editor, the tagRNA, the RT editing system, or the RT editing complex of any one of claims 35-36, wherein the Cas protein domain is a Cas12b.

41. The RT editor, the tagRNA, the RT editing system, or the RT editing complex of any one of claims 35-36, wherein the Cas protein domain is a Cas12a, Cas12b, Cas12c, Cas12d, Cas12e, Cas14a, Cas14b, Cas14c, Cas14d, Cas14e, Cas14f, Cas14g, Cas14h, Cas14u, Casϕ, Ssch1Cas9, Sro1Cas9, Sha4Cas9, SsuCas9, iSpyMacCas9, Ssi5Cas9, Ssi8Cas9, Ssci4Cas9, Shy1Cas9, Sag3Cas9, Slutr1Cas9, Ssch3Cas9, SpRYCas9, SpRYcCas9, Sma2Cas9, SsaCas9, EvoCjCas9, or iSpyMac.

42. The RT editor, the tagRNA, the RT editing system, or the RT editing complex of any one of claims 1-41, wherein the DNA polymerase domain is a reverse transcriptase, optionally the editing template is a reverse transcription template.

43. The RT editor, the tagRNA, the RT editing system, or the RT editing complex of claim 42, wherein the reverse transcriptase is a retrovirus reverse transcriptase.

44. The RT editor, the tagRNA, the RT editing system, or the RT editing complex of any one of claims 42-43, wherein the reverse transcriptase is a Moloney murine leukemia virus (MMLV) reverse transcriptase.

45. The RT editor, the tagRNA, the RT editing system, or the RT editing complex of any one of claims 1-44, wherein the DNA polymerase domain and the DNA binding domain, a DNA endonuclease domain are fused or linked to form a fusion protein.

46. The RT editor, the tagRNA, the RT editing system, or the RT editing complex of claim 45, wherein:
the DNA polymerase domain comprises a reverse transcriptase, optionally a reverse transcriptase comprising an amino acid sequence that is at least 80% identical to any one of SEQ ID NOs: 141, 250-269, 315-319, 899-909, and 1015-1018, optionally an amino acid sequence having one, two, three, four, or five mismatches relative to the sequence of any one of SEQ ID NOs: 141, 250-269, 315-319, 899-909, and 1015-1018; and/or
the DNA endonuclease domain comprises a Cas9 nickase, optionally a Cas9 nickase comprising an amino acid sequence that is at least 80% identical to any one of SEQ ID NOs: 142-163 and 270-314, optionally an amino acid sequence having one, two, three, four, or five mismatches relative to the sequence of any one of SEQ ID NOs: 142-163 and 270-314.

47. The RT editor, the tagRNA, the RT editing system, or the RT editing complex of any one of claims 1-46, wherein the editing template comprises: (i) at least 1, 2, 3, 4, 5, 6, 7, 8, 9, 10, 11, 12, 13, or 14 2′ O-methyl RNA base(s); and/or (ii) at least 1, 2, 3, 4, 5, 6, 7, 8, 9, 10, 11, 12, 13, or 14 2′ Fluoro RNA base(s).

48. The RT editor, the tagRNA, the RT editing system, or the RT editing complex of any one of claims 1-47, wherein the FBS comprises: (i) at least 1, 2, 3, 4, 5, 6, 7, or 8 2′ O-methyl RNA base(s); and/or (ii) at least 1, 2, 3, 4, 5, 6, 7, or 8 2′ Fluoro RNA base(s).

49. The RT editor, the tagRNA, the RT editing system, or the RT editing complex of any one of claims 1-48, wherein the scaffold sequence comprises a nucleotide sequence that is at least 80% identical to any one of SEQ ID NOs: 683-718, 894-898, and 964-1014, optionally a nucleotide sequence having one, two, three, four, or five mismatches relative to the sequence of any one of SEQ ID NOs: 683-718, 894-898, and 964-1014.

50. The RT editor, the tagRNA, the RT editing system, or the RT editing complex of any one of claims 1-49, wherein the scaffold sequence comprises one or more nucleotide substitutions, insertions, and/or deletions at one or more nucleotide positions relative to the parental scaffold sequence of SEQ ID NO: 683, optionally:
dinucleotide substitutions at nucleotide positions 5-6 and 34-35, optionally configured such that the nucleotide sequence at positions 5-6 is complementary to the nucleotide sequence at positions 34-35;
a deletion at nucleotide positions 55-87, 57-87, 59-87, 61-87, or 63-87;
a substitution at one or more of nucleotide positions 17, 18, 19, and 20, optionally a UUCG substitution at nucleotide positions 17-20;
a deletion at nucleotide positions 14-16 and 21-24;
a replacement of nucleotides at nucleotide positions 11-28 with GUUCGC; and/or
a deletion at nucleotide positions 12-16 and 21-26.

51. The RT editor, the tagRNA, the RT editing system, or the RT editing complex of any one of claims 1-50, wherein the scaffold sequence comprises one or more modifications and/or one or more nucleotide substitutions, insertions, and/or deletions at one or more nucleotide positions relative to the parental scaffold sequence of SEQ ID NO: 700, optionally:
a substitution at one or more of nucleotide positions 13, 18, 21, 40, 50, and 52-53, further optionally:
a U-to-C substitution at nucleotide position 13;
an A-to-G substitution at nucleotide position 18;
an A-to-C or an A-to-U substitution at nucleotide position 21;
a C-to-U or a C-to-A or a C-to-G substitution at nucleotide position 40;
a U-to-C or a U-to-A or a U-to-G substitution at nucleotide position 50;
an A-to-C or an A-to-U or an A-to-G substitution at nucleotide position 52;
and/or
an A-to-C or an A-to-U or an A-to-G substitution at nucleotide position 53;
the one or more modifications comprise nucleoside modification(s), sugar modification(s), modified internucleoside linkage(s), and/or backbone modification(s), optionally the scaffold sequence comprises (i) a 2′ O-methyl modification at one or more of nucleotide positions 1, 3-9, 12-27, 29-32, 36-38, 40, 44-45, 49-50, and 52-53, and/or (ii) a 2′-fluoro modification at one or more of nucleotide positions 1-4, 6, 8, 10-11, 20, 22-32, 35-38, 40-46, and 49-51; and/or
the scaffold sequence comprises any one of the sequences of SEQ ID NOs: 894-898 and 964-1014, or a sequence that exhibits at least about 85% identity to any one of the sequences of SEQ ID NOs: 894-898 and 964-1014.

52. The RT editor, the tagRNA, the RT editing system, or the RT editing complex of any one of claims 1-51, wherein the scaffold sequence of the tagRNA, the scaffold sequence of the egRNA, or both, comprises the sequence of any one of SEQ ID NOs: 1210-1266 or a sequence that exhibits at least about 85% identity to any one of SEQ ID NOs: 1210-1266.

53. The RT editor, the tagRNA, the RT editing system, or the RT editing complex of any one of claims 3-52, wherein:
the DNA polymerase domain comprises a reverse transcriptase, optionally a reverse transcriptase comprising one or more mutations, wherein at least one of the one or more mutations is at an amino acid position functionally equivalent to V101, N200, A208, G248, P330, L435, K445, and/or A623 relative to SEQ ID NO: 8.

54. The RT editor, the tagRNA, the RT editing system, or the RT editing complex of any one of claims 3-52, wherein:
the DNA polymerase domain comprises a reverse transcriptase comprising an amino acid sequence that is at least 80% identical to SEQ ID NO: 255, optionally the reverse transcriptase comprises one or more mutations,
optionally at least one of the one or more mutations is at amino acid position V98, N197, A205, S245, P327, V432, R442, and/or A623 relative to SEQ ID NO: 255,
further optionally the reverse transcriptase comprises one or more transition mutations selected from the group consisting of V98R, N197C, N197D, N197G, A205T, S245C, P327E, P327Q, V432K, R442T, and A623F relative to SEQ ID NO: 255.

55. The RT editor, the tagRNA, the RT editing system, or the RT editing complex of any one of claims 3-52, wherein:
the DNA polymerase domain comprises a reverse transcriptase comprising an amino acid sequence that is at least 80% identical to SEQ ID NO: 260, optionally the reverse transcriptase comprises one or more mutations,
optionally at least one of the one or more mutations is at amino acid position V106, N204, E212, G252, P334, L440, E450, and/or A629 relative to SEQ ID NO: 260,
further optionally the reverse transcriptase comprises one or more transition mutations selected from the group consisting of V106R, N204C, N204D, N204G, E212T, G252C, P334E, P334Q, L440K, E450T, and A629F relative to SEQ ID NO: 260.

56. The RT editor, the tagRNA, the RT editing system, or the RT editing complex of any one of claims 3-52, wherein the DNA polymerase domain:
comprises a reverse transcriptase comprising an amino acid sequence that is at least 80% identical to SEQ ID NO: 255, and wherein the reverse transcriptase comprises an N197C and/or V98R transition mutation relative to SEQ ID NO: 255; or
comprises a reverse transcriptase comprising an amino acid sequence that is at least 80% identical to SEQ ID NO: 260, and wherein the reverse transcriptase comprises an N204C and/or V106R transition mutation relative to SEQ ID NO: 260.

57. The RT editor, the tagRNA, the RT editing system, or the RT editing complex of any one of claims 3-56, wherein the RT editor further comprises an accessory domain, optionally the accessory domain comprises a single-strand binding (SSB) protein domain or a stabilon, further optionally the SSB protein domain is derived from RecA protein, Sso7d protein, or Sto7d protein.

58. The RT editor, the tagRNA, the RT editing system, or the RT editing complex of claim 57, wherein the accessory domain is situated at the N-terminus, the C-terminus, or at an internal location of the RT editor.

59. The RT editor, the tagRNA, the RT editing system, or the RT editing complex of any one of claims 57-58, wherein the RT editor comprising an accessory domain comprises an amino acid sequence that is at least 85% identical to any one of SEQ ID NOs: 1276-1298.

60. The RT editor, the tagRNA, the RT editing system, or the RT editing complex of any one of claims 57-58, wherein the SSB protein domain is derived from Sso7d and comprises one or more mutations at an amino acid position functionally equivalent to K12 and/or E35 of a wild type Sso7d amino acid sequence, optionally the one or more mutations comprise K12L and/or E35L relative to a wild type Sso7d sequence, further optionally, the SSB protein domain comprises an amino acid sequence that is at least 85% identical to SEQ ID NO: 1205.

61. The RT editor, the tagRNA, the RT editing system, or the RT editing complex of any one of claims 57-60, wherein the RT editor comprising the SSB protein domain comprises from N-terminus to C-terminus: [N-terminus of nCas9]-[linker]-[SSB protein domain]-[linker]-[RT]-[linker]-[C-terminus of nCas9], optionally the N-terminus of nCas9 comprises an amino acid sequence functionally equivalent to amino acids 1-1247 of SEQ ID NO: 6 and/or the C-terminus of nCas9 comprises an amino acid sequence functionally equivalent to amino acids 1248-1367 of SEQ ID NO: 6.

62. The RT editor, the tagRNA, the RT editing system, or the RT editing complex of claim 61, wherein the RT editor comprising the SSB protein domain comprises an amino acid sequence that is at least 85% identical to any one of SEQ ID NOs: 1207-1209.

63. A ribonucleoprotein (RNP) complex comprising the RT editing complex of any one of claims 32-62, or a component thereof.

64. A lipid nanoparticle (LNP) comprising the RT editing system of any one of claims 26-31 and 35-62, or a component thereof.

65. The LNP of claim 64, comprising the tagRNA and the nucleic acid encoding the RT editor.

66. The LNP of claim 65, wherein the nucleic acid encoding the RT editor is mRNA.

67. The LNP of any one of claims 64-66, further comprising the egRNA.

68. A polynucleotide encoding the RT editor of claim 1, the tagRNA of any one of claims 4-25 and 35-62, the RT editing system of any one of claims 26-31 and 35-56, or the RT editing complex of any one of claims 32-62.

69. The polynucleotide of claim 68, wherein the polynucleotide is an mRNA.

70. The polynucleotide of any one of claims 68-69, wherein the polynucleotide is operably linked to a regulatory element, optionally the regulatory element is an inducible regulatory element.

71. A vector comprising the polynucleotide of any one of claims 68-70.

72. The vector of claim 71, wherein the vector is an AAV vector.

73. An isolated cell comprising the RT editor of claim 1, the tagRNA of any one of claims 4-25 and 35-62, the RT editing system of any one of claims 26-31 and 35-62, the RT editing complex of any one of claims 32-62, the RNP of claim 63, the LNP of any one of claims 64-67, the polynucleotide of any one of claims 68-70, or the vector of any one of claims 71-72.

74. The cell of claim 73, wherein the cell is a mammalian cell, optionally a human cell.

75. The cell of any one of claims 73-74, wherein the cell is a primary cell.

76. The cell of any one of claims 73-75, wherein the cell is a hepatocyte.

77. The cell of any one of claims 73-76, wherein the cell is from a subject having a disease or disorder, optionally the disease or disorder is selected from the group comprising an autoimmune disease, a neurological disease or disorder, a cancer, an inflammatory disease, a cardiovascular disease, an infectious disease, a genetic disease, a trinucleotide repeat expansion disorder, a metabolic disease, or any combination thereof, optionally Wilson's disease, alpha1 antitrypsin deficiency disease, phenylketonuria, or hyperphenylalaninemia, further optionally the subject is a human.

78. A pharmaceutical composition comprising:
(i) the RT editor of claim 1, the tagRNA of any one of claims 4-25 and 35-62, the RT editing system of any one of claims 26-31 and 35-62, the RT editing complex of any one of claims 32-62, the RNP of claim 63, the LNP of any one of claims 64-67, the polynucleotide of any one of claims 68-70, the vector of any one of claims 71-72, or the cell of any one of claims 73-77; and
(ii) a pharmaceutically acceptable carrier.

79. A method for editing a double stranded target DNA, the method comprising contacting the double stranded target DNA with (i) the tagRNA of any one of claims 4-25 and 35-62 and a reverse transcriptase (RT) editor comprising a DNA binding domain, a DNA endonuclease domain and a DNA polymerase domain or (ii) the RT editing system of any one of claims 26-31 and 35-62, wherein the tagRNA directs the RT editor to incorporate the intended nucleotide edit in the double stranded target DNA, thereby editing the double stranded target DNA.

80. A method for editing a double stranded target DNA, comprising contacting the double stranded target DNA with the RT editing complex of any one of claims 32-62, wherein the tagRNA directs the RT editor to incorporate the intended nucleotide edit in the double stranded target DNA, thereby editing the double stranded target DNA.

81. The method of any one of claims 79-80, wherein the RT editor synthesizes a single stranded DNA encoded by the editing template, wherein the single stranded DNA replaces the editing target sequence and results in incorporation of the intended nucleotide edit into a region corresponding to the editing target in the double stranded target DNA.

82. The method of any one of claims 79-81, wherein the double stranded target DNA is in a cell.

83. The method of claim 82, wherein the cell is a mammalian cell, optionally a human cell.

84. The method of any one of claims 82-83, wherein the cell is a primary cell.

85. The method of any one of claims 82-84, wherein the cell is a hepatocyte.

86. The method of any one of claims 82-83, wherein the cell is a stem cell; optionally, an embryonic stem cell, an induced pluripotent stem cell, or an adult stem cell.

87. The method of any one of claims 82-86, wherein the cell is in a subject, optionally the subject is a human.

88. The method of any one of claims 82-86, wherein the cell is from a subject having a disease or disorder, optionally the disease or disorder is selected from the group comprising an autoimmune disease, a neurological disease or disorder, a cancer, an inflammatory disease, a cardiovascular disease, an infectious disease, a genetic disease, a trinucleotide repeat expansion disorder, a metabolic disease, or any combination thereof, optionally Wilson's disease, alpha1 antitrypsin deficiency disease, phenylketonuria, or hyperphenylalaninemia.

89. The method of claim 88, further comprising administering the cell to the subject after incorporation of the intended nucleotide edit.

90. A cell generated by the method of any one of claims 79-89.

91. A population of cells generated by the method of any one of claims 79-89.

92. A method for treating or preventing a disease or disorder in a subject in need thereof, the method comprising administering to the subject the RT editing system of any one of claims 26, 28-31 and 35-62, wherein the editing template comprises an intended nucleotide edit compared to a double stranded target DNA of the subject, wherein the tagRNA directs the RT editor to incorporate the intended nucleotide edit in the double stranded target DNA, and wherein incorporation of the intended nucleotide edit corrects a mutation in the double stranded target DNA associated with the disease or disorder, thereby treating or preventing the disease or disorder in the subject, optionally the disease or disorder is selected from the group comprising an autoimmune disease, a neurological disease or disorder, a cancer, an inflammatory disease, a cardiovascular disease, an infectious disease, a genetic disease, a trinucleotide repeat expansion disorder, a metabolic disease, or any combination thereof.

93. A method for treating or preventing a disease or disorder in a subject in need thereof, the method comprising administering to the subject the RT editing complex of any one of claims 33-62, the RNP of claim 63, the LNP of any one of claims 64-67, or the pharmaceutical composition of claim 78, wherein the editing template comprises an intended nucleotide edit compared to a double stranded target DNA of the subject, wherein the tagRNA directs the RT editor to incorporate the intended nucleotide edit in the double stranded target DNA, and wherein incorporation of the intended nucleotide edit corrects a mutation in the double stranded target DNA associated with the disease or disorder, thereby treating or preventing the disease or disorder in the subject, optionally the disease or disorder is selected from the group comprising an autoimmune disease, a neurological disease or disorder, a cancer, an inflammatory disease, a cardiovascular disease, an infectious disease, a genetic disease, a trinucleotide repeat expansion disorder, a metabolic disease, or any combination thereof.

94. A messenger RNA (mRNA) encoding a reverse transcriptase (RT) editor, optionally the RT editor of any one of claims 3-62.

95. The mRNA of claim 94, wherein the mRNA comprises one or more of a 5′-cap structure, a 5′-UTR, a 3′-UTR, and a nuclear localization sequence (NLS).

96. The mRNA of any one of claims 94-95, wherein the mRNA comprises (i) a 5′-cap, (ii) a 5′-untranslated region (UTR); (iii) an open reading frame (ORF) comprising a nucleotide sequence that encodes the RT editor; and (iv) a 3′ untranslated region (UTR).

97. The mRNA of any one of claims 94-96, wherein mRNA has a structure comprising or consisting of 5′-[5′UTR]-[NLS]-[nCas9]-[linker]-[RT]-[NLS]-[3′UTR and/or viral element]-[polyA sequence]-3′.

98. The mRNA of any one of claims 94-97, wherein one or more nucleosides of the mRNA sequence are chemically modified.

99. The mRNA of any one of claims 94-98, wherein the mRNA comprises one or more additional segmented polyA sequences configured to increase mRNA stability and/or half-life.

100. The mRNA of any one of claims 94-99, wherein the mRNA comprises one or more viral element sequences, optionally 3′ of the 3′-UTR.

101. The mRNA of claim 100, wherein the viral element comprises a woodchuck hepatitis virus post-transcriptional regulatory element (WPRE) sequence, optionally comprising the sequence of SEQ ID NO: 189, or a sequence that exhibits at least about 85% identity to SEQ ID NO: 189.

102. The mRNA of any one of claims 100-101, wherein the viral element comprises a eK5 sequence, optionally located 3′ of the 3′-UTR, optionally comprising the sequence of SEQ ID NO: 190, or a sequence that exhibits at least about 85% identity to SEQ ID NO: 190.

103. The mRNA of any one of claims 94-102, wherein the mRNA comprises one or more secondary structure motifs.

104. The mRNA of claim 103, wherein a secondary structure motif comprises a triple helix sequence, optionally a synthetic triple helix (STH) sequence.

105. The mRNA of claim 104, wherein the STH sequence comprises the sequence derived from a sequence element of a long non-coding RNA, optionally MALAT1.

106. The mRNA of any one of claims 104-105, wherein the STH sequence comprises any one of the sequences of SEQ ID NOs: 191-192 and 245-249, or a sequence that exhibits at least about 85% identity to any one of the sequences of SEQ ID NOs: 191-192 and 245-249.

107. The mRNA of any one of claims 94-106, wherein the mRNA comprises:
a polyA sequence comprising any one of the sequences of SEQ ID NOs: 182-187, or a sequence that exhibits at least about 85% identity to any one of the sequences of SEQ ID NOs: 182-187;
one or more 3′ UTR element sequences selected from the group comprising any one of the sequences of SEQ ID NOs: 188-190, or a sequence that exhibits at least about 85% identity to any one of the sequences of SEQ ID NOs: 188-190;
one or more 3′ UTR structural motifs selected from the group comprising any one of the sequences of SEQ ID NOs: 191-192, or a sequence that exhibits at least about 85% identity to any one of the sequences of SEQ ID NOs: 191-192;
a 5′UTR sequence comprising the sequence of any one of SEQ ID NOs: 193 and 910-933, or a sequence that exhibits at least about 85% identity to any one of SEQ ID NOs: 193 and 910-933; and/or
an RT editor sequence comprising the sequence of SEQ ID NO: 194, or a sequence that exhibits at least about 85% identity to SEQ ID NO: 194.

108. The mRNA of any one of claims 94-107, wherein the mRNA comprises a 3′ UTR comprising any one of the sequences of SEQ ID NOs: 195-219, or a sequence that exhibits at least about 85% identity to any one of the sequences of SEQ ID NOs: 195-219.

109. The mRNA of any one of claims 94-108, wherein the mRNA comprises:
one or more 5′ UTR element sequences selected from the group comprising any one of the sequences of SEQ ID NOs: 940-950 and 1075, or a sequence that exhibits at least about 85% identity to any one of the sequences of SEQ ID NOs: 940-950 and 1075;
one or more 3′ UTR element sequences selected from the group comprising any one of the sequences of SEQ ID NOs: 951-961 and 1076-1080, or a sequence that exhibits at least about 85% identity to any one of the sequences of SEQ ID NOs: 951-961 and 1076-1080; and/or
one or more additional elements selected from the group comprising any one of the sequences of SEQ ID NOs: 962-963, or a sequence that exhibits at least about 85% identity to any one of the sequences of SEQ ID NOs: 962-963, optionally the one or more additional elements are situated downstream of the 3′UTR and/or the polyA tail.

110. The mRNA of any one of claims 94-109, wherein the mRNA comprises one or more 5′-UTR element sequences selected from the group comprising any one of the sequences of SEQ ID NOs: 1081-1082, and wherein the second codon of the mRNA is a “gcc”.

111. The mRNA of any one of claims 94-110, wherein the mRNA is codon optimized for expression in human cells, optionally, the mRNA comprises the sequence of any one of SEQ ID NOs: 1086-1088.

112. A ribonucleoprotein (RNP) complex, a lipid nanoparticle (LNP), a vector, an isolated cell, or a pharmaceutical composition comprising the mRNA of any one of claims 94-111.

113. A polynucleotide encoding the mRNA of any one of claims 94-111.
